SEQUENCE LISTING (1 sequence)

SEQ ID NO: 1 - protein, 15 aa, Artificial Sequence
  Completely synthetic peptide
EQEDEPEGDYFEWLE

(19) United States
(12) Patent Application Publication
(10) Pub. No.: US 2013/0116231 A1
(43) Pub. Date: May 9, 2013

(54) TYROSINE KINASE INHIBITORS

(71) Applicants: Kevin Wilson, Newton, MA (US); Michael Altman, Needham, MA (US); Kathryn Lipford, Boston, MA (US); Catherine White, Newton Center, MA (US); Matthew Daniels, Cambridge, MA (US)

(72) Inventors: Kevin Wilson, Newton, MA (US); Michael Altman, Needham, MA (US); Kathryn Lipford, Boston, MA (US); Catherine White, Newton Center, MA (US); Matthew Daniels, Cambridge, MA (US)

(73) Assignee: Merck Sharp & Dohme Corp.

(21) Appl. No.: 13/809,436

(22) PCT Filed: Jul. 7, 2011
(86) PCT No.: PCT/US11/43114

§ 371 Date: Jan. 10, 2013

Related U.S. Application Data
(60) Provisional application No. 61/363,420, filed on Jul. 12, 2010.

(51) Int. Cl. C07D 411/14; C07D 403/14; C07D 403/04; C07D 401/14; C07D 401/12

(52) U.S. Cl. CPC C07D 411/14; C07D 401/14; C07D 401/12; C07D 403/04; C07D 403/14
USPC 514/210.18; 544/238; 514/252.04; 514/252.05; 514/252.02; 544/114; 514/235.2

(57) ABSTRACT

The present invention relates to 1,4-dihydropyridazinone derivatives, that are useful for treating cellular proliferative diseases, for treating disorders associated with MET activity, and for inhibiting the receptor tyrosine kinase MET. The invention also related to compositions which comprise these compounds, and methods of using them to treat cancer in mammals.

### BACKGROUND OF THE INVENTION

This invention relates to pyridazin-4(1H)-one compounds that are inhibitors of tyrosine kinases, in particular the receptor tyrosine kinase MET, and are useful in the treatment of cellular proliferative diseases, for example cancer, hyperplasias, restenosis, cardiac hypertrophy, immune disorders and inflammation.

Studies on signal transduction pathways have generated various promising molecular targets for therapeutic inhibition in cancer therapy. Receptor tyrosine kinases (RTK) represent an important class of such therapeutic targets. Recently, members of the MET proto-oncogene family, a subfamily of receptor tyrosine kinases, have drawn special attention to the association between invasion and metastasis. The MET family, including MET (also referred to as c-Met) and RON receptors, can function as oncogenes like most tyrosine kinases. MET has been shown to be overexpressed and/or mutated in a variety of malignancies. A number of MET activating mutations, many of which are located in the tyrosine kinase domain, have been detected in various solid tumors and have been implicated in invasion and metastasis of tumor cells.

The c-Met proto-oncogene encodes the MET receptor tyrosine kinase. The MET receptor is an approximately 190 kDa glycosylated dimeric complex composed of a 50 kDa alpha chain disulfide-linked to a 145 kDa beta chain. The alpha chain is found extracellularly while the beta chain contains extracellular, transmembrane and cytosolic domains. MET is synthesized as a precursor and is proteolytically cleaved to yield mature alpha and beta subunits. It displays structural similarities to semaphoring and plexins, a ligand-receptor family that is involved in cell-cell interaction.

The natural ligand for MET is hepatocyte growth factor (HGF), a disulfide linked heterodimeric member of the scatter factor family that is produced predominantly by mesenchymal cells and acts primarily on MET-expressing epithelial and endothelial cells in an endocrine and/or paraendocrine fashion. HGF has some homology to plasminogen.

It is known that stimulation of MET via hepatocyte growth factor (also known as scatter factor, HGF/SF) results in a plethora of biological and biochemical effects in the cell. Activation of c-Met signaling can lead to a wide array of cellular responses including proliferation, survival, angiogenesis, wound healing, tissue regeneration, scattering, motility, invasion and branching morphogenesis. HGF/MET signaling also plays a major role in the invasive growth that is found in most tissues, including cartilage, bone, blood vessels, and neurons.

Various c-Met mutations have been well described in multiple solid tumors and some hematologic malignancies. The prototypic c-Met mutation examples are seen in hereditary and sporadic human papillary renal carcinoma (Schmidt, L. et al., Nat. Tenet. 1997, 16, 68-73; Jeffers, M. et al., Proc. Nat. Acad. Sci. 1997, 94, 11445-11500). Other reported examples of c-Met mutations include ovarian cancer, childhood hepatocellular carcinoma, metastatic head and neck squamous cell carcinomas and gastric cancers. HGF/MET has been shown to inhibit anoikis, suspension-induced programmed cell death (apoptosis), in head and neck squamous cell carcinoma cells.

MET signaling is implicated in various cancers, especially renal. The nexus between MET and colorectal cancer has also been established. Analysis of c-Met expression during colorectal cancer progression showed that 50% of the carcinoma specimens analyzed expressed 5-50-fold higher levels of MET mRNA transcripts and protein versus the adjacent normal colonic mucosa. In addition, when compared to the primary tumor, 70% of colorectal cancer liver metastasis showed MET overexpression.

MET is also implicated in glioblastoma. High-grade malignant gliomas are the most common cancers of the central nervous system. Despite treatment with surgical resection, radiation therapy, and chemotherapy, the mean overall survival is <1.5 years, and few patients survive for >3 years. Human malignant gliomas frequently express both HGF and MET, which can establish an autocrine loop of biological significance. Glioma MET expression correlates with glioma grade, and an analysis of human tumor specimens showed that malignant gliomas have a 7-fold higher HGF content than low-grade gliomas. Multiple studies have demonstrated that human gliomas frequently co-express HGF and MET and that high levels of expression are associated with malignant progression. It was further shown that HGF-MET is able to activate Akt and protect glioma cell lines from apoptotic death, both in vitro and in vivo.

RON shares a similar structure, biochemical features, and biological properties with MET. Studies have shown RON overexpression in a significant fraction of breast carcinomas and colorectal adenocarcinomas, but not in normal breast epithelia or benign lesions. Cross-linking experiments have shown that RON and MET form a non-covalent complex on the cell surface and cooperate in intracellular signaling. RON and MET genes are significantly co-expressed in ovarian cancer cell motility and invasiveness. This suggests that co-expression of these two related receptors might confer a selective advantage to ovarian carcinoma cells during either tumor onset or progression.

A number of reviews on MET and its function as an oncogene have recently been published: Cancer and Metastasis Review 22:309-325 (2003); Nature Reviews/Molecular Cell Biology 4:915-925 (2003); Nature Reviews/Cancer 2:289-300 (2002).

Since dysregulation of the HGF/MET signaling has been implicated as a factor in tumorgenesis and disease progression in many tumors, different strategies for therapeutic inhibition of this important RTK molecule should be investigated. Specific small molecule inhibitors against HGF/MET signaling and against RON/MET signaling have important therapeutic value for the treatment of cancers in which Met activity contributes to the invasive/metastatic phenotype.

### SUMMARY OF THE INVENTION

The present invention relates to pyridazin-4(1H)-one derivatives, that are useful for treating cellular proliferative diseases, for treating disorders associated with MET activity, and for inhibiting the receptor tyrosine kinase MET. The compounds of the invention may be illustrated by the Formula I:

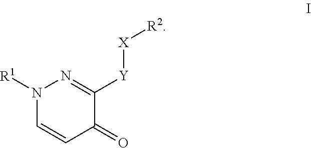

### DETAILED DESCRIPTION OF THE INVENTION

The compounds of this invention are useful in the inhibition of tyrosine kinses, in particular the receptor tyrosine kinase MET, and are illustrated by a compound of the formula:

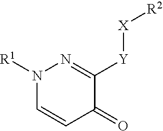

wherein X is CR⁴R^{4′}, C₂₋₃ alkenyl, O, NR⁵ or S;

### Y is CR³R^{3′}, O, NR⁵ or S;

R¹ is heteroaryl or aryl, wherein said heteroaryl and aryl groups are optionally substituted with one to three groups independently selected from the group consisting of halo, cyano, C₁₋₆ alkyl, (C₁₋₆ alkyl)R⁷, OR⁷, (C═O)NR⁵R⁶, heterocyclyl, aryl, heteroaryl and (heteroaryl)R⁶;
R² is heteroaryl or phenyl, wherein said heteroaryl and phenyl groups are optionally substituted with one to three groups independently selected from the group consisting of halo, cyano, oxo, C₁₋₆ alkyl, (C₁₋₆ alkyl)OR⁶, OR⁶, R⁷, OR⁷, O(C₁₋₆ alkyl)OR⁶, O(C₁₋₆ alkyl)R⁷, (C═O)R⁶, (C═O)OR⁶, (C═O)NHR⁶, (C═O)R⁷, NHR⁶, NH(C═O)OR⁶, NH(C═O)R⁷, NH(C═O)O(C₁₋₆ alkyl)OR⁶ and NO₂;
R³ is hydrogen, halo or C₁₋₆ alkyl,
R^{3′} is hydrogen, halo or C₁₋₆ alkyl,
R⁴ is hydrogen, halo, C₁₋₆ alkyl or (C═O)OR⁶,
R^{4′} is hydrogen, halo or C₁₋₆ alkyl,
R⁵ is hydrogen or C₁₋₆ alkyl,
R⁶ is hydrogen or C₁₋₆ alkyl, wherein said alkyl is optionally substituted with one to three groups independently selected from the group consisting of halo and hydroxyl;
R⁷ is hydrogen, heterocyclyl, aryl or heteroaryl, wherein said heterocyclyl and heteroaryl groups are optionally substituted with one to two groups independently selected from the group consisting of cyano, halo, hydroxyl, R⁶, R⁸, OR⁶, (C₁₋₆ alkyl)OR⁶, (C₁₋₆ alkyl)OR⁸, SO₂(C₁₋₆ alkyl), (C═O)R⁸;
R⁸ is heterocyclyl or heteroaryl, wherein said heterocyclyl group is optionally substituted with cyano, halo, hydroxyl, R⁶, OR⁶ or (C═O)OR⁶;
or a pharmaceutically acceptable salt thereof.

In a class of the invention, R¹ is heteroaryl, wherein said heteroaryl group is optionally substituted with one to three groups independently selected from the group consisting of halo, cyano and C₁₋₆ alkyl. In a subclass of the invention, R¹ is heteroaryl, wherein said heteroaryl group is optionally substituted with C₁₋₆ alkyl.

In a class of the invention, R² is heteroaryl or phenyl, wherein said heteroaryl and phenyl groups are optionally substituted with one to three groups independently selected from the group consisting of halo, oxo, C₁₋₆ alkyl, (C₁₋₆ alkyl)OR⁶, OR⁶, R⁷, OR⁷, O(C₁₋₆ alkyl)OR⁶, O(C₁₋₆ alkyl)R⁷, (C═O)R⁶, (C═O)OR⁶, (C═O)NHR⁶, (C═O)R⁷, NHR⁶, NH(C═O)OR⁶, NH(C═O)R⁷, NH(C═O)O(C₁₋₆ alkyl)OR⁶ and NO₂. In a subclass of the invention, R² is quinolinyl, wherein said quinolinyl is optionally substituted with one to three groups independently selected from the group consisting of halo, oxo, C₁₋₆ alkyl, (C₁₋₆ alkyl)OR⁶, OR⁶, R⁷, OR⁷, O(C₁₋₆ alkyl)OR⁶, O(C₁₋₆ alkyl)R⁷, (C═O)R⁶, (C═O)OR⁶, (C═O)NHR⁶, (C═O)R⁷, NHR⁶, NH(C═O)OR⁶, NH(C═O)R⁷, NH(C═O)O(C₁₋₆ alkyl)OR⁶ and NO₂.

In a class of the invention, R³ is hydrogen. In another class of the invention, R³ is methyl. In another class of the invention, R³ is (C═O)OR⁶.

In a class of the invention, R^{3′} is hydrogen.

In a class of the invention, R⁴ is hydrogen.

In a class of the invention, R^{4′} is hydrogen.

In a class of the invention, X is O. In another class of the invention X is CR⁴R^{4′}. In another class of the invention, X is NR⁵. In another class of the invention, X is S. In another class of the invention, X is C═CH₂.

In a class of the invention, Y is CR³R³. In another class of the invention Y is O. In another class of the invention, Y is NR⁵. In another class of the invention, Y is S.

Specific examples of the compounds of the instant invention include, but are not limited to:
1-(1-Methyl-1H-pyrazol-4-yl)-3-[(quinolin-6-yloxy)methyl]pyridazin-4(1H)-one;
3-[(isoquinolin-5-yloxy)methyl]-1-(1-methyl-1H-pyrazol-4-yl)pyridazin-4(1H)-one;
3-{4-oxo-3-[(quinolin-6-yloxy)methyl]pyridazin-1 (4H)-yl}benzonitrile;
3-fluoro-5-{4-oxo-3-[(quinolin-6-yloxy)methyl]pyridazin-1(4H)-yl}benzonitrile;
4-{4-oxo-3-[(quinolin-6-yloxy)methyl]pyridazin-1(4H)-yl}benzonitrile;
3-chloro-5-{4-oxo-3-[(quinolin-6-yloxy)methyl]pyridazin-1(4H)-yl}benzonitrile;
1-(3,5-difluorophenyl)-3-[(quinolin-6-yloxy)methyl]pyridazin-4(1H)-one;
1-(3,4-difluorophenyl)-3-[(quinolin-6-yloxy)methyl]pyridazin-4(1H)-one;
1-(1-methyl-1H-pyrazol-4-yl)-3-[(4-nitrophenoxy)methyl]pyridazin-4(1H)-one;
3-[(quinolin-6-yloxy)methyl]-1-(3,4,5-trifluorophenyl)pyridazin-4(1H)-one;
1-(1-methyl-1H-pyrazol-4-yl)-3-[(quinoxalin-6-yloxy)methyl]pyridazin-4(1H)-one;
ethyl 4-{[1-(1-methyl-1H-pyrazol-4-yl)-4-oxo-1,4-dihydropyridazin-3-yl]methoxy}benzoate;
1-(1-methyl-1H-pyrazol-4-yl)-3-{[(2-methylquinolin-6-yl)oxy]methyl}pyridazin-4(1H)-one;
6-{[1-(1-methyl-1H-pyrazol-4-yl)-4-oxo-1,4-dihydropyridazin-3-yl]methoxy}-1,3-benzothiazole-2-carbonitrile;
1-Methyl-N-(3-{[1-(1-methyl-1H-pyrazol-4-yl)-4-oxo-1,4-dihydropyridazin-3-yl]methoxy}phenyl)-1H-pyrazole-4-carboxamide;
N-methyl-4-{[1-(1-methyl-1H-pyrazol-4-yl)-4-oxo-1,4-dihydropyridazin-3-yl]methoxy}benzamide;
4-{[1-(1-methyl-1H-pyrazol-4-yl)-4-oxo-1,4-dihydropyridazin-3-yl]methoxy}benzamide;
4-{[1-(1-methyl-1H-pyrazol-4-yl)-4-oxo-1,4-dihydropyridazin-3-yl]methoxy}-N-(2,2,2-trifluoroethyl)benzamide;
1-(1-methyl-1H-pyrazol-4-yl)-3-{[(2-phenyl-1H-benzimidazol-5-yl)oxy]methyl}pyridazin-4(1H)-one;
1-(3-Bromophenyl)-3-[(quinolin-6-yloxy)methyl]pyridazin-4(1H)-one;
1-(5-bromopyridin-3-yl)-3-[(quinolin-6-yloxy)methyl]pyridazin-4(1H)-one;
1-(1-ethyl-1H-pyrazol-4-yl)-3-[(quinolin-6-yloxy)methyl]pyridazin-4(1H)-one;
3-{[(3-Ethoxyquinolin-6-yl)oxy]methyl}-1-(1-methyl-1H-pyrazol-4-yl)pyridazin-4(1H)-one;
3-({[3-(2-methoxyethoxy)quinolin-6-yl]oxy}methyl)-1-(1-methyl-1H-pyrazol-4-yl)pyridazin-4(1H)-one;
1-(1-methyl-1H-pyrazol-4-yl)-3-({[3-(tetrahydrofuran-3-ylmethoxy)quinolin-6-yl]oxy}methyl)pyridazin-4(1H)-one;
3-[({3-[(3-methyloxetan-3-yl)methoxy]quinolin-6-yl}oxy)methyl]-1-(1-methyl-1H-pyrazol-4-yl)pyridazin-4(1H)-one;
1-(1-methyl-1H-pyrazol-4-yl)-3-[(quinazolin-6-yloxy)methyl]pyridazin-4(1H)-one;
3-fluoro-5-[3-({[3-(2-methoxyethoxy)quinolin-6-yl]oxy}methyl)-4-oxopyridazin-1(4H)-yl]benzonitrile;
3-chloro-5-[3-({[3-(2-methoxyethoxy)quinolin-6-yl]oxy}methyl)-4-oxopyridazin-1(4H)-yl]benzonitrile;
1-(3,4-difluorophenyl)-3-({[3-(2-methoxyethoxy)quinolin-6-yl]oxy}methyl)pyridazin-4(1H)-one;
1-(3,5-difluorophenyl)-3-({[3-(2-methoxyethoxy)quinolin-6-yl]oxy}methyl)pyridazin-4(1H)-one;
3-{[(8-fluoroquinolin-6-yl)oxy]methyl}-1-(1-methyl-1H-pyrazol-4-yl)pyridazin-4(1H)-one;
3-({[4-(2-ethoxyethoxy)quinolin-6-yl]oxy}methyl)-1-(1-methyl-1H-pyrazol-4-yl)pyridazin-4(1H)-one;
3-({[4-(2-methoxyethoxy)quinolin-6-yl]oxy}methyl)-1-(1-methyl-1H-pyrazol-4-yl)pyridazin-4(1H)-one;
3-{[(3-methoxyquinolin-6-yl)oxy]methyl}-1-(1-methyl-1H-pyrazol-4-yl)pyridazin-4(1H)-one;
1-(1-ethyl-1H-pyrazol-4-yl)-3-({[3-(2-methoxyethoxy)quinolin-6-yl]oxy}methyl)pyridazin-4(1H)-one;
3-{[(3-fluoroquinolin-6-yl)oxy]methyl}-1-(1-methyl-1H-pyrazol-4-yl)pyridazin-4(1H)-one;
1-(3-bromophenyl)-3-({[3-(2-methoxyethoxy)quinolin-6-yl]oxy}methyl)pyridazin-4(1H)-one;
1-(1-methyl-1H-pyrazol-4-yl)-3-({[3-(4H-1,2,4-triazol-4-yl)quinolin-6-yl]oxy}methyl)pyridazin-4(1H)-one;
3-[({3-[4-(methoxymethyl)-1H-1,2,3-triazol-1-yl]quinolin-6-yl}oxy)methyl]-1-(1-methyl-1H-pyrazol-4-yl)pyridazin-4(1H)-one;
1-(1-methyl-1H-pyrazol-4-yl)-3-{[(3-{4-[(piperidin-4-yloxy)methyl]-1-(1-methyl-1H-pyrazol-4-yl)pyridazin-4(1H)-one;
1-(1-Methyl-1H-pyrazol-4-yl)-3-({[3-(1H-1,2,3-triazol-1-yl)quinolin-6-yl]oxy)methyl)pyridazin-4(1H)-one;
3-[(4-Ethoxyquinolin-6-yl)oxy]methyl}-1-(1-methyl-1H-pyrazol-4-yl)pyridazin-4(1H)-one;
3-[3-{[(3-ethoxyquinolin-6-yl)oxy]methyl}-4-oxopyridazin-1(4H)-yl]-5-fluorobenzonitrile;
3-chloro-5-[3-{[(3-ethoxyquinolin-6-yl)oxy]methyl}-4-oxopyridazin-1(4H)-yl]benzonitrile;
1-(3,4-difluorophenyl)-3-{([(3-ethoxyquinolin-6-yl)oxy]methyl}pyridazin-4(1H)-one;
1-(3,5-difluorophenyl)-3-{[(3-ethoxyquinolin-6-yl)oxy]methyl}pyridazin-4(1H)-one;
3-{[(4-Methoxyquinolin-6-yl)oxy]methyl}-1-(1-methyl-1H-pyrazol-4-yl)pyridazin-4(1H)-one;
3-{[(2-methoxyquinolin-6-yl)oxy]methyl}-1-(1-methyl-1H-pyrazol-4-yl)pyridazin-4(1H)-one;
1-(1-methyl-1H-pyrazol-4-yl)-3-{[(4-methylquinolin-6-yl)oxy]methyl}pyridazin-4(1H)-one;
3-{4-Oxo-3-[(quinolin-6-yloxy)methyl]pyridazin-1(4H)-yl}benzamide;
3-[3-({[3-(2-methoxyethoxy)quinolin-6-yl]oxy}methyl)-4-oxopyridazin-1(4H)-yl]-N,N-dimethylbenzamide;
1-(1-Methyl-1H-pyrazol-4-yl)-3-[2-(quinolin-6-yl)ethyl]pyridazin-4(1H)-one;
1-(1-methyl-1H-pyrazol-4-yl)-3-(2-quinolin-4-ylethyl)pyridazin-4(1H)-one;
1-(1-methyl-1H-pyrazol-4-yl)-3-[2-(1H-pyrrolo[2,3-b]pyridin-4-yl)ethyl]pyridazin-4(1H)-one;
methyl 4-{2-[1-(1-methyl-1H-pyrazol-4-yl)-4-oxo-1,4-dihydropyridazin-3-yl]ethyl}benzoate;
3-{2-[3-(2-methoxyethoxy)quinolin-6-yl]ethyl}-1-(1-methyl-1H-pyrazol-4-yl)pyridazin-4(1H)-one;
3-[2-(3-ethoxyquinolin-6-yl)ethyl]-1-(1-methyl-1H-pyrazol-4-yl)pyridazin-4(1H)-one;
3-[2-(4-methoxyquinolin-6-yl)ethyl]-1-(1-methyl-1H-pyrazol-4-yl)pyridazin-4(1H)-one;
1-(1-Methyl-1H-pyrazol-4-yl)-3-[2-(quinoxalin-6-yl)ethyl]pyridazin-4(1H)-one;
3-Hydroxy-6-{[1-(1-methyl-1H-pyrazol-4-yl)-4-oxo-1,4-dihydropyridazin-3-yl]methoxy}quinolinium chloride;
3-({[3-(2-Hydroxy-2-methylpropoxy)quinolin-6-yl]oxy}methyl)-1-(1-methyl-1H-pyrazol-4-yl)pyridazin-4(1H)-one;
1-(1-Methyl-1H-pyrazol-4-yl)-3-({[3-(oxetan-3-yloxy)quinolin-6-yl]oxy}methyl)pyridazin-4(1H)-one;
3-[({3-[(2H5)ethyloxy]quinolin-6-yl}oxy)methyl]-1-(1-methyl-1H-pyrazol-4-yl)pyridazin-4(1H)-one;
3-({[3-(2,2-difluoroethoxy)quinolin-6-yl]oxy}methyl)-1-(1-methyl-1H-pyrazol-4-yl)pyridazin-4(1H)-one;
3-({[3-(difluoromethoxy)quinolin-6-yl]oxy}methyl)-1-(1-methyl-1H-pyrazol-4-yl)pyridazin-4(1H)-one;
1-(1-ethyl-1H-pyrazol-4-yl)-3-({[3-(pyridin-2-yloxy)quinolin-6-yl]oxy}methyl)pyridazin-4(1H)-one;
3-({[3-(2,2-difluoroethoxy)quinolin-6-yl]oxy}methyl)-1-(1-ethyl-1H-pyrazol-4-yl)pyridazin-4(1H)-one;
1-(1-ethyl-1H-pyrazol-4-yl)-3-({[3-(tetrahydrofuran-3-yloxy)quinolin-6-yl]oxy}methyl)pyridazin-4(1H)-one;
1-(1-ethyl-1H-pyrazol-4-yl)-3-[({3-[(5-methyl-1,2,4-oxadiazol-3-yl)methoxy]quinolin-6-yl}oxy)methyl]pyridazin-4(1H)-one;
1-(1-Methyl-1H-pyrazol-4-yl)-3-{[(3-methylquinolin-6-yl)oxy]methyl}pyridazin-4(1H)-one;
3-{[(3-ethylquinolin-6-yl)oxy]methyl}-1-(1-methyl-1H-pyrazol-4-yl)pyridazin-4(1H)-one;
3-({[3-(methoxymethyl)quinolin-6-yl]oxy}methyl)-1-(1-methyl-1H-pyrazol-4-yl)pyridazin-4(1H)-one;
1-(1-Methyl-1H-pyrazol-4-yl)-3-{[(3-pyridin-3-ylquinolin-6-yl)oxy]methyl}pyridazin-4(1H)-one;
1-(1-methyl-1H-pyrazol-4-yl)-3-({[3-(1-methyl-1H-pyrazol-4-yl)quinolin-6-yl]oxy}methyl)pyridazin-4(1H)-one;
6-(1-ethyl-1H-pyrazol-4-yl)-2-[({3-[4-(methylsulfonyl)phenyl]quinolin-6-yl}oxy)methyl]pyridin-3(6H)-one;
1-(1-ethyl-1H-pyrazol-4-yl)-3-[({3-[1-(2-methoxyethyl)-1H-pyrazol-4-yl]quinolin-6-yl}oxy)methyl]pyridazin-4(1H)-one;
1-(1-ethyl-1H-pyrazol-4-yl)-3-({[3-(4-fluorophenyl)quinolin-6-yl]oxy}methyl)pyridazin-4(1H)-one;
1-(1-ethyl-1H-pyrazol-4-yl)-3-({[3-(4-methoxyphenyl)quinolin-6-yl]oxy}methyl)pyridazin-4(1H)-one;
1-(1-ethyl-1H-pyrazol-4-yl)-3-{[(3-pyridin-4-ylquinolin-6-yl)oxy]methyl}pyridazin-4(1H)-one;
5-(6-{[1-(1-ethyl-1H-pyrazol-4-yl)-4-oxo-1,4-dihydropyridazin-3-yl]methoxy}quinolin-3-yl)pyridine-2-carbonitrile;
1-(1-ethyl-1H-pyrazol-4-yl)-3-({[3-(6-methoxypyridin-3-yl)quinolin-6-yl]oxy}methyl)pyridazin-4(1H)-one;
1-(1-ethyl-1H-pyrazol-4-yl)-3-({[3-(2-fluorophenyl)quinolin-6-yl]oxy}methyl)pyridazin-4(1H)-one;
1-(1-ethyl-1H-pyrazol-4-yl)-3-({[3-(2-methylphenyl)quinolin-6-yl]oxy}methyl)pyridazin-4(1H)-one;
1-(1-ethyl-1H-pyrazol-4-yl)-3-{[(3-pyrimidin-5-ylquinolin-6-yl)oxy]methyl}pyridazin-4(1H)-one;
1-(1-ethyl-1H-pyrazol-4-yl)-3-({[3-(1,3-thiazol-4-yl)quinolin-6-yl]oxy}methyl)pyridazin-4(1H)-one;
1-(1-ethyl-1H-pyrazol-4-yl)-3-({[3-(6-morpholin-4-ylpyridin-3-yl)quinolin-6-yl]oxy}methyl)pyridazin-4(1H)-one;
3-({[3-(6-chloropyridin-3-yl)quinolin-6-yl]oxy}) methyl)-1-(1-ethyl-1H-pyrazol-4-yl)pyridazin-4(1H)-one;
1-(1-ethyl-1H-pyrazol-4-yl)-3-[({3-[4-(trifluoromethyl)phenyl]quinolin-6-yl}oxy)methyl]pyridazin-4(1H)-one;
1-(1-ethyl-1H-pyrazol-4-yl)-3-({[3-(3-fluorophenyl)quinolin-6-yl]oxy}methyl)pyridazin-4(1H)-one;
1-(1-ethyl-1H-pyrazol-4-yl)-3-{[(3-phenylquinolin-6-yl)oxy]methyl}pyridazin-4(1H)-one;
tert-Butyl-3-[4-(6-{[1-(1-methyl-1H-pyrazol-4-yl)-4-oxo-1,4-dihydropyridazin-3-yl]methoxy}quinolin-3-yl)-1H-pyrazol-1-yl]azetidine-1-carboxylate;
3-[({3-[(1)-1-(Azetidin-3-ylamino)prop-1-en-2-yl]quinolin-6-yl}oxy)methyl]-1-(1-methyl-1H-pyrazol-4-yl)pyridazin-4(1H)-one ammoniate;
1-(1-methyl-1H-pyrazol-4-yl)-3-[({3-[1-(piperidin-4-yl)-1H-pyrazol-4-yl]quinolin-6-yl}oxy)methyl]pyridazin-4(1H)-one;
Methyl 6-{[1-(1-methyl-1H-pyrazol-4-yl)-4-oxo-1,4-dihydropyridazin-3-yl]methoxy}quinoline-3-carboxylate;
1-(1-Methyl-1H-pyrazol-4-yl)-3-[({3-[1-(oxetan-3-yl)-1H-pyrazol-4-yl]quinolin-6-yl)}oxy)methyl]pyridazin-4(1H)-one;
3-{[(4-Chloroquinolin-6-yl)oxy]methyl}-1-(1-methyl-1H-pyrazol-4-yl)pyridazin-4(1H)-one;
3-{[(4-Ethylquinolin-6-yl)oxy]methyl}-1-(1-methyl-1H-pyrazol-4-yl)pyridazin-4(1H)-one;
3-({[3-(2-Methoxyethoxy)quinolin-6-yl]sulfanyl}methyl)-1-(1-methyl-1H-pyrazol-4-yl)pyridazin-4(1H)-one;
3-{[(3-ethoxyquinolin-6-yl)sulfanyl]methyl}-1-(1-methyl-1H-pyrazol-4-yl)pyridazin-4(1H)-one;
1-(1-Methyl-1H-pyrazol-4-yl)-3-[(quinolin-6-ylsulfanyl)methyl]pyridazin-4(1H)-one;
3-({[3-(2-Methoxyethoxy)quinolin-6-yl]amino}methyl)-1-(1-methyl-1H-pyrazol-4-yl)pyridazin-4(1H)-one;
3-{[(3-ethoxyquinolin-6-yl)amino]methyl}-1-(1-methyl-1H-pyrazol-4-yl)pyridazin-4(1H)-one;
1-(1-Methyl-1H-pyrazol-4-yl)-3-[(quinolin-6-ylamino)methyl]pyridazin-4(1H)-one;
3-[(1,3-Benzothiazol-6-yloxy)methyl]-1-(1-methyl-1H-pyrazol-4-yl)pyridazin-4(1H)-one;
3-({[3-(2-methoxyethoxy)quinoxalin-6-yl]oxy}methyl)-1-(1-methyl-1H-pyrazol-4-yl)pyridazin-4(1H)-one;
3-[(2,3-dihydro[1,4]dioxino[2,3-c]quinolin-9-yloxy)methyl]-1-(1-methyl-1H-pyrazol-4-yl)pyridazin-4(1H)-one;
1-(1-Methyl-1H-pyrazol-4-yl)-3-{[methyl(quinolin-6-yl)amino]methyl}pyridazin-4(1H)-one;
1-(1-methyl-1H-pyrazol-4-yl)-3-({[3-(morpholin-4-ylcarbonyl)quinolin-6-yl]oxy}methyl)pyridazin-4(1H)-one;
tert-Butyl (6-{[1-(1-methyl-1H-pyrazol-4-yl)-4-oxo-1,4-dihydropyridazin-3-yl]methoxy}quinolin-3-yl)carbamate;
1,3-bis(6-{[1-(1-methyl-1H-pyrazol-4-yl)-4-oxo-1,4-dihydropyridazin-3-yl]methoxy}quinolin-3-yl)urea;
3-{[(3-Aminoquinolin-6-yl)oxy]methyl}-1-(1-methyl-1H-pyrazol-4-yl)pyridazin-4(1H)-one;
1-(3-Bromophenyl)-3-[1-(quinolin-6-yloxy)ethyl]pyridazin-4(1H)-one;
1-(3-Bromophenyl)-3-[1-(quinolin-6-yloxy)ethyl]pyridazin-4(1H)-one;
3-((4-aminophenoxy)methyl)-1-(1-methyl-1H-pyrazol-4-yl)pyridazin-4(1H)-one;
3-((4-(4-hydroxybutylamino)phenoxy)methyl)-1-(1-methyl-1H-pyrazol-4-yl)pyridazin-4(1H)-one;
3-[2-(4-aminophenyl)ethyl]-1-(1-methyl-1H-pyrazol-4-yl)pyridazin-4(1H)-one;
3-(2-{4-[(4-hydroxybutyl)amino]phenyl}ethyl)-1-(1-methyl-1H-pyrazol-4-yl)pyridazin-4(1H)-one;
2-methoxyethyl (4-{[1-(1-methyl-1H-pyrazol-4-yl)-4-oxo-1,4-dihydropyridazin-3-yl]methoxy}phenyl)carbamate;
2-methoxyethyl (4-{2-[1-(1-methyl-1H-pyrazol-4-yl)-4-oxo-1,4-dihydropyridazin-3-yl]ethyl}phenyl)carbamate;
ethyl (4-{2-[1-(1-methyl-1H-pyrazol-4-yl)-4-oxo-1,4-dihydropyridazin-3-yl]ethyl}phenyl)carbamate;
ethyl (4-{[1-(1-methyl-1H-pyrazol-4-yl)-4-oxo-1,4-dihydropyridazin-3-yl]methoxy}phenyl)carbamate;
2-methylpropyl (4-{[1-(1-methyl-1H-pyrazol-4-yl)-4-oxo-1,4-dihydropyridazin-3-yl]methoxy}phenyl)carbamate;
1-(1-Methyl-1H-pyrazol-4-yl)-3-(2-quinolin-6-ylprop-2-en-1-yl)pyridazin-4(1H)-one;
-(1-Methyl-1H-pyrazol-4-yl)-3-(2-quinolin-6-ylpropyl)pyridazin-4(1H)-one;
-(1-Methyl-1H-pyrazol-4-yl)-3-(2-quinolin-6-ylpropyl)pyridazin-4(1H)-one;
1-(1-Methyl-1H-pyrazol-4-yl)-3-(quinolin-6-ylmethoxy)pyridazin-4(1H)-one;
1-(1-ethyl-1H-pyrazol-4-yl)-3-(quinolin-6-ylmethoxy)pyridazin-4(1H)-one;
1-(1-methyl-1H-pyrazol-4-yl)-3-({[3-(1-methyl-1H-pyrazol-4-yl)quinolin-6-yl]methoxy}pyridazin-4(1H)-one;
1-(1-methyl-1H-pyrazol-4-yl)-3-(1-quinolin-6-ylethoxy)pyridazin-4(1H)-one;
3-[1-(4-methoxyquinolin-6-yl)ethoxy]-1-(1-methyl-1H-pyrazol-4-yl)pyridazin-4(1H)-one;
1-(1-ethyl-1H-pyrazol-4-yl)-3-[1-(4-methoxyquinolin-6-yl)ethoxy]pyridazin-4(1H)-one;
3-[1-(3-ethoxyquinolin-6-yl)ethoxy]-1-(1-methyl-1H-pyrazol-4-yl)pyridazin-4(1H)-one;
3-{1-[3-(2-methoxyethoxy)quinolin-6-yl]ethoxy}-1-(1-methyl-1H-pyrazol-4-yl)pyridazin-4(1H)-one;
3-[1-(3-methoxyquinolin-6-yl)ethoxy]-1-(1-methyl-1H-pyrazol-4-yl)pyridazin-4(1H)-one;
1-(1-methyl-1H-pyrazol-4-yl)-3-{1-[3-(1-methyl-1H-pyrazol-4-yl)quinolin-6-yl]ethoxy}pyridazin-4(1H)-one;
3-[(4-methoxyquinolin-6-yl)methoxy]-1-(1-methyl-1H-pyrazol-4-yl)pyridazin-4(1H)-one;
1-(1-methyl-1H-pyrazol-4-yl)-3-[1-(quinolin-6-yl)ethoxy]pyridazin-4(1H)-one;
1-(1-methyl-1H-pyrazol-4-yl)-3-[1-(quinolin-6-yl)ethoxy]pyridazin-4(1H)-one;
3-[1-(3-methoxyquinolin-6-yl)ethoxy]-1-(1-methyl-1H-pyrazol-4-yl)pyridazin-4(1H)-one enantiomer 2;
3-[1-(3-ethoxyquinolin-6-yl)ethoxy]-1-(1-methyl-1H-pyrazol-4-yl)pyridazin-4(1H)-one enantiomer 1;
3-[1-(3-ethoxyquinolin-6-yl)ethoxy]-1-(1-methyl-1H-pyrazol-4-yl)pyridazin-4(1H)-one enantiomer 2;
3-{1-[3-(2-methoxyethoxy)quinolin-6-yl]ethoxy}-1-(1-methyl-1H-pyrazol-4-yl)pyridazin-4(1H)-one enantiomer 1;
3-{1-[3-(2-methoxyethoxy)quinolin-6-yl]ethoxy}-1-(1-methyl-1H-pyrazol-4-yl)pyridazin-4(1H)-one enantiomer 2;
3-[1-(4-methoxyquinolin-6-yl)ethoxy]-1-(1-methyl-1H-pyrazol-4-yl)pyridazin-4(1H)-one enantiomer 2;
1-(1-ethyl-1H-pyrazol-4-yl)-3-[1-(4-methoxyquinolin-6-yl)ethoxy]pyridazin-4(1H)-one enantiomer 2;
1-(1-methyl-1H-pyrazol-4-yl)-3-[(quinolin-6-ylmethyl)amino]pyridazin-4(1H)-one;
1-(1-methyl-1H-pyrazol-4-yl)-3-{[1-(quinolin-6-yl)ethyl]amino}pyridazin-4(1H)-one;
1-(1-methyl-1H-pyrazol-4-yl)-3-[(quinolin-6-ylmethyl)thio]pyridazin-4(1H)-one;
methyl {[1-(1-methyl-1H-pyrazol-4-yl)-4-oxo-1,4-dihydropyridazin-3-yl]oxy}(quinolin-6-yl)acetate;
   or a pharmaceutically acceptable salt thereof.

The compounds of the present invention may have asymmetric centers, chiral axes, and chiral planes (as described in: E. L. Eliel and S. H. Wilen, Stereochemistry of Carbon Compounds, John Wiley & Sons, New York, 1994, pages 1119-1190), and occur as racemates, racemic mixtures, and as individual diastereomers, with all possible isomers and mixtures thereof, including optical isomers, all such stereoisomers being included in the present invention. In addition, the compounds disclosed herein may exist as tautomers and both tautomeric forms are intended to be encompassed by the scope of the invention, even though only one tautomeric structure is depicted.

In the compounds of generic Formula I, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of generic Formula I. For example, different isotopic forms of hydrogen (H) include protium (1H) and deuterium (2H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds within generic Formula I can be prepared without undue experimentation by conventional techniques well known to those skilled in the art or by processes analogous to those described in the Schemes and Examples herein using appropriate isotopically-enriched reagents and/or intermediates.

When any variable (e.g. R⁶) occurs more than one time in any constituent, its definition on each occurrence is independent at every other occurrence. Also, combinations of substituents and variables are permissible only if such combinations result in stable compounds. Lines drawn into the ring systems from substituents represent that the indicated bond may be attached to any of the substitutable ring atoms. If the ring system is polycyclic, it is intended that the bond be attached to any of the suitable carbon atoms on the proximal ring only.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results. The phrase “optionally substituted with one or more substituents” should be taken to be equivalent to the phrase “optionally substituted with at least one substituent” and in such cases another embodiment will have from zero to three substituents.

As used herein, “alkyl” is intended to include both branched and straight-chain saturated aliphatic hydrocarbon groups having the specified number of carbon atoms. For example, C₁-C₁₀, as in “C₁-C₁₀ alkyl” is defined to include groups having 1, 2, 3, 4, 5, 6, 7, 8, 9 or 10 carbons in a linear or branched arrangement. For example, “C₁-C₁₀ alkyl” specifically includes methyl, ethyl, n-propyl, i-propyl, n-butyl, t-butyl, i-butyl, pentyl, hexyl, heptyl, octyl, nonyl, decyl, and so on. The term “cycloalkyl” means a monocyclic saturated aliphatic hydrocarbon group having the specified number of carbon atoms. For example, “cycloalkyl” includes cyclopropyl, methyl-cyclopropyl, 2,2-dimethyl-cyclobutyl, 2-ethyl-cyclopentyl, cyclohexyl, and so on. In an embodiment of the invention the term “cycloalkyl” includes the groups described immediately above and further includes monocyclic unsaturated aliphatic hydrocarbon groups. For example, “cycloalkyl” as defined in this embodiment includes cyclopropyl, methyl-cyclopropyl, 2,2-dimethyl-cyclobutyl, 2-ethyl-cyclopentyl, cyclohexyl, cyclopentenyl, cyclobutenyl and so on.

The term “haloalkyl” means an alkyl radical as defined above, unless otherwise specified, that is substituted with one to five, preferably one to three halogen. Representative examples include, but are not limited to trifluoromethyl, dichloroethyl, and the like.

As used herein, the term “alkenyl” refers to a non-aromatic hydrocarbon radical, straight or branched, containing from 2 to 10 carbon atoms and at least 1 carbon to carbon double bond. Preferably 1 carbon to carbon double bond is present, and up to 4 non-aromatic carbon-carbon double bonds may be present. Thus, “C₂-C₆ alkenyl” means an alkenyl radical having from 2 to 6 carbon atoms. Alkenyl groups include ethenyl, propenyl, butenyl and cyclohexenyl. As described above with respect to alkyl, the straight, branched or cyclic portion of the alkenyl group may contain double bonds and may be substituted if a substituted alkenyl group is indicated.

“Alkoxy” represents either a cyclic or non-cyclic alkyl group of indicated number of carbon atoms attached through an oxygen bridge. “Alkoxy” therefore encompasses the definitions of alkyl and cycloalkyl above.

In certain instances, substituents may be defined with a range of carbons that includes zero, such as (C₀-C₆)alkylene-aryl. If aryl is taken to be phenyl, this definition would include phenyl itself as well as —CH₂Ph, —CH₂CH₂Ph, CH(CH₃)CH₂CH(CH₃)Ph, and so on.

As used herein, “aryl” is intended to mean any stable monocyclic or bicyclic carbon ring of up to 7 atoms in each ring, wherein at least one ring is aromatic. Examples of such aryl elements include phenyl, naphthyl, tetrahydronaphthyl, indanyl and biphenyl. In cases where the aryl substituent is bicyclic and one ring is non-aromatic, it is understood that attachment is via the aromatic ring.

The term “heteroaryl,” as used herein, represents a stable monocyclic or bicyclic ring of up to 7 atoms in each ring, wherein at least one ring is aromatic and contains from 1 to 4 heteroatoms selected from the group consisting of O, N and S. Heteroaryl groups within the scope of this definition include but are not limited to: acridinyl, carbazolyl, cinnolinyl, quinoxalinyl, pyrrazolyl, indolyl, benzotriazolyl, furanyl, thienyl, benzothienyl, benzofuranyl, benzimidazolonyl, benzoxazolonyl, quinolinyl, isoquinolinyl, dihydroisoindolonyl, imidazopyridinyl, isoindolonyl, indazolyl, oxazolyl, oxadiazolyl, isoxazolyl, indolyl, pyrazinyl, pyridazinyl, pyridinyl, pyrimidinyl, pyrrolyl, tetrahydroquinoline. As with the definition of heterocycle below, “heteroaryl” is also understood to include the N-oxide derivative of any nitrogen-containing heteroaryl. In cases where the heteroaryl substituent is bicyclic and one ring is non-aromatic or contains no heteroatoms, it is understood that attachment is via the aromatic ring or via the heteroatom containing ring, respectively.

The term “heterocycle” or “heterocyclyl” as used herein is intended to mean a 3- to 10-membered aromatic or nonaromatic heterocycle containing from 1 to 4 heteroatoms selected from the group consisting of O, N and S, and includes bicyclic groups. For the purposes of this invention, the term “heterocyclic” is also considered to be synonymous with the terms “heterocycle” and “heterocyclyl” and is understood as also having the definitions set forth herein. “Heterocyclyl” therefore includes the above mentioned heteroaryls, as well as dihydro and tetrathydro analogs thereof. Further examples of “heterocyclyl” include, but are not limited to the following: azetidinyl, benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthpyridinyl, oxadiazolyl, oxooxazolidinyl, oxazolyl, oxazoline, oxopiperazinyl, oxopyrrolidinyl, oxomorpholinyl, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrahydropyranyl, tetrahydrofuranyl, tetrahydrothiopyranyl, tetrahydroisoquinolinyl, tetrazolyl, tetrazolopyridyl, thiadiazolyl, thiazolyl, thienyl, triazolyl, 1,4-dioxanyl, hexahydroazepinyl, piperazinyl, piperidinyl, pyridin-2-onyl, pyrrolidinyl, morpholinyl, thiomorpholinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, dioxidothiomorpholinyl, methylenedioxybenzoyl, tetrahydrofuranyl, and tetrahydrothienyl, and N-oxides thereof. Attachment of a heterocyclyl substituent can occur via a carbon atom or via a heteroatom.

As appreciated by those of skill in the art, “halo” or “halogen” as used herein is intended to include chloro, fluoro, bromo and iodo.

The alkyl, alkenyl, cycloalkyl, aryl, heteroaryl and heterocyclyl substituents may be substituted or unsubstituted, unless specifically defined otherwise. For example, a (C₁-C₆)alkyl may be substituted with one, two or three substituents selected from OH, oxo, halogen, alkoxy, dialkylamino, or heterocyclyl, such as morpholinyl, piperidinyl, and so on. In this case, if one substituent is oxo and the other is OH, the following are included in the definition: —C═O)CH₂CH(OH)CH₃, —(C═O)OH, —CH₂(OH)CH₂CH(O), and so on.

Included in the instant invention is the free form of compounds of the instant invention, as well as the pharmaceutically acceptable salts and stereoisomers thereof. The term “free form” refers to the amine compounds in non-salt form. The encompassed pharmaceutically acceptable salts not only include the salts exemplified for the specific compounds described herein, but also all the typical pharmaceutically acceptable salts of the free form of compounds of the instant invention. The free form of the specific salt compounds described may be isolated using techniques known in the art. For example, the free form may be regenerated by treating the salt with a suitable dilute aqueous base solution such as dilute aqueous NaOH, potassium carbonate, ammonia and sodium bicarbonate. The free forms may differ from their respective salt forms somewhat in certain physical properties, such as solubility in polar solvents, but the acid and base salts are otherwise pharmaceutically equivalent to their respective free forms for purposes of the invention.

The pharmaceutically acceptable salts of the instant compounds can be synthesized from the compounds of this invention which contain a basic or acidic moiety by conventional chemical methods. Generally, the salts of the basic compounds are prepared either by ion exchange chromatography or by reacting the free base with stoichiometric amounts or with an excess of the desired salt-forming inorganic or organic acid in a suitable solvent or various combinations of solvents. Similarly, the salts of the acidic compounds are formed by reactions with the appropriate inorganic or organic base.

Thus, pharmaceutically acceptable salts of the compounds of this invention include the conventional non-toxic salts of the compounds of this invention as formed by reacting a basic instant compound with an inorganic or organic acid. For example, conventional non-toxic salts include those derived from inorganic acids such as hydrochloric, hydrobromic, sulfuric, sulfamic, phosphoric, nitric and the like, as well as salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxy-benzoic, fumaric, toluenesulfonic, methanesulfonic, ethane disulfonic, oxalic, isethionic, trifluoroacetic and the like.

When the compound of the present invention is acidic, suitable “pharmaceutically acceptable salts” refers to salts prepared form pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc and the like. Particularly preferred are the ammonium, calcium, magnesium, potassium and sodium salts. Salts derived from pharmaceutically acceptable organic non-toxic bases include salts of primary, secondary and tertiary amines, substituted amines including naturally occurring substituted amines, cyclic amines and basic ion exchange resins, such as arginine, betaine caffeine, choline, N,N′-dibenzylethylenediamine, diethylamin, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine tripropylamine, tromethamine and the like. When the compound of the present invention is acidic, the term “free form” refers to the compound in its non-salt form, such that the acidic functionality is still protonated.

The preparation of the pharmaceutically acceptable salts described above and other typical pharmaceutically acceptable salts is more fully described by Berg et al., “Pharmaceutical Salts,” J. Pharm. Sci., 1977:66:1-19.

It will also be noted that the compounds of the present invention may potentially be internal salts or zwitterions, since under physiological conditions a deprotonated acidic moiety in the compound, such as a carboxyl group, may be anionic, and this electronic charge might then be balanced off internally against the cationic charge of a protonated or alkylated basic moiety, such as a quaternary nitrogen atom. An isolated compound having internally balance charges, and thus not associated with an intermolecular counterion, may also be considered the “free form” of a compound.

### Utilities

The compounds of the invention are useful to bind to and/or modulate the activity of a tyrosine kinase, in particular, a receptor tyrosine kinase. In an embodiment, the receptor tyrosine kinase is a member of the MET subfamily. In a further embodiment, the MET is human MET, although the activity of receptor tyrosine kinases from other organisms may also be modulated by the compounds of the present invention. In this context, modulate means either increasing or decreasing kinase activity of MET. In an embodiment, the compounds of the instant invention inhibit the kinase activity of MET.

The compounds of the invention find use in a variety of applications. As will be appreciated by those skilled in the art, the kinase activity of MET may be modulated in a variety of ways; that is, one can affect the phosphorylation/activation of MET either by modulating the initial phosphorylation of the protein or by modulating the autophosphorylation of the other active sites of the protein. Alternatively, the kinase activity of MET may be modulated by affecting the binding of a substrate of MET phosphorylation.

The compounds of the invention are used to treat or prevent cellular proliferation diseases. Disease states which can be treated by the methods and compositions provided herein include, but are not limited to, cancer (further discussed below), autoimmune disease, arthritis, graft rejection, inflammatory bowel disease, proliferation induced after medical procedures, including, but not limited to, surgery, angioplasty, and the like. It is appreciated that in some cases the cells may not be in a hyper- or hypoproliferation state (abnormal state) and still require treatment. Thus, in one embodiment, the invention herein includes application to cells or individuals which are afflicted or may eventually become afflicted with any one of these disorders or states.

The compounds, compositions and methods provided herein are particularly deemed useful for the treatment and prevention of cancer including solid tumors such as skin, breast, brain, cervical carcinomas, testicular carcinomas, etc. In an embodiment, the instant compounds are useful for treating cancer. In particular, cancers that may be treated by the compounds, compositions and methods of the invention include, but are not limited to: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor [nephroblastoma], lymphoma, leukemia,), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord (neurofibroma, meningioma, glioma, sarcoma); Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; Skin: malignant melanoma, basal cell carcinoma, squamous cell carcinoma, Karposi's sarcoma, moles dysplastic nevi, lipoma, angioma, dermatofibroma, keloids, psoriasis; and Adrenal glands: neuroblastoma. Thus, the term “cancerous cell” as provided herein, includes a cell afflicted by any one of the above-identified conditions. In an embodiment of the invention, cancers that may be treated by the compounds, compositions and methods of the invention include, in addition to the cancers listed above: Lung: bronchogenic carcinoma (non-small cell lung); Gastrointestinal: rectal, colorectal and colon; Genitourinary tract: kidney (papillary renal cell carcinoma); and Skin: head and neck squamous cell carcinoma.

In another embodiment, the compounds of the instant invention are useful for treating or preventing cancer selected from: head and neck squamous cell carcinomas, histiocytic lymphoma, lung adenocarcinoma, small cell lung cancer, non-small cell lung cancer, pancreatic cancer, papillary renal cell carcinoma, liver cancer, gastric cancer, colon cancer, multiple myeloma, glioblastomas and breast carcinoma. In yet another embodiment, the compounds of the instant invention are useful for treating or preventing cancer selected from: histiocytic lymphoma, lung adenocarcinoma, small cell lung cancer, pancreatic cancer, liver cancer, gastric cancer, colon cancer, multiple myeloma, glioblastomas and breast carcinoma. In still another embodiment, the compounds of the instant invention are useful for treating cancer selected from: histiocytic lymphoma, lung adenocarcinoma, small cell lung cancers, pancreatic cancer, liver cancer, gastric cancer, colon cancer, multiple myeloma, glioblastomas and breast carcinoma.

In another embodiment, the compounds of the instant invention are useful for the prevention or modulation of the metastases of cancer cells and cancer. In particular, the compounds of the instant invention are useful to prevent or modulate the metastases of ovarian cancer, childhood hepatocellular carcinoma, metastatic head and neck squamous cell carcinomas, gastric cancers, breast cancer, colorectal cancer, cervical cancer, lung cancer, nasopharyngeal cancer, pancreatic cancer, glioblastoma and sarcomas.

The compounds of this invention may be administered to mammals, preferably humans, either alone or in combination with pharmaceutically acceptable carriers, excipients or diluents, in a pharmaceutical composition, according to standard pharmaceutical practice. The compounds can be administered orally or parenterally, including the intravenous, intramuscular, intraperitoneal, subcutaneous, rectal and topical routes of administration.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, microcrystalline cellulose, sodium crosscarmellose, corn starch, or alginic acid; binding agents, for example starch, gelatin, polyvinyl-pyrrolidone or acacia, and lubricating agents, for example, magnesium stearate, stearic acid or talc. The tablets may be uncoated or they may be coated by known techniques to mask the unpleasant taste of the drug or delay disintegration and absorption in the gastrointestinal tract and thereby provide a sustained action over a longer period. For example, a water soluble taste masking material such as hydroxypropyl-methylcellulose or hydroxypropylcellulose, or a time delay material such as ethyl cellulose, cellulose acetate butyrate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethyleneglycol or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxymethylcellulose, methylcellulose, hydroxypropylmethyl-cellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as butylated hydroxyanisol or alpha-tocopherol.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present. These compositions may be preserved by the addition of an anti-oxidant such as ascorbic acid.

The pharmaceutical compositions of the invention may also be in the form of an oil-in-water emulsions. The oily phase may be a vegetable oil, for example olive oil or arachis oil, or a mineral oil, for example liquid paraffin or mixtures of these. Suitable emulsifying agents may be naturally occurring phosphatides, for example soy bean lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening, flavoring agents, preservatives and antioxidants.

Syrups and elixirs may be formulated with sweetening agents, for example glycerol, propylene glycol, sorbitol or sucrose. Such formulations may also contain a demulcent, a preservative, flavoring and coloring agents and antioxidant.

The pharmaceutical compositions may be in the form of a sterile injectable aqueous solutions. Among the acceptable vehicles and solvents that may be employed are water, Ringer's solution and isotonic sodium chloride solution.

The sterile injectable preparation may also be a sterile injectable oil-in-water microemulsion where the active ingredient is dissolved in the oily phase. For example, the active ingredient may be first dissolved in a mixture of soybean oil and lecithin. The oil solution then introduced into a water and glycerol mixture and processed to form a microemulation.

The injectable solutions or microemulsions may be introduced into a patient's blood stream by local bolus injection. Alternatively, it may be advantageous to administer the solution or microemulsion in such a way as to maintain a constant circulating concentration of the instant compound. In order to maintain such a constant concentration, a continuous intravenous delivery device may be utilized. An example of such a device is the Deltec CADD-PLUS™ model 5400 intravenous pump.

The pharmaceutical compositions may be in the form of a sterile injectable aqueous or oleagenous suspension for intramuscular and subcutaneous administration. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, for example as a solution in 1,3-butane diol. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Compounds of Formula I may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials include cocoa butter, glycerinated gelatin, hydrogenated vegetable oils, mixtures of polyethylene glycols of various molecular weights and fatty acid esters of polyethylene glycol.

For topical use, creams, ointments, jellies, solutions or suspensions, etc., containing the compound of Formula I are employed. (For purposes of this application, topical application shall include mouth washes and gargles.)

The compounds for the present invention can be administered in intranasal form via topical use of suitable intranasal vehicles and delivery devices, or via transdermal routes, using those forms of transdermal skin patches well known to those of ordinary skill in the art. To be administered in the form of a transdermal delivery system, the dosage administration will, of course, be continuous rather than intermittent throughout the dosage regimen. Compounds of the present invention may also be delivered as a suppository employing bases such as cocoa butter, glycerinated gelatin, hydrogenated vegetable oils, mixtures of polyethylene glycols of various molecular weights and fatty acid esters of polyethylene glycol.

The dosage regimen utilizing the compounds of the instant invention can be selected in accordance with a variety of factors including type, species, age, weight, sex and the type of cancer being treated; the severity (i.e., stage) of the cancer to be treated; the route of administration; the renal and hepatic function of the patient; and the particular compound or salt thereof employed. An ordinarily skilled physician or veterinarian can readily determine and prescribe the effective amount of the drug required to treat, for example, to prevent, inhibit (fully or partially) or arrest the progress of the disease.

In one exemplary application, a suitable amount of compound is administered to a mammal undergoing treatment for cancer. Administration occurs in an amount between about 0.1 mg/kg of body weight to about 60 mg/kg of body weight per day, preferably of between 0.5 mg/kg of body weight to about 40 mg/kg of body weight per day.

In a further example, compounds of the instant invention can be administered in a total daily dose of up to 1000 mg. Compounds of the instant invention can be administered once daily (QD), or divided into multiple daily doses such as twice daily (BID), and three times daily (TID). Compounds of the instant invention can be administered at a total daily dosage of up to 1000 mg, e.g., 200 mg, 300 mg, 400 mg, 600 mg, 800 mg or 1000 mg, which can be administered in one daily dose or can be divided into multiple daily doses as described above.

In addition, the administration can be continuous, i.e., every day, or intermittently. The terms “intermittent” or “intermittently” as used herein means stopping and starting at either regular or irregular intervals. For example, intermittent administration of a compound of the instant invention may be administration one to six days per week or it may mean administration in cycles (e.g., daily administration for two to eight consecutive weeks, then a rest period with no administration for up to one week) or it may mean administration on alternate days.

In addition, the compounds of the instant invention may be administered according to any of the schedules described above, consecutively for a few weeks, followed by a rest period. For example, the compounds of the instant invention may be administered according to any one of the schedules described above from two to eight weeks, followed by a rest period of one week, or twice daily at a dose of 100-500 mg for three to five days a week. In another particular embodiment, the compounds of the instant invention may be administered three times daily for two consecutive weeks, followed by one week of rest.

The instant compounds are also useful in combination with known therapeutic agents and anti-cancer agents. For example, instant compounds are useful in combination with known anti-cancer agents. Combinations of the presently disclosed compounds with other anti-cancer or chemotherapeutic agents are within the scope of the invention. Examples of such agents can be found in Cancer Principles and Practice of Oncology by V. T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the cancer involved. Such anti-cancer agents include, but are not limited to, the following: estrogen receptor modulators, androgen receptor modulators, retinoid receptor modulators, cytotoxic/cytostatic agents, antiproliferative agents, prenyl-protein transferase inhibitors, HMG-CoA reductase inhibitors and other angiogenesis inhibitors, inhibitors of cell proliferation and survival signaling, apoptosis inducing agents and agents that interfere with cell cycle checkpoints. The instant compounds are particularly useful when co-administered with radiation therapy.

In an embodiment, the instant compounds are also useful in combination with known anti-cancer agents including the following: estrogen receptor modulators, androgen receptor modulators, retinoid receptor modulators, cytotoxic agents, antiproliferative agents, prenyl-protein transferase inhibitors, HMG-CoA reductase inhibitors, HIV protease inhibitors, reverse transcriptase inhibitors, and other angiogenesis inhibitors.

“Estrogen receptor modulators” refers to compounds that interfere with or inhibit the binding of estrogen to the receptor, regardless of mechanism. Examples of estrogen receptor modulators include, but are not limited to, tamoxifen, raloxifene, idoxifene, LY353381, LY117081, toremifene, fulvestrant, 4-[7-(2,2-dimethyl-1-oxopropoxy-4-methyl-2-[4-[2-(1-piperidinyl)ethoxy]phenyl]-2H-1-benzopyran-3-yl]-phenyl-2,2-dimethylpropanoate, 4,4′-dihydroxybenzophenone-2,4-dinitrophenyl-hydrazone, and SH646.

“Androgen receptor modulators” refers to compounds which interfere or inhibit the binding of androgens to the receptor, regardless of mechanism. Examples of androgen receptor modulators include finasteride and other 5α-reductase inhibitors, nilutamide, flutamide, bicalutamide, liarozole, and abiraterone acetate.

“Retinoid receptor modulators” refers to compounds which interfere or inhibit the binding of retinoids to the receptor, regardless of mechanism. Examples of such retinoid receptor modulators include bexarotene, tretinoin, 13-cis-retinoic acid, 9-cis-retinoic acid, α-difluoromethylornithine, ILX23-7553, trans-N-(4′-hydroxyphenyl) retinamide, and N-4-carboxyphenyl retinamide.

“Cytotoxic/cytostatic agents” refer to compounds which cause cell death or inhibit cell proliferation primarily by interfering directly with the cell's functioning or inhibit or interfere with cell mytosis, including alkylating agents, tumor necrosis factors, intercalators, hypoxia activatable compounds, microtubule inhibitors/microtubule-stabilizing agents, inhibitors of mitotic kinesins, inhibitors of histone deacetylase, inhibitors of kinases involved in mitotic progression, antimetabolites; biological response modifiers; hormonal/anti-hormonal therapeutic agents, haematopoietic growth factors, monoclonal antibody targeted therapeutic agents, topoisomerase inhibitors, proteasome inhibitors and ubiquitin ligase inhibitors.

Examples of cytotoxic agents include, but are not limited to, sertenef, cachectin, ifosfamide, tasonermin, lonidamine, carboplatin, altretamine, prednimustine, dibromodulcitol, ranimustine, fotemustine, nedaplatin, oxaliplatin, temozolomide, heptaplatin, estramustine, improsulfan tosilate, trofosfamide, nimustine, dibrospidium chloride, pumitepa, lobaplatin, satraplatin, profiromycin, cisplatin, irofulven, dexifosfamide, cis-aminedichloro(2-methyl-pyridine)platinum, benzylguanine, glufosfamide, GPX100, (trans, trans, trans)-bis-mu-(hexane-1,6-diamine)-mu-[diamine-platinum(II)]bis[diamine(chloro)platinum (II)]tetrachloride, diarizidinylspermine, arsenic trioxide, 1-(11-dodecylamino-10-hydroxyundecyl)-3,7-dimethylxanthine, zorubicin, idarubicin, daunorubicin, bisantrene, mitoxantrone, pirarubicin, pinafide, valrubicin, amrubicin, antineoplaston, 3′-deamino-3′-morpholino-13-deoxo-10-hydroxycaminomycin, annamycin, galarubicin, elinafide, MEN10755, and 4-demethoxy-3-deamino-3-aziridinyl-4-methylsulphonyl-daunorubicin (see WO 00/50032).

An example of a hypoxia activatable compound is tirapazamine.

Examples of proteasome inhibitors include but are not limited to lactacystin and bortezomib.

Examples of microtubule inhibitors/microtubule-stabilising agents include paclitaxel, vindesine sulfate, 3′,4′-didehydro-4′-deoxy-8′-norvincaleukoblastine, docetaxol, rhizoxin, dolastatin, mivobulin isethionate, auristatin, cemadotin, RPR109881, BMS184476, vinflunine, cryptophycin, 2,3,4,5,6-pentafluoro-N-(3-fluoro-4-methoxyphenyl)benzene sulfonamide, anhydrovinblastine, N,N-dimethyl-L-valyl-L-valyl-N-methyl-L-valyl-L-prolyl-L-proline-t-butylamide, TDX258, the epothilones (see for example U.S. Pat. Nos. 6,284,781 and 6,288,237) and BMS188797.

Some examples of topoisomerase inhibitors are topotecan, hycaptamine, irinotecan, rubitecan, 6-ethoxypropionyl-3′,4′-O-exo-benzylidene-chartreusin, 9-methoxy-N,N-dimethyl-5-nitropyrazolo[3,4,5-kl]acridine-2-(6H) propanamine, 1-amino-9-ethyl-5-fluoro-2,3-dihydro-9-hydroxy-4-methyl-1H,12H-benzo[de]pyrano[3′,4′:b,7]-indolizino[1,2b]quinoline-10,13 (9H, 15H)dione, lurtotecan, 7-[2-(N-isopropylamino)ethyl]-(20S)camptothecin, BNP1350, BNPI100, BN80915, BN80942, etoposide phosphate, teniposide, sobuzoxane, 2′-dimethylamino-2′-deoxy-etoposide, GL331, N-[2-(dimethylamino)ethyl]-9-hydroxy-5,6-dimethyl-6H-pyrido[4,3-b]carbazole-1-carboxamide, asulacrine, (5a,5aB,8aa,9b)-9-[2-[N-[2-(dimethylamino)ethyl]-N-methylamino]ethyl]-5-[4-hydro0xy-3,5-dimethoxyphenyl]-5,5a,6,8,8a,9-hexohydrofuro(3′,4′:6,7)naphtho(2,3-d)-1,3-dioxol-6-one, 2,3-(methylenedioxy)-5-methyl-7-hydroxy-8-methoxybenzo[c]-phenanthridinium, 6,9-bis[(2-aminoethyl)amino]benzo[g]isoquinoline-5,10-dione, 5-(3-aminopropylamino)-7,10-dihydroxy-2-(2-hydroxyethylaminomethyl)-6H-pyrazolo[4,5,1-de]acridin-6-one, N-[1-[2(diethylamino)ethylamino]-7-methoxy-9-oxo-9H-thioxanthen-4-ylmethyl]formamide, N-(2-(dimethylamino)ethyl)acridine-4-carboxamide, 6-[[2-(dimethylamino)ethyl]amino]-3-hydroxy-7H-indeno[2,1-c]quinolin-7-one, and dimesna.

Examples of inhibitors of mitotic kinesins, and in particular the human mitotic kinesin KSP, are described in PCT Publications WO 01/30768, WO 01/98278, WO 03/050,064, WO 03/050,122, WO 03/049,527, WO 03/049,679, WO 03/049,678, WO04/039774, WO03/079973, WO03/099211, WO03/105855, WO03/106417, WO04/037171, WO04/058148, WO04/058700, WO04/126699, WO05/018638, WO05/019206, WO05/019205, WO05/018547, WO05/017190, US2005/0176776. In an embodiment inhibitors of mitotic kinesins include, but are not limited to inhibitors of KSP, inhibitors of MKLP1, inhibitors of CENP-E, inhibitors of MCAK, inhibitors of Kifl4, inhibitors of Mphosph1 and inhibitors of Rab6-KIFL.

Examples of “histone deacetylase inhibitors” include, but are not limited to, SAHA, TSA, oxamflatin, PXD101, MG98, valproic acid and scriptaid. Further reference to other histone deacetylase inhibitors may be found in the following manuscript; Miller, T. A. et al. J. Med. Chem. 46(24):5097-5116 (2003).

“Inhibitors of kinases involved in mitotic progression” include, but are not limited to, inhibitors of aurora kinase, inhibitors of Polo-like kinases (PLK) (in particular inhibitors of PLK-1), inhibitors of bub-1 and inhibitors of bub-R1.

“Antiproliferative agents” includes antisense RNA and DNA oligonucleotides such as G3139, ODN698, RVASKRAS, GEM231, and INX3001, and antimetabolites such as enocitabine, carmofur, tegafur, pentostatin, doxifluridine, trimetrexate, fludarabine, capecitabine, galocitabine, cytarabine ocfosfate, fosteabine sodium hydrate, raltitrexed, paltitrexid, emitefur, tiazofurin, decitabine, nolatrexed, pemetrexed, nelzarabine, 2′-deoxy-2′-methylidenecytidine, 2′-fluoromethylene-2′-deoxycytidine, N-[5-(2,3-dihydro-benzofuryl)sulfonyl]-N′-(3,4-dichlorophenyl)urea, N6-[4-deoxy-4-[N2-[2(E),4(E)-tetradecadienoyl]glycylamino]-L-glycero-B-L-manno-heptopyranosyl]adenine, aplidine, ecteinascidin, troxacitabine, 4-[2-amino-4-oxo-4,6,7,8-tetrahydro-3H-pyrimidino[5,4-b][1,4]thiazin-6-yl-(S)-ethyl]-2,5-thienoyl-L-glutamic acid, aminopterin, 5-fluorouracil, alanosine, 11-acetyl-8-(carbamoyloxymethyl)-4-formyl-6-methoxy-14-oxa-1,11-diazatetracyclo(7.4.1.0.0)-tetradeca-2,4,6-trien-9-yl acetic acid ester, swainsonine, lometrexol, dexrazoxane, methioninase, 2′-cyano-2′-deoxy-N4-palmitoyl-1-B-D-arabino furanosyl cytosine and 3-aminopyridine-2-carboxaldehyde thiosemicarbazone.

Examples of monoclonal antibody targeted therapeutic agents include those therapeutic agents which have cytotoxic agents or radioisotopes attached to a cancer cell specific or target cell specific monoclonal antibody. Examples include Bexxar.

“HMG-CoA reductase inhibitors” refers to inhibitors of 3-hydroxy-3-methylglutaryl-CoA reductase. Examples of HMG-CoA reductase inhibitors that may be used include but are not limited to lovastatin (MEVACOR®; see U.S. Pat. Nos. 4,231,938, 4,294,926 and 4,319,039), simvastatin (ZOCOR®; see U.S. Pat. Nos. 4,444,784, 4,820,850 and 4,916,239), pravastatin (PRAVACHOL®; see U.S. Pat. Nos. 4,346,227, 4,537,859, 4,410,629, 5,030,447 and 5,180,589), fluvastatin (LESCOL®; see U.S. Pat. Nos. 5,354,772, 4,911,165, 4,929,437, 5,189,164, 5,118,853, 5,290,946 and 5,356,896) and atorvastatin (LIPITOR®; see U.S. Pat. Nos. 5,273,995, 4,681,893, 5,489,691 and 5,342,952). The structural formulas of these and additional HMG-CoA reductase inhibitors that may be used in the instant methods are described at page 87 of M. Yalpani, “Cholesterol Lowering Drugs”, Chemistry & Industry, pp. 85-89 (5 Feb. 1996) and U.S. Pat. Nos. 4,782,084 and 4,885,314. The term HMG-CoA reductase inhibitor as used herein includes all pharmaceutically acceptable lactone and open-acid forms (i.e., where the lactone ring is opened to form the free acid) as well as salt and ester forms of compounds which have HMG-CoA reductase inhibitory activity, and therefor the use of such salts, esters, open-acid and lactone forms is included within the scope of this invention.

“Prenyl-protein transferase inhibitor” refers to a compound which inhibits any one or any combination of the prenyl-protein transferase enzymes, including farnesyl-protein transferase (FPTase), geranylgeranyl-protein transferase type I (GGPTase-I), and geranylgeranyl-protein transferase type-II (GGPTase-II, also called Rab GGPTase).

Examples of prenyl-protein transferase inhibitors can be found in the following publications and patents: WO 96/30343, WO 97/18813, WO 97/21701, WO 97/23478, WO 97/38665, WO 98/28980, WO 98/29119, WO 95/32987, U.S. Pat. No. 5,420,245, U.S. Pat. No. 5,523,430, U.S. Pat. No. 5,532,359, U.S. Pat. No. 5,510,510, U.S. Pat. No. 5,589,485, U.S. Pat. No. 5,602,098, European Patent Publ. 0 618 221, European Patent Publ. 0 675 112, European Patent Publ. 0 604 181, European Patent Publ. 0 696 593, WO 94/19357, WO 95/08542, WO 95/11917, WO 95/12612, WO 95/12572, WO 95/10514, U.S. Pat. No. 5,661,152, WO 95/10515, WO 95/10516, WO 95/24612, WO 95/34535, WO 95/25086, WO 96/05529, WO 96/06138, WO 96/06193, WO 96/16443, WO 96/21701, WO 96/21456, WO 96/22278, WO 96/24611, WO 96/24612, WO 96/05168, WO 96/05169, WO 96/00736, U.S. Pat. No. 5,571,792, WO 96/17861, WO 96/33159, WO 96/34850, WO 96/34851, WO 96/30017, WO 96/30018, WO 96/30362, WO 96/30363, WO 96/3 1111, WO 96/31477, WO 96/31478, WO 96/31501, WO 97/00252, WO 97/03047, WO 97/03050, WO 97/04785, WO 97/02920, WO 97/17070, WO 97/23478, WO 97/26246, WO 97/30053, WO 97/44350, WO 98/02436, and U.S. Pat. No. 5,532,359. For an example of the role of a prenyl-protein transferase inhibitor on angiogenesis see European J of Cancer, Vol. 35, No. 9, pp. 1394-1401 (1999).

“Angiogenesis inhibitors” refers to compounds that inhibit the formation of new blood vessels, regardless of mechanism. Examples of angiogenesis inhibitors include, but are not limited to, tyrosine kinase inhibitors, such as inhibitors of the tyrosine kinase receptors Flt-1 (VEGFR1) and Flk-1/KDR (VEGFR2), inhibitors of epidermal-derived, fibroblast-derived, or platelet derived growth factors, MMP (matrix metalloprotease) inhibitors, integrin blockers, interferon-α, interleukin-12, pentosan polysulfate, cyclooxygenase inhibitors, including nonsteroidal anti-inflammatories (NSAIDs) like aspirin and ibuprofen as well as selective cyclooxy-genase-2 inhibitors like celecoxib and rofecoxib (PNAS, Vol. 89, p. 7384 (1992); JNCI, Vol. 69, p. 475 (1982); Arch. Opthalmol., Vol. 108, p. 573 (1990); Anat. Rec., Vol. 238, p. 68 (1994); FEBS Letters, Vol. 372, p. 83 (1995); Clin, Orthop. Vol. 313, p. 76 (1995); J. Mol. Endocrinol., Vol. 16, p. 107 (1996); Jpn. J. Pharmacol., Vol. 75, p. 105 (1997); Cancer Res., Vol. 57, p. 1625 (1997); Cell, Vol. 93, p. 705 (1998); Intl. J. Mol. Med., Vol. 2, p. 715 (1998); J. Biol. Chem., Vol. 274, p. 9116 (1999)), steroidal anti-inflammatories (such as corticosteroids, mineralocorticoids, dexamethasone, prednisone, prednisolone, methylpred, betamethasone), carboxyamidotriazole, combretastatin A-4, squalamine, 6-O-chloroacetyl-carbonyl)-fumagillol, thalidomide, angiostatin, troponin-1, angiotensin 11 antagonists (see Fernandez et al., J. Lab. Clin. Med. 105:141-145 (1985)), and antibodies to VEGF (see, Nature Biotechnology, Vol. 17, pp. 963-968 (October 1999); Kim et al., Nature, 362, 841-844 (1993); WO 00/44777; and WO 00/61186).

Other therapeutic agents that modulate or inhibit angiogenesis and may also be used in combination with the compounds of the instant invention include agents that modulate or inhibit the coagulation and fibrinolysis systems (see review in Clin. Chem. La. Med. 38:679-692 (2000)). Examples of such agents that modulate or inhibit the coagulation and fibrinolysis pathways include, but are not limited to, heparin (see Thromb. Haemost. 80:10-23 (1998)), low molecular weight heparins and carboxypeptidase U inhibitors (also known as inhibitors of active thrombin activatable fibrinolysis inhibitor [TAFIa]) (see Thrombosis Res. 101:329-354 (2001)). TAFIa inhibitors have been described in PCT Publication WO 03/013,526 and U.S. Ser. No. 60/349,925 (filed Jan. 18, 2002).

“Agents that interfere with cell cycle checkpoints” refer to compounds that inhibit protein kinases that transduce cell cycle checkpoint signals, thereby sensitizing the cancer cell to DNA damaging agents. Such agents include inhibitors of ATR, ATM, the Chk1 and Chk2 kinases and cdk and cdc kinase inhibitors and are specifically exemplified by 7-hydroxystaurosporin, flavopiridol, CYC202 (Cyclacel) and BMS-387032.

“Agents that interfere with receptor tyrosine kinases (RTKs)” refer to compounds that inhibit RTKs and therefore mechanisms involved in oncogenesis and tumor progression. Such agents include inhibitors of c-Kit, Eph, PDGF, Flt3 and c-Met. Further agents include inhibitors of RTKs as described by Blume-Jensen and Hunter, Nature, 411:355-365, 2001.

“Inhibitors of cell proliferation and survival signaling pathway” refer to pharmaceutical agents that inhibit cell surface receptors and signal transduction cascades downstream of those surface receptors. Such agents include inhibitors of inhibitors of EGFR (for example gefitinib and erlotinib), inhibitors of ERB-2 (for example trastuzumab), inhibitors of IGFR, inhibitors of cytokine receptors, inhibitors of MET, inhibitors of PI3K (for example LY294002), serine/threonine kinases (including but not limited to inhibitors of Akt such as MK-2206 and those described in WO 02/083064, WO 02/083139, WO 02/083140, US 2004-0116432, WO 02/083138, US 2004-0102360, WO 03/086404, WO 03/086279, WO 03/086394, WO 03/084473, WO 03/086403, WO 2004/041162, WO 2004/096131, WO 2004/096129, WO 2004/096135, WO 2004/096130, WO 2005/100356, WO 2005/100344), inhibitors of Raf kinase (for example BAY-43-9006), inhibitors of MEK (for example CI-1040, AZD6244 and PD-098059) and inhibitors of mTOR (for example Ridaforolimus). Such agents include small molecule inhibitor compounds and antibody antagonists.

“Apoptosis inducing agents” include activators of TNF receptor family members (including the TRAIL receptors).

The invention also encompasses combinations with NSAID's which are selective COX-2 inhibitors. For purposes of this specification NSAID's which are selective inhibitors of COX-2 are defined as those which possess a specificity for inhibiting COX-2 over COX-1 of at least 100 fold as measured by the ratio of IC₅₀ for COX-2 over IC₅₀ for COX-1 evaluated by cell or microsomal assays. Such compounds include, but are not limited to those disclosed in U.S. Pat. No. 5,474,995, U.S. Pat. No. 5,861,419, U.S. Pat. No. 6,001,843, U.S. Pat. No. 6,020,343, U.S. Pat. No. 5,409,944, U.S. Pat. No. 5,436,265, U.S. Pat. No. 5,536,752, U.S. Pat. No. 5,550,142, U.S. Pat. No. 5,604,260, U.S. Pat. No. 5,698,584, U.S. Pat. No. 5,710,140, WO 94/15932, U.S. Pat. No. 5,344,991, U.S. Pat. No. 5,134,142, U.S. Pat. No. 5,380,738, U.S. Pat. No. 5,393,790, U.S. Pat. No. 5,466,823, U.S. Pat. No. 5,633,272, and U.S. Pat. No. 5,932,598, all of which are hereby incorporated by reference.

Inhibitors of COX-2 that are particularly useful in the instant method of treatment are: 3-phenyl-4-(4-(methylsulfonyl)phenyl)-2-(5H)-furanone; and 5-chloro-3-(4-methylsulfonyl)-phenyl-2-(2-methyl-5-pyridinyl)pyridine; or a pharmaceutically acceptable salt thereof.

Compounds that have been described as specific inhibitors of COX-2 and are therefore useful in the present invention include, but are not limited to: parecoxib, CELEBREX® and BEXTRA® or a pharmaceutically acceptable salt thereof.

Other examples of angiogenesis inhibitors include, but are not limited to, endostatin, ukrain, ranpirnase, IM862, 5-methoxy-4-[2-methyl-3-(3-methyl-2-butenyl)oxiranyl]-1-oxaspiro[2,5]oct-6-yl(chloroacetyl)carbamate, acetyldinanaline, 5-amino-1-[[3,5-dichloro-4-(4-chlorobenzoyl)-phenyl]methyl]-1H-1,2,3-triazole-4-carboxamide, CM101, squalamine, combretastatin, RPI4610, NX31838, sulfated mannopentaose phosphate, 7,7-(carbonyl-bis[imino-N-methyl-4,2-pyrrolocarbonylimino[N-methyl-4,2-pyrrole]-carbonylimino]-bis-(1,3-naphthalene disulfonate), and 3-[(2,4-dimethylpyrrol-5-yl)methylene]-2-indolinone (SU5416).

As used above, “integrin blockers” refers to compounds which selectively antagonize, inhibit or counteract binding of a physiological ligand to the αᵥβ₃ integrin, to compounds which selectively antagonize, inhibit or counteract binding of a physiological ligand to the αᵥβ5 integrin, to compounds which antagonize, inhibit or counteract binding of a physiological ligand to both the αᵥβ₃ integrin and the αᵥβ₃ integrin, and to compounds which antagonize, inhibit or counteract the activity of the particular integrin(s) expressed on capillary endothelial cells. The term also refers to antagonists of the αᵥβ₆, αᵥβ₈, α₁β₁, α₅β₁, α₆β₁ and α₆β₄ integrins. The term also refers to antagonists of any combination of αᵥβ₃, αᵥβ₅, αᵥβ₆, αᵥβ₈, α₁β₁, α₂β₁, α₅β₁, α₆β₁ and α₆β₄ integrins.

Some specific examples of tyrosine kinase inhibitors include N-(trifluoromethylphenyl)-5-methylisoxazol-4-carboxamide, 3-[(2,4-dimethylpyrrol-5-yl)methylidenyl)indolin-2-one, 17-(allylamino)-17-demethoxygeldanamycin, 4-(3-chloro-4-fluorophenylamino)-7-methoxy-6-[3-(4-morpholinyl)propoxyl]quinazoline, N-(3-ethynylphenyl)-6,7-bis(2-methoxyethoxy)-4-quinazolinamine, BIBX1382, 2,3,9,10,11,12-hexahydro-10-(hydroxymethyl)-10-hydroxy-9-methyl-9,12-epoxy-1H-diindolo[1,2,3-fg:3′,2′,1′-kl]pyrrolo[3,4-i][1,6]benzodiazocin-1-one, SH268, genistein, imatinib (STI571), CEP2563, 4-(3-chlorophenylamino)-5,6-dimethyl-7H-pyrrolo[2,3-d]pyrimidinemethane sulfonate, 4-(3-bromo-4-hydroxyphenyl)amino-6,7-dimethoxyquinazoline, 4-(4′-hydroxyphenyl)amino-6,7-dimethoxyquinazoline, SU6668, STI571A, N-4-chlorophenyl-4-(4-pyridylmethyl)-1-phthalazinamine, and EMD121974.

Combinations with compounds other than anti-cancer compounds are also encompassed in the instant methods. For example, combinations of the instantly claimed compounds with PPAR-γ (i.e., PPAR-gamma) agonists and PPAR-δ (i.e., PPAR-delta) agonists are useful in the treatment of certain malignancies. PPAR-γ and PPAR-δ are the nuclear peroxisome proliferator-activated receptors γ and δ. The expression of PPAR-γ on endothelial cells and its involvement in angiogenesis has been reported in the literature (see J. Cardiovasc. Pharmacol. 1998; 31:909-913; J. Biol. Chem. 1999; 274:9116-9121; Invest. Ophthalmol. Vis. Sci. 2000; 41:2309-2317). More recently, PPAR-γ agonists have been shown to inhibit the angiogenic response to VEGF in vitro; both troglitazone and rosiglitazone maleate inhibit the development of retinal neovascularization in mice. (Arch. Ophthamol 2001; 119:709-717). Examples of PPAR-γ agonists and PPAR-γ/α agonists include, but are not limited to, thiazolidinediones (such as DRF2725, CS-011, troglitazone, rosiglitazone, and pioglitazone), fenofibrate, gemfibrozil, clofibrate, GW2570, SB219994, AR-H039242, JTT-501, MCC-555, GW2331, GW409544, NN2344, KRP297, NP0110, DRF4158, NN622, GI262570, PNU182716, DRF552926, 2-[(5,7-dipropyl-3-trifluoromethyl-1,2-benzisoxazol-6-yl)oxy]-2-methylpropionic acid (disclosed in U.S. Ser. No. 09/782,856), and 2(R)-7-(3-(2-chloro-4-(4-fluorophenoxy)phenoxy)propoxy)-2-ethylchromane-2-carboxylic acid (disclosed in U.S. Ser. No. 60/235,708 and 60/244,697).

Another embodiment of the instant invention is the use of the presently disclosed compounds in combination with gene therapy for the treatment of cancer. For an overview of genetic strategies to treating cancer see Hall et al (Am J Hum Genet. 61:785-789, 1997) and Kufe et al (Cancer Medicine, 5th Ed, pp 876-889, BC Decker, Hamilton 2000). Gene therapy can be used to deliver any tumor suppressing gene. Examples of such genes include, but are not limited to, p53, which can be delivered via recombinant virus-mediated gene transfer (see U.S. Pat. No. 6,069,134, for example), a uPA/uPAR antagonist (“Adenovirus-Mediated Delivery of a uPA/uPAR Antagonist Suppresses Angiogenesis-Dependent Tumor Growth and Dissemination in Mice,” Gene Therapy, August 1998; 5(8):1105-13), and interferon gamma (J Immunol 2000; 164:217-222).

The compounds of the instant invention may also be administered in combination with an inhibitor of inherent multidrug resistance (MDR), in particular MDR associated with high levels of expression of transporter proteins. Such MDR inhibitors include inhibitors of p-glycoprotein (P-gp), such as LY335979, XR9576, OC144-093, R101922, VX853 and PSC833 (valspodar).

A compound of the present invention may be employed in conjunction with anti-emetic agents to treat nausea or emesis, including acute, delayed, late-phase, and anticipatory emesis, which may result from the use of a compound of the present invention, alone or with radiation therapy. For the prevention or treatment of emesis, a compound of the present invention may be used in conjunction with other anti-emetic agents, especially neurokinin-1 receptor antagonists, 5HT3 receptor antagonists, such as ondansetron, granisetron, tropisetron, and zatisetron, GABAB receptor agonists, such as baclofen, a corticosteroid such as Decadron (dexamethasone), Kenalog, Aristocort, Nasalide, Preferid, Benecorten or others such as disclosed in U.S. Pat. Nos. 2,789,118, 2,990,401, 3,048,581, 3,126,375, 3,929,768, 3,996,359, 3,928,326 and 3,749,712, an antidopaminergic, such as the phenothiazines (for example prochlorperazine, fluphenazine, thioridazine and mesoridazine), metoclopramide or dronabinol. In an embodiment, an anti-emesis agent selected from a neurokinin-1 receptor antagonist, a 5HT3 receptor antagonist and a corticosteroid is administered as an adjuvant for the treatment or prevention of emesis that may result upon administration of the instant compounds.

Neurokinin-1 receptor antagonists of use in conjunction with the compounds of the present invention are fully described, for example, in U.S. Pat. Nos. 5,162,339, 5,232,929, 5,242,930, 5,373,003, 5,387,595, 5,459,270, 5,494,926, 5,496,833, 5,637,699, 5,719,147; European Patent Publication Nos. EP 0 360 390, 0 394 989, 0 428 434, 0 429 366, 0 430 771, 0 436 334, 0 443 132, 0 482 539, 0 498 069, 0 499 313, 0 512 901, 0 512 902, 0 514 273, 0 514 274, 0 514 275, 0 514 276, 0 515 681, 0 517 589, 0 520 555, 0 522 808, 0 528 495, 0 532 456, 0 533 280, 0 536 817, 0 545 478, 0 558 156, 0 577 394, 0 585 913, 0 590 152, 0 599 538, 0 610 793, 0 634 402, 0 686 629, 0 693 489, 0 694 535, 0 699 655, 0 699 674, 0 707 006, 0 708 101, 0 709 375, 0 709 376, 0 714 891, 0 723 959, 0 733 632 and 0 776 893; PCT International Patent Publication Nos. WO 90/05525, 90/05729, 91/09844, 91/18899, 92/01688, 92/06079, 92/12151, 92/15585, 92/17449, 92/20661, 92/20676, 92/21677, 92/22569, 93/00330, 93/00331, 93/01159, 93/01165, 93/01169, 93/01170, 93/06099, 93/09116, 93/10073, 93/14084, 93/14113, 93/18023, 93/19064, 93/21155, 93/21181, 93/23380, 93/24465, 94/00440, 94/01402, 94/02461, 94/02595, 94/03429, 94/03445, 94/04494, 94/04496, 94/05625, 94/07843, 94/08997, 94/10165, 94/10167, 94/10168, 94/10170, 94/11368, 94/13639, 94/13663, 94/14767, 94/15903, 94/19320, 94/19323, 94/20500, 94/26735, 94/26740, 94/29309, 95/02595, 95/04040, 95/04042, 95/06645, 95/07886, 95/07908, 95/08549, 95/11880, 95/14017, 95/15311, 95/16679, 95/17382, 95/18124, 95/18129, 95/19344, 95/20575, 95/21819, 95/22525, 95/23798, 95/26338, 95/28418, 95/30674, 95/30687, 95/33744, 96/05181, 96/05193, 96/05203, 96/06094, 96/07649, 96/10562, 96/16939, 96/18643, 96/20197, 96/21661, 96/29304, 96/29317, 96/29326, 96/29328, 96/31214, 96/32385, 96/37489, 97/01553, 97/01554, 97/03066, 97/08144, 97/14671, 97/17362, 97/18206, 97/19084, 97/19942 and 97/21702; and in British Patent Publication Nos. 2 266 529, 2 268 931, 2 269 170, 2 269 590, 2 271 774, 2 292 144, 2 293 168, 2 293 169, and 2 302 689. The preparation of such compounds is fully described in the aforementioned patents and publications, which are incorporated herein by reference.

In an embodiment, the neurokinin-1 receptor antagonist for use in conjunction with the compounds of the present invention is selected from: 2-(R)-(1-(R)-(3,5-bis(trifluoromethyl)-phenyl)ethoxy)-3-(S)-(4-fluorophenyl)-4-(3-(5-oxo-1H,4H-1,2,4-triazolo)methyl)morpholine, or a pharmaceutically acceptable salt thereof, which is described in U.S. Pat. No. 5,719,147.

A compound of the instant invention may also be useful for treating or preventing cancer, including bone cancer, in combination with bisphosphonates (understood to include bisphosphonates, diphosphonates, bisphosphonic acids and diphosphonic acids). Examples of bisphosphonates include but are not limited to: etidronate (Didronel), pamidronate (Aredia), alendronate (Fosamax), risedronate (Actonel), zoledronate (Zometa), ibandronate (Boniva), incadronate or cimadronate, clodronate, EB-1053, minodronate, neridronate, piridronate and tiludronate including any and all pharmaceutically acceptable salts, derivatives, hydrates and mixtures thereof.

A compound of the instant invention may also be administered with an agent useful in the treatment of anemia. Such an anemia treatment agent is, for example, a continuous eythropoiesis receptor activator (such as epoetin alfa).

A compound of the instant invention may also be administered with an agent useful in the treatment of neutropenia. Such a neutropenia treatment agent is, for example, a hematopoietic growth factor which regulates the production and function of neutrophils such as a human granulocyte colony stimulating factor, (G-CSF). Examples of a G-CSF include filgrastim.

A compound of the instant invention may also be administered with an immunologic-enhancing drug, such as levamisole, isoprinosine and Zadaxin.

A compound of the instant invention may also be useful for treating or preventing cancer, including bone cancer, in combination with bisphosphonates (understood to include bisphosphonates, diphosphonates, bisphosphonic acids and diphosphonic acids). Examples of bisphosphonates include but are not limited to: etidronate (Didronel), pamidronate (Aredia), alendronate (Fosamax), risedronate (Actonel), zoledronate (Zometa), ibandronate (Boniva), incadronate or cimadronate, clodronate, EB-1053, minodronate, neridronate, piridronate and tiludronate including any and all pharmaceutically acceptable salts, derivatives, hydrates and mixtures thereof.

A compound of the instant invention may also be useful for treating or preventing breast cancer in combination with aromatase inhibitors. Examples of aromatase inhibitors include but are not limited to: anastrozole, letrozole and exemestane.

A compound of the instant invention may also be useful for treating or preventing cancer in combination with siRNA therapeutics.

The compounds of the instant invention may also be administered in combination with γ-secretase inhibitors and/or inhibitors of NOTCH signaling. Such inhibitors include compounds described in WO 01/90084, WO 02/30912, WO 01/70677, WO 03/013506, WO 02/36555, WO 03/093252, WO 03/093264, WO 03/093251, WO 03/093253, WO 2004/039800, WO 2004/039370, WO 2005/030731, WO 2005/014553, U.S. Ser. No. 10/957,251, WO 2004/089911, WO 02/081435, WO 02/081433, WO 03/018543, WO 2004/031137, WO 2004/031139, WO 2004/031138, WO 2004/101538, WO 2004/101539 and WO 02/47671 (including LY-450139).

A compound of the instant invention may also be useful for treating or preventing cancer in combination with PARP inhibitors.

A compound of the instant invention may also be useful for treating cancer in combination with the following therapeutic agents: abarelix (Plenaxis Depot®); aldesleukin (Prokine®); Aldesleukin (Proleukin®); Alemtuzumabb (Campath®); alitretinoin (Panretin®); allopurinol (Zyloprim®); altretamine (Hexylen®); amifostine (Ethyol®); anastrozole (Arimidex®); arsenic trioxide (Trisenox®); asparaginase (Elspar®); azacitidine (Vidaza®); bevacuzimab (Avastin®); bexarotene capsules (Targretin®); bexarotene gel (Targretin®); bleomycin (Blenoxane®); bortezomib (Velcade®); busulfan intravenous (Busulfex®); busulfan oral (Myleran®); calusterone (Methosarb®); capecitabine (Xeloda®); carboplatin (Paraplatin®); carmustine (BCNU®, BiCNU®); carmustine (Gliadel®); carmustine with Polifeprosan 20 Implant (Gliadel Wafer®); celecoxib (Celebrex®); cetuximab (Erbitux®); chlorambucil (Leukeran®); cisplatin (Platinol®); cladribine (Leustatin®, 2-CdA®); clofarabine (Clolar®); cyclophosphamide (Cytoxan®, Neosar®); cyclophosphamide (Cytoxan Injection®); cyclophosphamide (Cytoxan Tablet®); cytarabine (Cytosar-U®); cytarabine liposomal (DepoCyt®); dacarbazine (DTIC-Dome®); dactinomycin, actinomycin D (Cosmegen®); Darbepoetin alfa (Aranesp®); daunorubicin liposomal (DanuoXome®); daunorubicin, daunomycin (Daunorubicin®); daunorubicin, daunomycin (Cerubidine®); Denileukin diftitox (Ontak®); dexrazoxane (Zinecard®); docetaxel (Taxotere®); doxorubicin (Adriamycin PFS®); doxorubicin (Adriamycin®, Rubex®); doxorubicin (Adriamycin PFS Injection®); doxorubicin liposomal (Doxil®); DROMOSTANOLONE PROPIONATE (DROMOSTANOLONE®); DROMOSTANOLONE PROPIONATE (MASTERONE INJECTION®); Elliott's B Solution (Elliott's B Solution®); epirubicin (Ellence®); Epoetin alfa (Epogen®); erlotinib (Tarceva®); estramustine (Emcyt®); etoposide phosphate (Etopophos®); etoposide, VP-16 (Vepesid®); exemestane (Aromasin®); Filgrastim (Neupogen®); floxuridine (intraarterial) (FUDR®); fludarabine (Fludara®); fluorouracil, 5-FU (Adrucil®); fulvestrant (Faslodex®); gefitinib (Iressa®); gemcitabine (Gemzar®); gemtuzumab ozogamicin (Mylotarg®); goserelin acetate (Zoladex Implant®); goserelin acetate (Zoladex®); histrelin acetate (Histrelin Implant®); hydroxyurea (Hydrea®); Ibritumomab Tiuxetan (Zevalin®); idarubicin (Idamycin®); ifosfamide (IFEX®); imatinib mesylate (Gleevec®); interferon alfa 2a (Roferon A®); Interferon alfa-2b (Intron A®); irinotecan (Camptosar®); lenalidomide (Revlimid®); letrozole (Femara®); leucovorin (Wellcovorin®, Leucovorin®); Leuprolide Acetate (Eligard®); levamisole (Ergamisol®); lomustine, CCNU (CeeBU®); meclorethamine, nitrogen mustard (Mustargen®); megestrol acetate (Megace®); melphalan, L-PAM (Alkeran®); mercaptopurine, 6-MP (Purinethol®); mesna (Mesnex®); mesna (Mesnex Tabs®); methotrexate (Methotrexate®); methoxsalen (Uvadex®); mitomycin C (Mutamycin®); mitotane (Lysodren®); mitoxantrone (Novantrone®); nandrolone phenpropionate (Durabolin-50®); nelarabine (Arranon®); Nofetumomab (Verluma®); Oprelvekin (Neumega®); oxaliplatin (Eloxatin®); paclitaxel (Paxene®); paclitaxel (Taxol®); paclitaxel protein-bound particles (Abraxane®); palifermin (Kepivance®); pamidronate (Aredia®); pegademase (Adagen (Pegademase Bovine)®); pegaspargase (Oncaspar®); Pegfilgrastim (Neulasta®); pemetrexed disodium (Alimta®); pentostatin (Nipent®); pipobroman (Vercyte®); plicamycin, mithramycin (Mithracin®); porfimer sodium (Photofrin®); procarbazine (Matulane®); quinacrine (Atabrine®); Rasburicase (Elitek®); Rituximab (Rituxan®); sargramostim (Leukine®); Sargramostim (Prokine®); sorafenib (Nexavar®); streptozocin (Zanosar®); sunitinib maleate (Sutent®); talc (Sclerosol®); tamoxifen (Nolvadex®); temozolomide (Temodar®); teniposide, VM-26 (Vumon®); testolactone (Teslac®); thioguanine, 6-TG (Thioguanine®); thiotepa (Thioplex®); topotecan (Hycamtin®); toremifene (Fareston®); Tositumomab (Bexxar®); Tositumomab/1-131 tositumomab (Bexxar®); Trastuzumab (Herceptin®); tretinoin, ATRA (Vesanoid®); Uracil Mustard (Uracil Mustard Capsules®); valrubicin (Valstar®); vinblastine (Velban®); vincristine (Oncovin®); vinorelbine (Navelbine®); and zoledronate (Zometa®).

Thus, the scope of the instant invention encompasses the use of the instantly claimed compounds in combination with a second compound selected from: an estrogen receptor modulator, an androgen receptor modulator, retinoid receptor modulator, a cytotoxic/cytostatic agent, an antiproliferative agent, a prenyl-protein transferase inhibitor, an HMG-CoA reductase inhibitor, an HIV protease inhibitor, a reverse transcriptase inhibitor, an angiogenesis inhibitor, a PPAR-γ agonist, a PPAR-δ agonist, an inhibitor of inherent multidrug resistance, an anti-emetic agent, an agent useful in the treatment of anemia, an agent useful in the treatment of neutropenia, an immunologic-enhancing drug, an inhibitor of cell proliferation and survival signaling, an apoptosis inducing agent, a bisphosphonate, an aromatase inhibitor, an siRNA therapeutic γ-secretase inhibitors, agents that interfere with receptor tyrosine kinases (RTKs), an agent that interferes with a cell cycle checkpoint and any of the therapeutic agents listed above.

Any one or more of the specific dosages and dosage schedules of the compounds of the instant invention, may also be applicable to any one or more of the therapeutic agents to be used in the combination treatment (hereinafter referred to as the “second therapeutic agent”).

Moreover, the specific dosage and dosage schedule of this second therapeutic agent can further vary, and the optimal dose, dosing schedule and route of administration will be determined based upon the specific second therapeutic agent that is being used.

Of course, the route of administration of the compounds of the instant invention is independent of the route of administration of the second therapeutic agent. In an embodiment, the administration for a compound of the instant invention is oral administration. In another embodiment, the administration for a compound of the instant invention is intravenous administration. Thus, in accordance with these embodiments, a compound of the instant invention is administered orally or intravenously, and the second therapeutic agent can be administered orally, parenterally, intraperitoneally, intravenously, intraarterially, transdermally, sublingually, intramuscularly, rectally, transbuccally, intranasally, liposomally, via inhalation, vaginally, intraoccularly, via local delivery by catheter or stent, subcutaneously, intraadiposally, intraarticularly, intrathecally, or in a slow release dosage form.

In addition, a compound of the instant invention and second therapeutic agent may be administered by the same mode of administration, i.e., both agents administered e.g., orally, by IV. However, it is also within the scope of the present invention to administer a compound of the instant invention by one mode of administration, e.g., oral, and to administer the second therapeutic agent by another mode of administration, e.g., IV or any other ones of the administration modes described hereinabove.

The first treatment procedure, administration of a compound of the instant invention, can take place prior to the second treatment procedure, i.e., the second therapeutic agent, after the treatment with the second therapeutic agent, at the same time as the treatment with the second therapeutic agent, or a combination thereof. For example, a total treatment period can be decided for a compound of the instant invention. The second therapeutic agent can be administered prior to onset of treatment with a compound of the instant invention or following treatment with a compound of the instant invention. In addition, anti-cancer treatment can be administered during the period of administration of a compound of the instant invention but does not need to occur over the entire treatment period of a compound of the instant invention.

The term “administration” and variants thereof (e.g., “administering” a compound) in reference to a compound of the invention means introducing the compound or a prodrug of the compound into the system of the animal in need of treatment. When a compound of the invention or prodrug thereof is provided in combination with one or more other active agents (e.g., a cytotoxic agent, etc.), “administration” and its variants are each understood to include concurrent and sequential introduction of the compound or prodrug thereof and other agents.

As used herein, the term “composition” is intended to encompass a product comprising the specified ingredients in the specified amounts, as well as any product which results, directly or indirectly, from combination of the specified ingredients in the specified amounts.

The term “therapeutically effective amount” as used herein means that amount of active compound or pharmaceutical agent that elicits the biological or medicinal response in a tissue, system, animal or human that is being sought by a researcher, veterinarian, medical doctor or other clinician.

The term “treating cancer” or “treatment of cancer” refers to administration to a mammal afflicted with a cancerous condition and refers to an effect that alleviates the cancerous condition by killing the cancerous cells, but also to an effect that results in the inhibition of growth and/or metastasis of the cancer.

In an embodiment, the angiogenesis inhibitor to be used as the second compound is selected from a tyrosine kinase inhibitor, an inhibitor of epidermal-derived growth factor, an inhibitor of fibroblast-derived growth factor, an inhibitor of platelet derived growth factor, an MMP (matrix metalloprotease) inhibitor, an integrin blocker, interferon-α, interleukin-12, pentosan polysulfate, a cyclooxygenase inhibitor, carboxyamidotriazole, combretastatin A-4, squalamine, 6-O-chloroacetyl-carbonyl)-fumagillol, thalidomide, angiostatin, troponin-1, or an antibody to VEGF. In an embodiment, the estrogen receptor modulator is tamoxifen or raloxifene.

Also included in the scope of the claims is a method of treating cancer that comprises administering a therapeutically effective amount of a compound of Formula I in combination with radiation therapy and/or in combination with a compound selected from: an estrogen receptor modulator, an androgen receptor modulator, retinoid receptor modulator, a cytotoxic/cytostatic agent, an antiproliferative agent, a prenyl-protein transferase inhibitor, an HMG-CoA reductase inhibitor, an HIV protease inhibitor, a reverse transcriptase inhibitor, an angiogenesis inhibitor, a PPAR-γ agonist, a PPAR-δ agonist, an inhibitor of inherent multidrug resistance, an anti-emetic agent, an agent useful in the treatment of anemia, an agent useful in the treatment of neutropenia, an immunologic-enhancing drug, an inhibitor of cell proliferation and survival signaling, an apoptosis inducing agent, a bisphosphonate, an aromatase inhibitor, an siRNA therapeutic and an agent that interferes with a cell cycle checkpoint.

And yet another embodiment of the invention is a method of treating cancer that comprises administering a therapeutically effective amount of a compound of Formula I in combination with paclitaxel or trastuzumab.

The invention further encompasses a method of treating or preventing cancer that comprises administering a therapeutically effective amount of a compound of Formula I in combination with a COX-2 inhibitor.

The instant invention also includes a pharmaceutical composition useful for treating or preventing cancer that comprises a therapeutically effective amount of a compound of Formula I and a compound selected from: an estrogen receptor modulator, an androgen receptor modulator, a retinoid receptor modulator, a cytotoxic/cytostatic agent, an antiproliferative agent, a prenyl-protein transferase inhibitor, an HMG-CoA reductase inhibitor, an HIV protease inhibitor, a reverse transcriptase inhibitor, an angiogenesis inhibitor, a PPAR-γ agonist, a PPAR-6 agonist; an inhibitor of cell proliferation and survival signaling, a bisphosphonate, an aromatase inhibitor, an siRNA therapeutic and an agent that interferes with a cell cycle checkpoint.

Further included within the scope of the invention is a method of treating or preventing a disease in which angiogenesis is implicated, which is comprised of administering to a mammal in need of such treatment a therapeutically effective amount of a compound of the present invention. Other inhibitors of MET may also be administered for this method of treatment. Ocular neovascular diseases, which may result in certain forms of blindness, are examples of conditions where much of the resulting tissue damage can be attributed to aberrant infiltration of blood vessels in the eye. The undesirable infiltration can be triggered by ischemic retinopathy, such as that resulting from diabetic retinopathy, retinopathy of prematurity, retinal vein occlusions, etc., or by degenerative diseases, such as the choroidal neovascularization observed in age-related macular degeneration. Inhibiting the growth of blood vessels by administration of the present compounds would therefore prevent the infiltration of blood vessels and prevent or treat diseases where angiogenesis is implicated, such as ocular diseases like retinal vascularization, diabetic retinopathy, age-related macular degeneration, and the like.

Routes of systemic administration of the compounds of the present invention described above may be utilized in the treatment of such ocular neovascular diseases. Other routes of ocular administration may also be employed, such as topical, periocular, intravitreal and the like. Intravitreal implants coated with a drug:polymer matrix may also be employed.

Ophthalmic pharmaceutical compositions that are adapted for topical administration to the eye may be in the form of solutions, suspensions, ointments, creams or as a solid insert. Ophthalmic formulations of this compound may contain from 0.01 ppm to 1% and especially 0.1 ppm to 1% of medicament. For a single dose, from between 0.01 to 5000 ng, preferably 0.1 to 500 ng, and especially 1 to 100 ng of the compound can be applied to the human eye. Formulations useful for intravitreal administration are similar to saline solutions described previously for intravenous administration.

These and other aspects of the invention will be apparent from the teachings contained herein.

### SCHEMES AND EXAMPLES

The compounds of this invention may be prepared by employing reactions as shown in the following schemes, in addition to other standard manipulations that are known in the literature or exemplified in the experimental procedures. The illustrative schemes below, therefore, are not limited by the compounds listed or by any particular substituents employed for illustrative purposes. Substituent numbering as shown in the schemes does not necessarily correlate to that used in the claims and often, for clarity, a single substituent is shown attached to the compound where multiple substituents are allowed under the definitions of the instant invention hereinabove.

Examples provided are intended to assist in a further understanding of the invention. Particular materials employed, species and conditions are intended to be illustrative of the invention and not limiting of the reasonable scope thereof.

The abbreviations used herein have the following tabulated meanings. Abbreviations not tabulated below have their meanings as commonly used unless specifically stated otherwise.

| | |
|---|---|
| Ac = | acetyl |
| Bn = | benzyl |
| CAMP = | Cyclic adenosine-3′,5′-monophosphate |
| DCM = | dichloromethane |
| Et₂O = | diethyl ether |
| DIAD = | diisopropyl azodicarboxylate |
| DIPEA = | N,N-diisopropylethylamine |
| DMAP = | 4-(dimethylamino)pyridine |
| DME = | dimethoxyethane |
| DMF = | N,N-dimethylformamide |
| DMFDMA = | dimethylformamide dimethyl acetal |
| DPPA = | diphenylphosphoryl azide |
| DPPE = | ethane-1,2-diylbis(diphenylphosphane) |
| DPPF or dppf = | 1,1′-bis(diphenylphosphanyl) ferrocene |
| DPPP = | propane-1,3-diylbis(diphenylphosphane) |
| dtbpy = | 4,4′-di-tert-butyl-2,2′-bipyridine |
| EDC = | 3-(ethyliminomethyleneamino)-N,N-dimethyl- |
| | propan-1-amine |
| Et₃N = | triethylamine |
| EtOAc = | ethyl acetate |
| GST = | glutathione transferase |
| HMDS = | hexamethyldisilazide |
| HOBt = | 1-hydroxybenzotriazole |
| Hr = | Hour |
| JohnPhos = | biphenyl-2-yl(di-tert-butyl)phosphane |
| m-CPBA = | m-chloroperbenzoic acid |
| MeCN = | Acetonitrile |
| MeOH = | Methanol |
| Me₄^{t}BuXPhos = | di-tert-butyl[3,4,5,6-tetramethyl-2′,4′,6′- |
| | tri(propan-2-yl)biphenyl-2-yl]phosphane |
| MMPP = | monoperoxyphthalic acid |
| MPPM = | monoperoxyphthalic acid, magnesium salt 6H₂O |
| NMP = | N-methylpyrrolidinone |
| NSAID = | non-steroidal anti-inflammatory drug |
| OXONE ® = | 2KHSO₅•KHSO₄•K₂SO₄ |
| PL-HCO₃ = | polymer-supported bicarbonate |
| Pd/C = | palladium on carbon |
| Pd(PPh₃)₄ = | tetrakis(triphenylphosphine)palladium (0) |
| Pd₂(dba)₃ = | dipalladium (0) trisdibenzylideneacetone |
| Pd(OAc)₂ = | palladium (II) acetate |
| PdCl₂(dppf)•DCM = | 1,1′-bis(diphenylphosphino)ferrocene-palladium |
| | (II) dichloride dichloromethane complex |
| PDE = | phosphodiesterase |
| Ph = | phenyl |
| PPh₃ = | triphenylphosphine |
| r.t. = | room temperature |
| Rac. = | racemic |
| RuPhos = | [2′,6′-bis(propan-2-yloxy)biphenyl-2- |
| | yl](dicyclohexyl)phosphane |
| SEM = | 2-(trimethylsilyl)ethoxymethoxy |
| SFC = | supercritical fluid chromatography |
| SPA = | scintillation proximity assay |
| SPE = | solid phase extraction |
| SPhos = | dicyclohexyl(2′,6′-dimethoxybiphenyl-2- |
| | yl)phosphane |
| TBAF = | tetra-n-butylammonium fluoride |
| TFA = | trifluoroacetic acid |
| THF = | tetrahydrofuran |
| TIPSH = | triisopropylsilanethiol |
| TLC = | thin layer chromatography |
| TMAD = | 3-(dimethylcarbamoylimino)-1,1-dimethylurea |
| XPhos = | 2-dicyclohexylphosphino-2′,4′,6′- |
| | triisopropylbiphenyl |

### Alkyl Group Abbreviations

| | |
|---|---|
| Me = | methyl |
| Et = | ethyl |
| n-Pr = | normal propyl |
| i-Pr = | isopropyl |
| n-Bu = | normal butyl |
| i-Bu = | isobutyl |
| s-Bu = | secondary butyl |
| t-Bu = | tertiary butyl |
| c-Pr = | cyclopropyl |
| c-Bu = | cyclobutyl |
| c-Pen = | cyclopentyl |
| c-Hex = | cyclohexyl |

Methods of Synthesis

Substituted aryl or heteroaryl amine I is reacted with sodium nitrite in the presence of aqueous hydrochloric acid as solvent at or below 5° C. to provide a diazonium intermediate that is further reacted with tert-butyl acetoacetate in the presence of sodium acetate in a suitable solvent mixture such as ethanol/water at or below 5° C. to afford the corresponding diazo intermediate II (Scheme 1). Diazo intermediate II is heated in DMFDMA solvent at or around 100° C. to afford the corresponding substituted pyridazinone intermediate III. Substituted pyridazinone III is treated with an acid such as TFA in a suitable solvent such as DCM to afford the corresponding carboxylic acid intermediate IV. The acid IV is then reacted with isobutyl chloroformate in the presence of a suitable base such as N-methyl morpholine in an appropriate solvent such as DCM. The corresponding activated intermediate is then treated with a suitable reducing agent such as sodium borohydride in an appropriate cosolvent such as water at or around 0° C. to afford alcohol intermediate V. Alcohol V is then reacted with thionyl chloride at ambient temperature in a suitable solvent such as MeCN to afford chloride intermediate VI. Chloride intermediate VI is reacted with an appropriately substituted phenol using a suitable base such as potassium carbonate or aqueous sodium hydroxide and a suitable solvent such as DMF or DMSO to obtain ether VII. Alternatively, an appropriately substituted aryl halide is reacted with potassium hydroxide with a suitable catalyst system such as dipalladium (0) trisdibenzylideneacetone and Me₄^{t}BuXPhos in a suitable solvent system such as 1,4-dioxane and water at a temperature at or around 100-130° C., under either conventional heating or microwave irradiation. Chloride VI is then added to this solution of in situ generated phenol, and the resultant mixture is heated to a temperature at or around 60-100° C. to give ether VII. Alternatively, alcohol V is reacted with an appropriately substituted phenol using Mitsunobu conditions (i.e., PPh₃ and DIAD) in a suitable solvent such as THF at or around ambient temperature to give ether VII. Aryl nitrile VIII, a particular exemplification of VII, is treated with aqueous hydrogen peroxide and an appropriate base such as potassium carbonate in a suitable solvent such as DMSO at or around ambient temperature to give IX. Aryl bromide X, a particular exemplification of VII is reacted with carbon monoxide gas and dimethylamine in the presence of an appropriate catalyst system (i.e., palladium (II) acetate and 1,3-diphenylphosphinopropane) to obtain dimethylamide XI. Trimethylsilyl-protected triazole XII, prepared similarly to VII, is treated with a fluoride source such as potassium bifluoride in a solvent such as methanol to give deprotected triazole XIII.

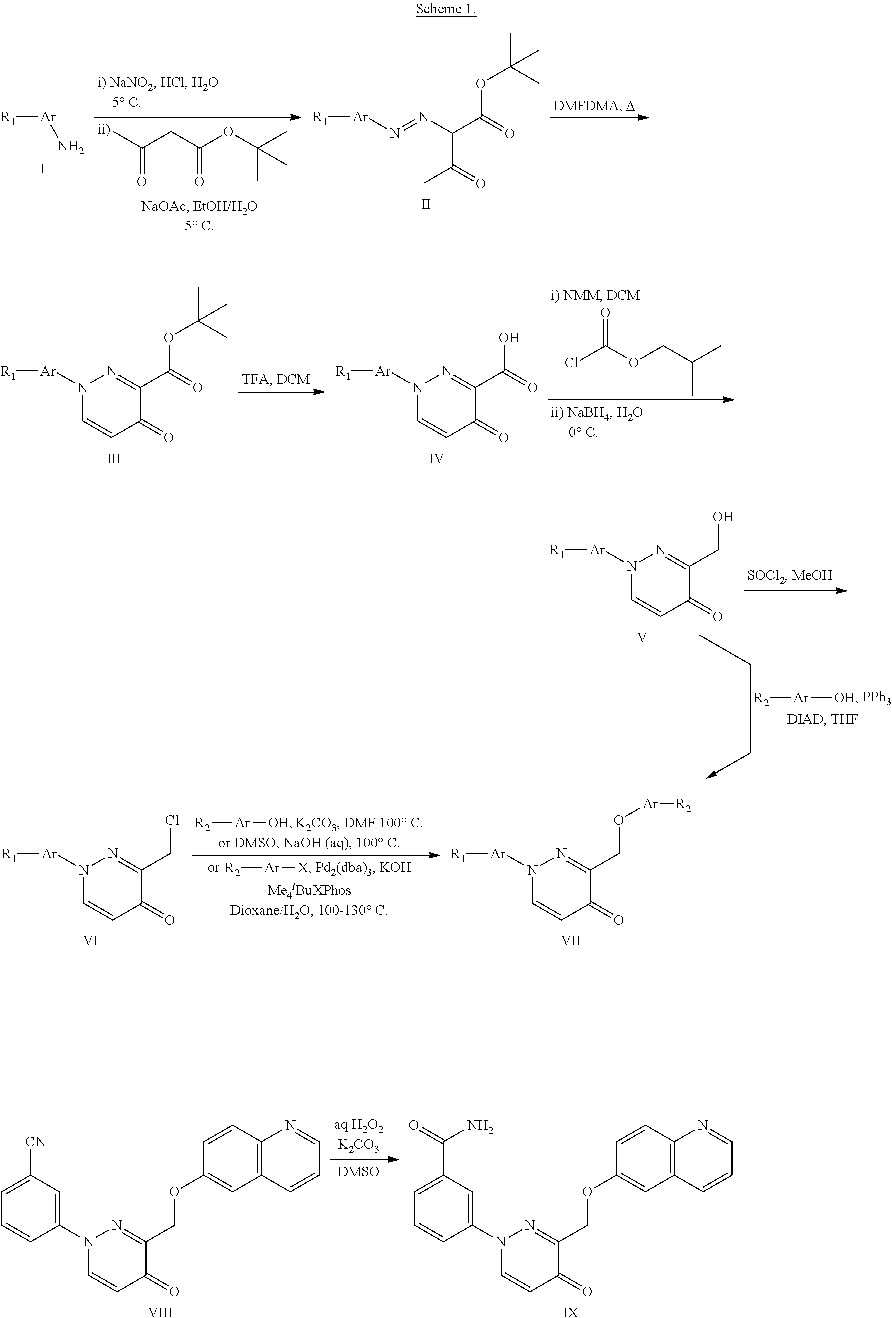

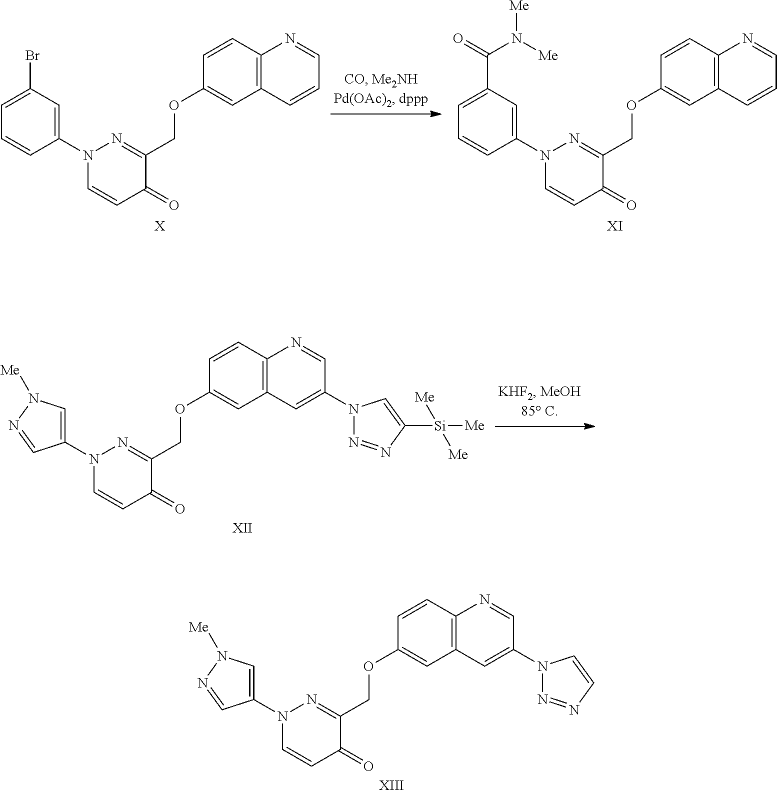

Non-commercially available aryl halides utilized in the preceding Buchwald ether synthesis may be prepared using the following methods (Aryl halide synthesis A-E):

Commercially available haloquinoline XIV is reacted with a borylation reagent such as bis(pinacolato)diboron using an appropriate catalyst (i.e., chloro(1,5-cyclooctadiene)iridium (I) dimer with a ligand such as dtbpy) in a solvent such as heptane at a temperature at or around 90° C. to give boronic ester XV (Method A). Intermediate XV is treated with a suitable oxidant such as Oxone in an appropriate solvent system such as acetone/water at a temperature at or around 0° C. to provide compound XVI. Hydroxyhaloquinoline XVI is reacted with an alkyl halide using a suitable base such as potassium carbonate in a suitable solvent such as DMF to give intermediate XVII.

Alternatively, 4-hydroxy-6-bromoquinoline XVIII is treated with a chlorination reagent such as phosphorous oxychloride to obtain XIX (Method B). 4-Chloro-6-bromoquinoline XIX is treated with a solution of appropriate sodium alkoxide in the same alcohol at a temperature at or around 100° C. to give intermediate XX. Alternatively, XIX is treated with a suitable base such as cesium carbonate in a solution of appropriate alcohol at a temperature at or around 100° C. to give XX.

Alternatively, haloquinoline XIV (X═Br) is treated with an oxidant such as m-CPBA in a solvent such as chloroform to obtain compound XXI (Method C). Quinoline N-oxide XXI is then reacted with a nitration reagent (i.e., silver nitrate and benzoyl chloride) in an appropriate solvent such as DCM to provide nitroquinoline N-oxide XXII, which is further treated with a reductant such as iron in an appropriate solvent system (i.e., water/ethanol/HCl) to provide compound XXIII. Aminoquinoline XXIII is treated with sodium nitrite in HF-pyridine to obtain dihaloquinoline XXIV.

Alternatively, commercially available carboxylic acid XXV is treated with DPPA in a solvent such as tert-butyl alcohol with a base such as triethyl amine at or around 100° C. to obtain XXVI (Method D). Treatment of protected amine XXVI with an acid such as HC in a solvent such as 1,4-dioxane gives aminoquinoline XXIII. XXIII is then heated neat with 1,2-diformylhydrazine at or around 160° C. to give 1,2,4-triazole XXVII. Alternatively, diazotization of XXIII with sodium nitrite at or around 0° C. in an appropriate solvent system such as THF/water with an appropriate acid (i.e., concentrated sulfuric acid) followed by reaction with a nucleophile such as sodium azide gives azide XXVIII. Reaction of XXVIII with an appropriately substituted alkyne in the presence of a suitable catalyst system such as copper (II) sulfate pentahydrate and sodium ascorbate in an appropriate solvent system such as Et₂O/DMF/H₂O gave 1,2,3-triazole XXIX.

### Aryl Halide Synthesis Method A

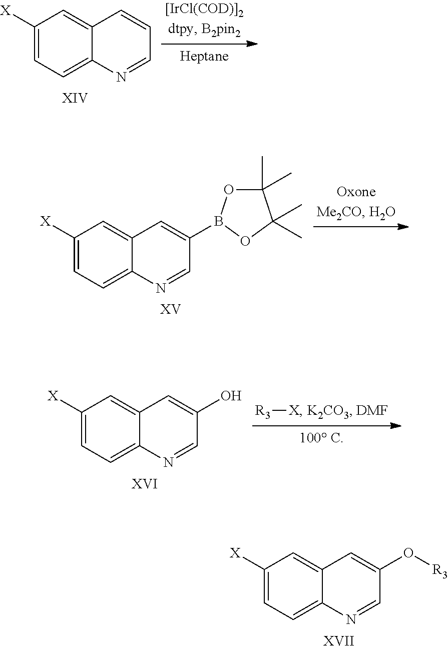

### Aryl Halide Synthesis Method B

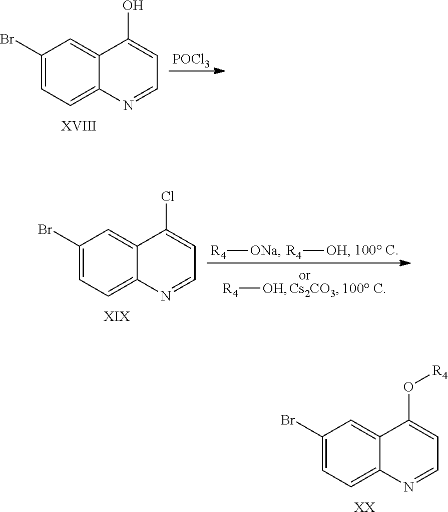

### Aryl Halide Synthesis Method C

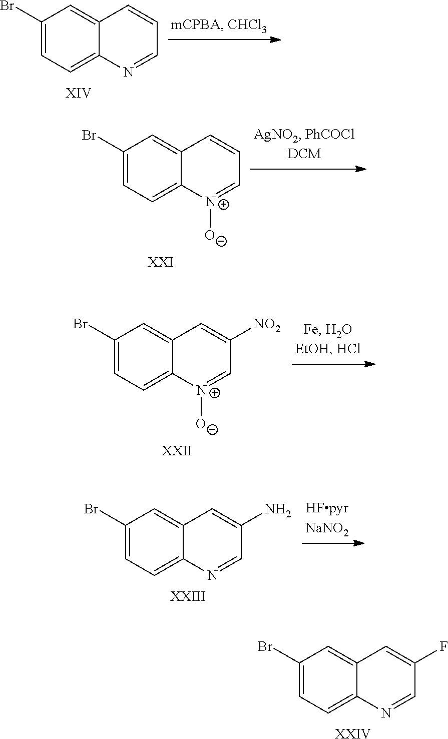

### Aryl Halide Synthesis Method D

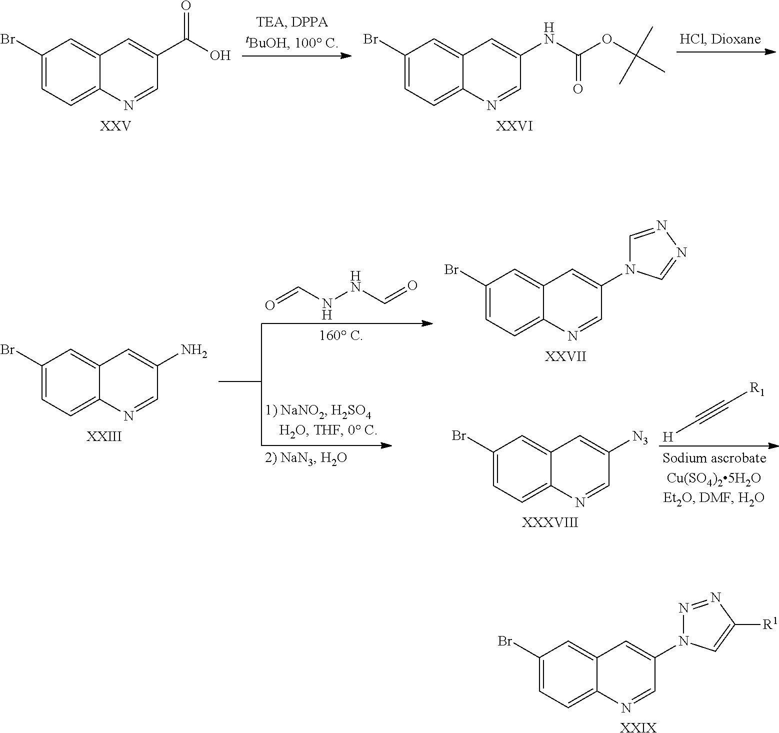

Chloride VI is treated with a phosphine such as triphenylphosphine in a suitable solvent such as DMF at or around 100° C. This solution is then treated with a suitable base such as potassium tert-butoxide and an appropriately substituted aryl aldehyde to afford XXX (Scheme 2). Alkene XXX is then reacted with hydrogen at one atmosphere of pressure (or alternatively at higher hydrogen pressure in a bomb apparatus) with an appropriate palladium catalyst (i.e., 10% Pd/C) in a solvent such as methanol at or around ambient temperature to afford XXXI.

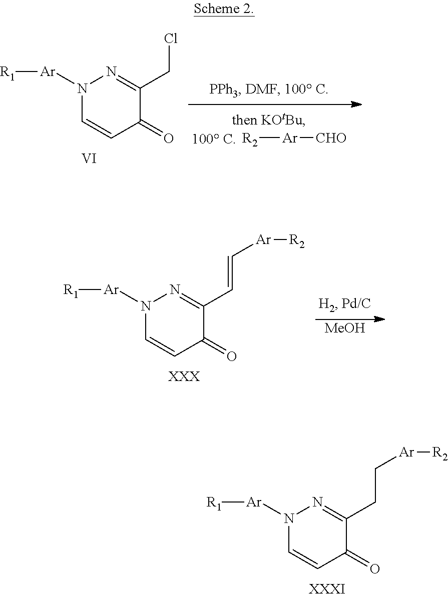

Commercially unavailable aryl aldehydes are prepared by reacting an aryl halide XVII or XX (prepared as described in Methods A and B) with potassium (2-trimethylsilylethoxy)methyl trifluoroborate using a suitable catalyst system, such as palladium(II) acetate and RuPhos or DTBPF, and suitable base such as cesium carbonate in a suitable solvent system such as 1,4-dioxane and water at or around 100° C. to give intermediate XXXII (Method E). Ether XXXII is then treated with boron trifluoride diethyl etherate in a suitable solvent such as DCM, followed by reaction with Dess-Martin periodinane in a suitable solvent such as DCM to give aldehyde XXXIII.

### Aryl Aldehyde Synthesis Method E

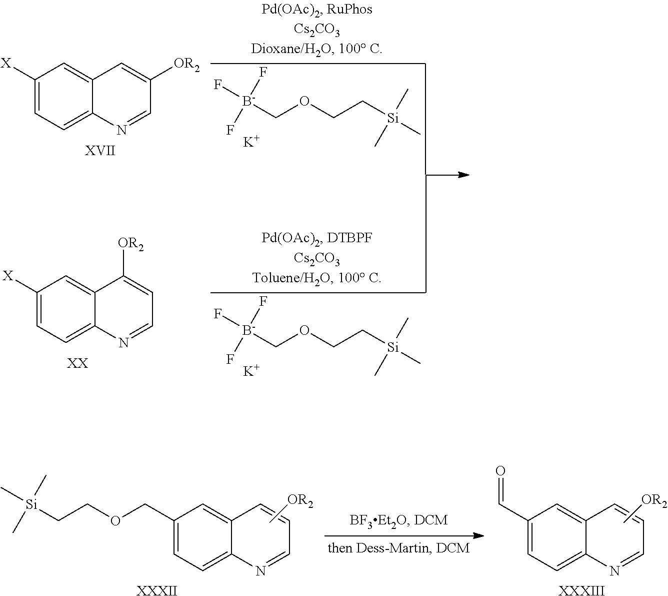

Chloride VI is treated with a phosphine such as triphenylphosphine in a suitable solvent such as DMF at or around 100° C. This solution is treated with a suitable base such as potassium tert-butoxide and quinoxaline-6-carbaldehyde and heated at or around 180° C. using microwave irradiation to afford XXXIV (Scheme 3). Quinoxaline XXXIV is hydrogenated at balloon pressure with an appropriate palladium catalyst (i.e., 10% Pd/C) in a suitable solvent system such as DCM/ethanol at or around ambient temperature to afford XXXV. Amine XXXV is stirred in a suitable solvent such as THF and treated with an oxidizing agent such as ceric ammonium nitrate at or around ambient temperature to provide quinoxaline XXXVI, a particular exemplification of XXXVI.

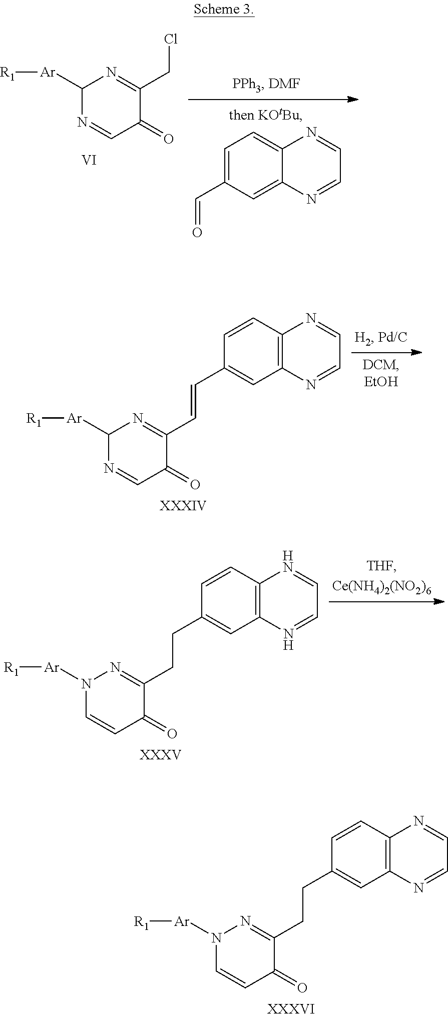

3-Hydroxy-6-haloquinoline XVI is deprotonated with a base such as sodium hydride in a suitable solvent system (i.e., DMF/THF) then alkylated with SEM-Cl to give XXXVII (Scheme 4). Aryl halide XXXVII is hydroxylated with potassium hydroxide and a suitable catalyst system such as dipalladium (0) trisdibenzylideneacetone in a suitable solvent system such as 1,4-dioxane and water at a temperature at or around 100° C. Addition of chloride VI to the crude solution and heating to a temperature at or around 100° C. gives XXXVIII. XXXVIII is deprotected with a suitable acid (i.e., HCl) in an appropriate solvent such as ethanol to give the hydrochloride salt XXXIX. XXXIX is then treated with isobutylene oxide in a suitable solvent such as DMF with a suitable base such as potassium carbonate at a temperature at or around 150° C. to give XL. Alternatively, XXXIX is reacted with an appropriately substituted alkyl halide and a suitable base such as potassium carbonate in a suitable solvent such as DMF at a temperature at or around 100-150° C. to give XLI.

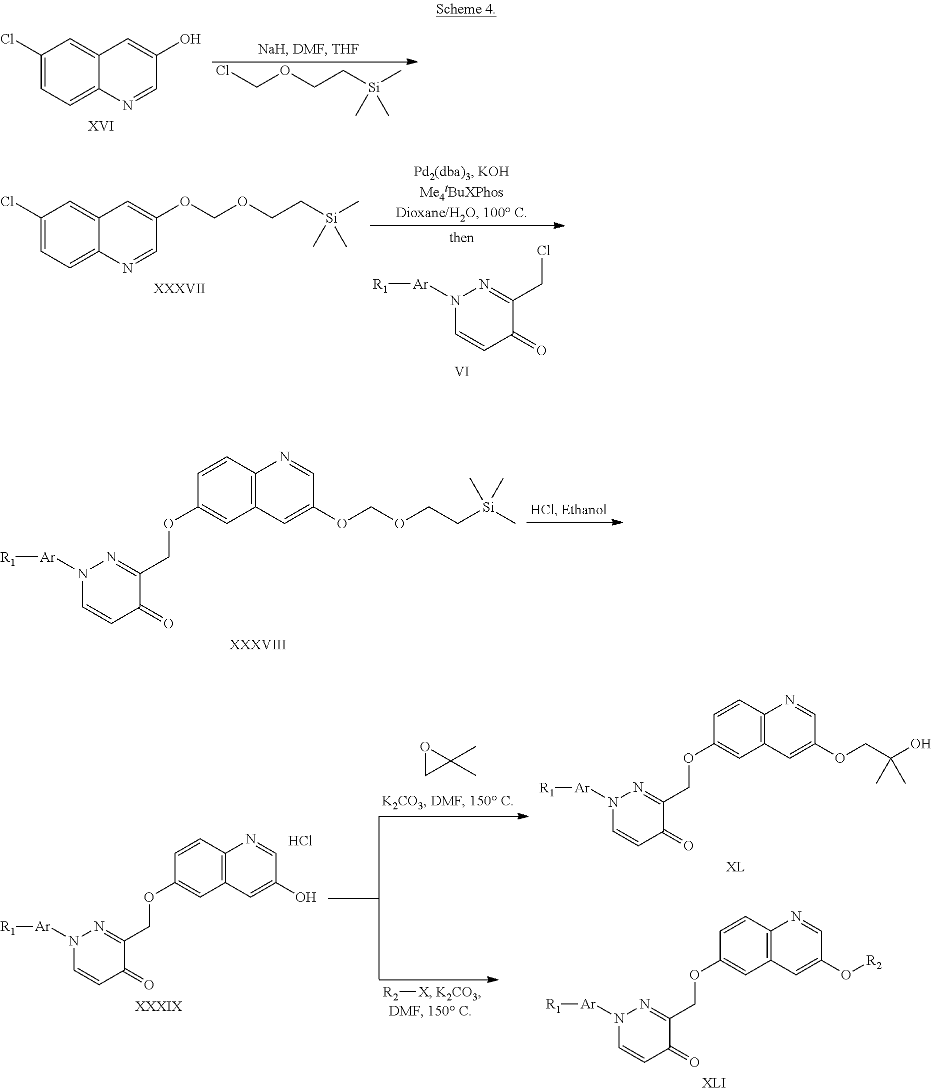

Intermediate XXXIX is stirred in a suitable solvent such as THF with a suitable trifluoromethanesulfonylation reagent (i.e., N-phenylbis(trifluoromethyl)sulfonimide) and a suitable base such as N,N-diisopropylethylamine at or around ambient temperature to obtain triflate XLII (Scheme 5). XLII is then reacted with an appropriately substituted organoboron compound (i.e., a potassium organotrifluoroborate salt, a boronic acid or a pinacol boronic ester) in a Suzuki-type coupling, using a suitable catalyst system (i.e., dipalladium (0) trisdibenzylideneacetone and SPhos or palladium (II) acetate and tricyclohexylphosphine) and base (i.e., cesium carbonate or potassium phosphate tribasic) in a suitable solvent or mixture of solvents such as 1,4-dioxane or toluene and water, at or around a temperature of 100° C., to obtain XLIII. Alternatively, XLII is reacted with carbon monoxide at one atmosphere of pressure with a suitable catalyst system (such as palladium (II) acetate and DPPP) in a suitable solvent system such as DMF/methanol with a suitable base such as N,N-diisopropylethylamine at or around 70° C. to give XLIV. In the particular case of compounds such as XLV, prepared in a similar manner as XLIII, deprotection of the tert-butyl carbamate to the amine XLVI is achieved by treatment with a suitable acid such as HCl in an appropriate solvent system such as MeOH/1,4-dioxane. Alternatively, compound XLII is reacted with a suitable borylation reagent such as bis(pinacolato)diboron using a suitable catalyst system (i.e., dipalladium (0) trisdibenzylideneacetone and XPhos) with a suitable base such as potassium acetate in an appropriate solvent such as 1,4-dioxane at or around 100° C. to obtain boronic acid XLVII. Meanwhile, iodide XLVIII is reacted with 3-iodooxetane and a base such as cesium carbonate in an appropriate solvent such as DMF at or around a temperature of 150° C. to give iodide XLIX, which is then coupled onto boronic acid XLVII using a suitable catalyst system (i.e., dipalladium (0) trisdibenzylideneacetone and XPhos) with an appropriate base such as cesium carbonate in a suitable solvent such as 1,4-dioxane at or around 100° C. to provide compound L.

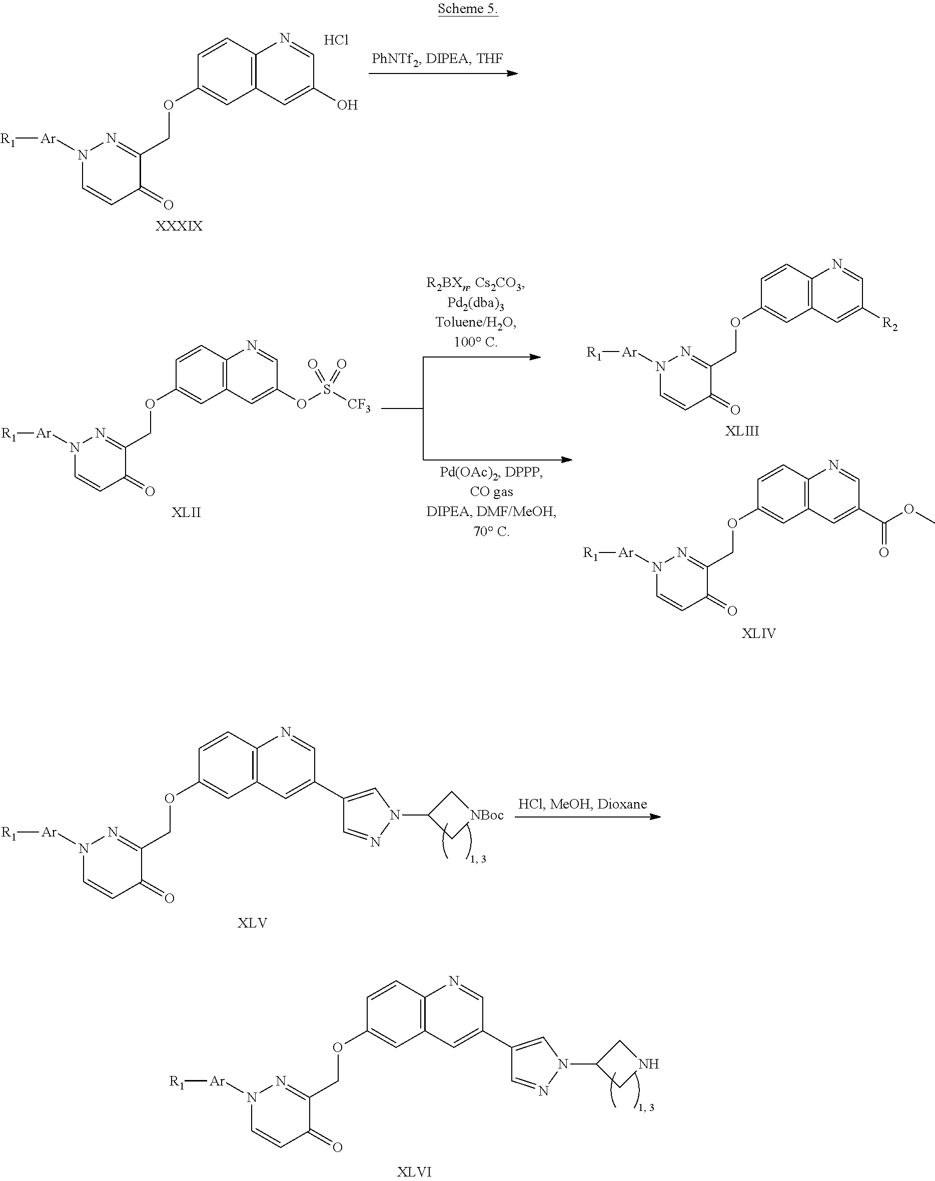

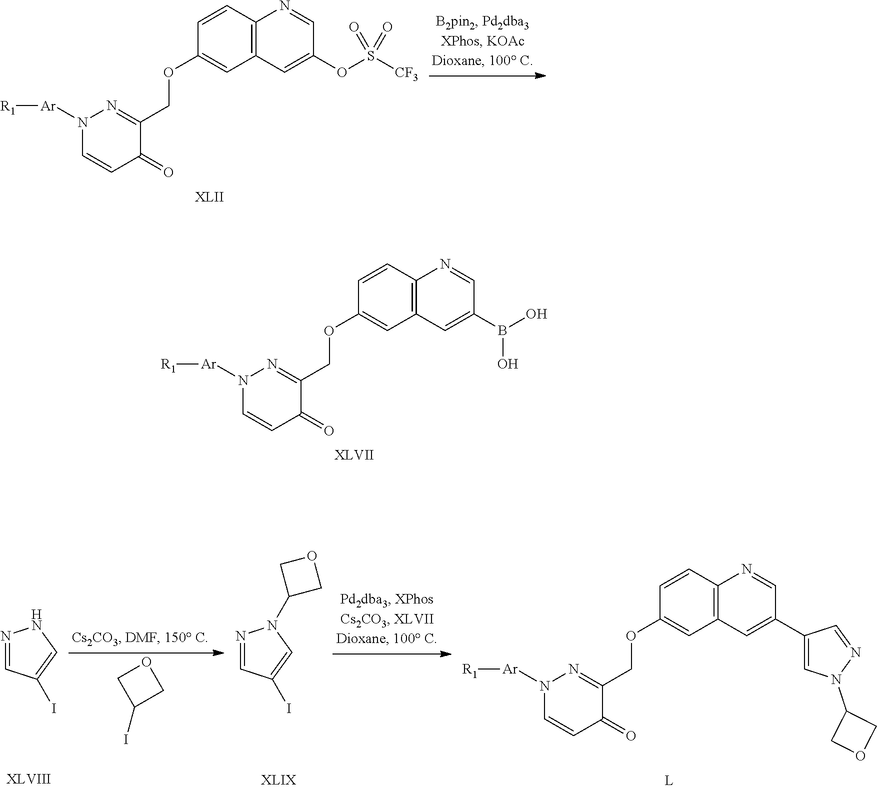

Non commercially available boronates used in the synthesis of XLIII may be obtained via reaction of commercially available aryl boronates such as LI with alkyl halides such as LII and a suitable base such as cesium carbonate in a suitable solvent such as DMF at or around 150° C., giving compounds such as LIII (Method F).

### Aryl Halide Synthesis Method F

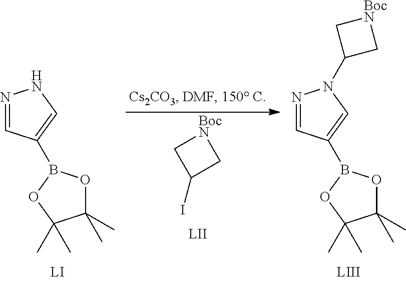 [SMILES: CC1(C)OB(c2cn[nH]c2)OC1(C)C ; CC(C)(C)OC(=O)N1CC(I)C1 ; CC(C)(C)OC(=O)N1CC(n2cc(B3OC(C)(C)C(C)(C)O3)cn2)C1]

6-Bromo-4-chloroquinoline XIX is reacted with potassium hydroxide using a suitable catalyst system such as dipalladium (0) trisdibenzylideneacetone and Me₄^{t}BuXPhos in a suitable solvent system such as 1,4-dioxane and water at a temperature at or around 60° C., then treated with alkyl chloride VI at a temperature at or around 60° C. to give LIV (Scheme 6). LIV is reacted with a suitably substituted organoboron derivative, using a suitable catalyst system (i.e., dipalladium (0) trisdibenzylideneacetone and SPhos) and base (i.e., cesium carbonate) in a suitable solvent or mixture of solvents such as toluene and/or water, at or around a temperature of 100° C. to obtain LV.

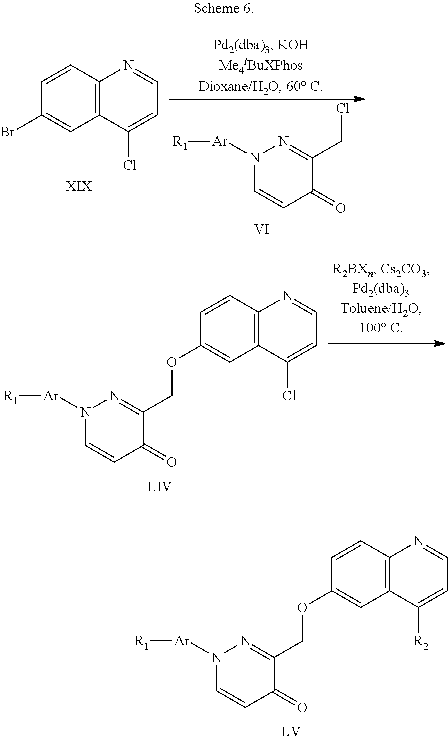

Aryl halide LVI is reacted with a suitable catalyst system (i.e., dipalladium (0) trisdibenzylideneacetone and Me₄^{t}BuXPhos) and potassium hydroxide in an appropriate solvent system such as 1,4-dioxane and water at a temperature at or around 100° C. (Scheme 8). An appropriate thiocarbamoyl chloride such as N,N-dimethylthiocarbamoyl chloride is then added to the reaction mixture and heating is continued at or around 100° C. to give LVII. LVII, dissolved in a suitable solvent such as NMP, is heated to 250° C. under microwave irradiation, followed by treatment with a suitable base (i.e., aqueous sodium hydroxide) at a temperature at or around 100° C. VI is then added to the reaction mixture, and heating continued at or around 100° C. to produce thioether LVIII. Alternatively, LVI is reacted with an appropriate sulfur source such as triisopropylsilane thiol using a suitable catalyst system (i.e., tetrakis(triphenylphosphine)palladium (0)) and a suitable base such as cesium carbonate in a suitable solvent such as toluene at a temperature at or around 100° C. to give LIX. LIX is then reacted with VI using a suitable base such as potassium carbonate in a suitable solvent such as DMF at or around 100° C. to give LVIII.

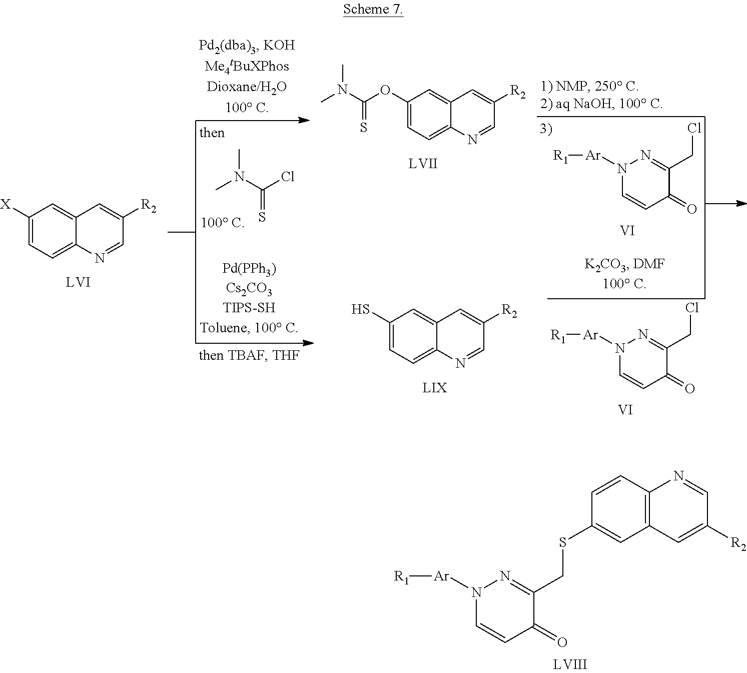

The appropriately substituted aryl halide LVI, if not commercially available may be obtained through Method A. Alternatively, commercially available boronic acid LX is stirred with a suitable iodination reagent (i.e., N-iodosuccinimide) in a suitable solvent such as acetonitrile to give a particular exemplification of LVI such that X═I and R═H (Method G), which can also be used in Scheme 8.

### Method G

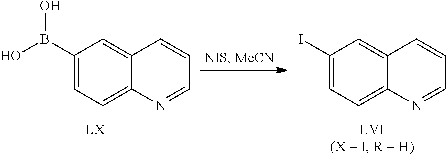 [SMILES: OB(O)c1ccc2ncccc2c1 ; Ic1ccc2ncccc2c1]

Aryl halide LVI is reacted with lithium hexamethyldisilazide using a suitable catalyst system (i.e., dipalladium (0) trisdibenzylideneacetone and JohnPhos) in a suitable solvent such as THF at or around 100° C. to give amine LXI after treatment with an acid such as aqueous HCl (Scheme 8). LXI is then heated with VI and an appropriate base such as potassium carbonate in a suitable solvent such as DMF to provide LXII. Alternatively, LXI may be a commercially available aryl amine.

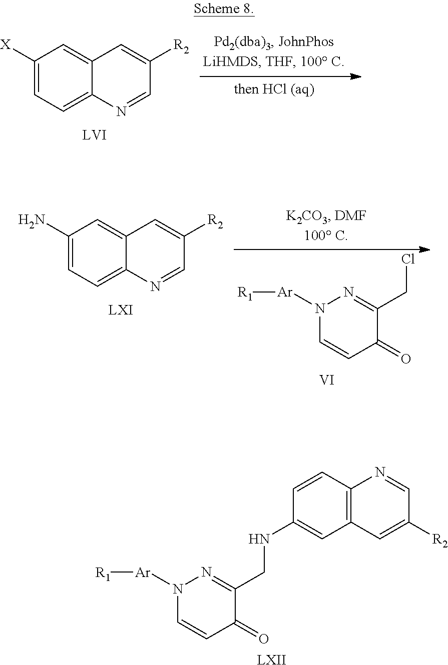

Aryl halide LXIII is treated with a suitable catalyst (i.e., 1,1′-diphenylphosphinoferrocene palladium (II) dichloride dichloromethane adduct), a suitable base (i.e., potassium acetate) and a suitable borylation reagent (i.e., bis(pinacoloto)diboron) in a suitable solvent such as 1,4-dioxane at a temperature at or around 100° C. to give boronate LXIV (Scheme 9). LXIV is stirred with a suitable base (i.e., aqueous sodium hydroxide) and an appropriate oxidizer such as aqueous hydrogen peroxide in a suitable solvent such as THF at or around ambient temperature, and then treated with VI at or around a temperature of 60° C. to give LXV.

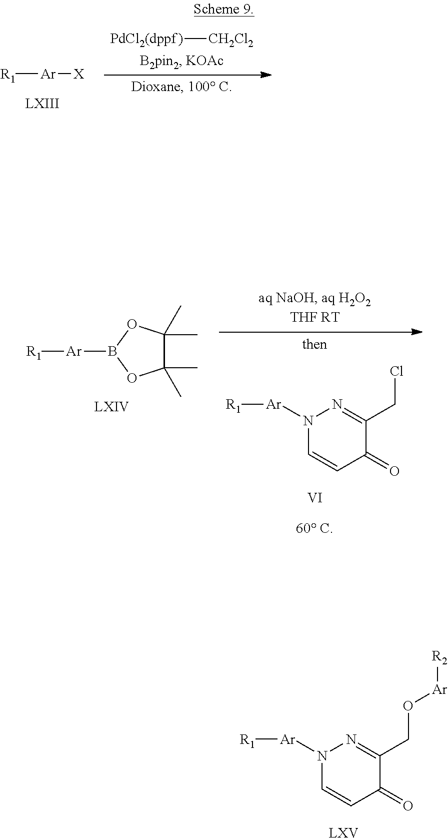

Non-commercially available aryl halides LXIII used above may be obtained as follows:

Haloquinoxaline LXVI is treated with an appropriate alcohol such as 2-methoxyethanol and a suitable base such as cesium carbonate at or around 100° C. to obtain compound LXVII (Method H).

Alternatively, LXVIII is reacted with an iodination reagent such as N-iodosuccinimide in an appropriate solvent such as acetic acid at or around 60° C. to obtain compound LXIX (Method I). Haloquinolinol LXIX is treated with a chlorination reagent such as phosphorus oxychloride at or around 100° C. to obtain polyhaloquinoline LXX. Compound LXX is reacted with an appropriate diol such as ethylene glycol using a suitable catalyst such as copper (I) iodide with a suitable base (i.e., cesium carbonate) at or around 100° C. to obtain tricyclic 1,4-dioxane LXXI.

### Aryl Halide Synthesis Method H

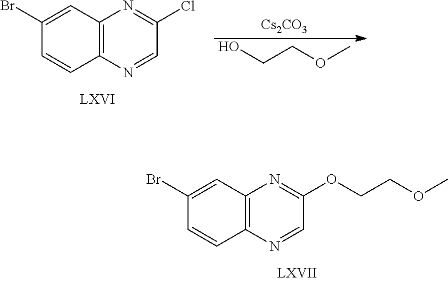

### Aryl Halide Synthesis Method I

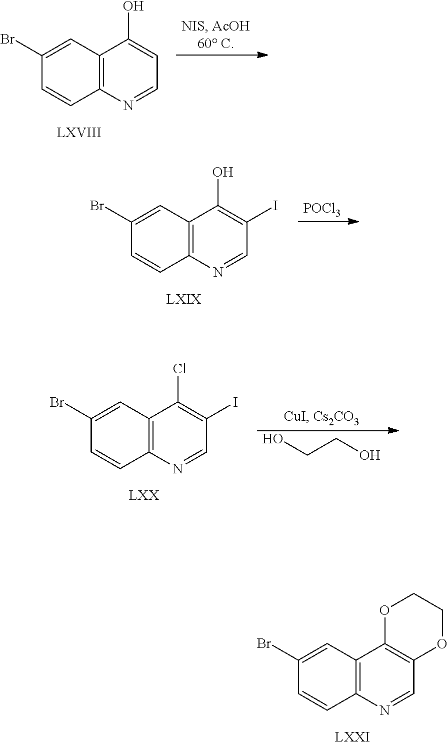

Alkyl chloride VI is reacted with an appropriately substituted amine such as methylamine in a suitable solvent such THF at a temperature at or around 80° C. to give LXXII (Scheme 10). LXXII is then reacted with an appropriately substituted aryl halide using a suitable catalyst system (i.e., dipalladium (0) trisdibenzylideneacetone and JohnPhos) and base (i.e., sodium tert-butoxide) in a suitable solvent such as DME at a temperature at or around 40° C. to give LXXIII.

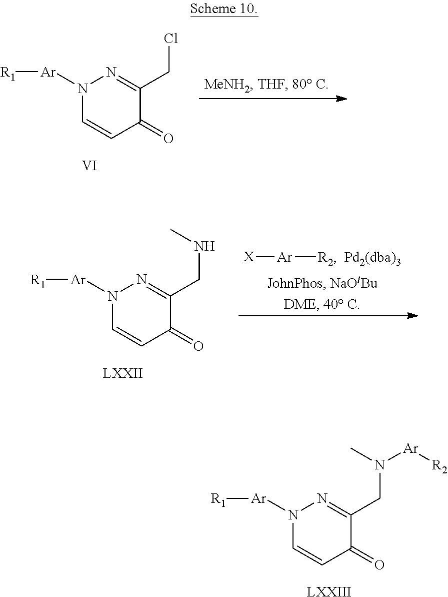

Methyl ester XLIV is treated with an appropriate base such as lithium hydroxide in a suitable solvent system such as THF and water at or around 100° C. to give LXXIV (Scheme 11). LXXIV is then stirred with an appropriate amine such as morpholine with suitable amide coupling reagents (i.e., HOBT, EDC and DIPEA) in a suitable solvent such as DMF at or around 100° C. to give amide LXXV.

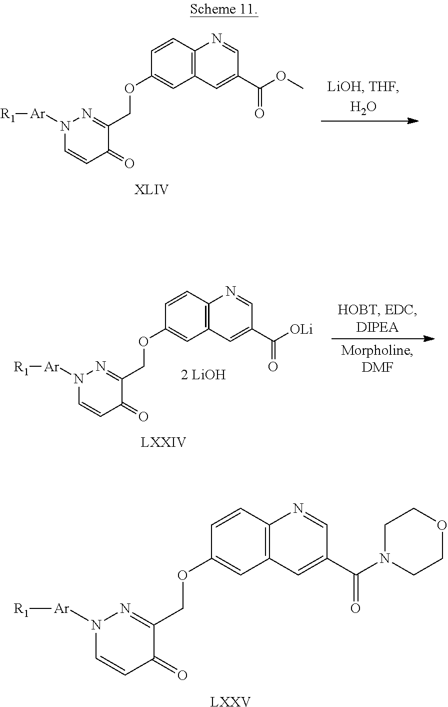

LXXIV, as the free acid, is treated with diphenylphosphorylazide and a suitable base such as triethylamine in tert-butyl alcohol at or around 100° C. to give a combination of LXXVI and LXXVII (Scheme 12). LXXVI is stirred in a suitable acid and solvent combination such as HCl and 1,4-dioxane at or around ambient temperature to give LXXVIII.

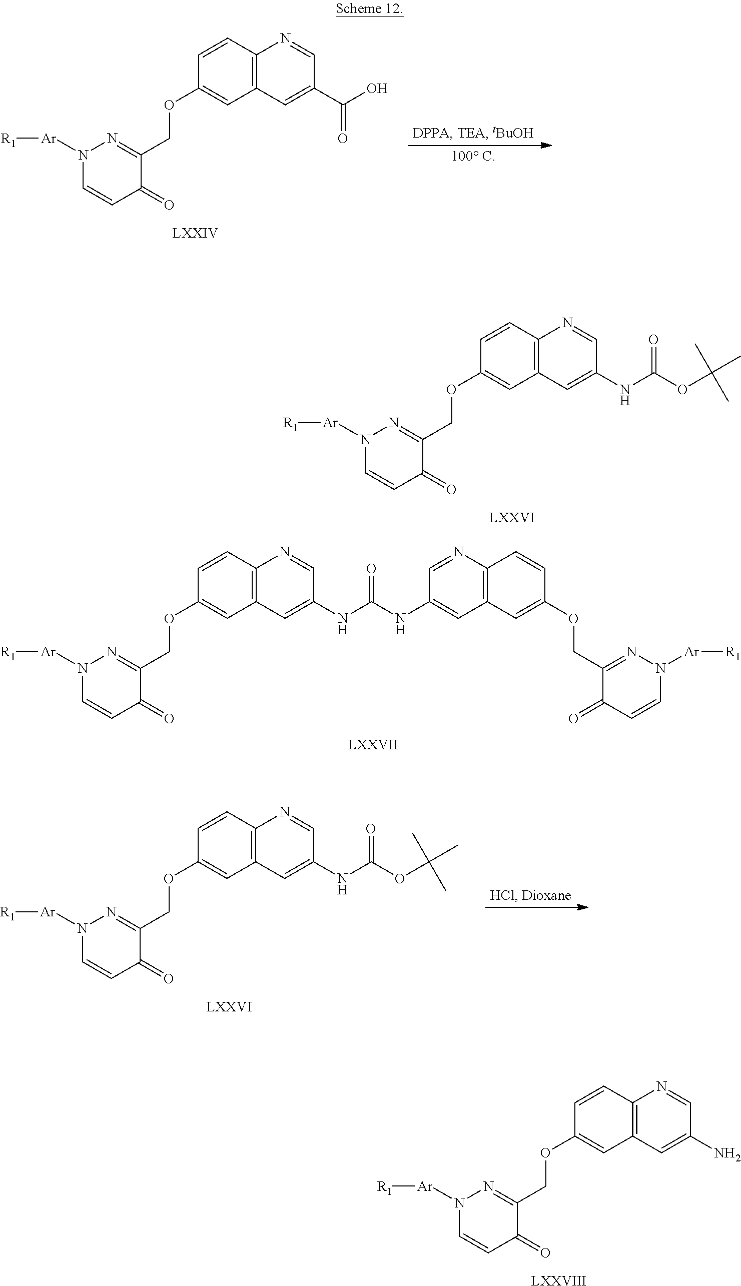

Carboxylic acid IV is stirred with N,O-dimethylhydroxylamine hydrochloride, suitable base such as N,N-diisopropylethylamine and suitable coupling reagents such as EDC and HOBt in a suitable solvent such as DMF at or around ambient temperature to give LXXIX (Scheme 13). LXXIX is treated at a temperature at or around −78° C. with methylmagnesium halide in a suitable solvent such as THF to give LXXX. LXXX is stirred with a reducing agent such as sodium borohydride in a suitable solvent such as methanol at or around ambient temperature, giving LXXXI. LXXXI is then reacted under appropriate Mitsunobu conditions (i.e., triphenylphosphine and DIAD in THF) with an appropriately substituted phenol at or around ambient temperature to give racemic ether LXXXII. LXXXII is then separated by appropriate chromatography (i.e., chiral SFC) to separate LXXXII into its two enantiomers, LXXXIII and LXXXIV.

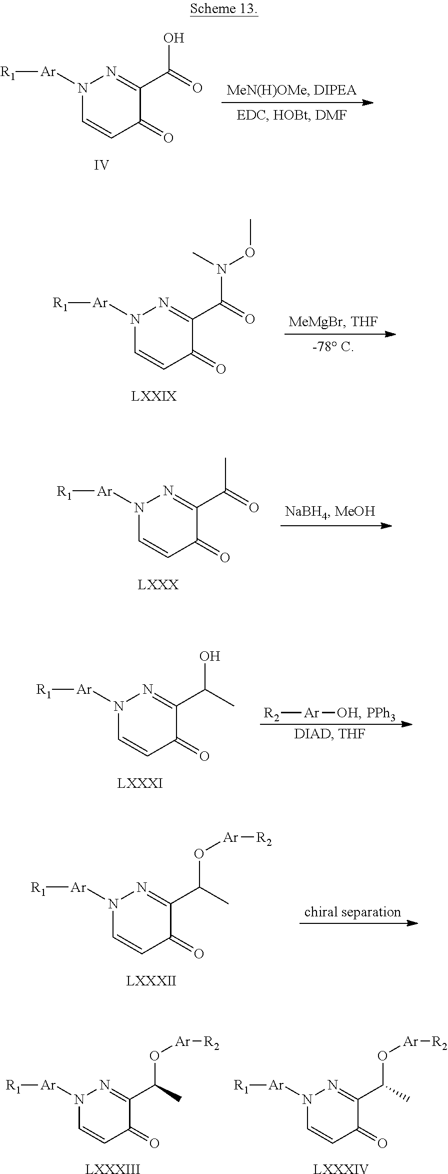

Compound LXXXV, a particular exemplification of VII or XXX such that X is either CH₂ or O, is hydrogenated under standard Parr shaker conditions (i.e., 45 psi of hydrogen gas over 10% Pd/C) in a suitable solvent system such as ethyl acetate and THF to give both LXXXVI and LXXXVII (Scheme 14). LXXXVI is then stirred with an appropriately substituted chloroformate ester and a suitable base such as N,N-diisopropylethylamine in a suitable solvent such as THF at or around ambient temperature to give LXXXVIII.

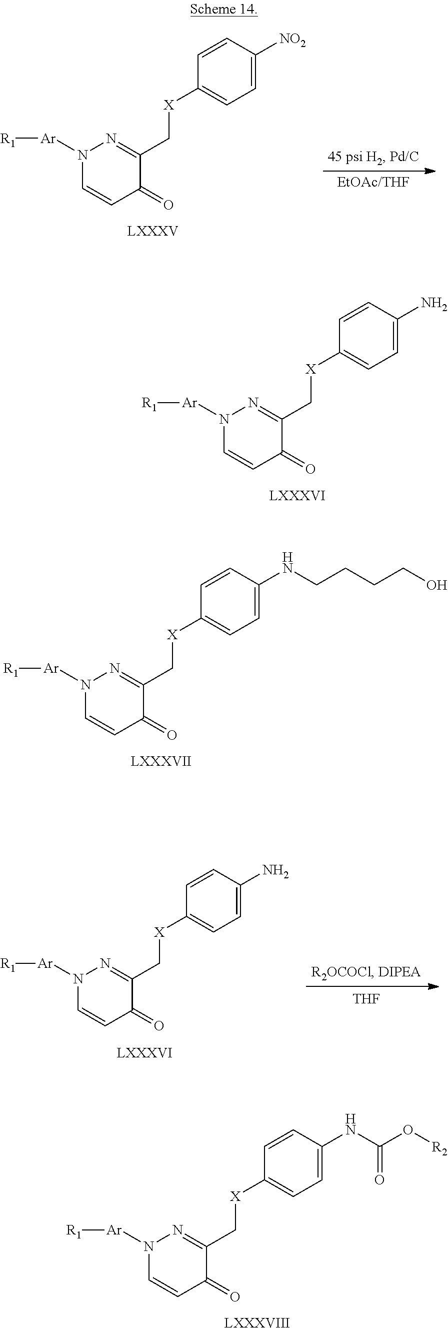

Compound LXXXIX is treated at or around −78° C. with an appropriate base (i.e., lithium hexamethyldisilazide) and trifluoromethanesulfonation reagent (i.e., 2-[N,N-bis(trifluoromethylsulfonyl)amino]pyridine) in a suitable solvent such as THF to generate vinyl trifluoromethanesulfonate XC (Scheme 15). Compound XC is reacted with an appropriate borylation reagent such as bis(pinacolato)diboron in a suitable solvent such as toluene with an appropriate catalyst system (i.e., triphenylphosphine and bis(triphenylphosphine)palladium (II) chloride) and base (i.e., sodium phenoxide) at or around 55° C. to obtain boronate XCI. XCI is coupled via a Suzuki reaction to compound VI using a suitable catalyst system such as 1,1′-diphenylphosphinoferrocene palladium (II) chloride with an appropriate base (i.e., potassium phosphate tribasic) and solvent (i.e., DME) at or around 100° C. to provide compound XCII. Olefin XCII is reduced under a balloon atmosphere of hydrogen in a solvent such as methanol with a suitable catalyst (i.e., Pd/C) to provide racemic compound XCIII, which is then separated into its enantiomers via appropriate chromatography such as chiral SFC to provide XCIV and XCV.

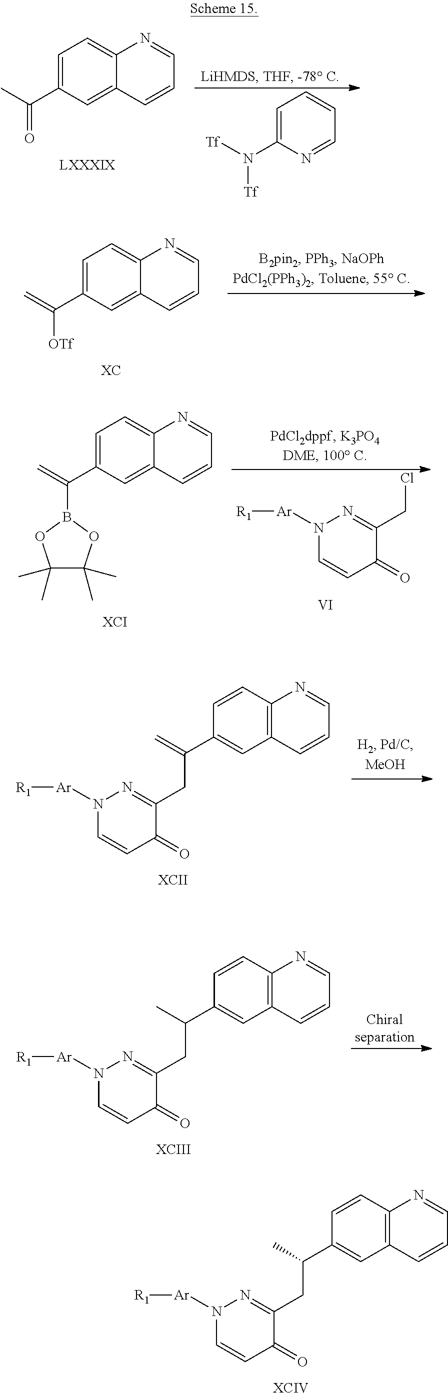

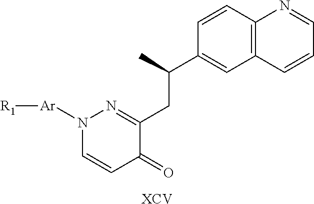

Compound XCVI is reacted with DPPA in tert-butanol with a suitable solvent such as triethylamine at reflux temperature to obtain compound XCVII. XCVII is deprotected by treatment with an acid such as HCl in solvent such as 1,4-dioxane to provide heterocycle XCVIII. Compound XCVIII chlorinated with sodium nitrite in concentrated HCl at or around 0° C. to give XCIX. Chloride XCIX is stirred with an alcohol such as C and a base such as sodium hydride in an appropriate solvent such as DMF to yield compound CI. Alternatively, XCIX is heated with XXXIII and cesium fluoride in a suitable solvent such as DMF at or around 200° C. to also provide CI. Compounds with the formula CI can be separated, when appropriate, with a suitable method of chromatography such as chiral SFC to provide the individual enantiomers CII and CIII.

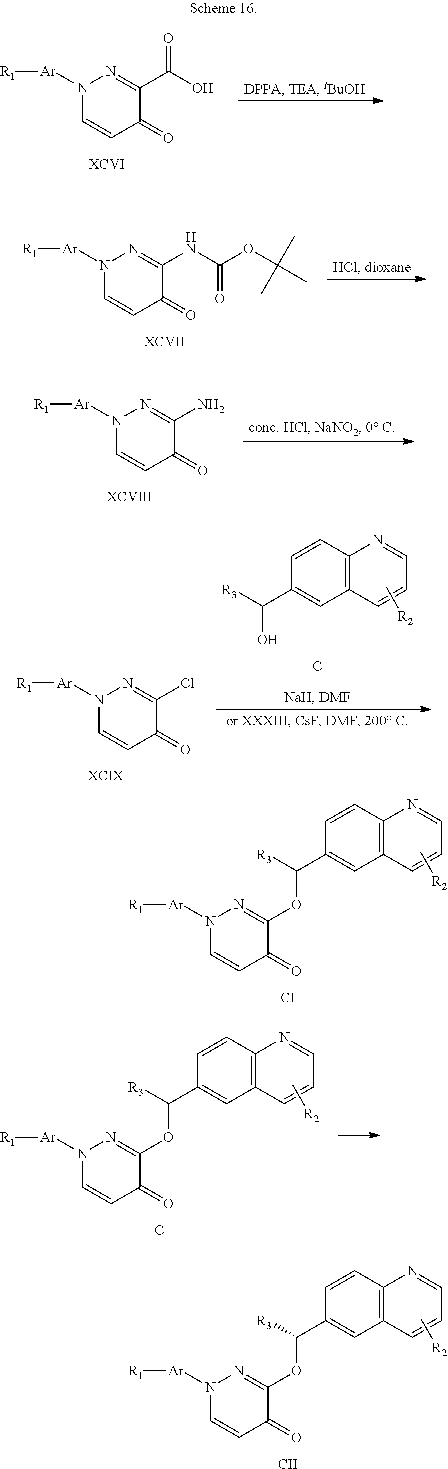

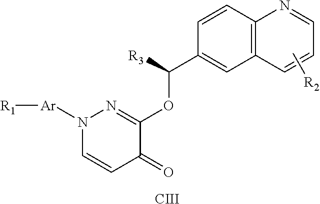

Non-commercially available compounds C can be generated as follows:

Aryl halide XVII or XX is reacted with a vinyl ether such as butyl vinyl ether using an appropriate base (i.e., N-cyclohexyl-N-methylcyclohexanamine) and catalyst system (i.e., palladium acetate and 1,3-bis(diphenylphosphino)propane) in a solvent such as DMSO followed by hydrolysis with a suitable acid such as aqueous HCl to provide ketone CIX (Method J). CIX is treated with a reducing agent such as sodium borohydride in a solvent such as methanol to provide alcohol CX, a particular exemplification of alcohol C.

Alternatively, alcohol C (where R₃═H), either commercially available or prepared as shown in Method J, is reacted with a borylation reagent such as bis(pinacolato)diboron in a solvent such as CPME using an appropriate catalyst system (i.e., dtbpy and bis(1,5-cyclooctadiene)di-μ-methoxydiiridium (I)) at or around 100° C. and then treated with an aryl halide such as N-methyl-4-iodopyrazole in situ using an appropriate catalyst and base (i.e., 1,1′-diphenylphosphinoferrocene dichloropalladium (II) and potassium hydroxide in water) to provide alcohol CXI, another particular exemplification of C (Method K).

### Alcohol Synthesis Method J

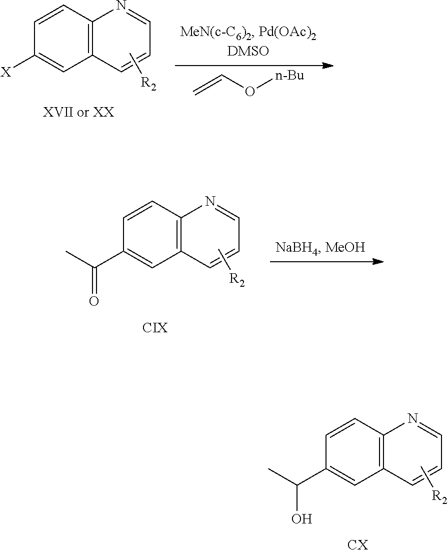

### Alcohol Synthesis Method K

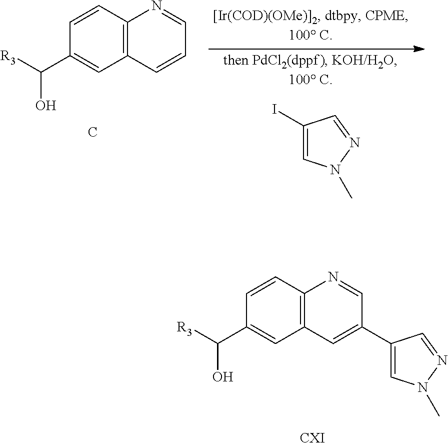

Compound XCVII is reacted with compound C under appropriate Mitsunobu conditions (i.e., triphenylphosphine and tetramethylazodicarboxamide in THF) to obtain compound CIV, which is subsequently deprotected with an acid such as TFA in a solvent such as DCM to provide compound CV (Scheme 17).

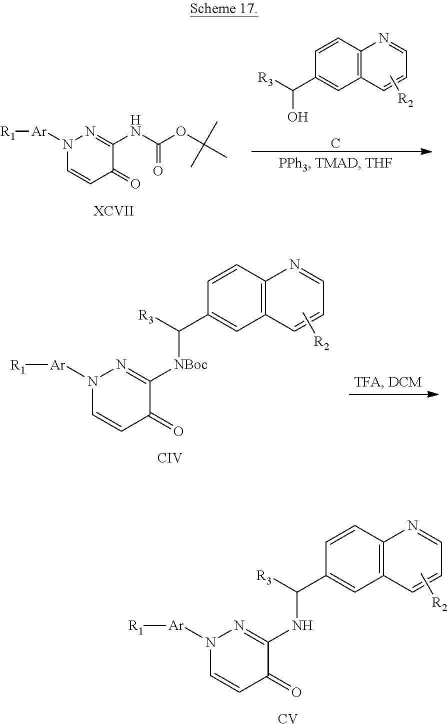

Compound CVI (a particular exemplification of C) is treated with thioacetic acid using suitable Mitsunobu conditions (i.e., triphenylphosphine and di-tert-butylazodicarboxylate in THF) to give compound CVII (Scheme 18). Treatment of thioester CVII with a base such as aqueous sodium hydroxide in a suitable solvent system such as methanol and THF followed by treatment of the formed intermediate with XCIX in a solvent such as DMF gives thioether CIII.

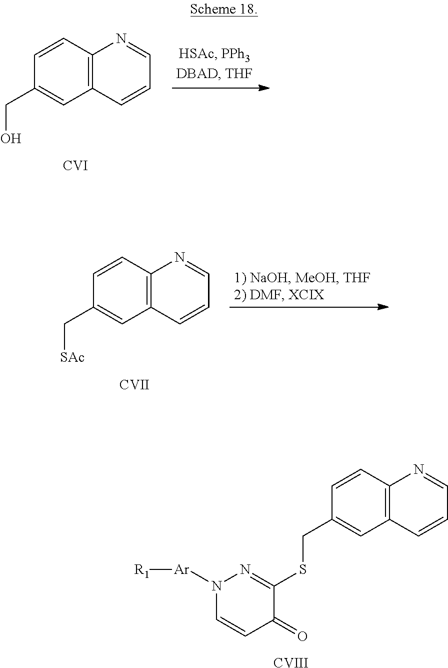

Ester CXII is treated with a base such as sodium hydride in a suitable solvent system such as THF/DMF to obtain the conjugate base of CXII, which is then reacted with compound VI to provide compound CXIII (Scheme 19).

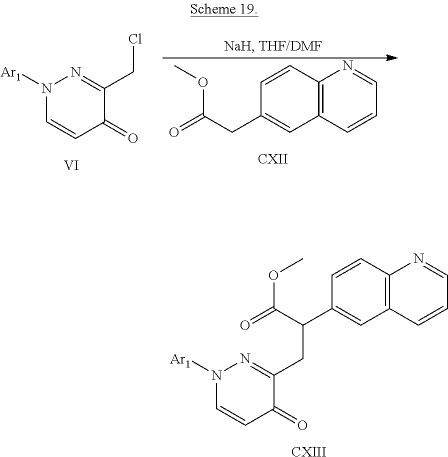

The invention will now be illustrated in the following non-limiting Examples in which, unless otherwise stated:

All the end products of the formula I were analyzed by NMR, LCMS.
Intermediates were analyzed by NMR and/or TLC and/or LCMS.
Most compounds were purified by silica gel flash chromatography, reverse-phase HPLC, recrystallization and/or swish (suspension in a solvent followed by filtration of the solid).
The course of the reactions were followed by thin layer chromatography (TLC) and/or LCMS and reaction times are given for illustration only.

### Scheme #1

### Intermediate #1

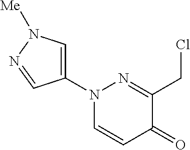 [SMILES: Cn1cc(-n2ccc(=O)c(CCl)n2)cn1]

### 3-(Chloromethyl)-1-(1-methyl-1H-pyrazol-4-yl)pyridazin-4(1H)-one

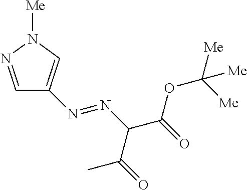 [SMILES: CC(=O)C(/N=N/c1cnn(C)c1)C(=O)OC(C)(C)C]

### Step 1. tert-Butyl 2-[1-methyl-1H-pyrazol-4-yl)diazenyl]-3-oxobutanoate

1-Methyl-1H-pyrazol-4-amine (17.0 g, 175 mmol) was dissolved in concentrated HCl (50 mL)/water (260 mL) and cooled to 0° C. A solution of sodium nitrite (12.7 g, 184 mmol) in water (180 mL) was added dropwise while maintaining the internal temperature at <4° C. On complete addition, the mixture was stirred at 0° C. for 20 minutes. The resulting diazonium chloride solution was added dropwise to a solution of tert-butyl acetoacetate (29.0 mL, 175 mmol) and sodium acetate (187 g, 2280 mmol) in water (220 mL)/ethanol (220 mL) at 0° C. The resulting mixture was stirred at 0° C. for 15 minutes. Saturated aqueous sodium bicarbonate solution was added and the products extracted into EtOAc (3×). The combined organic extracts were dried over sodium sulfate, filtered and concentrated in vacuo to give tert-butyl 2-[(1-methyl-1H-pyrazol-4-yl)diazenyl]-3-oxobutanoate.

LRMS (ESI) calc'd for C12H19N4O3 [M+H]⁺: 267. Found: 267.

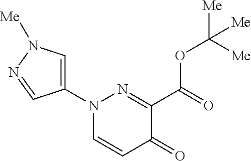 [SMILES: Cn1cc(-n2ccc(=O)c(C(=O)OC(C)(C)C)n2)cn1]

### Step 2. tert-Butyl 1-(1-methyl-1H-pyrazol-4-yl)-4-oxo-1,4-dihydropyridazine-3-carboxylate

tert-Butyl 2-[(1-methyl-1H-pyrazol-4-yl)diazenyl]-3-oxobutanoate (47.0 g, 176 mmol) was stirred in refluxing DMFDMA (350 mL) for 1 hour. Ambient temperature was attained before cooling the reaction mixture in the freezer overnight. The solvent was decanted off, Et₂O was added and the solid collected by filtration and washed with Et₂O followed by water to give tert-butyl 1-(1-methyl-1H-pyrazol-4-yl)-4-oxo-1,4-dihydropyridazine-3-carboxylate.

LRMS (ESI) calc'd for C13H17N4O3 [M+H]⁺: 277. Found: 277.

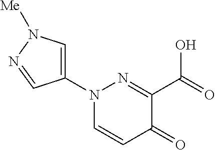 [SMILES: Cn1cc(-n2ccc(=O)c(C(=O)O)n2)cn1]

### Step 3. 1-(1-Methyl-1H-pyrazol-4-yl)-4-oxo-1,4-dihydropyridazine-3-carboxylic acid

tert-Butyl 1-(1-methyl-1H-pyrazol-4-yl)-4-oxo-1,4-dihydropyridazine-3-carboxylate (35.1 g, 127 mmol) was stirred in DCM (580 mL)/TFA (58 mL) at ambient temperature for 2 hours. The solvent was removed in vacuo and the residue triturated in Et₂O to give 1-(1-methyl-1H-pyrazol-4-yl)-4-oxo-1,4-dihydropyridazine-3-carboxylic acid.

LRMS (ESI) calc'd for C9H9N4O3 [M+H]⁺: 221. Found: 221.

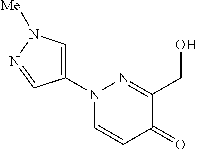 [SMILES: Cn1cc(-n2ccc(=O)c(CO)n2)cn1]

### Step 4. 3-(Hydroxymethyl)-1-(1-methyl-1H-pyrazol-4-yl)pyridazin-4(1H)-one

1-(1-Methyl-1H-pyrazol-4-yl)-4-oxo-1,4-dihydropyridazine-3-carboxylic acid (27.4 g, 125 mmol) was taken up in THF (1250 mL) and cooled to 0° C. Isobutyl chloroformate (19.6 mL, 149 mmol) was added, followed by N-methylmorpholine (16.4 mL, 149 mmol) and the resulting mixture stirred at 0° C. for 1 hour. A solution of sodium borohydride (14.1 g, 374 mmol) in water (75 mL) was prepared and immediately added to the reaction mixture at such a rate so as to avoid bubbling over. After 1 hour at 0° C., additional water was added and the solvent removed in vacuo onto silica gel. Purification of the residue by silica gel flash chromatography (MPLC, gradient elution, 0-10% MeOH/DCM) gave 3-(hydroxymethyl)-1-(1-methyl-1H-pyrazol-4-yl)pyridazin-4(1H)-one.

LRMS (ESI) calc'd for C9H11N4O2 [M+H]⁺: 207. Found: 207.

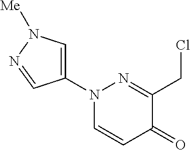 [SMILES: Cn1cc(-n2ccc(=O)c(CCl)n2)cn1]

### Step 5. 3-(Chloromethyl)-1-(1-methyl-1H-pyrazol-4-yl)pyridazin-4(1H)-one

3-(Hydroxymethyl)-1-(1-methyl-1H-pyrazol-4-yl)pyridazin-4(1H)-one (20.2 g, 98.0 mmol) was taken up in MeCN (980 mL). Thionyl chloride (35.7 mL, 489 mmol) was added dropwise and the resulting mixture stirred at ambient temperature for 3 hours. The reaction mixture was concentrated in vacuo onto silica gel and purified by silica gel flash chromatography (MPLC, gradient elution, 0-10% MeOH/DCM). The isolated product was taken up in 10% MeOH/DCM and washed with saturated aqueous sodium bicarbonate solution, dried over magnesium sulfate, filtered and concentrated in vacuo. The residue was triturated in Et₂O to give 3-(chloromethyl)-1-(1-methyl-1H-pyrazol-4-yl)pyridazin-4(1H)-one.

LRMS (ESI) calc'd for C9H10ClN4O [M+H]⁺: 225. Found: 225.

The following intermediates were prepared according to Scheme #1 following similar procedures described for Intermediate #1, which can be achieved by those of ordinary skill in the art of organic synthesis.

| | | | Exact |
|---|---|---|---|
| | | | Mass |
| Inter- | | | [M + |
| mediate | Structure | IUPAC Name | H]⁺ |
| 2 | 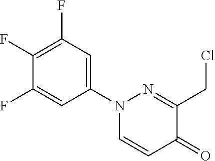 [SMILES: O=c1ccn(-c2cc(F)c(F)c(F)c2)nc1CCl] | 3- (chloromethyl)- 1-(3,4,5- trifluorophenyl) pyridazin- 4(1H)-one | Calc'd 275, found 275 |
| 3 | 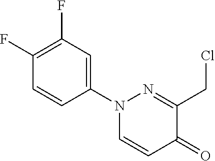 [SMILES: O=c1ccn(-c2ccc(F)c(F)c2)nc1CCl] | 3- (chloromethyl)- 1-(3,4- difluorophenyl) pyridazin- 4(1H)-one | Calc'd 257, found 257 |
| 4 | 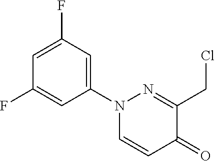 [SMILES: O=c1ccn(-c2cc(F)cc(F)c2)nc1CCl] | 3- (chloromethyl)- 1-(3,5- difluorophenyl) pyridazin- 4(1H)-one | Calc'd 257, found 257 |
| 5 | 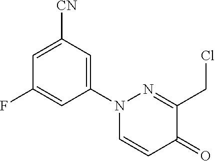 [SMILES: N#Cc1cc(F)cc(-n2ccc(=O)c(CCl)n2)c1] | 3-chloro-5-[3- (chloromethyl)- 4-oxopyridazin- 1(4H)-yl] benzonitrile | Calc'd 264 found 264 |
| 6 | 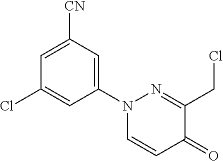 [SMILES: N#Cc1cc(Cl)cc(-n2ccc(=O)c(CCl)n2)c1] | 3-fluoro-5-[3- (chloromethyl)- 4-oxopyridazin- 1(4H)- yl]benzonitrile | Calc'd 280, found 280 |
| 7 | 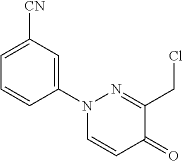 [SMILES: N#Cc1cccc(-n2ccc(=O)c(CCl)n2)c1] | 3-[3- (chloromethyl)- 4-oxopyridazin- 1(4H)-yl] benzonitrile | Calc'd 246, found 246 |
| 8 | 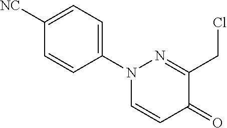 [SMILES: N#Cc1ccc(-n2ccc(=O)c(CCl)n2)cc1] | 4-[3- (chloromethyl)- 4-oxopyridazin- 1(4H)-yl] benzonitrile | Calc'd 246, found 246 |
| 9 | 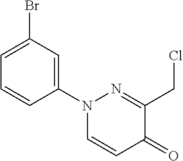 [SMILES: O=c1ccn(-c2cccc(Br)c2)nc1CCl] | 1-(3- bromophenyl)- 3- (chloromethyl) pyridazin- 4(1H)-one | Calc'd 299, found 299 |
| 10 | 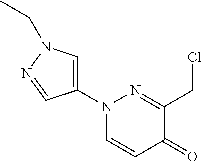 [SMILES: CCn1cc(-n2ccc(=O)c(CCl)n2)cn1] | 3- (chloromethyl)- 1-(1-ethyl-1H- pyrazol-4- yl)pyridazin- 4(1H)-one | Calc'd 239, Found 239 |

### Method A

### Intermediate #11

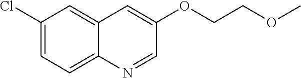 [SMILES: COCCOc1cnc2ccc(Cl)cc2c1]

### 6-Chloro-3-(2-methoxyethoxy)quinoline

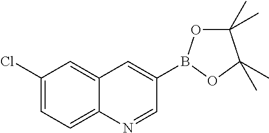 [SMILES: CC1(C)OB(c2cnc3ccc(Cl)cc3c2)OC1(C)C]

### Step 1. 6-Chloro-3-(4,4,5,5-tetramethyl-1,3,2-dioxaborolan-2-yl)quinoline

A round bottom flask was charged with 4,4′-di-tert-butyl-2,2′-bipyridine (1.05 g, 3.91 mmol), chloro(1,5-cyclooctadiene)iridium (I) dimer (1.30 g, 1.96 mmol) and heptane (326 mL) and sparged with a stream of nitrogen for 20 min while stirring at room temperature. 6-Chloroquinoline (16.0 g, 98 mmol) and bis(pinacolato)diboron (32.3 g, 127 mmol) were added and the reaction mixture was heated at 90° C. for 4 hours then stirred at room temperature overnight. The reaction mixture was diluted with 100 mL of ethyl acetate and the organics were separated, washed with 5% sodium carbonate (40 mL) followed by brine (40 mL), dried over sodium sulfate, filtered and concentrated in vacuo to give crude 6-chloro-3-(4,4,5,5-tetramethyl-1,3,2-dioxaborolan-2-yl)quinoline which was carried on without purification.

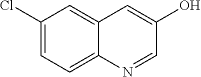 [SMILES: Oc1cnc2ccc(Cl)cc2c1]

### Step 2. 6-Chloroquinolin-3-ol

Crude 6-chloro-3-(4,4,5,5-tetramethyl-1,3,2-dioxaborolan-2-yl)quinolin (28 g, 97 mmol) was dissolved in acetone (320 mL) and stirred in an ice bath. Oxone (59.4 g, 97 mmol) was dissolved in water (320 mL) and added via addition funnel over 1.5 hours (the reaction temperature was monitored internally and kept below 5° C.). After completion of addition, the reaction was stirred in the ice bath for 30 minutes, quenched with 20 mL of 5% sodium hydrosulfite solution, stirred for 10 minutes and filtered. The filtrate was diluted with 100 mL of water and extracted with ethyl acetate (2×100 mL). The combined organics were washed with brine, dried over sodium sulfate, filtered, concentrated in vacuo and purified via silica gel flash chromatography (MPLC, gradient elution, 0-100% EtOAc/hexanes). The resulting solid was triturated with hexanes to give a portion of 6-chloroquinolin-3-ol. Meanwhile, the remaining aqueous fractions were made basic with solid sodium carbonate, at which point a large amount of precipitate was observed to form which was extracted with ethyl acetate (3×100 mL) and the combined organics were dried over sodium sulfate, filtered, concentrated in vacuo and purified by silica gel flash chromatography (MPLC, gradient elution, 0-100% EtOAc/hexanes). The resulting oily solid was triturated with hexanes to give a further portion of 6-chloroquinolin-3-ol.

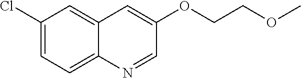 [SMILES: COCCOc1cnc2ccc(Cl)cc2c1]

### Step 3. 6-Chloro-3-(2-methoxyethoxy)quinoline

An oven-dried, nitrogen cooled 20 mL scintillation vial was charged with 36-chloroquinolin-3-ol (1.00 g, 5.57 mmol) and potassium carbonate (1.15 g, 8.32 mmol), sealed under nitrogen, charged with a solution of 2-bromoethyl methyl ether (0.85 g, 6.12 mmol) in DMF (15 mL) and heated to 100° C. for 17 hours. After cooling to ambient temperature, the reaction mixture was diluted with water, extracted with EtOAc (3×), washed with brine (2×), dried over magnesium sulfate, filtered and concentrated in vacuo. Water was added to the residue and stirred for several hours to give a precipitate which was collected by filtration and dried via lyophilizer to obtain 6-chloro-3-(2-methoxyethoxy)quinoline.

LRMS (ESI) calc'd for C12H13ClNO2 [M+H]⁺: 238. Found: 238.

The following examples were prepared according to Method A following similar procedures described for Intermediate #11, which can be achieved by those of ordinary skill in the art of organic synthesis.

| | | | Exact Mass |
|---|---|---|---|
| Intermediate | Structure | IUPAC Name | [M + H]⁺ |
| 12 | 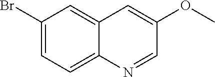 | 6-bromo-3- methoxyquinoline | Calc'd 238, Found 238 |
| 13 | 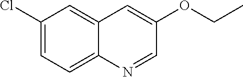 | 6-chloro-3- ethoxyquinoline | Calc'd 208, Found 208 |
| 14 | 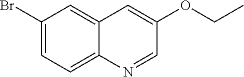 | 6-bromo-3- ethoxyquinoline | Calc'd 252, Found 252 |
| 15 | 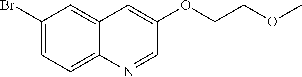 [SMILES: COCCOc1cnc2ccc(Br)cc2c1] | 6-bromo-3-(2- methoxyethoxy) quinoline | Calc'd 282, found 282 |
| 16 | 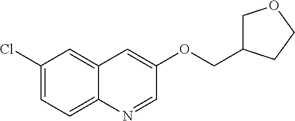 [SMILES: Clc1ccc2ncc(OCC3CCOC3)cc2c1] | 6-chloro-3- (tetrahydrofuran-3- ylmethoxy)quinoline | Calc'd 264, Found 264 |
| 17 | 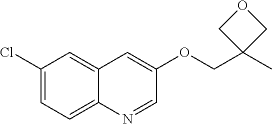 [SMILES: CC1(COc2cnc3ccc(Cl)cc3c2)COC1] | 6-chloro-3-[(3- methyloxetan-3- yl)methoxy]quinoline | Calc'd 264, Found 264 |

### Method B

### Intermediate #18

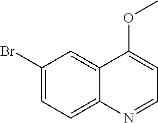

### 6-Bromo-4-methoxyquinoline

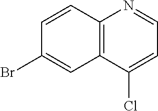 [SMILES: Clc1ccnc2ccc(Br)cc12]

### Step 1. 6-Bromo-4-chloroquinoline

A 20 mL scintillation vial was charged with 6-bromoquinolin-4-ol (0.98 g, 4.4 mmol) and phosphorus (V) oxychloride (10 mL). The reaction mixture was stirred for 19 hours at ambient temperature and carefully poured into a mixture of saturated aqueous sodium bicarbonate solution and ice, extracted into ethyl acetate (3×), washed with brine, dried over magnesium sulfate, filtered and concentrated in vacuo. The residue was purified by silica gel flash chromatography (MPLC, gradient elution, 0-50% EtOAc/hexanes) to give 6-bromo-4-chloroquinoline.

LRMS (ESI) calc'd for C9H6BrClN [M+H]⁺: 242. Found: 242.

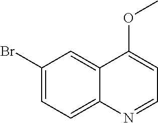

### Step 2. 6-Bromo-4-methoxyquinoline

A 5 mL microwave vial was charged with 6-bromo-4-chloroquinoline (0.15 g, 0.62 mmol) and a 25 wt % solution of sodium methoxide in methanol (2.0 mL, 8.8 mmol), sealed and heated to 100° C. for 60 minutes under microwave irradiation. After cooling, the solvent was removed in vacuo, the residue washed with water and dried via lyophilizer to obtain 6-bromo-4-methoxyquinoline.

LRMS (ESI) calc'd for C10H9BrNO [M+H]⁺: 238. Found: 238.

The following example was prepared according to Method B following similar procedures described for Intermediate #18, which can be achieved by those of ordinary skill in the art of organic synthesis.

| Inter- | | | Exact Mass |
|---|---|---|---|
| mediate | Structure | IUPAC Name | [M + H]⁺ |
| 19 | 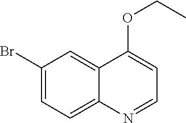 | 6-bromo-4- ethoxyquinoline | Calc'd 252, Found 252 |

### Method B

### Intermediate #20

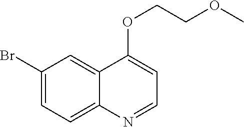 [SMILES: COCCOc1ccnc2ccc(Br)cc12]

### 6-Bromo-4-(2-methoxyethoxy)quinoline

### Step 1. 6-Bromo-4-(2-methoxyethoxy)quinoline

A 5 mL microwave vial was charged with 6-bromo-4-chloroquinoline (0.10 g, 0.41 mmol), cesium carbonate (0.20 g, 0.61 mmol) and 2-ethoxyethanol (2 mL), sealed and heated to 100° C. for 60 minutes under microwave irradiation. After cooling and removing the solvent in vacuo, the residue was taken up in water, extracted with EtOAc (3×), washed with brine, dried over magnesium sulfate, filtered and concentrated to obtain 6-bromo-4-(2-methoxyethoxy)quinoline.

LRMS (ESI) calc'd for C12H13BrNO2 [M+H]⁺: 282. Found: 282.

The following example was prepared according to Method B following similar procedures described for Intermediate #20, which can be achieved by those of ordinary skill in the art of organic synthesis.

| | | | Exact |
|---|---|---|---|
| Inter- | | | Mass |
| me- | | IUPAC | [M + |
| diate | Structure | Name | H]⁺ |
| 21 | 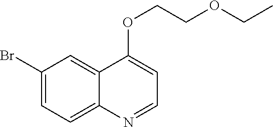 [SMILES: CCOCCOc1ccnc2ccc(Br)cc12] | 6-bromo- 4-(2- ethoxy- ethoxy) quinoline | Calc'd 296, Found 296 |

### Method C

### Intermediate #22

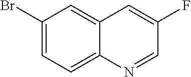 [SMILES: Fc1cnc2ccc(Br)cc2c1]

### 6-Bromo-3-fluoroquinoline

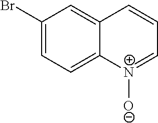 [SMILES: [O-][n+]1cccc2cc(Br)ccc21]

### Step 1. 6-Bromoquinoline-1-oxide

In a 10 L 4-necked round-bottom flask, a solution of 6-bromoquinoline (500 g, 2.40 mol) in chloroform (5 L) was charged with m-CPBA (831 g, 4.80 mol) and stirred overnight at 60° C. The reaction mixture was cooled, washed with aqueous potassium carbonate (3×3 L) and brine (2×1 L), dried over sodium sulfate, filtered and concentrated in vacuo. The residue was washed with petroleum ether to obtain 6-bromoquinoline-1-oxide.

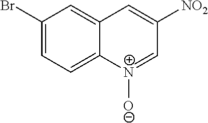 [SMILES: O=[N+]([O-])c1cc2cc(Br)ccc2[n+]([O-])c1]

### Step 2. 6-Bromo-3-nitroquinoline-1-oxide

A 5 L 4-necked round-bottom flask was charged with silver nitrate (501 g, 2.95 mol) and DCM (1 L). Benzoyl chloride (225 g, 1.60 mol) was added dropwise while stirring at 0° C. over 30 min and the reaction mixture was stirred for 180 min at room temperature. The solid formed was filtered, and the resulting filtrate was added dropwise to a refluxing solution of 6-bromoquinoline-1-oxide (300 g, 1.34 mol) in dichloromethane (2 L). The reaction mixture was refluxed overnight in an oil bath and the resulting solution was cooled, washed with saturated aqueous sodium bicarbonate solution (2×2 L) and brine (2×1 L), dried over sodium sulfate, filtered and concentrated in vacuo to obtain 6-bromo-3-nitroquinoline-1-oxide which was used without further purification.

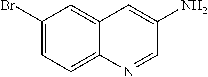 [SMILES: Nc1cnc2ccc(Br)cc2c1]

### Step 3. 6-Bromoquinolin-3-amine

A 5 L 4-necked round-bottom flask was charged with a solution of 6-bromo-3-nitroquinoline-1-oxide (160 g, 595 mmol) in 1:1 ethanol:water (2000 mL), heated to 70° C., charged with iron (160 g, 2.86 mol) in several batches at 70° C., charged with concentrated HCl (10 mL) dropwise and stirred for 3 h at 70° C. The reaction mixture was cooled, filtered and concentrated in vacuo to remove most of the ethanol. The residual solution was extracted with ethyl acetate (3×1 L) and the combined organic layers were dried over sodium sulfate, filtered and concentrated in vacuo to obtain 6-bromoquinolin-3-amine.

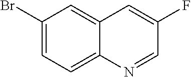 [SMILES: Fc1cnc2ccc(Br)cc2c1]

### Step 4. 6-Bromo-3-fluoroquinoline

A 3 L 4-necked round-bottom flask was charged with a solution of 6-bromoquinolin-3-amine (120 g, 538 mmol) in 70% HF*pyridine (1.2 L). Sodium nitrite (56 g, 810 mmol) was added in several batches at 0° C. over 20 min. The resulting solution was stirred overnight at 10-20° C. The reaction mixture was adjusted to pH 7-8 with aqueous sodium bicarbonate and extracted with ethyl acetate (3×1 L). The organic layers were combined, dried over sodium sulfate, filtered, concentrated in vacuo and purified by silica gel chromatography (1:20 EtOAc/petroleum ether) to obtain 6-bromo-3-fluoroquinoline.

LRMS (ESI) calc'd for C9H6BrFN [M+H]⁺: 226. Found: 226.

### Method D

### Intermediate #23

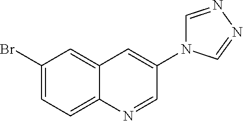 [SMILES: Brc1ccc2ncc(-n3cnnc3)cc2c1]

### 6-Bromo-3-(4H-1,2,4-triazol-4-yl)quinoline

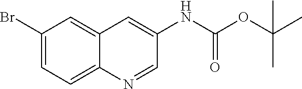 [SMILES: CC(C)(C)OC(=O)Nc1cnc2ccc(Br)cc2c1]

### Step 1. tert-Butyl (6-bromoquinolin-3-yl)carbamate

Triethylamine (0.087 mL, 0.627 mmol) and diphenylphosphoryl azide (0.105 mL, 0.488 mmol) were added to a solution of 6-bromoquinoline-3-carboxylic acid (100 mg, 0.397 mmol) in tert-butyl alcohol (1.5 mL) (Note: the flask was not tightly sealed in order to allow for gas evolution) and the mixture was allowed to stir at 100° C. overnight. Room temperature was attained and the reaction mixture was diluted with water and extracted into EtOAc. The organic layer was washed with saturated aqueous sodium bicarbonate followed by brine, dried over sodium sulfate, filtered, concentrated in vacuo and purified by silica gel flash chromatography (MPLC, gradient elution, 25-100% EtOAc/hexanes) to give tert-butyl (6-bromoquinolin-3-yl)carbamate.

LRMS (ESI) calc'd for C14H16BrN2O2 [M+H]⁺: 323. Found 323.

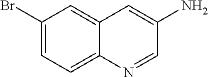 [SMILES: Nc1cnc2ccc(Br)cc2c1]

### Step 2. 6-Bromoquinolin-3-amine

A solution of tert-butyl (6-bromoquinolin-3-yl)carbamate (804 mg, 2.49 mmol) in 1,4-dioxane (8 mL) was charged with 4M HCl in 1,4-dioxane (4 mL) and stirred overnight at ambient temperature. Additional 4M HCl in 1,4-dioxane (4 mL) was added and the mixture was heated at 60° C. for 8 hours. The reaction mixture was then charged with 2 mL of MeOH and heated at reflux for 12 hours, cooled to room temperature and concentrated in vacuo to give 6-bromoquinolin-3-amine.

LRMS (ESI) calc'd for C9H8BrN2 [M+H]⁺: 223. Found 223.

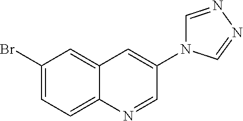 [SMILES: Brc1ccc2ncc(-n3cnnc3)cc2c1]

### Step 3. 6-Bromo-3-(4H-1,2,4-triazol-4-yl)quinoline

6-Bromoquinolin-3-amine (33 mg, 0.15 mmol) and 1,2-diformylhydrazine (14.3 mg, 0.163 mmol) were added to a 20 mL glass vial equipped with a stir bar. The vial was evacuated of air and filled with nitrogen gas and covered with a septum. The solids were allowed to stir neat at 160° C. for 3 days. Room temperature was then attained and the residue was loaded onto silica gel and purified by silica gel flash chromatography (MPLC, gradient elution, 0-20% MeOH/DCM) to give 6-bromo-3-(4H-1,2,4-triazol-4-yl)quinoline.

LRMS (ESI) calc'd for C11H8BrN4 [M+H]⁺: 275. Found 275.

### Method D

### Intermediate #24

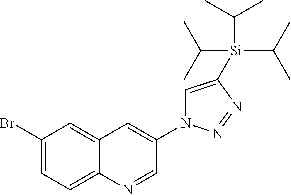 [SMILES: CC(C)[Si](c1cn(-c2cnc3ccc(Br)cc3c2)nn1)(C(C)C)C(C)C]

### 6-Bromo-3-[4-(tripropan-2-ylsilyl)-1H-1,2,3-triazol-1-yl]quinoline

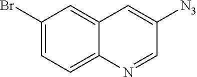 [SMILES: [N-]=[N+]=Nc1cnc2ccc(Br)cc2c1]

### Step 1. 3-Azido-6-bromoquinoline

A solution of 6-bromoquinolin-3-amine (Method D, Intermediate #23, Step 2, 50 mg, 0.22 mmol) in THF (4 mL) was cooled to 0° C. while stirring under nitrogen gas, charged with a solution of sodium nitrite (15.5 mg, 0.224 mmol) in water (0.01 mL) mixture dropwise followed by concentrated sulfuric acid (0.2 mL) and stirred at ambient temperature for 30 minutes. The reaction mixture was charged with a solution of sodium azide (17.5 mg, 0.269 mmol) in water (0.2 mL) while venting the generated nitrogen gas. stirred 20 minutes and then diluted with EtOAc and water. The reaction mixture was extracted into EtOAc (3×) and the combined organic layers were washed with brine, dried over sodium sulfate, and concentrated in vacuo, making sure to not heat the water bath above 25° C., to give 3-azido-6-bromoquinoline.

LRMS (ESI) calc'd for C9H6BrN4 [M+H]⁺: 249. Found 249.

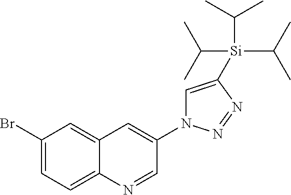 [SMILES: CC(C)[Si](c1cn(-c2cnc3ccc(Br)cc3c2)nn1)(C(C)C)C(C)C]

### Step 2. 6-Bromo-3-[4-(tripropan-2-ylsilyl)-1H-1,2,3-triazol-1-yl]quinoline

A mixture of 3-azido-6-bromoquinoline (60 mg, 0.24 mmol), ethynyl(tripropan-2-yl)silane (0.054 mL, 0.241 mmol) and sodium ascorbate (19.1 mg, 0.096 mmol) in DMF (1 mL) and diethyl ether (1 mL) under a nitrogen atmosphere was charged with a solution of copper (II) sulfate pentahydrate (12.0 mg, 0.048 mmol) in water (0.10 mL). The reaction was allowed to stir overnight at ambient temperature at which point EtOAc and saturated aqueous ammonium chloride were added. The reaction mixture was extracted into EtOAc (3×) and the combined organics were washed with brine, dried over sodium sulfate, filtered, concentrated in vacuo and purified by silica gel flash chromatography (MPLC, gradient elution, 0-35% EtOAc/hexanes) to obtain 6-bromo-3-[4-(tripropan-2-ylsilyl)-1H-1,2,3-triazol-1-yl]quinoline.

LRMS (ESI) calc'd for C20H28BrN4Si [M+H]⁺: 431. Found 431.

The following intermediates were prepared according to Method E following similar procedures described for Intermediate #24, which can be achieved by those of ordinary skill in the art of organic synthesis.

| | | | Exact Mass |
|---|---|---|---|
| Intermediate | Structure | IUPAC Name | [M + H]⁺ |
| 25 | 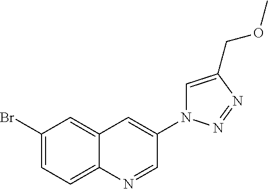 [SMILES: COCc1cn(-c2cnc3ccc(Br)cc3c2)nn1] | 6-bromo-3-[4- (methoxymethyl)-1H-1,2,3- triazol-1-yl]quinoline | Calc'd 319, found 319 |
| 26 | 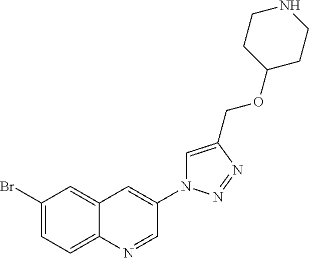 [SMILES: Brc1ccc2ncc(-n3cc(COC4CCNCC4)nn3)cc2c1] | 6-bromo-3-{4-[(piperidin- 4-yloxy)methyl]-1H-1,2,3- triazol-1-yl}quinoline | Calc'd 388, found 388 |

### Method E

### Intermediate #27

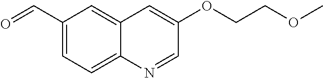 [SMILES: COCCOc1cnc2ccc(C=O)cc2c1]

### 3-(2-Methoxyethoxy)quinoline-6-carbaldehyde

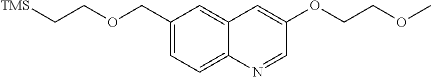 [SMILES: COCCOc1cnc2ccc(COCC[Si](C)(C)C)cc2c1]

### Step 1. 3-(2-Methoxyethoxy)-6-{[2-(trimethylsilyl)ethoxy]methyl}quinoline

6-Chloro-3-(2-methoxyethoxy)quinoline (Intermediate #11, 100 mg, 0.421 mmol), potassium (2-(trimethylsilyl)ethoxy)methyl trifluoroborate (120. mg, 0.505 mmol), palladium (II) acetate (4.7 mg, 0.021 mmol), RuPhos (19.6 mg, 0.042 mmol) and cesium carbonate (411 mg, 1.26 mmol) were loaded into a flask and taken up in 1,4-dioxane (3.8 mL) and water (0.38 mL). The flask was evacuated and back-filled with nitrogen and subsequently heated at 100° C. overnight. After cooling, the crude reaction mixture was diluted with EtOAc, filtered through Celite and concentrated in vacuo onto silica gel. Purification by silica gel flash chromatography (MPLC, gradient elution, 0-100% EtOAc/hexanes) gave 3-(2-methoxyethoxy)-6-{[2-(trimethylsilyl)ethoxy]methyl}quinoline.

LRMS (ESI) calc'd for C18H28NO3Si [M+H]⁺: 334. Found: 334.

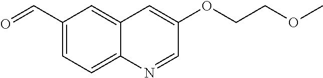 [SMILES: COCCOc1cnc2ccc(C=O)cc2c1]

### Step 2. 3-(2-Methoxyethoxy)quinoline-6-carbaldehyde

3-(2-Methoxyethoxy)-6-{[2-(trimethylsilyl)ethoxy]methyl}quinoline (277 mg, 0.831 mmol) was taken up in DCM (8.3 mL), boron trifluoride diethyl etherate (316 μL, 2.49 mmol) was added and the reaction mixture was stirred at ambient temperature overnight. The crude reaction mixture was concentrated in vacuo. The residue was taken up and stirred in DCM (4.2 mL). Dess-Martin periodinane (388 mg, 0.915 mmol) was added and the solution was warmed to ambient temperature. The crude reaction mixture was filtered through Celite which was rinsed with EtOAc. The filtrate was concentrated in vacuo and purified by silica gel flash chromatography (MPLC, gradient elution, 0-15% MeOH/DCM) to give 3-(2-methoxyethoxy)quinoline-6-carbaldehyde.

LRMS (ESI) calc'd for C13H14NO3 [M+H]⁺: 232. Found: 232.

The following example was prepared according to Method F following similar procedures described for Intermediate #27 using Intermediate #13, which can be achieved by those of ordinary skill in the art of organic synthesis.

| In- | | | Exact |
|---|---|---|---|
| ter- | | | Mass |
| me- | | | [M + |
| diate | Structure | IUPAC Name | H]⁺ |
| 28 | 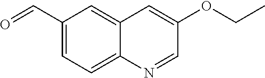 | 3-ethoxy- quinoline-6- carbaldehyde | Calc'd 202, Found 202 |

### Method E

### Intermediate #29

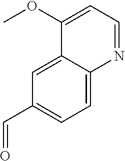

### 4-Methoxyquinoline-6-carbaldehyde

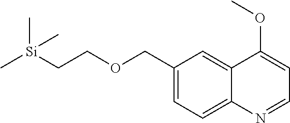 [SMILES: COc1ccnc2ccc(COCC[Si](C)(C)C)cc12]

### Step 1. 4-Methoxy-6-{[2-(trimethylsilyl)ethoxy]methyl}quinoline

A microwave vial was charged with 6-bromo-4-methoxyquinoline (377 mg, 1.58 mmol), potassium (2-trimethylsilyl)-ethoxymethyl trifluoroborate (451 mg, 1.90 mmol), cesium carbonate (1032 mg, 3.17 mmol), DTBPF (30.0 mg, 0.063 mmol), palladium (II) acetate (7 mg, 0.03 mmol), toluene (6.6 mL), and water (1.3 mL) and heated to 100° C. for 19 hours. The crude reaction mixture was cooled to room temperature, filtered through a column pre-packed with Celite, concentrated in vacuo and purified by silica gel flash chromatography (MPLC, gradient elution, 0-15% DCM/MeOH) to obtain 4-methoxy-6-{[2-(trimethylsilyl)ethoxy]methyl}quinoline.

LRMS (ESI) calc'd for C16H24NO2Si [M+H]⁺: 290. Found 290.

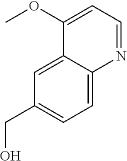

### Step 2. (4-Methoxyquinolin-6-yl)methanol

4-Methoxy-6-{[2-(trimethylsilyl)ethoxy]methyl}quinoline (248 mg, 0.855 mmol) was taken up in DCM (8.6 mL), trifluoro(1,1′-oxydiethane)boron (322 μL, 2.57 mmol) was added and the reaction mixture was stirred for 23 hours at room temperature. The crude reaction mixture was dissolved in ethyl acetate, filtered through a column pre-packed with Celite, concentrated onto silica gel in vacuo and purified by silica gel flash chromatography (MPLC, gradient elution 0-15% DCM/MeOH) to obtain (4-methoxyquinolin-6-yl)methanol. LRMS (ESI) calc'd for C11H12NO2 [M+H]⁺: 190. Found 190.

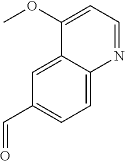

### Step 3. 4-Methoxyquinoline-6-carbaldehyde

A microwave vial equipped with a stir bar was charged with (4-methoxyquinolin-6-yl)methanol (68.5 mg, 0.362 mmol) and DCM (1.8 mL), cooled to 0° C., charged with Dess-Martin periodinane (174 mg, 0.398 mmol) and warmed to room temperature while stirring for 20 hours. The crude reaction mixture was dissolved in ethyl acetate and filtered through a column pre-packed with Celite. The combined organics were concentrated in vacuo to obtain 4-methoxyquinoline-6-carbaldehyde.

LRMS (ESI) calc'd for C11H10NO2 [M+H]⁺: 188. Found 188.

### Method F

### Intermediate #30

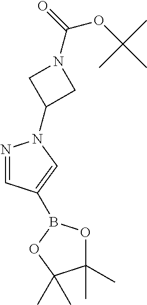 [SMILES: CC(C)(C)OC(=O)N1CC(n2cc(B3OC(C)(C)C(C)(C)O3)cn2)C1]

### tert-Butyl-3-[4-(4,4,5,5-tetramethyl-1,3,2-dioxaborolan-2-yl)-1H-pyrazol-1-yl]azetidine-1-carboxylate

### Step 1. tert-Butyl-3-[4-(4,4,5,5-tetramethyl-1,3,2-dioxaborolan-2-yl)-1H-pyrazol-1-yl]azetidine-1-carboxylate

A 20 mL microwave vial was charged with tert-butyl 3-iodoazetidine-1-carboxylate (2.19 g, 7.73 mmol), cesium carbonate (2.02 g, 6.18 mmol), 4-(4,4,5,5-tetramethyl-1,3,2-dioxaborolan-2-yl)-1H-pyrazole (1.0 g, 5.2 mmol), and DMF (15 mL), sealed with a septum, purged in vacuo, backfilling with argon (3×, releasing excess pressure by venting to balloon) and heated to 150° C. for 25 min. The reaction mixture was diluted with 50 mL EtOAc, 50 mL Et₂O and 30 mL water. The layers were separated and the organics were washed with 30 mL water (2×) followed by 30 mL brine, dried over sodium sulfate, filtered, concentrated in vacuo and purified by silica gel flash chromatography (MPLC, gradient elution, 0.5-10% DCM/MeOH) to give tert-butyl-3-[4-(4,4,5,5-tetramethyl-1,3,2-dioxaborolan-2-yl)-1H-pyrazol-1-yl]azetidine-1-carboxylate.

LRMS (ESI) calc'd for C17H29BN3O4 [M+H]⁺: 350. Found 350.

### Method G

### Intermediate #31

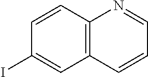 [SMILES: Ic1ccc2ncccc2c1]

### 6-Iodoquinoline

### Step 1. 6-Iodoquinoline

Quinolin-6-ylboronic acid (0.88 g, 5.9 mmol) and N-iodosuccinimide (1.15 g, 5.09 mmol) were placed in a round bottom flask wrapped in aluminum foil and taken up in MeCN (20 mL). The flask was evacuated and back-filled with nitrogen gas (3×) and heated to reflux for 3 hours. Ambient temperature was attained and the solvent removed in vacuo onto silica gel. The residue was purified by silica gel flash chromatography (MPLC, gradient elution, 0-20% EtOAc-toluene) to give 6-iodoquinoline.

LRMS (ESI) calc'd for C9H7NI [M+H]⁺: 256. Found: 256.

### Method H

### Intermediate #32

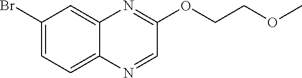 [SMILES: COCCOc1cnc2ccc(Br)cc2n1]

### 7-Bromo-2-(2-methoxyethoxy)quinoxaline

### Step 1. 7-Bromo-2-(2-methoxyethoxy)quinoxaline

A 5 mL microwave vial was charged with 2-chloro-7-bromoquinoxaline (0.10 g, 0.41 mmol), cesium carbonate (0.50 g, 1.5 mmol) and 2-methoxyethanol (2 mL), sealed and heated to 100° C. for 1 hour under microwave irradiation. After cooling to room temperature the reaction mixture was poured into water, extracted with EtOAc (3×) and the combined organics were washed with 2N NaOH (2×) followed by brine, dried over magnesium sulfate, filtered, concentrated in vacuo and dried via lyophilizer to obtain 7-bromo-2-(2-methoxyethoxy)quinoxaline.

LRMS (ESI) calc'd for C11H12BrN2O2 [M+H]⁺: 283. Found: 283.

### Method I

### Intermediate #33

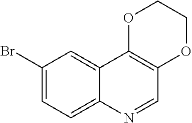 [SMILES: Brc1ccc2ncc3c(c2c1)OCCO3]

### 9-Bromo-2,3-dihydro[1,4]dioxino[2,3-c]quinoline

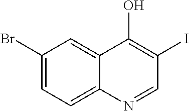 [SMILES: Oc1c(I)cnc2ccc(Br)cc12]

### Step 1. 6-Bromo-3-iodoquinolin-4-ol

6-Bromo-quinolin-4-ol (139.4 g, 0.622 mol) in acetic acid (3600 mL) was treated with N-iodosuccinimide (139.9 g, 0.503 mol) and the mixture was heated at 60° C. for 2 hr, cooled and concentrated in vacuo. Excess sodium bicarbonate solution was added and the solid formed was collected and washed with water (2×2.5 L) and acetone (2×2.5 L) to obtain 6-bromo-3-iodoquinolin-4-ol.

LRMS (ESI) calc'd for C9H6BrINO [M+H]⁺: 350. Found: 350.

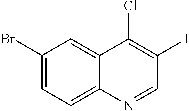 [SMILES: Clc1c(I)cnc2ccc(Br)cc12]

### Step 2. 6-Bromo-4-chloro-3-iodoquinoline

A mixture of 6-bromo-3-iodoquinolin-4-ol (0.47 g, 1.34 mmol) in POCl₃ (5 mL, 53.6 mmol) was heated to 100° C. for 16 hours in a sealed microwave vial, cooled to room temperature, carefully poured onto saturated aqueous sodium bicarbonate over ice and filtered to collect 6-bromo-4-chloro-3-iodoquinoline.

LRMS (ESI) calc'd for C9H5BrClIN [M+H]⁺: 368. Found: 368.

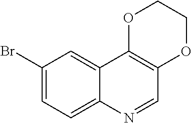 [SMILES: Brc1ccc2ncc3c(c2c1)OCCO3]

### Step 3. 9-Bromo-2,3-dihydro[1,4]dioxino[2,3-c]quinoline

In an oven-dried, nitrogen cooled 5 mL microwave vial was placed 6-bromo-4-chloro-3-iodoquinoline (359.6 mg, 0.976 mmol), cesium carbonate (0.94 g, 2.9 mmol) and copper (I) iodide (0.04 g, 0.2 mmol). The vial was sealed under nitrogen and ethylene glycol (2.0 mL) was added. The reaction mixture was heated to 100° C. for 2 hours under microwave irradiation, cooled to room temperature and extracted with EtOAc (3×). The combined organics were dried over magnesium sulfate, filtered, concentrated in vacuo and purified by silica gel flash chromatography (MPLC, gradient elution, 0-20% MeOH/DCM) to obtain 9-bromo-2,3-dihydro[1,4]dioxino[2,3-c]quinoline.

LRMS (ESI) calc'd for C11H9BrNO2 [M+H]⁺: 266. Found: 266.

### Scheme #15

### Intermediate #34

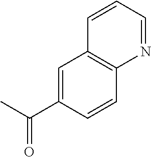

### 1-Quinolin-6-ylethanone

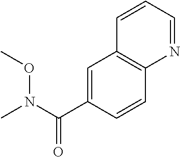 [SMILES: CON(C)C(=O)c1ccc2ncccc2c1]

### Step 1. N-Methoxy-N-methylquinoline-6-carboxamide

A 150 mL pressure vial equipped with a stir bar was charged with quinoline-6-carboxylic acid (1.5 g, 8.7 mmol), N,O-dimethylhydroxylamine hydrochloride (1.7 g, 17 mmol), diisopropyl ethyl amine (6.0 mL, 34.6 mmol), EDC (2.5 g, 13 mmol), HOBT (2.0 g, 13 mmol), and THF (43 mL), sealed and stirred for 18 hours at room temperature. Saturated aqueous sodium bicarbonate was added and the products were extracted with ethyl acetate. The combined organics were washed with brine, dried over magnesium sulfate, filtered, concentrated in vacuo and purified by silica gel flash chromatography (MPLC, gradient elution, 0-10% MeOH/DCM) to obtain N-methoxy-N-methylquinoline-6-carboxamide.

LRMS (ESI) calc'd for C12H13N2O2 [M+H]⁺: 217. Found: 217.

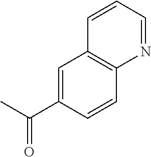

### Step 2. 1-(Quinolin-6-yl)ethanone

N-Methoxy-N-methylquinoline-6-carboxamide (1.6 g, 7.5 mmol) was taken up in THF (37 mL), cooled to −78° C., charged with methyl magnesium bromide (7.5 mL, 22 mmol) and stirred at −78° C. for 10 minutes before warming to room temperature and stirring for 2 hours. The reaction mixture was cooled to −78° C. and quenched with 1 N HCl, neutralized with solid sodium carbonate and extracted with ethyl acetate. The combined organic extracts were dried over magnesium sulfate, filtered, concentrated in vacuo and purified by silica gel flash chromatography (MPLC, gradient elution, 0-15% MeOH/DCM) to obtain 1-(quinolin-6-yl)ethanone.

LRMS (ESI) calc'd for C11H10NO [M+H]⁺: 172. Found: 172.

### Scheme #16

### Intermediate #35

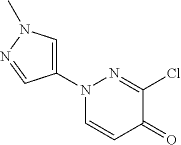

### 3-Chloro-1-(1-methyl-1H-pyrazol-4-yl)pyridazin-4(1H)-one

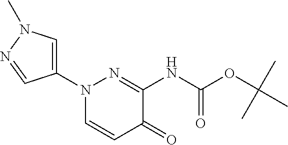 [SMILES: Cn1cc(-n2ccc(=O)c(NC(=O)OC(C)(C)C)n2)cn1]

### Step 1. tert-Butyl [1-(1-methyl-1H-pyrazol-4-yl)-4-oxo-1,4-dihydropyridazin-3-yl]carbamate

A 250 mL round bottom flask was charged with 1-(1-methyl-1H-pyrazol-4-yl)-4-oxo-1,4-dihydropyridazine-3-carboxylic acid (Scheme #1, Intermediate #1, Step 3, 5.0 g, 23 mmol), tert-butyl alcohol (40 mL) and triethylamine (5.0 mL, 369 mmol). Diphenylphosphorylazide (6.0 mL, 28 mmol) was added dropwise and a reflux condenser was attached. The reaction mixture was heated to reflux for 17.5 hours, cooled to room temperature, diluted with water and extracted with EtOAc (3×). The organics were washed successively with saturated aqueous sodium bicarbonate (3×), water (2×) and brine, dried over magnesium sulfate, filtered, concentrated in vacuo and purified by silica gel flash chromatography (MPLC, gradient elution, 0-20% MeOH/DCM) to obtain tert-butyl [1-(1-methyl-1H-pyrazol-4-yl)-4-oxo-1,4-dihydropyridazin-3-yl]carbamate.

LRMS (ESI) calc'd for C13H18N5O3 [M+H]⁺: 292. Found: 292.

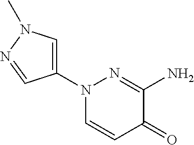

### Step 2. 3-Amino-1-(1-methyl-1H-pyrazol-4-yl)pyridazin-4(1H)-one

To a stirring solution of tert-butyl [1-(1-methyl-1H-pyrazol-4-yl)-4-oxo-1,4-dihydropyridazin-3-yl]carbamate (5.31 g, 18.2 mmol) in 1,4-dioxane (65 mL) was added 4N HCl in 1,4-dioxane (100 mL, 400 mmol). The reaction mixture was stirred for 67 hours at room temperature and then filtered to collect the precipitate which was washed with ethyl acetate followed by hexanes, taken up in methanol, concentrated onto silica gel and purified by silica gel flash chromatography (MPLC, gradient elution, 0-20% MeOH/DCM) to obtain 3-amino-1-(1-methyl-1H-pyrazol-4-yl)pyridazin-4(1H)-one.

LRMS (ESI) calc'd for C8H10N5O [M+H]⁺: 192. Found: 192.

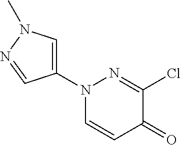

### Step 3. 3-Chloro-1-(1-methyl-1H-pyrazol-4-yl)pyridazin-4(1H)-one

To a suspension of 3-amino-1-(1-methyl-1H-pyrazol-4-yl)pyridazin-4(1H)-one (50 mg, 0.26 mmol) in concentrated hydrochloric acid (2 mL) at 0° C. was added dropwise a solution of sodium nitrite (18.9 mg, 0.274 mmol) in water (0.2 mL). The reaction mixture was stirred at 0° C. for 110 min then carefully quenched with 2N NaOH until basic and extracted with DCM (3×). The combined organics were washed with brine, dried over magnesium sulfate, filtered and concentrated in vacuo to give 3-chloro-1-(1-methyl-1H-pyrazol-4-yl)pyridazin-4(1H)-one.

LRMS (ESI) calc'd for C8H8ClN4O [M+H]⁺: 211. Found: 211.

The following intermediates were prepared according to Scheme #16 following similar procedures described for Intermediate #35, which can be achieved by those of ordinary skill in the art of organic synthesis.

| | | | Exact |
|---|---|---|---|
| Interme- | | | Mass |
| diate | Structure | IUPAC Name | [M + H]⁺ |
| 36 | 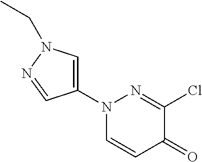 | 3-chloro-1- (1-ethyl-1H- pyrazol-4-yl) pyridazin- 4(1H)-one | Calc'd 225, found 225 |

### Method J

### Intermediate #37

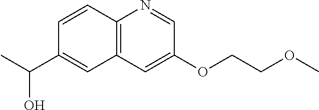 [SMILES: COCCOc1cnc2ccc(C(C)O)cc2c1]

### 1-[3-(2-Methoxyethoxy)quinolin-6-yl]ethanol

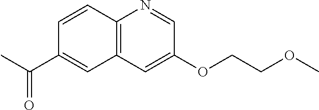 [SMILES: COCCOc1cnc2ccc(C(C)=O)cc2c1]

### Step 1. 1-[3-(2-Methoxyethoxy)quinolin-6-yl]ethanone

A microwave vial was charged with 1,3-bis(diphenylphosphino)propane (18.3 mg, 0.044 mmol), N-cyclohexyl-N-methylcyclohexanamine (208 mg, 1.06 mmol), palladium (II) acetate (5.0 mg, 0.022 mmol), 6-bromo-3-(2-methoxyethoxy)quinoline (Intermediate #15, 250 mg, 0.886 mmol), butyl vinyl ether (230 μL, 1.77 mmol), and DMSO (8.9 mL), sealed and heated to 115° C. for 17.5 hours. The reaction mixture was cooled to room temperature, charged with 1 M HCl. stirred for 30 minutes and washed with ethyl acetate and water. The combined organics were concentrated in vacuo and purified via silica gel flash chromatography (MPLC, gradient elution, 0-15% DCM/MeOH) to obtain 1-[3-(2-methoxyethoxy)quinolin-6-yl]ethanone.

LRMS (ESI) calc'd for C14H16NO3 [M+H]⁺: 246. Found: 246.

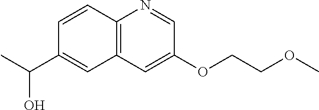 [SMILES: COCCOc1cnc2ccc(C(C)O)cc2c1]

### Step 2. 1-[3-(2-Methoxyethoxy)quinolin-6-yl]ethanol

A solution of 1-[3-(2-methoxyethoxy)quinolin-6-yl]ethanone (217 mg, 0.885 mmol) in MeOH (2949 μL) was charged with sodium borohydride (36.8 mg, 0.973 mmol), stirred at room temperature for 5 minutes, diluted with DCM, filtered through a column pre-packed with Celite, concentrated in vacuo and purified by silica gel flash chromatography (MPLC, gradient elution, 0-15% MeOH/DCM) to obtain 1-[3-(2-methoxyethoxy)quinolin-6-yl]ethanol.

LRMS (ESI) calc'd for C14H18NO3 [M+H]⁺: 248. Found: 248.

The following intermediates were prepared according to Method J following similar procedures described for Intermediate #37 using Intermediates #12, 14, 18 and 34, which can be achieved by those of ordinary skill in the art of organic synthesis.

| | | | Exact |
|---|---|---|---|
| Inter- | | | Mass |
| me- | | IUPAC | [M + |
| diate | Structure | Name | H]⁺ |
| 38 | 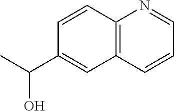 | 1-(quinolin- 6-yl)ethanol | Calc'd 174, found 174 |
| 39 | 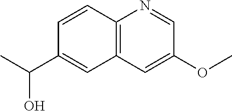 | 1-(3- methoxy- quinolin-6- yl)ethanol | Calc'd 204, found 204 |
| 40 | 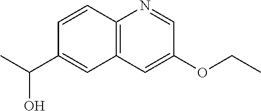 [SMILES: CCOc1cnc2ccc(C(C)O)cc2c1] | 1-(3-ethoxy- quinolin-6- yl)ethanol | Calc'd 218, found 218 |
| 41 | 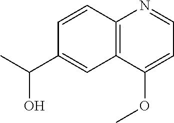 | 1-(4- methoxy- quinolin-6- yl)ethanol | Calc'd 204, found 204 |

### Method K

### Intermediate #42

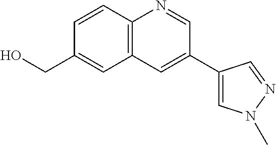 [SMILES: Cn1cc(-c2cnc3ccc(CO)cc3c2)cn1]

### [3-(1-Methyl-1H-pyrazol-4-yl)quinolin-6-yl]methanol

### Step 1. [3-(1-Methyl-1H-pyrazol-4-yl)quinolin-6-yl]methanol

An oven-dried, nitrogen cooled 2 mL microwave vial was charged with bis(pinacolato)diboron (0.30 g, 1.2 mmol), 4,4′-di-tert-butyl-2,2′-dipyridyl (0.04 g, 0.1 mmol) and bis(1,5-cyclooctadiene)di-μ-methoxydiiridium (I) (0.04 g, 0.06 mmol), sealed under a nitrogen atmosphere, charged with CPME (0.5 mL) and stirred at room temperature. Meanwhile, a separate oven-dried, nitrogen cooled 2 mL microwave vial was charged with 6-hydroxymethylquinoline (100 mg, 0.628 mmol), sealed under a nitrogen atmosphere and charged with CPME (0.5 mL). After the first solution stirred for 20 min, it was added to the quinoline-containing vial via syringe and the combined mixture was heated to 100° C. for 15.5 hours. At this point, the reaction mixture was charged with a degassed mixture of N-methyl-4-iodopyrazole (0.30 g, 1.4 mmol), PdCl₂(dppf)-CH₂Cl₂ adduct (0.05 g, 0.06 mmol) and potassium hydroxide (0.18 g, 3.2 mmol) in water (0.5 mL) and CPME (0.5 mL) and stirred at 100° C. for 3 hours. After cooling to room temperature, the reaction mixture was filtered through Celite, concentrated in vacuo and purified by silica gel flash chromatography (MPLC, gradient elution, 0-20% MeOH/DCM, then repurified with 0-20% MeOH/EtOAc) to obtain [3-(1-methyl-1H-pyrazol-4-yl)quinolin-6-yl]methanol.

LRMS (ESI) calc'd for C14H14N3O [M+H]⁺: 240. Found: 240.

The following intermediates were prepared according to Method K following similar procedures described for Intermediate #42 using Intermediate #38, which can be achieved by those of ordinary skill in the art of organic synthesis.

| | | | Exact |
|---|---|---|---|
| Inter- | | | Mass |
| me- | | IUPAC | [M + |
| diate | Structure | Name | H]⁺ |
| 43 | 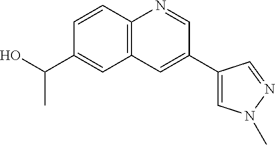 [SMILES: CC(O)c1ccc2ncc(-c3cnn(C)c3)cc2c1] | 1-[3-(1- methyl- 1H- pyrazol- 4-yl) quinolin- 6-yl] ethanol | Calc'd 254, found 254 |

### General Methods

### Scheme #1

### Example #1

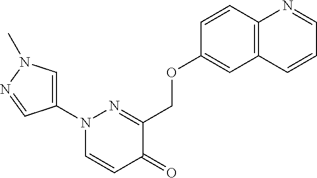 [SMILES: Cn1cc(-n2ccc(=O)c(COc3ccc4ncccc4c3)n2)cn1]

1-(1-Methyl-1H-pyrazol-4-yl)-3-[(quinolin-6-yloxy)methyl]pyridazin-4(1H)-one

### Step 1. 1-(1-Methyl-1H-pyrazol-4-yl)-3-[(quinolin-6-yloxy)methyl]pyridazin-4(1H)-one

A mixture of 3-(chloromethyl)-1-(1-methyl-1H-pyrazol-4-yl)pyridazin-4(1H)-one (Intermediate #1, 0.30 g, 1.3 mmol), potassium carbonate (0.25 g, 1.8 mmol) and 6-hydroxyquinoline (0.21 g, 1.4 mmol) in DMF (2 mL) was heated to 100° C. for 2 hours. After cooling to ambient temperature, water was added and the mixture was filtered. The precipitate was washed with water then dried via lyophilizer to obtain 1-(1-methyl-1H-pyrazol-4-yl)-3-[(quinolin-6-yloxy)methyl]pyridazin-4(1H)-one.

LRMS (ESI) calc'd for C18H16N5O2 [M+H]⁺: 334. Found: 334.

The following examples were prepared according to Scheme #1 following similar procedures described for Example #1 using Intermediates #1-8, which can be achieved by those of ordinary skill in the art of organic synthesis.

| | | | Exact Mass |
|---|---|---|---|
| Example | Structure | IUPAC Name | [M + H]⁺ |
| 2 | 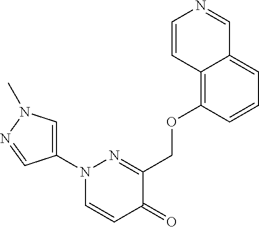 [SMILES: Cn1cc(-n2ccc(=O)c(COc3cccc4cnccc34)n2)cn1] | 3-[(isoquinolin-5- yloxy)methyl]-1-(1- methyl-1H-pyrazol-4- yl)pyridazin-4(1H)- one | Calc'd 334, found 334 |
| 3 | 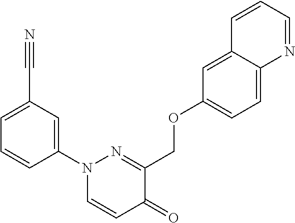 [SMILES: N#Cc1cccc(-n2ccc(=O)c(COc3ccc4ncccc4c3)n2)c1] | 3-{4-oxo-3- [(quinolin-6- yloxy)methyl] pyridazin-1(4H)- yl}benzonitrile | Calc'd 355, found 355 |
| 4 | 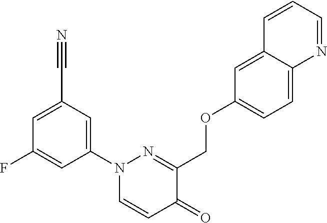 [SMILES: N#Cc1cc(F)cc(-n2ccc(=O)c(COc3ccc4ncccc4c3)n2)c1] | 3-fluoro-5-{4-oxo-3- [(quinolin-6- yloxy)methyl] pyridazin-1(4H)- yl}benzonitrile | Calc'd 373, found 373 |
| 5 | 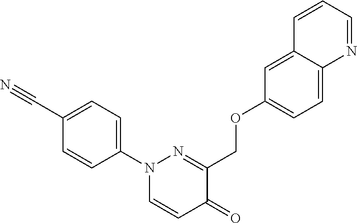 [SMILES: N#Cc1ccc(-n2ccc(=O)c(COc3ccc4ncccc4c3)n2)cc1] | 4-{4-oxo-3- [(quinolin-6- yloxy)methyl] pyridazin-1(4H)- yl}benzonitrile | Calc'd 355, found 355 |
| 6 | 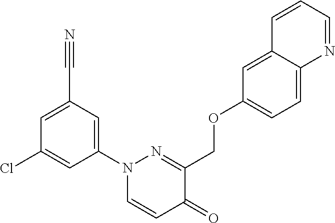 [SMILES: N#Cc1cc(Cl)cc(-n2ccc(=O)c(COc3ccc4ncccc4c3)n2)c1] | 3-chloro-5-{4-oxo-3- [(quinolin-6- yloxy)methyl] pyridazin-1(4H)- yl}benzonitrile | Calc'd 389, found 389 |
| 7 | 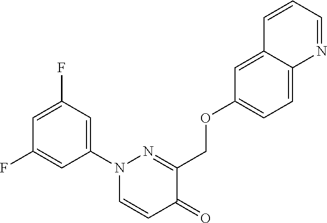 [SMILES: O=c1ccn(-c2cc(F)cc(F)c2)nc1COc1ccc2ncccc2c1] | 1-(3,5- difluorophenyl)-3- [(quinolin-6- yloxy)methyl] pyridazin-4(1H)-one | Calc'd 366, found 366 |
| 8 | 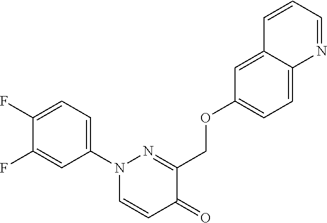 [SMILES: O=c1ccn(-c2ccc(F)c(F)c2)nc1COc1ccc2ncccc2c1] | 1-(3,4- difluorophenyl)-3- [(quinolin-6- yloxy)methyl] pyridazin-4(1H)-one | Calc'd 366, found 366 |
| 9 | 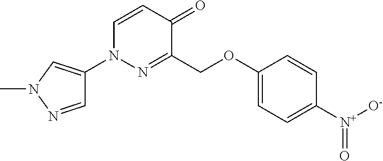 [SMILES: Cn1cc(-n2ccc(=O)c(COc3ccc([N+](=O)[O-])cc3)n2)cn1] | 1-(1-methyl-1H- pyrazol-4-yl)-3-[(4- nitrophenoxy)methyl] pyridazin-4(1H)-one | Calc'd 328, found 328 |
| 10 | 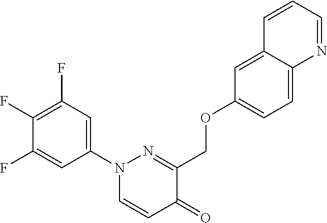 [SMILES: O=c1ccn(-c2cc(F)c(F)c(F)c2)nc1COc1ccc2ncccc2c1] | 3-[(quinolin-6- yloxy)methyl]-1- (3,4,5- trifluorophenyl) pyridazin-4(1H)-one | Calc'd 384, found 384 |
| 11 | 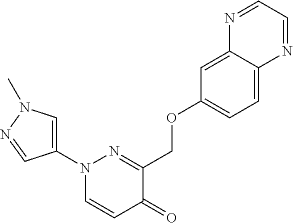 [SMILES: Cn1cc(-n2ccc(=O)c(COc3ccc4nccnc4c3)n2)cn1] | 1-(1-methyl-1H- pyrazol-4-yl)-3- [(quinoxalin-6- yloxy)methyl] pyridazin-4(1H)-one | Calc'd 435, found 335 |
| 12 | 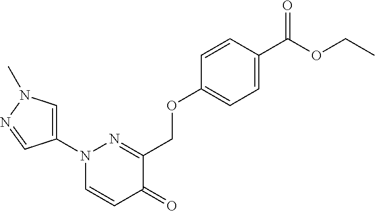 [SMILES: CCOC(=O)c1ccc(OCc2nn(-c3cnn(C)c3)ccc2=O)cc1] | ethyl 4-{[1-(1- methyl-1H-pyrazol-4- yl)-4-oxo-1,4- dihydropyridazin-3- yl]methoxy}benzoate | Calc'd 355, found 355 |
| 13 | 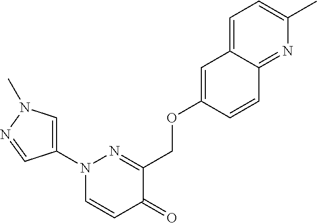 [SMILES: Cc1ccc2cc(OCc3nn(-c4cnn(C)c4)ccc3=O)ccc2n1] | 1-(1-methyl-1H- pyrazol-4-yl)-3-{[2- methylquinolin-6- yl)oxy]methyl} pyridazin-4(1H)-one | Calc'd 348, found 348 |
| 14 | 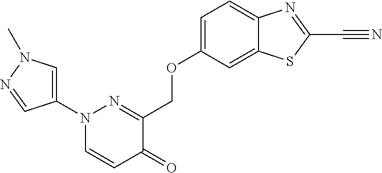 [SMILES: Cn1cc(-n2ccc(=O)c(COc3ccc4nc(C#N)sc4c3)n2)cn1] | 6-{[1-(1-methyl-1H- pyrazol-4-yl)-4-oxo- 1,4-dihydropyridazin- 3-yl]methoxy}-1,3- benzothiazole-2- carbonitrile | Calc'd 365, found 365 |

### Scheme #1

### Example #15

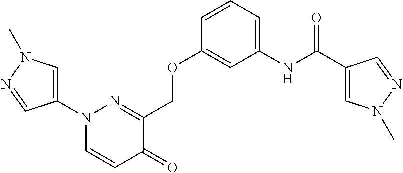 [SMILES: Cn1cc(C(=O)Nc2cccc(OCc3nn(-c4cnn(C)c4)ccc3=O)c2)cn1]

### 1-Methyl-N-(3-{[1-(1-methyl-1H-pyrazol-4-yl)-4-oxo-1,4-dihydropyridazin-3-yl]methoxy}phenyl)-1H-pyrazole-4-carboxamide

### Step 1. 1-Methyl-N-(3-{[1-(1-methyl-1H-pyrazol-4-yl)-4-oxo-1,4-dihydropyridazin-3-yl]methoxy}phenyl-1H-pyrazole-4-carboxamide

A stirring solution of 3-(chloromethyl)-1-(1-methyl-1H-pyrazol-4-yl)pyridazin-4(1H)-one (Intermediate #1, 40 mg, 0.18 mmol) and N-(3-hydroxyphenyl)-1-methyl-1H-pyrazole-4-carboxamide (40 mg, 0.18 mmol) in DMSO (0.5 mL) was charged with 1N sodium hydroxide (36 μL, 0.36 mmol), sealed and heated to 100° C. for 18 hours. After cooling to room temperature, the reaction mixture was quenched with TFA (30 μL, 0.39 mmol), diluted with DMSO and purified by mass-triggered reverse-phase HPLC to obtain 1-methyl-N-(3-{[1-(1-methyl-1H-pyrazol-4-yl)-4-oxo-1,4-dihydropyridazin-3-yl]methoxy}phenyl)-1H-pyrazole-4-carboxamide.

LRMS (ESI) calc'd for C20H20N7O3 [M+H]⁺: 475. Found: 475.

The following examples were prepared according to Scheme #1 following similar procedures described for Example #15 using Intermediate #1, which can be achieved by those of ordinary skill in the at of organic synthesis.

| | | | Exact Mass |
|---|---|---|---|
| Example | Structure | IUPAC Name | [M + H]⁺ |
| 16 | 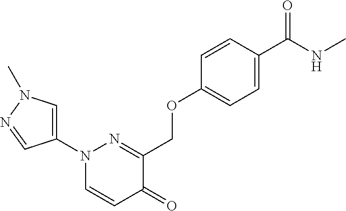 [SMILES: CNC(=O)c1ccc(OCc2nn(-c3cnn(C)c3)ccc2=O)cc1] | N-methyl-4-{[1-(1- methyl-1H-pyrazol-4- yl)-4-oxo-1,4- dihydropyridazin-3- yl]methoxy}benzamide | Calc'd 340, found 340 |
| 17 | 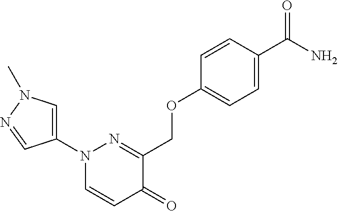 [SMILES: Cn1cc(-n2ccc(=O)c(COc3ccc(C(N)=O)cc3)n2)cn1] | N-{[1-(1-methyl-1H- pyrazol-4-yl)-4-oxo- 1,4-dihydropyridazin- 3-yl]methoxy} benzamide | Calc'd 326, found 326 |
| 18 | 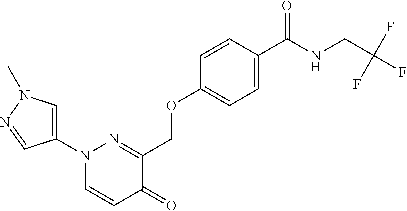 [SMILES: Cn1cc(-n2ccc(=O)c(COc3ccc(C(=O)NCC(F)(F)F)cc3)n2)cn1] | 4-{[1-(1-methyl-1H- pyrazol-4-yl)-4-oxo- 1,4-dihydropyridazin- 3-yl]methoxy}-N- (2,2,2-trifluoroethyl) benzamide | Calc'd 408, found 408 |
| 19 | 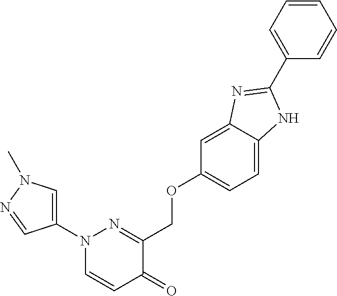 [SMILES: Cn1cc(-n2ccc(=O)c(COc3ccc4[nH]c(-c5ccccc5)nc4c3)n2)cn1] | 1-(1-methyl-1H- pyrazol-4-yl)-3-{[(2- phenyl-1H- benzimidazol-5-yl) oxy]methyl}pyridazin- 4(1H)-one | Calc'd 399, found 399 |

### Scheme #1

### Example #20

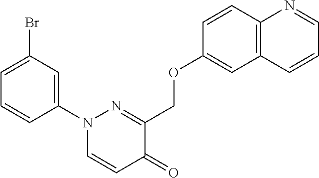 [SMILES: O=c1ccn(-c2cccc(Br)c2)nc1COc1ccc2ncccc2c1]

### 1-(3-Bromophenyl)-3-[(quinolin-6-yloxy)methyl]pyridazin-4(1H)-one

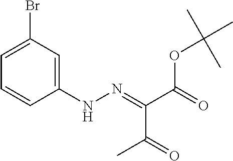 [SMILES: CC(=O)/C(=N\Nc1cccc(Br)c1)C(=O)OC(C)(C)C]

### Step 1. tert-Butyl(2E)-2-[2-(3-bromophenyl)hydrazinylidene]-3-oxobutanoate

3-Bromoaniline (5.0 g, 29 mmol), concentrated HCl (7.27 mL, 87 mmol) and 25 mL of water were combined in a round bottom flask and cooled to 0° C. in an acetone/ice bath. Sodium nitrite (2.21 g, 32.0 mmol) dissolved in 20 mL of water was added dropwise, keeping the temperature of the reaction mixture below 3° C. The reaction mixture was then stirred in an ice bath for an additional 20 minutes. This cold solution was added to a mixture of tert-butylacetoacetate (4.82 mL, 29.1 mmol), ethanol (32.9 mL), sodium acetate (16.7 g, 203 mmol) and 30 mL of water at 0-5° C. over 10 min. The brown suspension was stirred for 30 min. Saturated aqueous sodium bicarbonate solution (20 mL) was slowly added and the solution transferred to a separatory funnel with 50 mL of water. The aqueous layer was extracted with EtOAc (2×100 mL). The organics were combined, then washed with brine, dried over sodium sulfate and filtered. Evaporation of the solvent from the filtrate gave tert-butyl (2E)-2-[2-(3-bromophenyl)hydrazinylidene]-3-oxobutanoate.

LRMS (ESI) calc'd for C14H18BrN2O3 [M+H]⁺: 341. Found: 341.

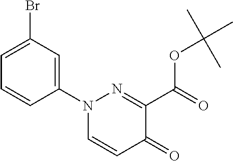 [SMILES: CC(C)(C)OC(=O)c1nn(-c2cccc(Br)c2)ccc1=O]

### Step 2. tert-Butyl 1-(3-bromophenyl)-4-oxo-1,4-dihydropyridazine-3-carboxylate

tert-Butyl (2E)-2-[2-(3-bromophenyl)hydrazinylidene]-3-oxobutanoate (103 g, 302 mmol) and DMFDMA (202 mL, 1510 mmol) were combined in a 100 mL round bottom flask under nitrogen and heated to 90° C. (internal temperature) for 3 hours. After cooling to ambient temperature, 100 mL of hexanes was added, followed by a portionwise addition of 250 mL of 10% aqueous ammonium chloride solution (gas evolution, exothermic) followed by 250 mL of water. After stirring the suspension at ambient temperature for 1 hour, the mixture was filtered and the filter cake washed with water (200 mL) and hexanes (100 mL). Drying overnight gave tert-butyl 1-(3-bromophenyl)-4-oxo-1,4-dihydropyridazine-3-carboxylate.

LRMS (ESI) calc'd for C15H16BrN2O3 [M+H]⁺: 351. Found: 351.

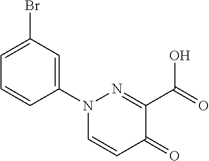 [SMILES: O=C(O)c1nn(-c2cccc(Br)c2)ccc1=O]

### Step 3. 1-(3-bromophenyl)-4-oxo-1,4-dihydropyridazine-3-carboxylic acid

tert-Butyl 1-(3-bromophenyl)-4-oxo-1,4-dihydropyridazine-3-carboxylate (75.0 g, 214 mmol) was dissolved in DCM (534 mL) and stirred at ambient temperature under nitrogen. TFA (82 mL) was added via addition funnel over 20 minutes and the resulting dark solution stirred at ambient temperature for 2 hours. The reaction mixture was concentrated in vacuo to a volume of approximately 50 mL, poured into a mixture of 200 mL of water and 200 mL of Et₂O and stirred vigorously for 1 hour. The precipitate that formed was collected via filtration, and the filter cake washed with 100 mL of water and 100 mL of Et₂O (2×). Drying in vacuo gave 1-(3-bromophenyl)-4-oxo-1,4-dihydropyridazine-3-carboxylic acid.

LRMS (ESI) calc'd for C11H8BrN2O3 [M+H]⁺: 295. Found: 295.

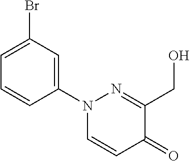 [SMILES: O=c1ccn(-c2cccc(Br)c2)nc1CO]

### Step 4. 1-(3-Bromophenyl)-3-(hydroxymethyl)pyridazin-4(1H)-one

1-(3-Bromophenyl)-4-oxo-1,4-dihydropyridazine-3-carboxylic acid (10.0 g, 33.9 mmol) was suspended in DCM (150 mL) and cooled in an ice/acetone bath. Isobutylchloroformate (5.34 mL, 40.7 mmol) was added via syringe followed by addition of N-methylmorpholine (5.22 mL, 47.4 mmol) via pipette over 20 minutes, keeping the temperature below 2° C. The reaction mixture was stirred in an ice bath for 1 hour. Sodium borohydride (3.2 g, 85 mmol) was suspended in THF (40 mL) and water (4.58 mL) and stirred for 25 minutes at ambient temperature. This suspension was then added to the reaction mixture over the course of minutes, keeping the temperature below 6° C. (significant gas evolution, exothermic), then stirred with cooling for 1 hour. At this point, 50 mL of 10% aqueous ammonium chloride solution was added and the mixture was diluted with 200 mL of DCM, filtered through Celite and rinsed with 100 mL of DCM. The combined organic layers were transferred to a separatory funnel, washed with water and brine, dried over sodium sulfate, filtered, concentrated in vacuo onto silica gel and purified by silica gel flash chromatography (ISCO, 10-90% MeCN/EtOAc) to obtain 1-(3-bromophenyl)-3-(hydroxymethyl)pyridazin-4(1H)-one as a solid.

LRMS (ESI) calc'd for C11H10BrN2O2 [M+H]⁺: 281. Found: 281.

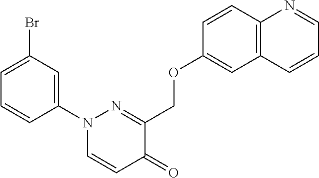 [SMILES: O=c1ccn(-c2cccc(Br)c2)nc1COc1ccc2ncccc2c1]

### Step 1. 1-(3-Bromophenyl)-3-(quinolin-6-yloxy)methyl pyridazin-4(1H)-one

An oven-dried, nitrogen cooled flask was charged with 1-(3-bromophenyl)-3-(hydroxymethyl)pyridazin-4(1H)-one (0.10 g, 0.34 mmol), triphenylphosphine (0.14 g, 0.53 mmol), 6-hydroxyquinoline (0.08 g, 0.6 mmol) and THF (2 mL) followed by DIAD (0.10 mL, 0.51 mmol). The flask was sealed and stirred at ambient temperature for 21 hours. The reaction mixture was concentrated in vacuo and the crude residue was purified by silica gel flash chromatography (MPLC, gradient elution, 0-15% MeOH/EtOAc) to obtain 1-(3-bromophenyl)-3-[(quinolin-6-yloxy)methyl]pyridazin-4(1H)-one.

LRMS (ESI) calc'd for C20H15BrN3O2 [M+H]⁺: 408. Found: 408.

The following examples were prepared according to Scheme #1 following similar procedures described for Example #20, which can be achieved by those of ordinary skill in the art of organic synthesis.

| | | | Exact Mass |
|---|---|---|---|
| Example | Structure | IUPAC Name | [M + H]⁺ |
| 21 | 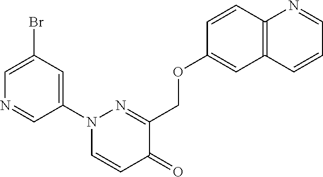 [SMILES: O=c1ccn(-c2cncc(Br)c2)nc1COc1ccc2ncccc2c1] | 1-(5-bromopyridin-3- yl)-3-[(quinolin-6- yloxy)methyl] pyridazin-4(1H)-one | Calc'd 409, found 409 |
| 22 | 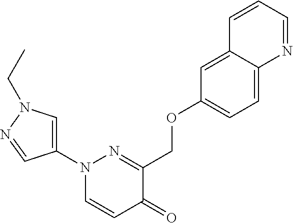 [SMILES: CCn1cc(-n2ccc(=O)c(COc3ccc4ncccc4c3)n2)cn1] | 1-(1-ethyl-1H- pyrazol-4-yl)-3- [(quinolin-6- yloxy)methyl] pyridazin-1(4H)-one | Calc'd 348, found 348 |

### Scheme #1

### Example #23

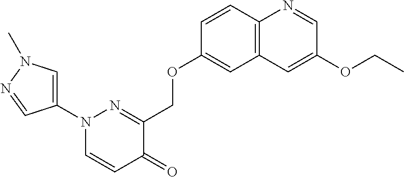 [SMILES: CCOc1cnc2ccc(OCc3nn(-c4cnn(C)c4)ccc3=O)cc2c1]

### 3-{[(3-Ethoxyquinolin-6-yl)oxy]methyl}-1-(1-methyl-1H-pyrazol-4-yl)pyridazin-4(1H)-one

### Step 1. 3-{[(3-Ethoxyquinolin-6-yl)oxy]methyl}-1-(1-methyl-1H-pyrazol-4-yl)pyridazin-4(1H)-one

A 5 mL microwave vial containing 6-chloro-3-ethoxyquinoline (Intermediate #13, 0.11 g, 0.53 mmol) was charged with dipalladium (0) trisdibenzylideneacetone (2.5 mg, 2.7 mmol), Me₄^{t}BuXPhos (5.0 mg, 10 μmol) and freshly ground potassium hydroxide (0.09 g, 2 mmol). The vial was sealed with a septum, evacuated and back-filled with argon (3×) and charged with 1,4-dioxane (0.5 mL) and degassed water (0.5 mL) (degassed by placing water in vacuo and sonicating for ˜30 seconds). The reaction mixture was heated to 100° C. for 15 hours. After cooling to ambient temperature, 3-(chloromethyl)-1-(1-methyl-1H-pyrazol-4-yl)pyridazin-4(1H)-one (Intermediate #1, 0.14 g, 0.62 mmol) was added and the vial was heated to 100° C. for 2 hours. After cooling to ambient temperature, the reaction mixture was filtered through Celite and eluted with EtOAc. The filtrate was concentrated in vacuo and purified by silica gel flash chromatography (MPLC, gradient elution, 0-50% MeOH/EtOAc) to obtain 3-{[(3-ethoxyquinolin-6-yl)oxy]methyl}-1-(1-methyl-1H-pyrazol-4-yl)pyridazin-4(1H)-one.

LRMS (ESI) calc'd for C20H20N5O3 [M+H]⁺: 378. Found: 378.

The following examples were prepared according to Scheme #1 following similar procedures described for Example #23 using Intermediates #1, 3-6, 9-17, 20-23, and 25-26, which can be achieved by those of ordinary skill in the art of organic synthesis.

| | | | Exact Mass |
|---|---|---|---|
| Example | Structure | IUPAC Name | [M + H]⁺ |
| 24 | 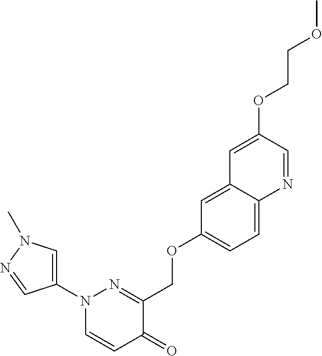 [SMILES: COCCOc1cnc2ccc(OCc3nn(-c4cnn(C)c4)ccc3=O)cc2c1] | 3-({[3-(2- methoxyethoxy) quinolin-6- yl]oxy}methyl)-1- (1-methyl-1H- pyrazol-4- yl)pyridazin- 4(1H)-one | Calc'd 408, found 408 |
| 25 | 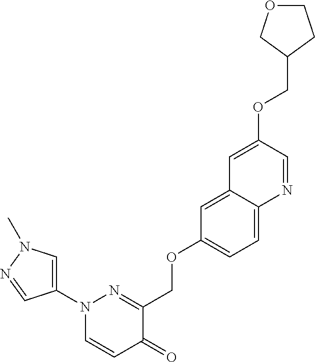 [SMILES: Cn1cc(-n2ccc(=O)c(COc3ccc4ncc(OCC5CCOC5)cc4c3)n2)cn1] | 1-(1-methyl-1H- pyrazol-4-yl)-3- ({[3- (tetrahydrofuran- 3-ylmethoxy) quinolin-6- yl]oxy}methyl) pyridazin-4(1H)- one | Calc'd 434, found 434 |
| 26 | 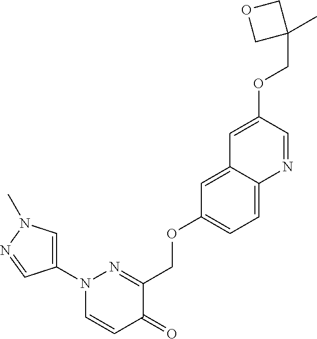 [SMILES: Cn1cc(-n2ccc(=O)c(COc3ccc4ncc(OCC5(C)COC5)cc4c3)n2)cn1] | 3-[({3-[(3- methyloxetan-3- yl)methoxy] quinolin-6- yl}oxy)methyl]-1- (1-methyl-1H- pyrazol-4- yl)pyridazin- 4(1H)-one | Calc'd 434, found 434 |
| 27 | 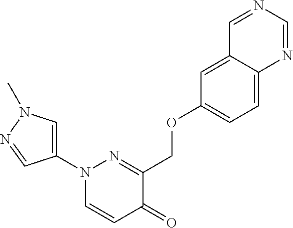 [SMILES: Cn1cc(-n2ccc(=O)c(COc3ccc4ncncc4c3)n2)cn1] | 1-(1-methyl-1H- pyrazol-4-yl)-3- [(quinazolin-6- yloxy)methyl] pyridazin-4(1H)- one | Calc'd 335, found 335 |
| 28 | 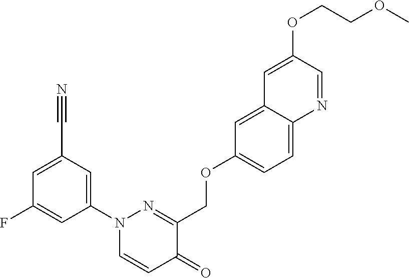 [SMILES: COCCOc1cnc2ccc(OCc3nn(-c4cc(F)cc(C#N)c4)ccc3=O)cc2c1] | 3-fluoro-5-[3-({[3- (2-methoxyethoxy) quinolin-6- yl]oxy}methyl)- 4-oxopyridazin- 1(4H)- yl]benzonitrile | Calc'd 447, found 447 |
| 29 | 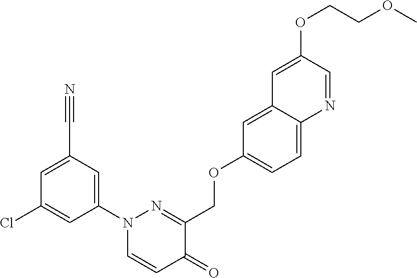 [SMILES: COCCOc1cnc2ccc(OCc3nn(-c4cc(Cl)cc(C#N)c4)ccc3=O)cc2c1] | 3-chloro-5-[3- ({[3-(2- methoxyethoxy) quinolin-6- yl]oxy}methyl)-4- oxopyridazin- 1(4H)- yl]benzonitrile | Calc'd 463, found 463 |
| 30 | 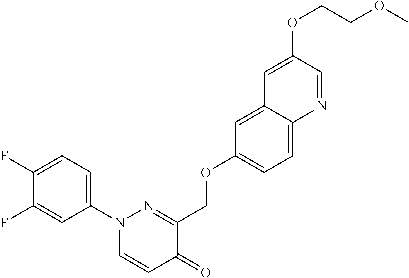 [SMILES: COCCOc1cnc2ccc(OCc3nn(-c4ccc(F)c(F)c4)ccc3=O)cc2c1] | 1-(3,4- difluorophenyl)-3- ({[3-(2- methoxyethoxy) quinolin-6- yl]oxy}methyl) pyridazin-4(1H)- one | Calc'd 440, found 440 |
| 31 | 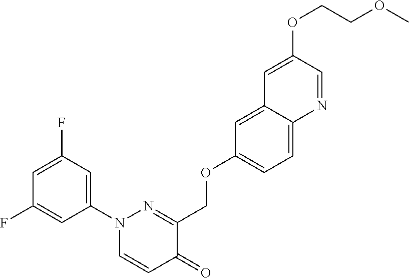 [SMILES: COCCOc1cnc2ccc(OCc3nn(-c4cc(F)cc(F)c4)ccc3=O)cc2c1] | 1-(3,5- difluorophenyl)-3- ({[3-(2- methoxyethoxy) quinolin-6- yl]oxy}methyl) pyridazin-4(1H)- one | Calc'd 440, found 440 |
| 32 | 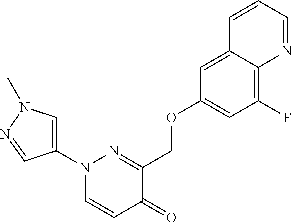 [SMILES: Cn1cc(-n2ccc(=O)c(COc3cc(F)c4ncccc4c3)n2)cn1] | 3-{[(8- fluoroquinolin-6- yl)oxy]methyl}-1- (1-methyl-1H- pyrazol-4- yl)pyridazin- 4(1H)-one | Calc'd 352, found 352 |
| 33 | 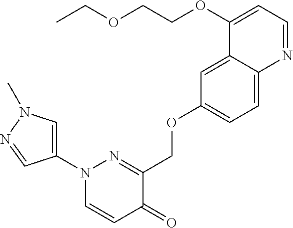 [SMILES: CCOCCOc1ccnc2ccc(OCc3nn(-c4cnn(C)c4)ccc3=O)cc12] | 3-({[4-(2- ethoxyethoxy) quinolin-6- yl]oxy}methyl)- 1-(1-methyl-1H- pyrazol-4- yl)pyridazin- 4(1H)-one | Calc'd 422, found 422 |
| 34 | 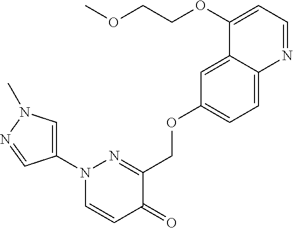 [SMILES: COCCOc1ccnc2ccc(OCc3nn(-c4cnn(C)c4)ccc3=O)cc12] | 3-({[4-(2- methoxyethoxy) quinolin-6- yl]oxy}methyl)-1- (1-methyl-1H- pyrazol-4- yl)pyridazin- 4(1H)-one | Calc'd 408, found 408 |
| 35 | 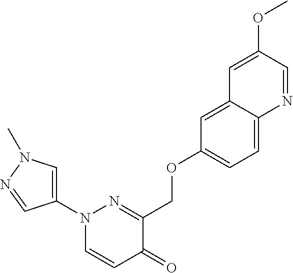 [SMILES: COc1cnc2ccc(OCc3nn(-c4cnn(C)c4)ccc3=O)cc2c1] | 3-{[(3- methoxyquinolin- 6-yl)oxy]methyl}- 1-(1-methyl-1H- pyrazol-4- yl)pyridazin- 4(1H)-one | Calc'd 364, found 364 |
| 36 | 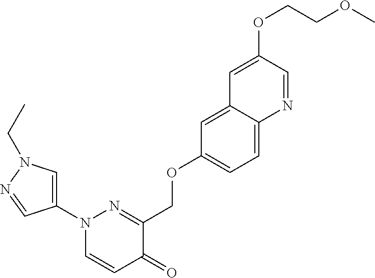 [SMILES: CCn1cc(-n2ccc(=O)c(COc3ccc4ncc(OCCOC)cc4c3)n2)cn1] | 1-(1-ethyl-1H- pyrazol-4-yl)-3- ({[3-(2- methoxyethoxy) quinolin-6- yl]oxy}methyl) pyridazin-4(1H)- one | Calc'd 422, found 422 |
| 37 | 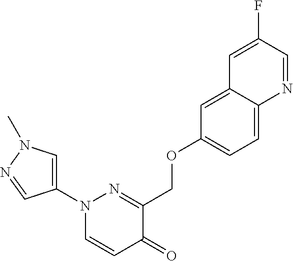 [SMILES: Cn1cc(-n2ccc(=O)c(COc3ccc4ncc(F)cc4c3)n2)cn1] | 3-{[(3- fluoroquinolin-6- yl)oxy]methyl}-1- (1-methyl-1H- pyrazol-4- yl)pyridazin- 4(1H)-one | Calc'd 352, found 352 |
| 38 | 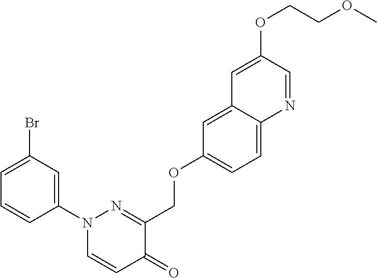 [SMILES: COCCOc1cnc2ccc(OCc3nn(-c4cccc(Br)c4)ccc3=O)cc2c1] | 1-(3- bromophenyl)-3- ({[3-(2- methoxyethoxy) quinolin-6- yl]oxy}methyl) pyridazin-4(1H)- one | Calc'd 482, found 482 |
| 39 | 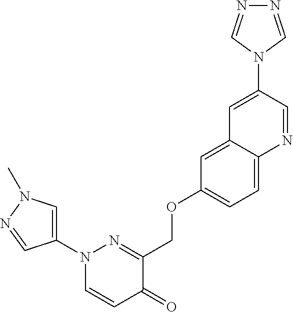 [SMILES: Cn1cc(-n2ccc(=O)c(COc3ccc4ncc(-n5cnnc5)cc4c3)n2)cn1] | 1-(1-methyl-1H- pyrazol-4-yl)-3- ({[3-(4H-1,2,4- triazol-4- yl)quinolin-6- yl]oxy}methyl) pyridazin-4(1H)- one | Calc'd 401, found 401 |
| 40 | 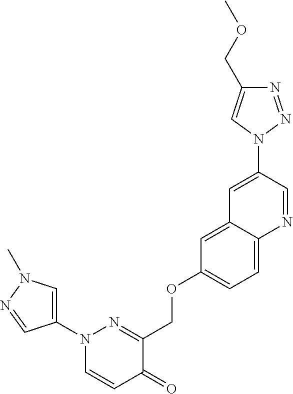 [SMILES: COCc1cn(-c2cnc3ccc(OCc4nn(-c5cnn(C)c5)ccc4=O)cc3c2)nn1] | 3-[({3-[4- (methoxymethyl)- 1H-1,2,3-triazol-1- yl]quinolin-6- yl}oxy)methyl]-1- (1-methyl-1H- pyrazol-4- yl)pyridazin- 4(1H)-one | Calc'd 445, found 445 |
| 41 | 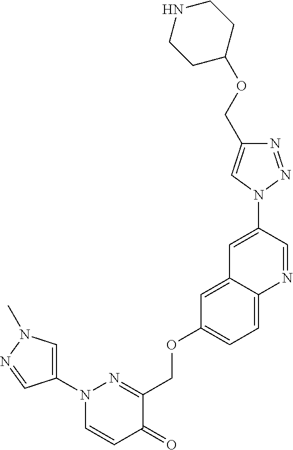 [SMILES: Cn1cc(-n2ccc(=O)c(COc3ccc4ncc(-n5cc(COC6CCNCC6)nn5)cc4c3)n2)cn1] | 1-(1-methyl-1H- pyrazol-4-yl)-3- {[(3-{4- [(piperidin-4- yloxy)methyl]-1H- 1,2,3-triazol-1- yl}quinolin-6- yl)oxy]methyl} pyridazin-4(1H)- one | Calc'd 514, found 514 |

### Scheme #1

### Example #42

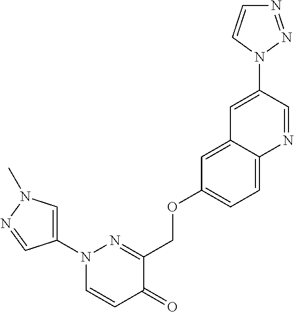 [SMILES: Cn1cc(-n2ccc(=O)c(COc3ccc4ncc(-n5ccnn5)cc4c3)n2)cn1]

### 1-(1-Methyl-1H-pyrazol-4-yl)-3-({[3-(1H-1,2,3-triazol-1-yl)quinolin-6-yl]oxy}methyl)pyridazin-4(1H)-one

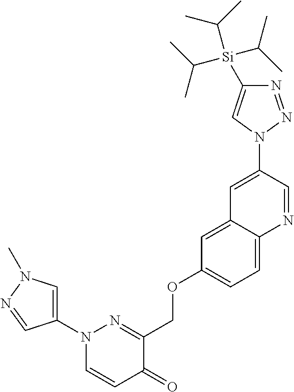 [SMILES: CC(C)[Si](c1cn(-c2cnc3ccc(OCc4nn(-c5cnn(C)c5)ccc4=O)cc3c2)nn1)(C(C)C)C(C)C]

### Step 1. 1-(1-Methyl-1H-pyrazol-4-yl-3-[({3-[4-(tripropan-2-ylsilyl)-1H1,2,3-triazol-1-yl]quinolin-6-yl}oxy)methyl]pyridazin-4(1H)-one

A 5 mL microwave vial was charged with 6-bromo-3-[4-(tripropan-2-ylsilyl)-1H-1,2,3-triazol-1-yl]quinoline (Intermediate #24, 80 mg, 0.185 mmol), dipalladium (0) trisdibenzylideneacetone (1.7 mg, 1.9 μmol), Me₄^{t}BuXPhos (1.8 mg, 3.7 μmol), and freshly ground KOH (33.2 mg, 0.592 mmol), sealed, evacuated and back filled with nitrogen gas (3×), charged with 1,4-dioxane (185 μL) and degassed water (185 μL) (degassed by sealing in a microwave vial, evacuating air while in a sonicator bath for 30 seconds) and heated at 100° C. for 3 hours. The reaction mixture was then cooled to room temperature, charged with 3-(chloromethyl)-1-(1-methyl-1H-pyrazol-4-yl)pyridazin-4(1H)-one (Intermediate #1, 51.2 mg, 0.228 mmol), resealed and the heated at 100° C. overnight. After cooling to room temperature, the reaction mixture was filtered over Celite and eluted with EtOAc. The filtrate was concentrated onto silica gel in vacuo and purified by silica gel flash chromatography (MPLC, gradient elution, 0-10% MeOH/EtOAc) to obtain 1-(1-methyl-1H-pyrazol-4-yl)-3-[({3-[4-(tripropan-2-ylsilyl)-1H-1,2,3-triazol-1-yl]quinolin-6-yl}oxy)methyl]pyridazin-4(1H)-one.

LRMS (ESI) calc'd for C29H37N8O2Si [M+H]⁺: 557. Found 557.

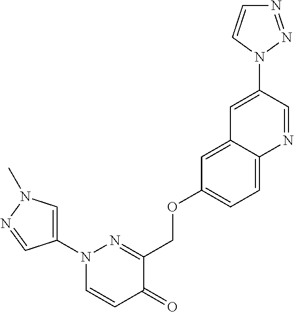 [SMILES: Cn1cc(-n2ccc(=O)c(COc3ccc4ncc(-n5ccnn5)cc4c3)n2)cn1]

### Step 2. 1-(1-Methyl-1H-pyrazol-4-yl)-3-({[3-(1H-1,2,3-triazol-1-yl)quinolin-6-yl]oxy}methyl)pyridazin-4(1H)-one

1-(1-Methyl-1H-pyrazol-4-yl)-3-[({3-[4-(tripropan-2-ylsilyl)-1H-1,2,3-triazol-1-yl]quinolin-6-yl}oxy)methyl]pyridazin-4(1H)-one (50 mg, 0.090 mmol) was taken up in methanol (4.0 mL), charged with 4M KHF₂ (1 mL) and stirred at 85° C. overnight. The mixture was concentrated in vacuo onto silica gel and purified by silica gel flash chromatography (MPLC, gradient elution, 0-15% MeOH/DCM) to obtain 1-(1-methyl-1H-pyrazol-4-yl)-3-({[3-(1H-1,2,3-triazol-1-yl)quinolin-6-yl]oxy}methyl)pyridazin-4(1H)-one.

LRMS (ESI) calc'd for C20H17N8O2 [M+H]⁺: 401. Found 401.

### Scheme #1

### Example #43

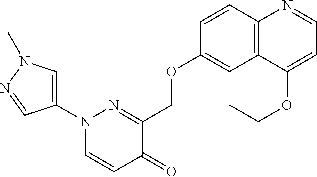 [SMILES: CCOc1ccnc2ccc(OCc3nn(-c4cnn(C)c4)ccc3=O)cc12]

### 3-{[(4-Ethoxyquinolin-6-yl)oxy]methyl}-1-(1-methyl-1H-pyrazol-4-yl)pyridazin-4(1H)-one

### Step 1. 3-{[(4-Ethoxyquinolin-6-yl)oxy]methyl}-1-(1-methyl-1H-pyrazol-4-yl)pyridazin-4(1H)-one

A 5 mL microwave vial containing 6-bromo-3-ethoxyquinoline (Intermediate #19, 0.11 g, 0.53 mmol) was charged with dipalladium (0) trisdibenzylideneacetone (2.5 mg, 2.7 μmol), Me₄^{t}BuXPhos (5.0 mg, 10 μmol) and freshly ground potassium hydroxide (0.09 g, 2 mmol). The vial was sealed with a septum, evacuated and back-filled with nitrogen and charged with 1,4-dioxane (0.5 mL) and degassed water (0.5 mL) (degassed by placing water in vacuo and sonicating for ˜30 seconds). The reaction mixture was heated to 100° C. for 19 hours followed by heating to 130° C. for 1 hour under microwave irradiation. After cooling to ambient temperature, 3-(chloromethyl)-1-(1-methyl-1H-pyrazol-4-yl)pyridazin-4(1H)-one (Intermediate #1, 0.14 g, 0.62 mmol) was added and the vial was heated to 100° C. for 18 hours. After cooling to ambient temperature, the reaction mixture was filtered through Celite and eluted with EtOAc. The filtrate was concentrated in vacuo and purified by silica gel flash chromatography (MPLC, gradient elution, 0-20% MeOH/EtOAc) followed by reverse-phase HPLC (15-50% MeCN/water, 0.1% TFA, freebased with PL-HCO3 cartridges) to obtain 3-{[(4-ethoxyquinolin-6-yl)oxy]methyl}-1-(1-methyl-1H-pyrazol-4-yl)pyridazin-4(1H)-one.

LRMS (ESI) calc'd for C20H20N5O3 [M+H]: 378. Found: 378.

The following examples were prepared according to Scheme #1 following similar procedures described for Example #43 using Intermediates #3-6 and 13-14, which can be achieved by those of ordinary skill in the art of organic synthesis.

| | | | Exact Mass |
|---|---|---|---|
| Example | Structure | IUPAC Name | [M + H]⁺ |
| 44 | 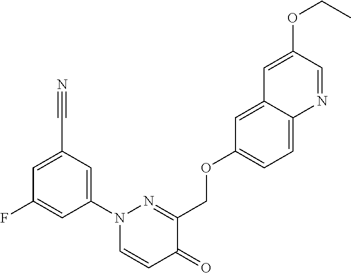 [SMILES: CCOc1cnc2ccc(OCc3nn(-c4cc(F)cc(C#N)c4)ccc3=O)cc2c1] | 3-[3-{[(3- ethoxyquinolin-6- yl)oxy]methyl}-4- oxopyridazin-1(4H)- yl]-5- fluorobenzonitrile | Calc'd 417, found 417 |
| 45 | 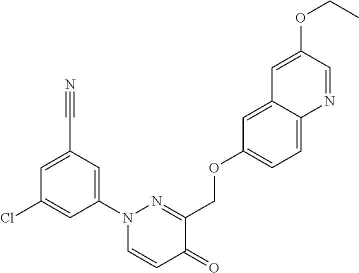 [SMILES: CCOc1cnc2ccc(OCc3nn(-c4cc(Cl)cc(C#N)c4)ccc3=O)cc2c1] | 3-chloro-5-[3-{[(3- ethoxyquinolin-6- yl)oxy]methyl}-4- oxopyridazin-1(4H)- yl]benzonitrile | Calc'd 433, found 433 |
| 46 | 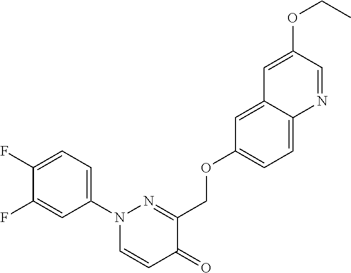 [SMILES: CCOc1cnc2ccc(OCc3nn(-c4ccc(F)c(F)c4)ccc3=O)cc2c1] | 1-(3,4- difluorophenyl)-3- {[(3-ethoxyquinolin- 6- yl)oxy]methyl} pyridazin-4(1H)-one | Calc'd 410, found 410 |
| 47 | 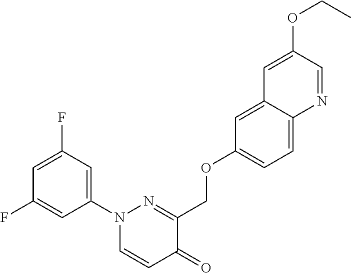 [SMILES: CCOc1cnc2ccc(OCc3nn(-c4cc(F)cc(F)c4)ccc3=O)cc2c1] | 1-(3,5- difluorophenyl)-3- {[(3-ethoxyquinolin- 6-yl)oxy]methyl} pyridazin-4(1H)-one | Calc'd 410, found 410 |

### Scheme #1

### Example #48

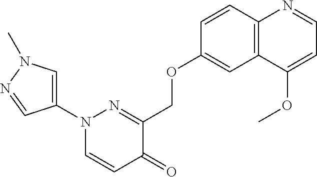 [SMILES: COc1ccnc2ccc(OCc3nn(-c4cnn(C)c4)ccc3=O)cc12]

### 3-{[(4-Methoxyquinolin-6-yl)oxy]methyl}-1-(1-methyl-1H-pyrazol-4-yl)pyridazin-4(1H)-one

### Step 1. 3-{[(4-Methoxyquinolin-6-yl)oxy]methyl}-1-(1-methyl-1H-pyrazol-4-yl)pyridazin-4(1H)-one

An oven-dried, nitrogen cooled 5 mL microwave vial was charged with 6-bromo-3-methoxyquinoline (Intermediate #18, 0.11 g, 0.53 mmol), dipalladium (0) trisdibenzylideneacetone (2.5 mg, 2.7 μmol), Me₄^{t}BuXPhos (5.0 mg, 10 μmol), freshly ground potassium hydroxide (0.09 g, 2 mmol), 1,4-dioxane (0.5 mL) and degassed water (0.5 mL) (degassed by placing water in vacuo and sonicating for −30 seconds), sealed with a septum and heated to 130° C. for 1 hour under microwave irradiation. After cooling to ambient temperature, 3-(chloromethyl)-1-(1-methyl-1H-pyrazol-4-yl)pyridazin-4(1H)-one (Intermediate #1, 0.14 g, 0.62 mmol) was added and the vial was heated to 100° C. for 17.5 hours. After cooling to ambient temperature, the reaction mixture was filtered through Celite and eluted with EtOAc. The filtrate was concentrated in vacuo and purified by silica gel flash chromatography (MPLC, gradient elution, 0-20% MeOH/EtOAc) to obtain 3-{[(4-methoxyquinolin-6-yl)oxy]methyl}-1-(1-methyl-1H-pyrazol-4-yl)pyridazin-4(1H)-one.

LRMS (ESI) calc'd for C19H18N5O3 [M+H]⁺: 364. Found: 364.

The following examples were prepared according to Scheme #1 following similar procedures described for Example #48 using Intermediate #1, which can be achieved by those of ordinary skill in the art of organic synthesis.

| | | | Exact Mass |
|---|---|---|---|
| Example | Structure | IUPAC Name | [M + H]⁺ |
| 49 | 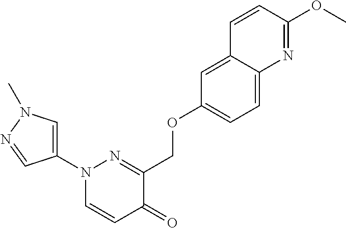 [SMILES: COc1ccc2cc(OCc3nn(-c4cnn(C)c4)ccc3=O)ccc2n1] | 3-{[(2- methoxyquinolin-6- yl)oxy]methyl}-1-(1- methyl-1H-pyrazol-4- yl)pyridazin-4(1H)-one | Calc'd 364, found 364 |
| 50 | 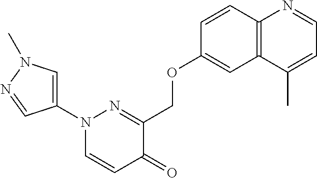 [SMILES: Cc1ccnc2ccc(OCc3nn(-c4cnn(C)c4)ccc3=O)cc12] | 1-(1-methyl-1H- pyrazol-4-yl)-3-{[(4- methylquinolin-6- yl)oxy]methyl}pyridazin- 4(1H)-one | Calc'd 348, found 348 |

### Scheme #1

### Example #51

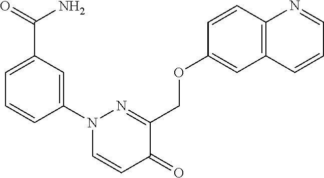 [SMILES: NC(=O)c1cccc(-n2ccc(=O)c(COc3ccc4ncccc4c3)n2)c1]

### 3-{4-Oxo-3-[(quinolin-6-yloxy)methyl]pyridazin-1(4H)-yl}benzamide

### Step 1. 3-{4-Oxo-3-[(quinolin-6-yloxy)methyl]pyridazin-1(4H)-yl}benzamide

A stirring mixture of 3-{4-oxo-3-[(quinolin-6-yloxy)methyl]pyridazin-1(4H)-yl}benzonitrile (Example #3, 62.3 mg, 0.176 mmol) and potassium carbonate (0.05 g, 0.4 mmol) in DMSO (1 mL) was charged with 30% hydrogen peroxide (180 μL, 1.8 mmol) and stirred at ambient temperature for 100 min. The reaction mixture was diluted with water and extracted into EtOAc (3×). The combined organics were washed with water and brine, dried over magnesium sulfate, filtered, concentrated in vacuo, and purified by silica gel flash chromatography (MPLC, gradient elution, 0-20% MeOH/DCM) followed by reverse-phase HPLC (10-100% MeCN/water, 0.1% TFA, freebased with PL-HCO3 SPE cartridges) to obtain 3-{4-oxo-3-[(quinolin-6-yloxy)methyl]pyridazin-1(4H)-yl}benzamide.

LRMS (ESI) calc'd for C21H17N4O3 [M+H]⁺: 373. Found: 373.

### Scheme #1

### Example #52

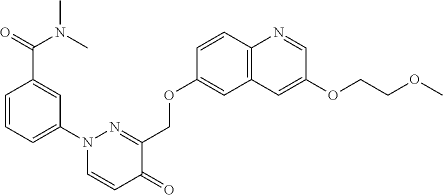 [SMILES: COCCOc1cnc2ccc(OCc3nn(-c4cccc(C(=O)N(C)C)c4)ccc3=O)cc2c1]

### 3-[3-({[3-(2-methoxyethoxy)quinolin-6-yl]oxy}methyl)-4-oxopyridazin-1(4H)-yl]-N,N-dimethylbenzamide

### Step 1. 3-[3-({[3-(2-methoxyethoxy)quinolin-6-yl]oxy}methyl)-4-oxopyridazin-1(4H)-yl]-N,N-dimethylbenzamide

A 2 mL microwave vial was charged with 1-(3-bromophenyl)-3-({[3-(2-methoxyethoxy)quinolin-6-yl]oxy}methyl)pyridazin-4(1H)-one (Example #38, 21.4 mg, 0.044 mmol), palladium (II) acetate (0.5 mg, 2 mmol) and DPPP (1.0 mg, 2.4 mol), sealed with a septum, charged with DMF (0.40 mL) and purged with a stream of carbon monoxide gas. Dimethylamine was then added (0.10 mL, 0.20 mmol) and the reaction mixture was heated to 85° C. overnight under a carbon monoxide atmosphere. Additional dimethylamine (0.10 mL, 0.20 mmol) was added and the reaction mixture was heated to 85° C. for 30 min under a carbon monoxide atmosphere. Heating was then continued under a stream of carbon monoxide gas for 5 hours. The reaction mixture was cooled to room temperature dissolved in DMSO and purified by mass-triggered reverse-phase HPLC to obtain 3-[3-({[3-(2-methoxyethoxy)quinolin-6-yl]oxy}) methyl)-4-oxopyridazin-1(4H)-yl]-N,N-dimethylbenzamide.

LRMS (ESI) calc'd for C26H27N4O5 [M+H]⁺: 475. Found: 475.

### Scheme #2

### Example #53

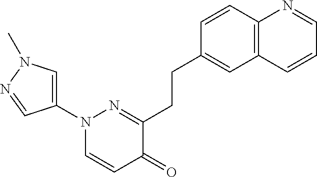 [SMILES: Cn1cc(-n2ccc(=O)c(CCc3ccc4ncccc4c3)n2)cn1]

### 1-(1-Methyl-1H-pyrazol-4-yl)-3-[2-(quinolin-6-yl)ethyl]pyridazin-4(1H)-one

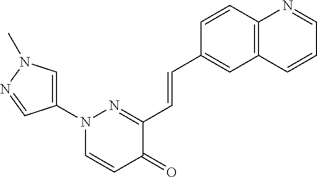 [SMILES: Cn1cc(-n2ccc(=O)c(/C=C/c3ccc4ncccc4c3)n2)cn1]

### Step 1. 1-(1-Methyl-1H-pyrazol-4-yl)-3-[(E)-2-(quinolin-6-yl)ethenyl]pyridazin-4(1H)-one

An oven-dried, nitrogen cooled 5 mL microwave vial was charged with 3-(chloromethyl)-1-(1-methyl-1H-pyrazol-4-yl)pyridazin-4(1H)-one (Intermediate #1, 0.10 g, 0.45 mmol) and triphenylphosphine (0.12 g, 0.46 mmol), sealed under a nitrogen atmosphere, charged with DMF (2 mL) and heated to 100° C. for 4 hours. After cooling to 0° C., the reaction mixture was charged with a 1.78 M solution of potassium tert-butoxide in THF (0.30 mL, 0.53 mmol) followed by quinoline-4-carboxaldehyde (90. mg, 0.57 mmol) and heated to 100° C. for 80 min. After cooling to ambient temperature, the reaction mixture was diluted with water and extracted with DCM/MeOH (3×). The combined organic extracts were dried over magnesium sulfate, filtered, concentrated in vacuo and purified by silica gel flash chromatography (MPLC, gradient elution, 5-40% MeOH/EtOAc) to obtain 1-(1-methyl-1H-pyrazol-4-yl)-3-[(E)-2-(quinolin-6-yl)ethenyl]pyridazin-4(1H)-one.

LRMS (ESI) calc'd for C19H16N5O [M+H]⁺: 330. Found: 330.

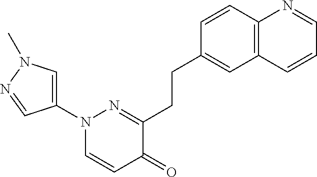 [SMILES: Cn1cc(-n2ccc(=O)c(CCc3ccc4ncccc4c3)n2)cn1]

### Step 2. 1-(1-Methyl-1H-pyrazol-4-yl)-3-[2-(quinolin-6-yl)ethyl]pyridazin-4(1H)-one

A solution of 1-(1-methyl-1H-pyrazol-4-yl)-3-[(E)-2-(quinolin-6-yl)ethenyl]pyridazin-4(1H)-one (87.8 mg, 0.267 mmol) in MeOH (10 mL) was charged with 10% Pd/C (0.04 g, 0.04 mmol). The atmosphere was removed in vacuo, backfilling with a balloon of hydrogen gas (3×). The reaction was stirred at ambient temperature under one atmosphere of hydrogen for 80 min, filtered through Celite, eluted with DCM, concentrated in vacuo and purified by silica gel flash chromatography (MPLC, gradient elution, 10-40% MeOH/EtOAc) to obtain 1-(1-methyl-1H-pyrazol-4-yl)-3-[2-(quinolin-6-yl)ethyl]pyridazin-4(1H)-one.

LRMS (ESI) calc'd for C19H18N5O [M+H]⁺: 332. Found: 332.

The following examples were prepared according to Scheme #2 following similar procedures described for Example #53 using Intermediates #1 and #27-29, which can be achieved by those of ordinary skill in the art of organic synthesis.

| | | | Exact Mass |
|---|---|---|---|
| Example | Structure | IUPAC Name | [M + H]+ |
| 54 | 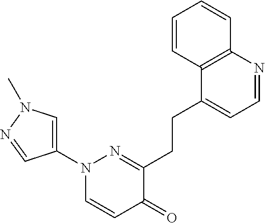 [SMILES: Cn1cc(-n2ccc(=O)c(CCc3ccnc4ccccc34)n2)cn1] | 1-(1-methyl-1H- pyrazol-4-yl)-3-(2- quinolin-4- ylethyl)pyridazin- 4(1H)-one | Calc'd 332, found 332 |
| 55 | 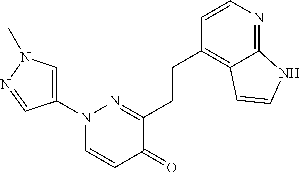 [SMILES: Cn1cc(-n2ccc(=O)c(CCc3ccnc4[nH]ccc34)n2)cn1] | 1-(1-methyl-1H- pyrazol-4-yl)-3-[2- (1H-pyrrolo[2,3- b]pyridin-4- yl)ethyl]pyridazin- 4(1H)-one | Calc'd 321, found 321 |
| 56 | 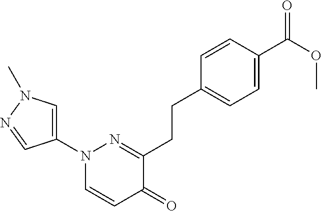 [SMILES: COC(=O)c1ccc(CCc2nn(-c3cnn(C)c3)ccc2=O)cc1] | methyl 4-{2-[1-(1- methyl-1H-pyrazol-4- yl)-4-oxo-1,4- dihydropyridazin-3- yl]ethyl}benzoate | Calc'd 339, found 339 |
| 57 | 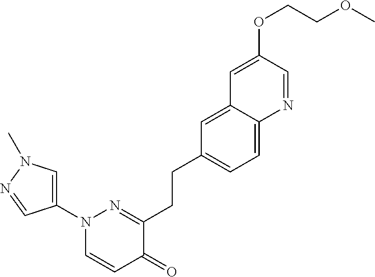 [SMILES: COCCOc1cnc2ccc(CCc3nn(-c4cnn(C)c4)ccc3=O)cc2c1] | 3-{2-[3-(2-methoxy- ethoxy)quinolin- 6-yl]ethyl}-1-(1- methyl-1H-pyrazol-4- yl)pyridazin-4(1H)- one | Calc'd 406, found 406 |
| 58 | 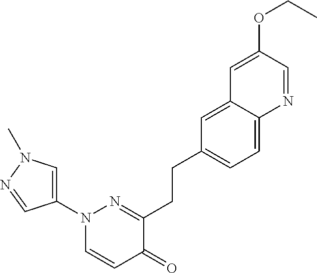 [SMILES: CCOc1cnc2ccc(CCc3nn(-c4cnn(C)c4)ccc3=O)cc2c1] | 3-[2-(3- ethoxyquinolin-6- yl)ethyl]-1-(1-methyl- 1H-pyrazol-4- yl)pyridazin-4(1H)- one | Calcd'd 376, found 376 |
| 59 | 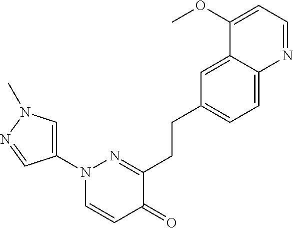 [SMILES: COc1ccnc2ccc(CCc3nn(-c4cnn(C)c4)ccc3=O)cc12] | 3-[2-(4- methoxyquinolin-6- yl)ethyl]-1-(1-methyl- 1H-pyrazol-4- yl)pyridazin-4(1H)- one | Calc'd 362, found 362 |

### Scheme #3

### Example #60

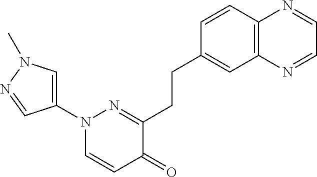 [SMILES: Cn1cc(-n2ccc(=O)c(CCc3ccc4nccnc4c3)n2)cn1]

### 1-(1-Methyl-1H-pyrazol-4-yl)-3-[2-(quinoxalin-6-yl)ethyl]pyridazin-4(1H)-one

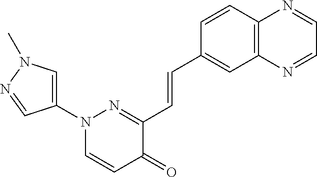 [SMILES: Cn1cc(-n2ccc(=O)c(/C=C/c3ccc4nccnc4c3)n2)cn1]

### Step 1. 1-(1-Methyl-1H-pyrazol-4-yl)-3-[(E)-2-(quinoxalin-6-yl)ethenyl]pyridazin-4(1H)-one

An oven dried, nitrogen cooled 5 mL microwave vial was charged with 3-(chloromethyl)-1-(1-methyl-1H-pyrazol-4-yl)pyridazin-4(1H)-one (Intermediate #1, 150 mg, 0.668 mmol) and triphenylphosphine (180 mg, 0.688 mmol), sealed under nitrogen, charged with DMF (2.7 mL) and heated at 100° C. overnight. The reaction mixture was cooled to 0° C., charged with a 1.78 M solution of potassium tert-butoxide in THF (450 μL, 0.801 mmol) followed by a solution of quinoxaline-6-carbaldehyde (136 mg, 0.861 mmol) in THF (0.67 mL) and heated at 180° C. for 60 minutes under microwave irradiation. The crude reaction mixture was filtered through Celite, eluted with EtOAc, concentrated in vacuo onto silica gel and purified by silica gel flash chromatography (MPLC, gradient elution, 0-25% MeOH/DCM) to give 1-(1-methyl-1H-pyrazol-4-yl)-3-[(E)-2-(quinoxalin-6-yl)ethenyl]pyridazin-4(1H)-one.

LRMS (ESI) calc'd for C18H15N6O [M+H]⁺: 331. Found: 331.

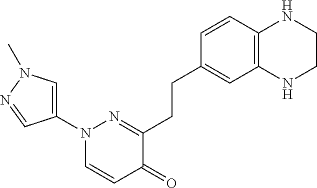 [SMILES: Cn1cc(-n2ccc(=O)c(CCc3ccc4c(c3)NCCN4)n2)cn1]

### Step 2. 1-(1-Methyl-1H-pyrazol-4-yl)-3-[2-(1,2,3,4-tetrahydroquinoxalin-6-yl)ethyl]pyridazin-4(1H)-one

To an oven dried, nitrogen cooled microwave vial was added 1-(1-methyl-1H-pyrazol-4-yl)-3-[(E)-2-(quinoxalin-6-yl)ethenyl]pyridazin-4(1H)-one (60.4 mg, 0.183 mmol) and 10% Pd/C (58.4 mg, 0.055 mmol). The vial was sealed and DCM (914 μL) and EtOH (914 μL) were added. The reaction was stirred under one atmosphere of hydrogen gas at ambient temperature overnight. The crude reaction mixture was diluted with a 1:1 solution of DCM:EtOH, filtered through Celite, concentrated in vacuo onto silica gel and purified by silica gel flash chromatography (MPLC, gradient elution, 0-100% EtOAc/hexanes followed by 0-15% MeOH/DCM) to obtain 1-(1-methyl-1H-pyrazol-4-yl)-3-[2-(1,2,3,4-tetrahydroquinoxalin-6-yl)ethyl]pyridazin-4(1H)-one.

LRMS (ESI) calc'd for C18H21N6O [M+H]⁺: 337. Found: 337.

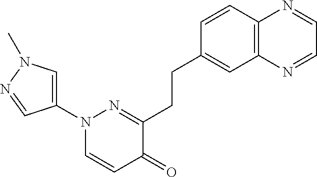 [SMILES: Cn1cc(-n2ccc(=O)c(CCc3ccc4nccnc4c3)n2)cn1]

### Step 3. 1-(1-Methyl-1H-pyrazol-4-yl)-3-[2-(quinoxalin-6-yl)ethyl]pyridazin-4(1H)-one

An oven-dried, nitrogen cooled vial was charged with 1-(1-methyl-1H-pyrazol-4-yl)-3-[2-(1,2,3,4-tetrahydroquinoxalin-6-yl)ethyl]pyridazin-4(1H)-one (5.2 mg, 0.015 mmol) and THF (0.3 mL), cooled to −78° C., charged with ceric ammonium nitrate (25.4 mg, 0.046 mmol) and stirred overnight, allowing to warm to ambient temperature. After cooling to −78° C., additional ceric ammonium nitrate (12.7 mg, 0.023 mmol) was added and stirring was continued for 1.5 hours while warming to ambient temperature. The crude reaction mixture was concentrated in vacuo onto silica gel and purified by silica gel flash chromatography (MPLC, gradient elution, 0-15% MeOH/DCM) to obtain gave the title compound as an oil which was dissolved in acetonitrile and water, frozen and dried on the lyophilizer overnight to give 1-(1-methyl-1H-pyrazol-4-yl)-3-[2-(quinoxalin-6-yl)ethyl]pyridazin-4(1H)-one as a solid.

LRMS (ESI) calc'd for C18H17N6O [M+H]⁺: 333. Found: 333.

### Scheme #4

### Example #61

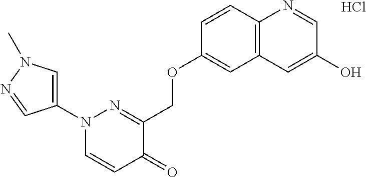 [SMILES: Cn1cc(-n2ccc(=O)c(COc3ccc4ncc(O)cc4c3)n2)cn1 ; Cl]

### 3-Hydroxy-6-{[1-(1-methyl-1H-pyrazol-4-yl)-4-oxo-1,4-dihydropyridazin-3-yl]methoxy}quinolinium chloride

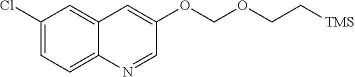 [SMILES: C[Si](C)(C)CCOCOc1cnc2ccc(Cl)cc2c1]

### Step 1. 6-Chloro-3-{[2-(trimethylsilyl)ethoxy]methoxy}quinoline

6-Chloroquinolin-3-ol (Intermediate #11, Step 2, 2.00 g, 11.1 mmol) was dissolved in DMF (10 mL) and THF (10 mL) followed by the portionwise addition of sodium hydride (0.534 g, 13.4 mmol). After 20 minutes at ambient temperature, SEM-Cl (2.37 mL, 13.4 mmol) was added and the mixture was stirred at ambient temperature for 4 hours. Ethyl acetate and saturated aqueous ammonium chloride solution were added to the reaction mixture, the layers were separated and the aqueous fraction was extracted with ethyl acetate (3×). The combined organic layers were washed with brine, dried over sodium sulfate, filtered, concentrated in vacuo and purified by silica gel flash chromatography (MPLC, gradient elution, 0-30% EtOAc/hexanes) to give 6-chloro-3-{[2-(trimethylsilyl)ethoxy]methoxy}quinoline.

LRMS (ESI) calc'd for C15H21ClNO2Si [M+H]⁺: 310. Found: 310.

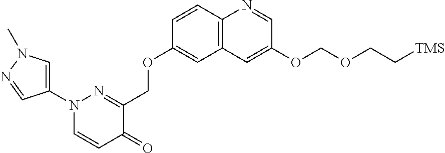 [SMILES: Cn1cc(-n2ccc(=O)c(COc3ccc4ncc(OCOCC[Si](C)(C)C)cc4c3)n2)cn1]

### Step 2. 1-(1-Methyl-1H-pyrazol-4-yl)-3-{[(3-{[(2-(trimethylsilyl)ethoxy]methoxy}quinolin-6-yl)oxy]methyl}pyridazin-4(1H)-one

A 200 mL pressure flask was charged with 6-chloro-3-{[2-(trimethylsilyl)ethoxy]methoxy}quinoline (3.1 g, 9.0 mmol), dipalladium (0) trisdibenzylideneacetone (0.082 g, 0.090 mmol), Me₄^{t}BuXPhos (86 mg, 0.18 mmol), freshly ground potassium hydroxide (1.60 g, 28.6 mmol) 1,4-dioxane (20 mL) and degassed water (2 mL), purged with nitrogen gas for 30 minutes, sealed and heated to 100° C. overnight. After cooling to ambient temperature, the reaction mixture was charged with 3-(chloromethyl)-1-(1-methyl-1H-pyrazol-4-yl)pyridazin-4(1H)-one (Intermediate #1, 2.47 g, 8.95 mmol), and heated at 100° C. for 2.5 hours. After cooling to ambient temperature, the mixture was filtered through Celite, concentrated in vacuo onto silica gel and purified by silica gel flash chromatography (MPLC, gradient elution, 0-10% MeOH/EtOAc) to give 1-(1-methyl-1H-pyrazol-4-yl)-3-{[(3-{[2-(trimethylsilyl)ethoxy]methoxy}quinolin-6-yl)oxy]methyl}pyridazin-4(1H)-one.

LRMS (ESI) calc'd for C24H30N5O4Si [M+H]⁺: 480. Found: 480.

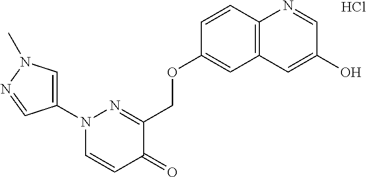 [SMILES: Cn1cc(-n2ccc(=O)c(COc3ccc4ncc(O)cc4c3)n2)cn1 ; Cl]

### Step 3. 3-Hydroxy-6-{[1-(1-methyl-1H-pyrazol-4-yl)-4-oxo-1,4-dihydropyridazin-3-yl]methoxy}quinolinium chloride

1-(1-Methyl-1H-pyrazol-4-yl)-3-{[(3-{[2-(trimethylsilyl)ethoxy]methoxy}quinolin-6-yl)oxy]methyl}pyridazin-4(1H)-one (3.59 g, 7.49 mmol) was stirred in a mixture of EtOH (8 mL) and 2 N HCl (8 mL) at ambient temperature overnight. The reaction mixture was concentrated in vacuo to give 3-hydroxy-6-{[1-(1-methyl-1H-pyrazol-4-yl)-4-oxo-1,4-dihydropyridazin-3-yl]methoxy}quinolinium chloride.

LRMS (ESI) calc'd for C18H16N5O3 [M+H]⁺: 350. Found: 350.

### Scheme #4

### Example #62

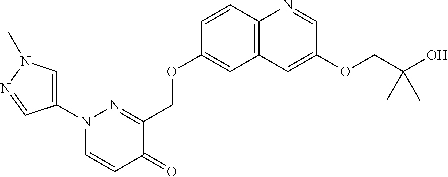 [SMILES: Cn1cc(-n2ccc(=O)c(COc3ccc4ncc(OCC(C)(C)O)cc4c3)n2)cn1]

### 3-({[3-(2-Hydroxy-2-methylpropoxy)quinolin-6-yl]oxy}methyl)-1-(1-methyl-1H-pyrazol-4-yl)pyridazin-4(1H)-one

### Step 1. 3-({[3-(2-Hydroxy-2-methylpropoxy)quinolin-6-yl]oxy}methyl)-1-(1-methyl-1H-pyrazol-4-yl)pyridazin-4(1H)-one

A 5 mL microwave vial was charged with 3-hydroxy-6-{[1-(1-methyl-1H-pyrazol-4-yl)-4-oxo-1,4-dihydropyridazin-3-yl]methoxy}quinolinium chloride (Example #61, 50 mg, 0.13 mmol), isobutylene oxide (0.057 mL, 0.65 mmol) and potassium carbonate (71.6 mg, 0.518 mmol), evacuated and back-filled with nitrogen gas, charged with DMF (2 mL) and heated to 150° C. for 30 minutes under microwave irradiation. Additional potassium carbonate (71.6 mg, 0.518 mmol) and isobutylene oxide (0.057 mL, 0.65 mmol) were added and the reaction was heated to 150° C. for an additional 30 minutes under microwave irradiation. After cooling to ambient temperature, the reaction mixture was filtered through Celite, concentrated in vacuo (using high vacuum to ensure removal of DMF), purified by reverse-phase HPLC (5-50% MeCN/water, 0.1% TFA) and neutralized with PL-HCO3 SPE cartridge to obtain 3-({[3-(2-hydroxy-2-methylpropoxy)quinolin-6-yl]oxy}methyl)-1-(1-methyl-1H-pyrazol-4-yl)pyridazin-4(1H)-one.

LRMS (ESI) calc'd for C22H24N5O4 [M+H]⁺: 422. Found: 422.

### Scheme #4

### Example #63

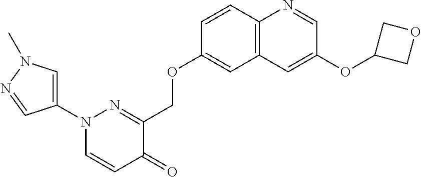 [SMILES: Cn1cc(-n2ccc(=O)c(COc3ccc4ncc(OC5COC5)cc4c3)n2)cn1]

### 1-(1-Methyl-1H-pyrazol-4-yl)-3-({[3-(oxetan-3-yloxy)quinolin-6-yl]oxy}methyl)pyridazin-4(1H)-one

### Step 1. 1-(1-Methyl-1H-pyrazol-4-yl)-3-({[3-(oxetan-3-yloxy)quinolin-6-yl]oxy}methyl)pyridazin-4(1H)-one

A 5 mL microwave vial was charged with 3-hydroxy-6-{[1-(1-methyl-1H-pyrazol-4-yl)-4-oxo-1,4-dihydropyridazin-3-yl]methoxy}quinolinium chloride (Example #61, 50. mg, 0.13 mmol), 3-iodooxetane (119 mg, 0.648 mmol) and potassium carbonate (71.6 mg, 0.518 mmol), sealed, evacuated and back-filled with nitrogen gas, charged with DMF (2 mL) and heated to 150° C. for 30 minutes under microwave irradiation. After cooling to ambient temperature, the reaction mixture was filtered through Celite, concentrated in vacuo (using high vacuum to ensure removal of DMF), purified by reverse-phase HPLC (5-50% MeCN/water, 0.1% TFA) and neutralized with PL-HCO3 SPE cartridge to obtain 1-(1-methyl-1H-pyrazol-4-yl)-3-({[3-(oxetan-3-yloxy)quinolin-6-yl]oxy}methyl)pyridazin-4(1H)-one.

LRMS (ESI) calc'd for C21H20N5O4 [M+H]⁺: 406. Found: 406.

The following examples were prepared according to Scheme #4 following similar procedures described for Example #63, which can be achieved by those of ordinary skill in the art of organic synthesis.

| | | | Exact Mass |
|---|---|---|---|
| Example | Structure | IUPAC Name | [M + H]+ |
| 64 | 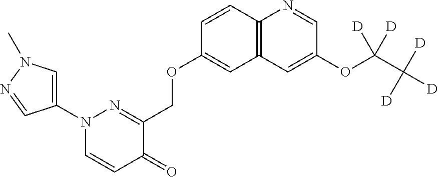 [SMILES: [2H]C([2H])([2H])C([2H])([2H])Oc1cnc2ccc(OCc3nn(-c4cnn(C)c4)ccc3=O)cc2c1] | 3-[({3- [(²H₅)ethyloxy]quino- lin-6- yl}oxy)methyl]-1- (1-methyl-1H- pyrazol-4- yl)pyridazin- 4(1H)-one | Calc'd 383, found 383 |
| 65 | 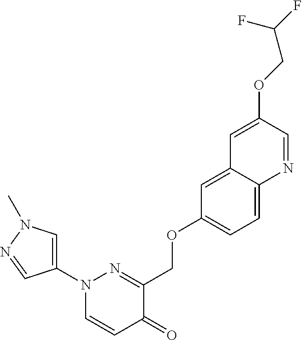 [SMILES: Cn1cc(-n2ccc(=O)c(COc3ccc4ncc(OCC(F)F)cc4c3)n2)cn1] | 3-({[3-(2,2- difluoroethoxy)quino- lin-6- yl]oxy}methyl)-1- (1-methyl-1H- pyrazol-4- yl)pyridazin- 4(1H)-one | Calc'd 414, found 414 |
| 66 | 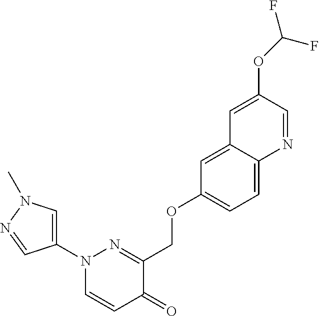 [SMILES: Cn1cc(-n2ccc(=O)c(COc3ccc4ncc(OC(F)F)cc4c3)n2)cn1] | 3-({[3- (difluoromethoxy) quinolin-6- yl]oxy}methyl)-1- (1-methyl-1H- pyrazol-4- yl)pyridazin- 4(1H)-one | Calc'd 400, found 400 |
| 67 | 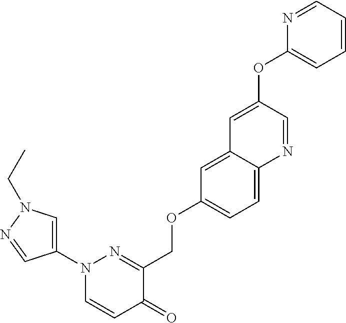 [SMILES: CCn1cc(-n2ccc(=O)c(COc3ccc4ncc(Oc5ccccn5)cc4c3)n2)cn1] | 1-(1-ethyl-1H- pyrazol-4-yl)-3- ({[3-(pyridin-2- yloxy)quinolin-6- yl]oxy}methyl)pyri- dazin-4(1H)-one | Calc'd 441, found 441 |
| 68 | 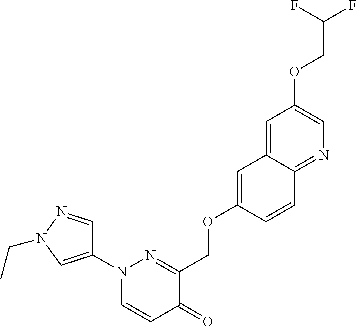 [SMILES: CCn1cc(-n2ccc(=O)c(COc3ccc4ncc(OCC(F)F)cc4c3)n2)cn1] | 3-({[3-(2,2- difluoroethoxy)quino- lin-6- yl]oxy}methyl)-1- (1-ethyl-1H- pyrazol-4- yl)pyridazin- 4(1H)-one | Calc'd 428, found 428 |
| 69 | 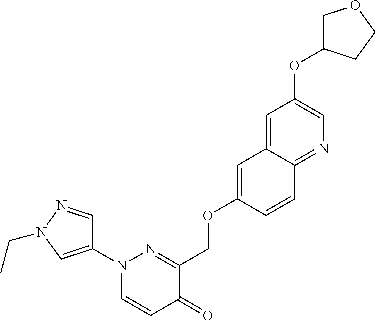 [SMILES: CCn1cc(-n2ccc(=O)c(COc3ccc4ncc(OC5CCOC5)cc4c3)n2)cn1] | 1-(1-ethyl-1H- pyrazol-4-yl)-3- ({[3- (tetrahydrofuran- 3-yloxy)quinolin- 6- yl]oxy}methyl)pyri- dazin-4(1H)-one | Calc'd 434, found 434 |
| 70 | 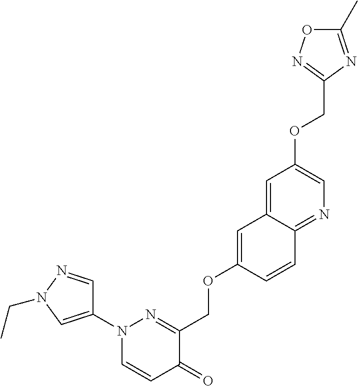 [SMILES: CCn1cc(-n2ccc(=O)c(COc3ccc4ncc(OCc5noc(C)n5)cc4c3)n2)cn1] | 1-(1-ethyl-1H- pyrazol-4-yl)-3- [({3-[(5-methyl- 1,2,4-oxadiazol- 3-yl)methoxy] quinolin-6-yl} oxy)methyl]pyri- dazin-4(1H)-one | Calc'd 460, found 460 |

### Scheme #5

### Example #71

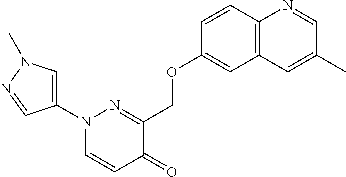 [SMILES: Cc1cnc2ccc(OCc3nn(-c4cnn(C)c4)ccc3=O)cc2c1]

### 1-(1-Methyl-1H-pyrazol-4-yl)-3-{[(3-methylquinolin-6-yl)oxy]methyl}pyridazin-4(1H)-one

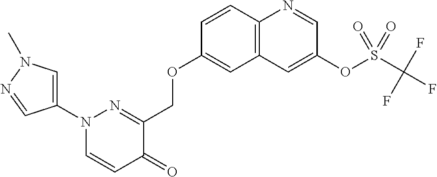 [SMILES: Cn1cc(-n2ccc(=O)c(COc3ccc4ncc(OS(=O)(=O)C(F)(F)F)cc4c3)n2)cn1]

### Step 1. 6-{[1-(1-Methyl-1H-pyrazol-4-yl)-4-oxo-1,4-dihydropyridazin-3-yl]methoxy}quinolin-3-yl trifluoromethanesulfonate

A suspension of 3-hydroxy-6-{[1-(1-methyl-1H-pyrazol-4-yl)-4-oxo-1,4-dihydropyridazin-3-yl]methoxy}quinolinium chloride (Example #61, 1.00 g, 2.59 mmol) in THF (13 mL) was charged with N-phenylbis(trifluoromethyl)sulfonimide (1.02 g, 2.86 mmol) and DIPEA (1.0 mL, 5.7 mmol), stirred at ambient temperature for 6.5 days, poured into saturated aqueous ammonium chloride solution and extracted into EtOAc (3×). The combined organics were washed with saturated aqueous sodium bicarbonate solution and brine, dried over magnesium sulfate, filtered, concentrated in vacuo and purified by silica gel flash chromatography (MPLC, gradient elution, 0-15% MeOH/EtOAc) to obtain 6-{[1-(1-methyl-1H-pyrazol-4-yl)-4-oxo-1,4-dihydropyridazin-3-yl]methoxy}quinolin-3-yl trifluoromethanesulfonate.

LRMS (ESI) calc'd for C19H15F3N5O5S [M+H]⁺: 482. Found: 482.

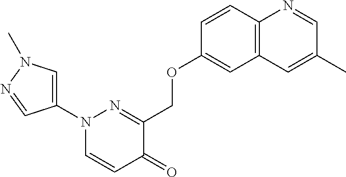 [SMILES: Cc1cnc2ccc(OCc3nn(-c4cnn(C)c4)ccc3=O)cc2c1]

### Step 2. 1-(1-Methyl-1H-pyrazol-4-yl)-3-{[(3-methylquinolin-6-yl)oxy]methyl}pyridazin-4(1H)-one

An oven-dried, nitrogen cooled 2 mL microwave vial was charged with 6-{[1-(1-methyl-1H-pyrazol-4-yl)-4-oxo-1,4-dihydropyridazin-3-yl]methoxy}quinolin-3-yl trifluoromethanesulfonate (40. mg, 0.083 mmol), dipalladium (0) trisdibenzylideneacetone (1.5 mg, 1.6 μmol), SPhos (2.7 mg, 6.6 mmol), cesium carbonate (80. mg, 0.25 mmol) and potassium methyltrifluoroborate (15.2 mg, 0.125 mmol), sealed under nitrogen, charged with toluene (0.5 mL) and degassed water (0.05 mL) and heated to 100° C. for 18 hours. After cooling to ambient temperature, the reaction mixture was filtered through Celite, eluted with EtOAc, concentrated in vacuo and purified by silica gel flash chromatography (MPLC, gradient elution, 0-20% MeOH/DCM) to obtain 1-(1-methyl-1H-pyrazol-4-yl)-3-{[(3-methylquinolin-6-yl)oxy]methyl}pyridazin-4(1H)-one.

LRMS (ESI) calc'd for C19H18N5O2 [M+H]⁺: 348. Found: 348.

The following examples were prepared according to Scheme #5 following similar procedures described for Example #71, which can be achieved by those of ordinary skill in the art of organic synthesis.

| | | | Exact Mass |
|---|---|---|---|
| Example | Structure | IUPAC Name | [M + H]+ |
| 72 | 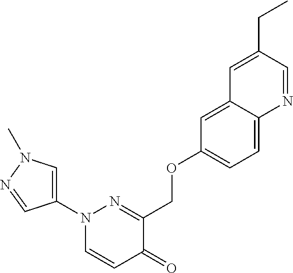 [SMILES: CCc1cnc2ccc(OCc3nn(-c4cnn(C)c4)ccc3=O)cc2c1] | 3-{[(3-ethylquinolin- 6-yl)oxy]methyl}-1- (1-methyl-1H- pyrazol-4- yl)pyridazin-4(1H)- one | Calc'd 362, found 362 |
| 73 | 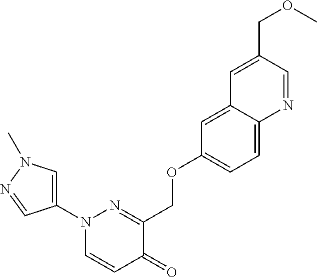 [SMILES: COCc1cnc2ccc(OCc3nn(-c4cnn(C)c4)ccc3=O)cc2c1] | 3-({[3- (methoxymethyl)quino- lin-6- yl]oxy}methyl)-1-(1- methyl-1H-pyrazol-4- yl)pyridazin-4(1H)- one | Calc'd 378, found 378 |

### Scheme #5

### Example #74

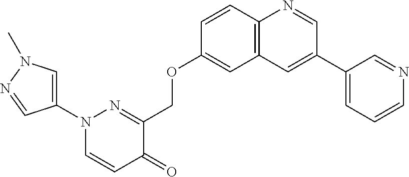 [SMILES: Cn1cc(-n2ccc(=O)c(COc3ccc4ncc(-c5cccnc5)cc4c3)n2)cn1]

### 1-(1-Methyl-1H-pyrazol-4-yl)-3-{[(3-pyridin-3-ylquinolin-6-yl)oxy]methyl}pyridazin-4(1H)-one

### Step 1. 1-(1-Methyl-1H-pyrazol-4-yl)-3-{[(3-pyridin-3-ylquinolin-6-yl)oxy]methyl}pyridazin-4(1H)-one

A 5 mL microwave vial was charged with 6-{[-(1-Methyl-1H-pyrazol-4-yl)-4-oxo-1,4-dihydropyridazin-3-yl]methoxy}quinolin-3-yl trifluoromethanesulfonate (Scheme #5, Example #71, Step 1, 55 mg, 0.11 mmol), pyridin-3-ylboronic acid (28.1 mg, 0.228 mmol), palladium (II) acetate (2.6 mg, 0.011 mmol), tricyclohexylphosphine (6.4 mg, 0.023 mmol) and potassium phosphate tribasic (72.8 mg, 0.343 mmol), evacuated, back-filled with nitrogen gas (3×) and charged with 1,4-dioxane (2.5 mL). The reaction mixture was heated at 100° C. for 3 hours, filtered through Celite, concentrated in vacuo onto silica gel and purified by silica gel flash chromatography (MPLC, gradient elution, 0-15% MeOH/EtOAc) to give 1-(1-methyl-1H-pyrazol-4-yl)-3-{[(3-pyridin-3-ylquinolin-6-yl)oxy]methyl}pyridazin-4(1H)-one.

LRMS (ESI) calc'd for C23H19N6O2 [M+H]⁺: 411. Found: 411.

The following example was prepared according to Scheme #5 following similar procedures described for Example #74 which can be achieved by those of ordinary skill in the art of organic synthesis.

| | | | Exact Mass |
|---|---|---|---|
| Example | Structure | IUPAC Name | [M + H]+ |
| 75 | 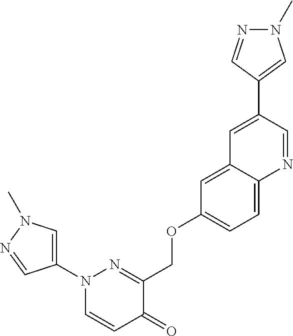 [SMILES: Cn1cc(-c2cnc3ccc(OCc4nn(-c5cnn(C)c5)ccc4=O)cc3c2)cn1] | 1-(1-methyl-1H- pyrazol-4-yl)-3-({[3- (1-methyl-1H- pyrazol-4- yl)quinolin-6- yl]oxy}methyl)pyri- dazin-4(1H)-one | Calc'd 414, found 414 |

### Scheme #5

### Example #76

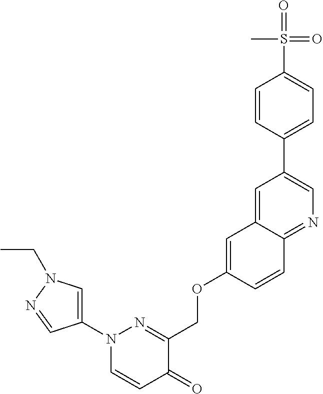 [SMILES: CCn1cc(-n2ccc(=O)c(COc3ccc4ncc(-c5ccc(S(C)(=O)=O)cc5)cc4c3)n2)cn1]

### 1-(1-Ethyl-1H-pyrazol-4-yl)-3-[({3-[4-(methylsulfonyl)phenyl]quinolin-6-yl}oxy)methyl]pyridazin-4(1H)-one

### Step 1. 1-(1-Ethyl-1H-pyrazol-4-yl)-3-[({3-[4-(methylsulfonyl)phenyl]quinolin-6-yl}oxy)methyl]pyridazin-4(1H)-one

A microwave vial was charged with 6-{[1-(1-ethyl-1H-pyrazol-4-yl)-4-oxo-1,4-dihydropyridazin-3-yl]methoxy}quinolin-3-yl trifluoromethanesulfonate (Prepared as described for Scheme #5, Example #71, Step 1 from Intermediate #10, 50 mg, 0.1 mmol), potassium fluoride (17 mg, 0.3 mmol), [4-(methylsulfonyl)phenyl]boronic acid (20 mg, 0.1 mmol), RuPhos (9.3 mg, 0.02 mmol), 1,4-dioxane (0.9 mL), and water (0.1 mL). Dipalladium (0) trisdibenzylideneacetone (9.2 mg, 10 μm) was added last and the reaction mixture was purged with a stream of nitrogen gas for 5 minutes then heated to 80° C. for 18 hours. After cooling to room temperature, the crude reaction mixture was filtered through a column pre-packed with Celite and the filtrate was concentrated in vacuo and purified by mass-directed reverse-phase HPLC to obtain the formate salt of 1-(1-ethyl-1H-pyrazol-4-yl)-3-[({3-[4-(methylsulfonyl)phenyl]quinolin-6-yl}oxy)methyl]pyridazin-4(1H)-one.

LRMS (ESI) calc'd for C26H24N5O4S [M+H]⁺: 502. Found: 502.

The following examples were prepared as the formate salts according to Scheme #5 following similar procedures described for Example #76 which can be achieved by those of ordinary skill in the art of organic synthesis.

| | | | Exact |
|---|---|---|---|
| | | | Mass |
| Example | Structure | IUPAC Name | [M + H]+ |
| 77 | 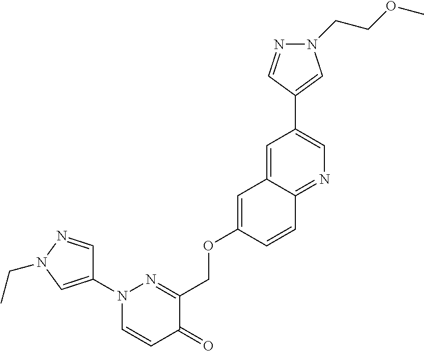 [SMILES: CCn1cc(-n2ccc(=O)c(COc3ccc4ncc(-c5cnn(CCOC)c5)cc4c3)n2)cn1] | 1-(1-ethyl-1H- pyrazol-4-yl)-3-[({3- [1-(2-methoxyethyl)- 1H-pyrazol-4- yl]quinolin-6- yl}oxy)methyl]pyridazin- 4(1H)-one | Calc'd 472, found 472 |
| 78 | 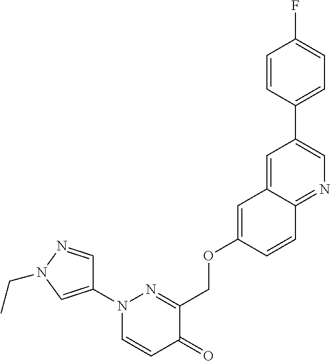 [SMILES: CCn1cc(-n2ccc(=O)c(COc3ccc4ncc(-c5ccc(F)cc5)cc4c3)n2)cn1] | 1-(1-ethyl-1H- pyrazol-4-yl)-3-({[3- (4- fluorophenyl)quinolin- 6- yl]oxy}methyl)pyridazin- 4(1H)-one | Calc'd 442, found 442 |
| 79 | 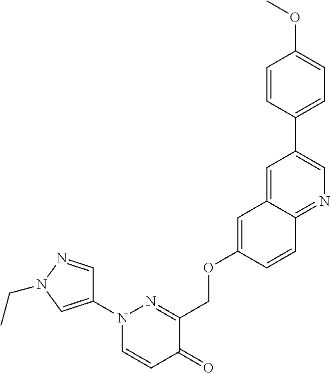 [SMILES: CCn1cc(-n2ccc(=O)c(COc3ccc4ncc(-c5ccc(OC)cc5)cc4c3)n2)cn1] | 1-(1-ethyl-1H- pyrazol-4-yl)-3-({[3- (4- methoxyphenyl)quinolin- 6- yl]oxy}methyl)pyridazin- 4(1H)-one | Calc'd 454, found 454 |
| 80 | 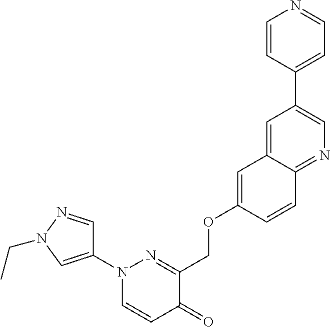 [SMILES: CCn1cc(-n2ccc(=O)c(COc3ccc4ncc(-c5ccncc5)cc4c3)n2)cn1] | 1-(1-ethyl-1H- pyrazol-4-yl)-3-{[(3- pyridin-4-ylquinolin- 6- yl)oxy]methyl}pyridazin- 4(1H)-one | Calc'd 425, found 425 |
| 81 | 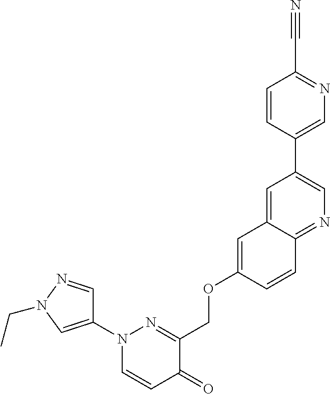 [SMILES: CCn1cc(-n2ccc(=O)c(COc3ccc4ncc(-c5ccc(C#N)nc5)cc4c3)n2)cn1] | 5-(6-{[1-(1-ethyl-1H- pyrazol-4-yl)-4-oxo- 1,4-dihydropyridazin- 3- yl]methoxy}quinolin- 3-yl)pyridine-2- carbonitrile | Calc'd 450, found 450 |
| 82 | 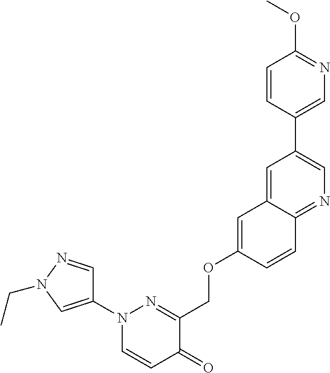 [SMILES: CCn1cc(-n2ccc(=O)c(COc3ccc4ncc(-c5ccc(OC)nc5)cc4c3)n2)cn1] | 1-(1-ethyl-1H- pyrazol-4-yl)-3-({[3- (6-methoxypyridin-3- yl)quinolin-6- yl]oxy}methyl)pyridazin- 4(1H)-one | Calc'd 455, found 455 |
| 83 | 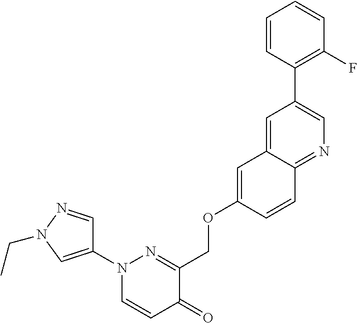 [SMILES: CCn1cc(-n2ccc(=O)c(COc3ccc4ncc(-c5ccccc5F)cc4c3)n2)cn1] | 1-(1-ethyl-1H- pyrazol-4-yl)-3-({[3- (2- fluorophenyl)quinolin- 6- yl]oxy}methyl)pyridazin- 4(1H)-one | Calc'd 442, found 442 |
| 84 | 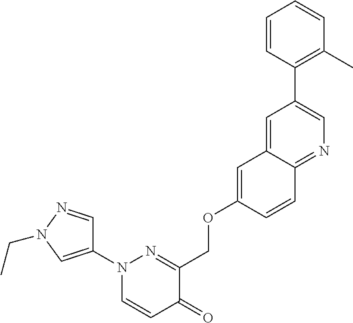 [SMILES: CCn1cc(-n2ccc(=O)c(COc3ccc4ncc(-c5ccccc5C)cc4c3)n2)cn1] | 1-(1-ethyl-1H- pyrazol-4-yl)-3-({[3- (2- methylphenyl)quinolin- 6- yl]oxy}methyl)pyridazin- 4(1H)-one | Calc'd 438, found 438 |
| 85 | 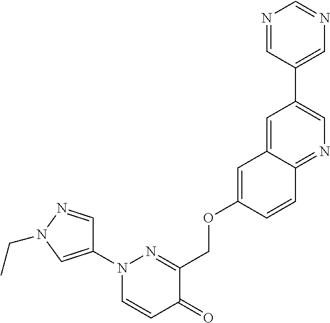 [SMILES: CCn1cc(-n2ccc(=O)c(COc3ccc4ncc(-c5cncnc5)cc4c3)n2)cn1] | 1-(1-ethyl-1H- pyrazol-4-yl)-3-{[(3- pyrimidin-5- ylquinolin-6- yl)oxy]methyl}pyridazin- 4(1H)-one | Calc'd 426, found 426 |
| 86 | 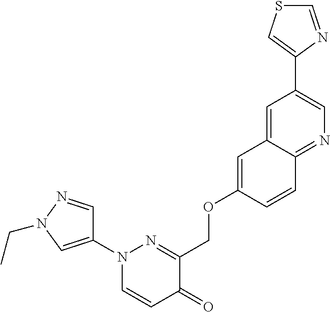 [SMILES: CCn1cc(-n2ccc(=O)c(COc3ccc4ncc(-c5cscn5)cc4c3)n2)cn1] | 1-(1-ethyl-1H- pyrazol-4-yl)-3-({[3- (1,3-thiazol-4- yl)quinolin-6- yl]oxy}methyl)pyridazin- 4(1H)-one | Calc'd 431, found 431 |
| 87 | 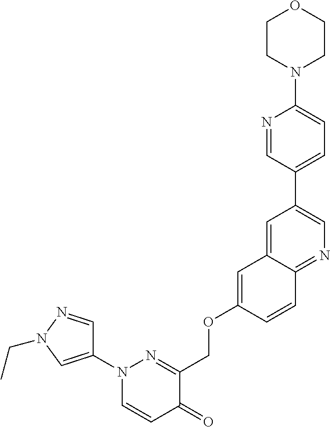 [SMILES: CCn1cc(-n2ccc(=O)c(COc3ccc4ncc(-c5ccc(N6CCOCC6)nc5)cc4c3)n2)cn1] | 1-(1-ethyl-1H- pyrazol-4-yl)-3-({[3- (6-morpholin-4- ylpyridin-3- yl)quinolin-6- yl]oxy}methyl)pyridazin- 4(1H)-one | Calc'd 510, found 510 |
| 88 | 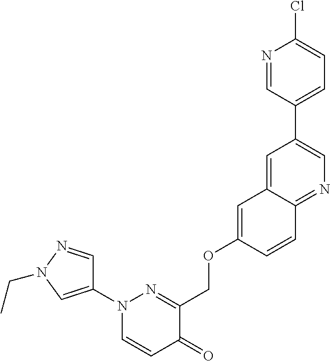 [SMILES: CCn1cc(-n2ccc(=O)c(COc3ccc4ncc(-c5ccc(Cl)nc5)cc4c3)n2)cn1] | 3-({[3-(6- chloropyridin-3- yl)quinolin-6- yl]oxy}methyl)-1-(1- ethyl-1H-pyrazol-4- yl)pyridazin-4(1H)- one | Calc'd 459, found 459 |
| 89 | 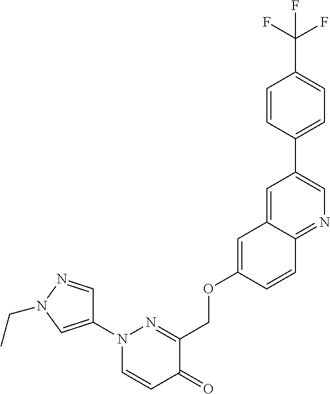 [SMILES: CCn1cc(-n2ccc(=O)c(COc3ccc4ncc(-c5ccc(C(F)(F)F)cc5)cc4c3)n2)cn1] | 1-(1-ethyl-1H- pyrazol-4-yl)-3-[({3- [4- (trifluoromethyl)phenyl] quinolin-6- yl}oxy)methyl]pyridazin- 4(1H)-one | Calc'd 492, found 492 |
| 90 | 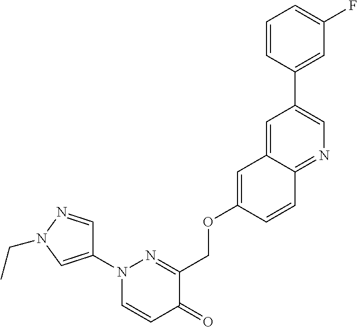 [SMILES: CCn1cc(-n2ccc(=O)c(COc3ccc4ncc(-c5cccc(F)c5)cc4c3)n2)cn1] | 1-(1-ethyl-1H- p yrazol-4-yl)-3-({[3- (3- fluorophenyl)quinolin- 6- yl]oxy}methyl)pyridazin- 4(1H)-one | Calc'd 442, found 442 |
| 91 | 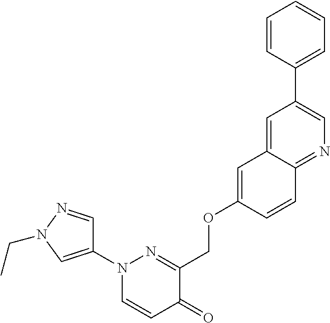 [SMILES: CCn1cc(-n2ccc(=O)c(COc3ccc4ncc(-c5ccccc5)cc4c3)n2)cn1] | 1-(1-ethyl-1H- pyrazol-4-yl)-3-{[(3- phenylquinolin-6- yl)oxy]methyl}pyridazin- 4(1H)-one | Calc'd 424, found 424 |

### Scheme #5

### Example #92

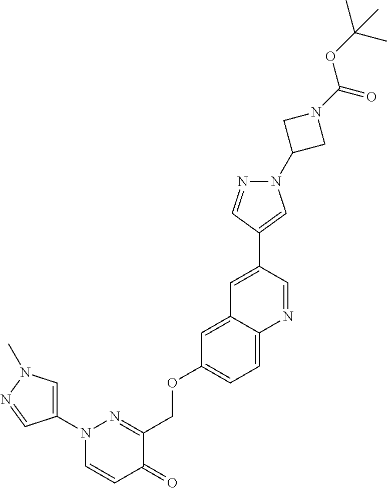 [SMILES: Cn1cc(-n2ccc(=O)c(COc3ccc4ncc(-c5cnn(C6CN(C(=O)OC(C)(C)C)C6)c5)cc4c3)n2)cn1]

### tert-Butyl 3-[4-(6-{[1-(1-methyl-1H-pyrazol-4-yl)-4-oxo-4-dihydropyridazin-3-yl]methoxy}quinolin-3-yl)-1H-pyrazol-1-yl]azetidine-1-carboxylate

### Step 1. tert-Butyl 3-[4-(6-{[1-(1-methyl-1H-pyrazol-4-yl)-4-oxo-1,4-dihydropyridazin-3-yl]methoxy}quinolin-3-yl)-1H-pyrazol-1-yl]azetidine-1-carboxylate

A 2 mL microwave vial was charged with 6-{[1-(1-Methyl-1H-pyrazol-4-yl)-4-oxo-1,4-dihydropyridazin-3-yl]methoxy}quinolin-3-yl trifluoromethanesulfonate (Scheme #5, Example #71, Step #1, 100 mg, 0.208 mmol), tert-butyl-3-[4-(4,4,5,5-tetramethyl-1,3,2-dioxaborolan-2-yl)-1H-pyrazol-1-yl]azetidine-1-carboxylate (Intermediate #30, 110 mg, 0.291 mmol), Pd₂(dba)₃ (9.5 mg, 10 μmol), XPhos (7.9 mg, 0.017 mmol), and cesium carbonate (271 mg, 0.831 mmol), sealed, evacuated and back-filled with nitrogen gas (3×), charged with 1,4-dioxane (2 mL) and heated at 100° C. for 2 hours. After cooling to room temperature, the reaction mixture was filtered through Celite and eluted with DCM and EtOAc. The filtrate was concentrated in vacuo onto silica gel and purified by silica gel flash chromatography (MPLC, gradient elution 0-15% MeOH/DCM) to obtain tert-butyl 3-[4-(6-{[1-(1-methyl-1H-pyrazol-4-yl)-4-oxo-1,4-dihydropyridazin-3-yl]methoxy}quinolin-3-yl)-1H-pyrazol-1-yl]azetidine-1-carboxylate.

LRMS (ESI) calc'd for C29H31N8O4 [M+H]⁺: 555. Found 555.

### Scheme #5

### Example #93

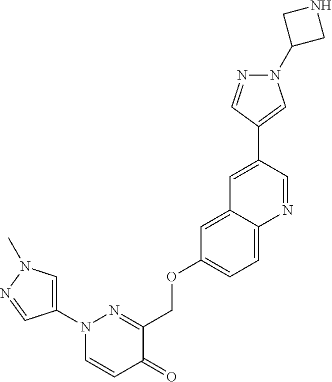 [SMILES: Cn1cc(-n2ccc(=O)c(COc3ccc4ncc(-c5cnn(C6CNC6)c5)cc4c3)n2)cn1]

### 3-[({3-[1-(Azetidin-3-yl)-1H-pyrazol-4-yl]quinolin-6-yl}oxy)methyl]-t-(1-methyl-1H-pyrazol-4-yl)pyridazin-4(1H)-one

### Step 1. 3-[({3-[1-(Azetidin-3-yl)-1H-pyrazol-4-yl]quinolin-6-yl}oxy)methyl]-1-(1-methyl-1H-pyrazol-4-yl)pyridazin-4(1H)-one

A stirring solution of tert-butyl 3-[4-(6-{[1-(1-methyl-1H-pyrazol-4-yl)-4-oxo-1,4-dihydropyridazin-3-yl]methoxy}quinolin-3-yl)-1H-pyrazol-1-yl]azetidine-1-carboxylate (Scheme #5, Example #92, 90 mg, 0.162 mmol) methanol (1 mL) and 1,4-dioxane (1 mL) was charged with 4N HCl in 1,4-dioxane (1 mL) and stirred at ambient temperature for 48 hours. The reaction mixture was concentrated in vacuo, taken up in 10:1 DCM/methanol and neutralized with PL-HCO3 bicarbonate frits to obtain 3-[({3-[1-(azetidin-3-yl)-1H-pyrazol-4-yl]quinolin-6-yl}oxy)methyl]-1-(1-methyl-1H-pyrazol-4-yl)pyridazin-4(1H)-one.

LRMS (ESI) calc'd for C24H23N8O2 [M+H]: 455. Found 455.

The following example was prepared according to Scheme #5 following similar procedures described for Example #93 which can be achieved by those of ordinary skill in the art of organic synthesis.

| | | | Exact Mass |
|---|---|---|---|
| Example | Structure | IUPAC Name | [M + H]+ |
| 94 | 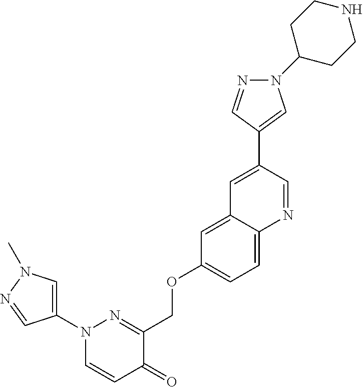 [SMILES: Cn1cc(-n2ccc(=O)c(COc3ccc4ncc(-c5cnn(C6CCNCC6)c5)cc4c3)n2)cn1] | 1-(1-methyl-1H- pyrazol-4-yl)-3-[({3- [1-(piperidin-4-yl)- 1H-pyrazol-4- yl]quinolin-6- yl}oxy)methyl]pyri- dazin-4(1H)-one | Calc'd 483, found 483 |

### Scheme #5

### Example #95

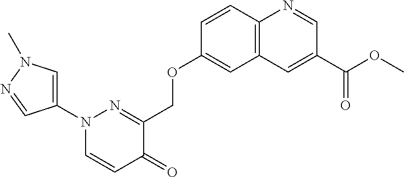 [SMILES: COC(=O)c1cnc2ccc(OCc3nn(-c4cnn(C)c4)ccc3=O)cc2c1]

### Methyl 6-{[1-(1-methyl-1H-pyrazol-4-yl)-4-oxo-1,4-dihydropyridazin-3-yl]methoxy}quinoline-3-carboxylate

### Step 1. Methyl 6-{[1-(1-methyl-1H-pyrazol-4-yl-4-oxo-1,4-dihydropyridazin-3-yl]methoxy}quinoline-3-carboxylate

6-{[1-(1-Methyl-1H-pyrazol-4-yl)-4-oxo-1,4-dihydropyridazin-3-yl]methoxy}quinolin-3-yl trifluoromethanesulfonate (Scheme #5, Example #71, Step 1, 100. mg, 0.208 mmol), palladium (II) acetate (2.3 mg, 10. μmol) and DPPP (4.3 mg, 10. μmol) were taken up in DMF (0.6 mL) and MeOH (0.3 mL). The reaction mixture was purged with a stream of carbon monoxide gas for 5 minutes, charged with DIPEA (0.073 mL, 0.415 mmol) and heated at 70° C. under a balloon of CO overnight. The reaction mixture was concentrated in vacuo onto silica gel and purified by silica gel flash chromatography (MPLC, gradient elution, 0-10% MeOH/DCM). The product solid was triturated with DCM to give methyl 6-{[1-(1-methyl-1H-pyrazol-4-yl)-4-oxo-1,4-dihydropyridazin-3-yl]methoxy}quinoline-3-carboxylate.

LRMS (ESI) calc'd for C20H18N5O4 [M+H]⁺: 392. Found: 392.

### Scheme #5

### Example #96

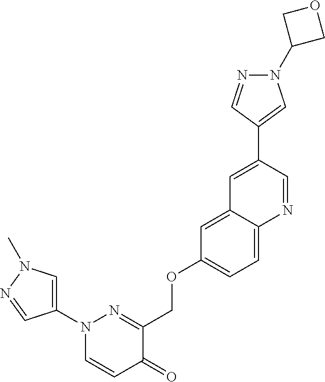 [SMILES: Cn1cc(-n2ccc(=O)c(COc3ccc4ncc(-c5cnn(C6COC6)c5)cc4c3)n2)cn1]

### 1-(1-Methyl-1H-pyrazol-4-yl)-3-[({3-[1-(oxetan-3-yl)-1H-pyrazol-4-yl]quinolin-6-yl}oxy)methyl]pyridazin-4(1H)-one

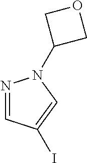

### Step 1. 4-Iodo-1-(oxetan-3-yl)-1H-pyrazole

A 20 mL microwave vial was charged with 3-iodo-oxetane (1.0 g, 5.4 mmol), 4-iodo-1H-pyrazole (1.16 g, 5.98 mmol), and Cs₂CO₃ (1.95 g, 5.98 mmol), sealed, evacuated and back-filled with nitrogen gas (3×), charged with DMF (16 mL) and heated at 150° C. for 20 min under microwave irradiation. Saturated aqueous ammonium chloride was added to the reaction mixture which was extracted with ethyl acetate (3×). The combined organic layers were washed with brine, dried over magnesium sulfate, filtered, concentrated in vacuo and purified by silica gel flash chromatography (MPLC, gradient elution; 0-40% EtOAc/hexanes) to obtain 4-iodo-1-(oxetan-3-yl)-1H-pyrazole.

LRMS (ESI) calc'd for C6H81N2O [M+H]⁺: 251. Found 251.

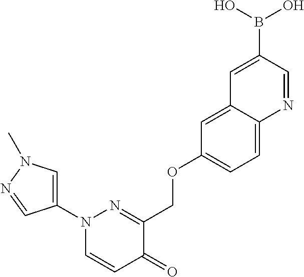 [SMILES: Cn1cc(-n2ccc(=O)c(COc3ccc4ncc(B(O)O)cc4c3)n2)cn1]

### Step 2. (6-{[1-(1-Methyl-1H-pyrazol-4-yl)-4-oxo-1,4-dihdropyridazin-3-yl]methoxy}quinolin-3-yl)boronic acid

6-{[1-(1-Methyl-1H-pyrazol-4-yl)-4-oxo-1,4-dihydropyridazin-3-yl]methoxy}quinolin-3-yl trifluoromethanesulfonate (Scheme #5, Example #71, Step 1, 100 mg, 0.208 mmol), bis(pinacolato)diboron (79 mg, 0.31 mmol), dipalladium (0) trisdibenzylideneacetone (9.5 mg, 10 μmol), XPhos (7.9 mg, 0.017 mmol), and potassium acetate (61.2 mg, 0.623 mmol) were combined in a 5 mL microwave vial which was then evacuated and back filled with nitrogen gas (3×) before adding 1,4-dioxane (4 mL). The reaction mixture was heated at 100° C. for 2 hours then filtered over Celite and eluted with EtOAc. The filtrate was concentrated in vacuo onto silica gel and purified by silica gel flash chromatography (MPLC, gradient elution, 0-15% MeOH/DCM) to obtain (6-{[1-(1-methyl-1H-pyrazol-4-yl)-4-oxo-1,4-dihydropyridazin-3-yl]methoxy}quinolin-3-yl)boronic acid.

LRMS (ESI) calc'd for C18H17BN5O4 [M+H]⁺: 378. Found 378.

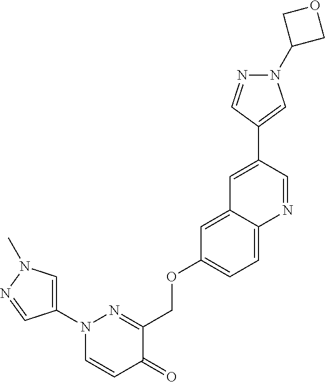 [SMILES: Cn1cc(-n2ccc(=O)c(COc3ccc4ncc(-c5cnn(C6COC6)c5)cc4c3)n2)cn1]

### Step 3. 1-(1-Methyl-1H-pyrazol-4-yl)-3-[({3-[1-(oxetan-3-yl)-1H-pyrazol-4-yl]quinolin-6-yl}oxy)methyl]pyridazin-4(1H)-one

A 2 mL microwave vial was charged with (6-{([1-(1-methyl-1H-pyrazol-4-yl)-4-oxo-1,4-dihydropyridazin-3-yl]methoxy}quinolin-3-yl)boronic acid (60 mg, 0.16 mmol), 4-iodo-1-(oxetan-3-yl)-1H-pyrazole (55.7 mg, 0.223 mmol), dipalladium (0) trisdibenzylideneacetone (7.3 mg, 7.95 μmol), XPhos (6.1 mg, 0.013 mmol), and cesium carbonate (207 mg, 0.636 mmol), sealed, evacuated and back-filled with nitrogen gas (3×), charged with 1,4-dioxane (2 mL) and heated at 100° C. for 2 hours. After cooling to room temperature, the mixture was filtered over Celite, eluted with EtOAc and DCM, concentrated in vacuo onto silica gel and purified by silica gel flash chromatography (MPLC, gradient elution, 0-15% MeOH/DCM) to obtain 1-(1-methyl-1H-pyrazol-4-yl)-3-[({3-[1-(oxetan-3-yl)-1H-pyrazol-4-yl]quinolin-6-yl}oxy)methyl]pyridazin-4(1H)-one.

LRMS (ESI) calc'd for C24H22N7O3 [M+H]⁺: 456. Found 456.

### Scheme #6

### Example #97

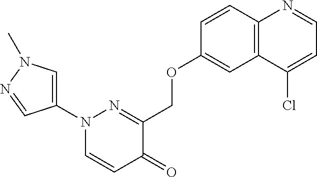 [SMILES: Cn1cc(-n2ccc(=O)c(COc3ccc4nccc(Cl)c4c3)n2)cn1]

### 3-{[(4-Chloroquinolin-6-yl)oxy]methyl)}-1-(1-methyl-1H-pyrazol-4-yl)pyridazin-4(1H)-one

### Step 1. 3-{[(4-Chloroquinolin-6-yl)oxy]methyl}-1-(1-methyl-1H-pyrazol-4-yl)pyridazin-4(1H)-one

A 5 mL microwave vial was charged with 4-chloro-6-bromoquinoline (Intermediate #18, Step 1, 0.10 g, 0.41 mmol), dipalladium (0) trisdibenzylideneacetone (9.3 mg, 10 μmol), Me₄^{t}BuXPhos (19 mg, 0.040 mmol), freshly ground potassium hydroxide (90 mg, 1.6 mmol), 1,4-dioxane (0.5 mL) and degassed water (0.5 mL) (degassed by placing water in vacuo and sonicating for −30 seconds). The reaction mixture was stirred at ambient temperature for 64 hours followed by 60° C. for 4.5 hours. After cooling to ambient temperature, the reaction mixture was charged with 3-(chloromethyl)-1-(1-methyl-1H-pyrazol-4-yl)pyridazin-4(1H)-one (Intermediate #1, 0.10 g, 0.45 mmol) and heated to 60° C. for 2 hours. After cooling to ambient temperature the reaction mixture was filtered through Celite, eluted with EtOAc, concentrated in vacuo and purified by silica gel flash chromatography (MPLC, gradient elution, 0-20% MeOH/EtOAc) followed by reverse-phase HPLC (15-50% MeCN/water, 0.1% TFA), freebasing with PL-HCO₃ cartridges to obtain 3-{[(4-chloroquinolin-6-yl)oxy]methyl}-1-(1-methyl-1H-pyrazol-4-yl)pyridazin-4(1H)-one.

LRMS (ESI) calc'd for C18H15ClN5O2 [M+H]⁺: 368. Found: 368.

### Scheme #6

### Example #98

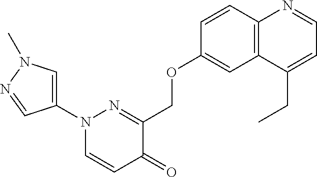 [SMILES: CCc1ccnc2ccc(OCc3nn(-c4cnn(C)c4)ccc3=O)cc12]

### 3-{[(4-Ethylquinolin-6-yl)oxy]methyl}-1-(1-methyl-1H-pyrazol-4-yl)pyridazin-4(1H)-one

### Step 1. 3-{[(4-Ethylquinolin-6-yl)oxy]methyl}-1-(1-methyl-1H-pyrazol-4-yl)pyridazin-4(1H)-one

An oven-dried, nitrogen cooled 2 mL microwave vial was charged with 3-{[(4-chloroquinolin-6-yl)oxy]methyl}-1-(1-methyl-1H-pyrazol-4-yl)pyridazin-4(1H)-one (Example #97, 43.6 mg, 0.119 mmol), dipalladium (0) trisdibenzylideneacetone (2.2 mg, 2.4 μmol), SPhos (3.9 mg, 9.5 μmol), cesium carbonate (0.12 g, 0.37 mmol) and potassium ethyltrifluoroborate (24 mg, 0.18 mmol), sealed under nitrogen, charged with toluene (0.5 mL) and degassed water (0.05 mL) and heated to 100° C. for 18 hours. After cooling to ambient temperature, the reaction mixture was filtered through Celite, eluted with EtOAc, concentrated in vacuo and purified by silica gel flash chromatography (MPLC, gradient elution, 0-20% MeOH/DCM) to obtain 3-{[(4-ethylquinolin-6-yl)oxy]methyl}-1-(1-methyl-1H-pyrazol-4-yl)pyridazin-4(1H)-one.

LRMS (ESI) calc'd for C20H20N5O2 [M+H]⁺: 362. Found: 362.

### Scheme #7

### Example #99

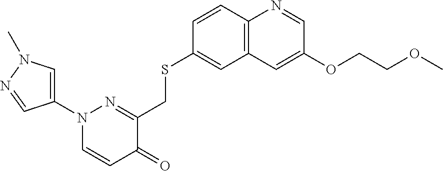 [SMILES: COCCOc1cnc2ccc(SCc3nn(-c4cnn(C)c4)ccc3=O)cc2c1]

### 3-({[3-(2-Methoxyethoxy)quinolin-6-yl]sulfanyl}methyl)-1-(1-methyl-1H-pyrazol-4-yl)pyridazin-4(1H)-one

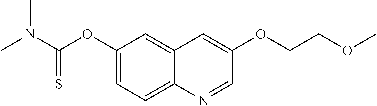

### Step 1. O-[3-(2-Methoxyethoxy)quinolin-6-yl]dimethylcarbamothioate

A 5 mL microwave vial was charged with 6-chloro-3-(2-methoxyethoxy)quinoline (Intermediate #11, 0.10 g, 0.42 mmol), dipalladium (0) trisdibenzylideneacetone (2.0 mg, 2.2 μmol), Me₄^{t}BuXPhos (4.0 mg, 8.3 μmol) and freshly ground potassium hydroxide (0.08 g, 1. mmol). The vial was sealed with a septum and its atmosphere purged in vacuo, backfilling with nitrogen. 1,4-dioxane (1 mL) and degassed water (1 mL) (degassed by placing water in vacuo and sonicating for ˜30 seconds) were added and the reaction was heated to 100° C. for 18 hours. After cooling to ambient temperature, the reaction mixture was charged with N,N-dimethylthiocarbamoyl chloride (0.16 g, 1.29 mmol) and heated to 100° C. for 10 min. The vial was removed from heat, stirred at ambient temperature overnight, filtered through Celite, eluted with EtOAc, concentrated in vacuo and purified by silica gel flash chromatography (MPLC, gradient elution, 0-20% MeOH/DCM followed by 25-100% EtOAc/hexanes) to give O-[3-(2-methoxyethoxy)quinolin-6-yl]dimethylcarbamothioate.

LRMS (ESI) calc'd for C15H19N2O3S [M+H]⁺: 307. Found: 307.

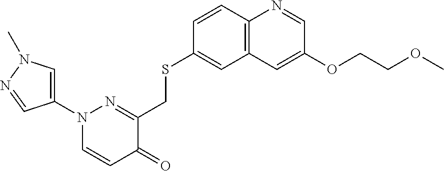 [SMILES: COCCOc1cnc2ccc(SCc3nn(-c4cnn(C)c4)ccc3=O)cc2c1]

### Step 2. 3-({[3-(2-Methoxyethoxy)quinolin-6-yl]sulfanyl}methyl)-1-(1-methyl-1H-pyrazol-4-yl)pyridazin-4(1H)-one

A solution of O-[3-(2-methoxyethoxy)quinolin-6-yl]dimethylcarbamothioate (54.7 mg, 0.179 mmol) in NMP (2 mL) was heated to 250° C. under microwave irradiation. After cooling to ambient temperature, 2 N sodium hydroxide solution (0.1 mL, 0.2 mmol) was added and the reaction mixture was heated to 100° C. for 1.5 hours. Additional 2 N sodium hydroxide solution (0.2 mL, 0.4 mmol) was added and the reaction mixture was heated to 100° C. for 24 hours. The reaction mixture was cooled to ambient temperature, charged with 3-(chloromethyl)-1-(1-methyl-1H-pyrazol-4-yl)pyridazin-4(1H)-one (Intermediate #1, 0.05 g, 0.2 mmol) and heated to 100° C. for 2 hours. After cooling to ambient temperature, the reaction mixture was filtered through Celite, eluted with EtOAc, washed with brine, dried over magnesium sulfate, filtered, concentrated in vacuo and purified by silica gel flash chromatography (MPLC, gradient elution, 0-20% MeOH/EtOAc) to obtain 3-({[3-(2-methoxyethoxy)quinolin-6-yl]sulfanyl}methyl)-1-(1-methyl-1H-pyrazol-4-yl)pyridazin-4(1H)-one.

LRMS (ESI) calc'd for C21H22N5O3S [M+H]⁺: 424. Found: 424.

The following example was prepared according to Scheme #8 following similar procedures described for Example #99, using Intermediate #13, which can be achieved by those of ordinary skill in the art of organic synthesis.

| | | | Exact Mass |
|---|---|---|---|
| Example | Structure | IUPAC Name | [M + H]+ |
| 100 | 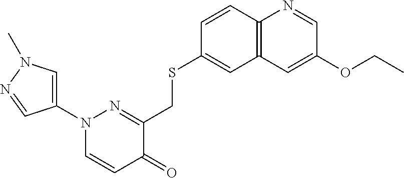 [SMILES: CCOc1cnc2ccc(SCc3nn(-c4cnn(C)c4)ccc3=O)cc2c1] | 3-{[(3- ethoxyquinolin-6- yl)sulfanyl]methyl}- 1-(1-methyl-1H- pyrazol-4- yl)pyridazin-4(1H)- one | Calc'd 394, found 394 |

### Scheme #7

### Example #101

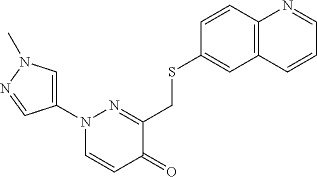 [SMILES: Cn1cc(-n2ccc(=O)c(CSc3ccc4ncccc4c3)n2)cn1]

### 1-(1-Methyl-1H-pyrazol-4-yl)-3-[(quinolin-6-ylsulfanyl)methyl]pyridazin-4(1H)-one

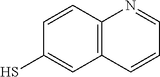 [SMILES: Sc1ccc2ncccc2c1]

### Step 1. Quinoline-6-thiol

In an oven-dried, nitrogen cooled 5 mL microwave vial was placed 6-iodoquinoline (Intermediate #31, 0.40 g, 1.6 mmol), tetrakis(triphenylphosphine)palladium (0) (0.09 g, 0.08 mmol) and cesium carbonate (0.67 g, 2.1 mmol). The vial was sealed under nitrogen and a degassed solution of triisopropylsilanethiol (0.44 mL, 2.0 mmol) in toluene (3 mL) was added. The reaction was heated to 100° C. for 2 hours under microwave irradiation then cooled to ambient temperature. A solution of TBAF in THF (2.1 mL, 2.1 mmol) was added and the reaction mixture was stirred for 2 hours at ambient temperature. The reaction was quenched with saturated aqueous ammonium chloride solution then extracted with EtOAc (3×). The combined organics were dried over magnesium sulfate, filtered and concentrated to give quinoline-6-thiol.

LRMS (ESI) calc'd for C9H8NS [M+H]⁺: 162. Found: 162.

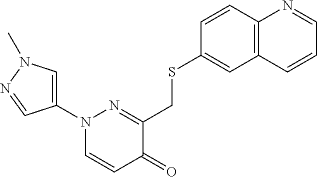 [SMILES: Cn1cc(-n2ccc(=O)c(CSc3ccc4ncccc4c3)n2)cn1]

### Step 2. 1-(1-Methyl-1H-pyrazol-4-yl)-3-[(quinolin-6-ylsulfanyl)methyl]pyridazin-4(1H)-one

In a 20 mL microwave vial was placed quinoline-6-thiol (253 mg, 1.57 mmol), potassium carbonate (0.30 g, 2.2 mmol), 3-(chloromethyl)-1-(1-methyl-1H-pyrazol-4-yl)pyridazin-4(1H)-one (Intermediate #1, 0.37 g, 1.6 mmol) and DMF (10 mL). The vial was sealed with a septum and heated to 100° C. for 2 hours under microwave irradiation. After cooling to ambient temperature, water was added to quench the reaction mixture. The reaction mixture was filtered to remove the precipitate that had formed. The filtrate was concentrated in vacuo then triturated with water. The precipitate was collected via vacuum filtration, washed with water and dried via lyophilizer to obtain 1-(1-methyl-1H-pyrazol-4-yl)-3-[(quinolin-6-ylsulfanyl)methyl]pyridazin-4(1H)-one as a white solid.

LRMS (ESI) calc'd for C18H16N50S [M+H]⁺: 350. Found: 350.

### Scheme #8

### Example #102

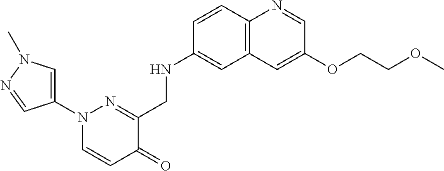 [SMILES: COCCOc1cnc2ccc(NCc3nn(-c4cnn(C)c4)ccc3=O)cc2c1]

### 3-({[3-(2-Methoxyethoxy)quinolin-6-yl]amino}methyl)-1-(1-methyl-1H-pyrazol-4-yl)pyridazin-4(1H)-one

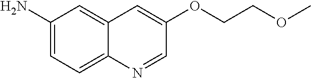 [SMILES: COCCOc1cnc2ccc(N)cc2c1]

### Step 1. 3-(2-Methoxyethoxy)quinolin-6-amine

In an oven-dried, nitrogen cooled 5 mL microwave vial was placed 6-chloro-3-(2-methoxyethoxy)quinoline (Intermediate #11, 0.10 g, 0.42 mmol), dipalladium (0) trisdibenzylideneacetone (19 mg, 0.021 mmol), and JohnPhos (15 mg, 0.050 mmol). The reaction was sealed under nitrogen, THF (2 mL) and 1 M LiHMDS in THF (0.51 mL, 0.51 mmol) were added and the reaction heated to 10° C. for 16 hours. Ambient temperature was attained, at which point 3 N HCl in MeOH (0.15 mL, 0.45 mmol) was added. This mixture was passed through a PL-HCO3 SPE cartridge. Concentration of the filtrate in vacuo gave 3-(2-methoxyethoxy)quinolin-6-amine.

LRMS (ESI) calc'd for C12H15N2O2 [M+H]⁺: 219. Found: 219.

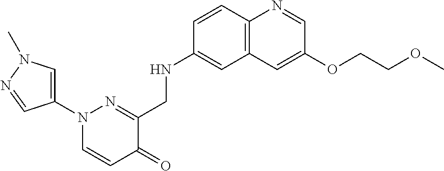 [SMILES: COCCOc1cnc2ccc(NCc3nn(-c4cnn(C)c4)ccc3=O)cc2c1]

### Step 2. 3-({[3-(2-Methoxyethoxy)quinolin-6-yl]amino}methyl)-1-(1-methyl-1H-pyrazol-4-yl)pyridazin-4(1H)-one

In a 5 mL microwave vial was placed 3-(chloromethyl)-1-(1-methyl-1H-pyrazol-4-yl)pyridazin-4(1H)-one (Intermediate #1, 0.08 g, 0.4 mmol), 3-(2-methoxyethoxy)quinolin-6-amine (92 mg, 0.42 mmol), potassium carbonate (0.10 g, 0.72 mmol) and DMF (2 mL). The reaction was sealed and heated to 100° C. for 2 hours. The reaction was allowed to cool to ambient temperature, at which point addition of water gave a precipitate which was removed by filtration. Concentration of the filtrate in vacuo and purification by reverse-phase HPLC (15-50% MeCN/water, 0.1% TFA, freebased with PL-HCO3 cartridges) gave 3-({[3-(2-methoxyethoxy)quinolin-6-yl]amino}methyl)-1-(1-methyl-1H-pyrazol-4-yl)pyridazin-4(1H)-one as an orange solid.

LRMS (ESI) calc'd for C21H23N6O3 [M+H]⁺: 407. Found: 407.

The following example was prepared according to Scheme #7 following similar procedures described for Example #102, using Intermediate #13, which can be achieved by those of ordinary skill in the art of organic synthesis.

| | | | Exact Mass |
|---|---|---|---|
| Example | Structure | IUPAC Name | [M + H]+ |
| 103 | 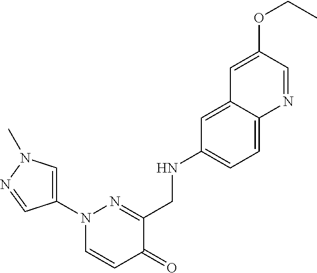 [SMILES: CCOc1cnc2ccc(NCc3nn(-c4cnn(C)c4)ccc3=O)cc2c1] | 3-{[(3- ethoxyquinolin-6- yl)amino]methyl}-1- (1-methyl-1H- pyrazol-4- yl)pyridazin-4(1H)- one | Calc'd 377, found 377 |

### Scheme #8

### Example #104

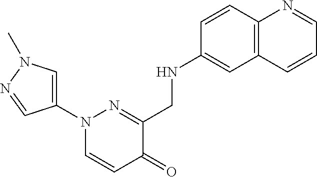 [SMILES: Cn1cc(-n2ccc(=O)c(CNc3ccc4ncccc4c3)n2)cn1]

### 1-(1-Methyl-1H-pyrazol-4-yl)-3-[(quinolin-6-ylamino)methyl]pyridazin-4(1H)-one

### Step 1. 1-(1-Methyl-1H-pyrazol-4-yl)-3-[(quinolin-6-ylamino)methyl]pyridazin-4(1H)-one

In an oven-dried, nitrogen cooled 5 mL microwave vial was placed 3-(chloromethyl)-1-(1-methyl-1H-pyrazol-4-yl)pyridazin-4(1H)-one (Intermediate #1, 0.20 g, 0.89 mmol), potassium carbonate (0.20 g, 1.4 mmol) and 6-aminoquinoline (0.20 g, 1.4 mmol). The reaction mixture was sealed under nitrogen and DMF (2 mL) was added. The reaction mixture was stirred at 100° C. for 16.5 hours. Ambient temperature was attained and water was added. The mixture was then concentrated in vacuo onto silica gel. Purification by silica gel flash chromatography (MPLC, gradient elution, 0-20% MeOH/DCM) followed by recrystallization from methanol gave 1-(1-methyl-1H-pyrazol-4-yl)-3-[(quinolin-6-ylamino)methyl]pyridazin-4(1H)-one as a white solid.

LRMS (ESI) calc'd for C18H17N6O [M+H]⁺: 333. Found: 333.

### Scheme #9

### Example #105

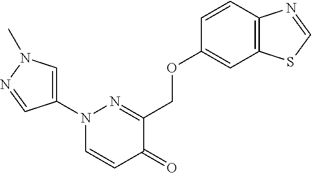 [SMILES: Cn1cc(-n2ccc(=O)c(COc3ccc4ncsc4c3)n2)cn1]

### 3-[(1,3-Benzothiazol-6-yloxy)methyl]-1-(1-methyl-1H-pyrazol-4-yl)pyridazin-4(1H)-one

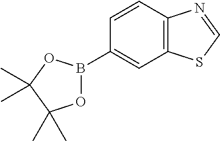 [SMILES: CC1(C)OB(c2ccc3ncsc3c2)OC1(C)C]

### Step 1. 6-(4,4,5,5-Tetramethyl-1,3,2-dioxaborolan-2-yl)-1,3-benzothiazole

In an oven-dried, nitrogen cooled 5 mL microwave vial was placed 6-bromo-1,3-benzothiazole (0.10 g, 0.47 mmol), PdCl₂(dppf).DCM (0.02 g, 0.02 mmol), bis(pinacoloto)diboron (0.13 g, 0.51 mmol) and potassium acetate (0.14 g, 1.4 mmol). The vial was sealed under nitrogen and 1,4-dioxane (1 mL) was added. The reaction mixture was heated to 100° C. for 18 hours. After cooling to ambient temperature, the reaction mixture filtered through Celite, eluting with EtOAc. The filtrate was concentrated in vacuo and purified by silica gel flash chromatography (MPLC, gradient elution, 0-50% EtOAc/hexanes) to obtain 6-(4,4,5,5-tetramethyl-1,3,2-dioxaborolan-2-yl)-1,3-benzothiazole as a white solid.

LRMS (ESI) calc'd for C13H17BNO2S [M+H]⁺: 262. Found: 262.

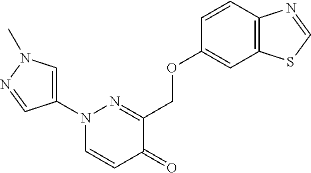 [SMILES: Cn1cc(-n2ccc(=O)c(COc3ccc4ncsc4c3)n2)cn1]

### Step 2. 3-[(1,3-Benzothiazol-6-yloxy)methyl]-1-(1-methyl-1H-pyrazol-4-yl)pyridazin-4(1H)-one as a white solid

To a stirring solution of 6-(4,4,5,5-tetramethyl-1,3,2-dioxaborolan-2-yl)-1,3-benzothiazole (81 mg, 0.31 mmol) in THF (3 mL) was added 1 N sodium hydroxide (0.5 mL, 0.5 mmol) followed by 30% hydrogen peroxide (35 μL, 0.34 mmol). The reaction mixture was stirred at ambient temperature for 18 hours. 3-(Chloromethyl)-1-(1-methyl-1H-pyrazol-4-yl)pyridazin-4(1H)-one (Intermediate #1, 0.09 g, 0.4 mmol) was added and the reaction mixture was stirred at ambient temperature for 5 hours followed by heating to 60° C. for 70 min. The solvent was removed in vacuo and purified by reverse-phase HPLC (25-60% MeCN/water, 0.1% TFA, freebased via PL-HCO3 SPE cartridges) to obtain 3-[(1,3-benzothiazol-6-yloxy)methyl]-1-(1-methyl-1H-pyrazol-4-yl)pyridazin-4(1H)-one as a white solid.

LRMS (ESI) calc'd for C16H14N5O2S [M+H]⁺: 340. Found: 340.

The following examples were prepared according to Scheme #9 following similar procedures described for Example #105, using Intermediates #1, and 32-33, which can be achieved by those of ordinary skill in the art of organic synthesis.

| | | | Exact Mass |
|---|---|---|---|
| Example | Structure | IUPAC Name | [M + H]+ |
| 106 | 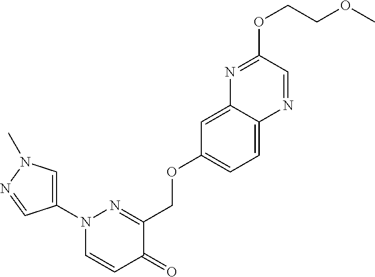 [SMILES: COCCOc1cnc2ccc(OCc3nn(-c4cnn(C)c4)ccc3=O)cc2n1] | 3-({[3-(2- methoxyethoxy)quin- oxalin-6- yl]oxy}methyl)-1-(1- methyl-1H-pyrazol-4- yl)pyridazin-4(1H)- one | Calc'd 409, found 409 |
| 107 | 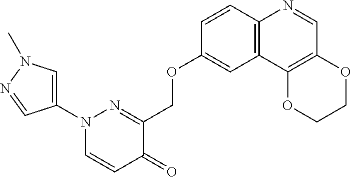 [SMILES: Cn1cc(-n2ccc(=O)c(COc3ccc4ncc5c(c4c3)OCCO5)n2)cn1] | 3-[(2,3- dihydro[1,4]dioxino[2,3- c]quinolin-9- yloxy)methyl]-1-(1- methyl-1H-pyrazol-4- yl)pyridazin-4(1H)- one | Calc'd 392, found 392 |

### Scheme #10

### Example #108

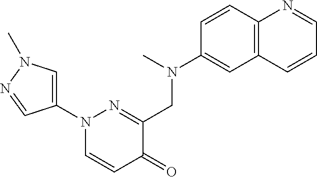 [SMILES: CN(Cc1nn(-c2cnn(C)c2)ccc1=O)c1ccc2ncccc2c1]

### 1-(1-Methyl-1H-pyrazol-4-yl)-3-{[methyl(quinolin-6-yl)amino]methyl}pyridazin-4(1H)-one

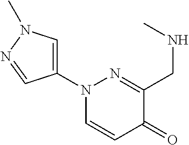 [SMILES: CNCc1nn(-c2cnn(C)c2)ccc1=O]

### Step 1. 3-[(Methylamino)methyl]-1-(1-methyl-1H-pyrazol-4-yl)pyridazin-4(1H)-one

A stirring solution of 3-(chloromethyl)-1-(1-methyl-1H-pyrazol-4-yl)pyridazin-4(1H)-one (Intermediate #1, 0.10 g, 0.45 mmol) and 2 M methylamine in THF (2 mL, 4 mmol) was heated to 80° C. for 65 hours. The crude reaction mixture was concentrated in vacuo onto silica gel and purified by silica gel flash chromatography (MPLC, gradient elution, 0-100% MeOH/DCM). The combined fractions were dried over magnesium sulfate, filtered and concentrated in vacuo to obtain 3-[(methylamino)methyl]-1-(1-methyl-1H-pyrazol-4-yl)pyridazin-4(1H)-one as an orange solid.

LRMS (ESI) calc'd for C10H14N5O [M+H]⁺: 220. Found: 220.

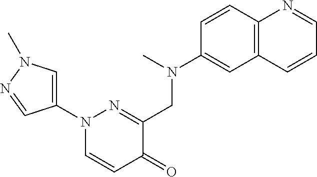 [SMILES: CN(Cc1nn(-c2cnn(C)c2)ccc1=O)c1ccc2ncccc2c1]

### Step 2. 1-(1-Methyl-1H-pyrazol-4-yl)-3-{[(methyl(quinolin-6-yl)amino]methyl}pyridazin-4(1H)-one

In a 5 mL microwave vial was placed 3-[(methylamino)methyl]-1-(1-methyl-1H-pyrazol-4-yl)pyridazin-4(1H)-one (66.0 mg, 0.301 mmol), dipalladium (0) trisdibenzylideneacetone (13.8 mg, 0.015 mmol), JohnPhos (13.5 mg, 0.045 mmol), sodium tert-butoxide (35 mg, 0.36 mmol) and 6-chloroquinoline (0.06 g, 0.4 mmol). The vial was sealed and its atmosphere was evacuated, backfilling with nitrogen (3×). DME (1.5 mL) was added and the reaction was heated to 40° C. overnight. The reaction was cooled to ambient temperature and filtered through Celite, eluting with EtOAc. The filtrate was concentrated in vacuo and purified by reverse-phase HPLC (15-50% MeCN/water, 0.1% TFA) to provide 1-(1-methyl-1H-pyrazol-4-yl)-3-{[methyl(quinolin-6-yl)amino]methyl}pyridazin-4(1H)-one as a lemon-yellow solid.

LRMS (ESI) calc'd for C19H19N6O [M+H]: 347. Found: 347.

### Scheme #11

### Example #109

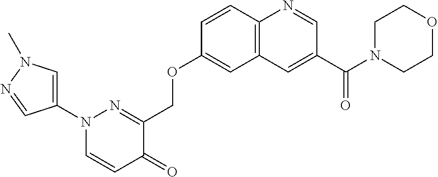 [SMILES: Cn1cc(-n2ccc(=O)c(COc3ccc4ncc(C(=O)N5CCOCC5)cc4c3)n2)cn1]

### 1-(1-methyl-1H-pyrazol-4-yl)-3-({[3-(morpholin-4-ylcarbonyl)quinolin-6-yl]oxy}methyl)pyridazin-4(1H)-one

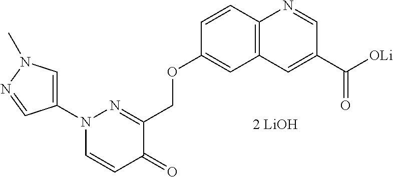

### Step 1. Lithium 6-{[1-(1-methyl-1H-pyrazol-4-yl)-4-oxo-1,4-dihydropyridazin-3-yl]methoxy}quinoline-3-carboxylate

A suspension of methyl 6-{[1-(1-methyl-1H-pyrazol-4-yl)-4-oxo-1,4-dihydropyridazin-3-yl]methoxy}quinoline-3-carboxylate (Example #95, 273.9 mg, 0.700 mmol) and lithium hydroxide (0.05 g, 2 mmol) in THF (1 mL) and water (1 mL) was heated to 100° C. for 2 hours. The reaction mixture was cooled to ambient temperature and concentrated in vacuo to obtain crude lithium 6-{[1-(1-methyl-1H-pyrazol-4-yl)-4-oxo-1,4-dihydropyridazin-3-yl]methoxy}quinoline-3-carboxylate as a white solid (two equivalents of lithium hydroxide remaining).

LRMS (ESI) calc'd for C19H16N5O4 (free acid) [M+H]: 378. Found: 378.

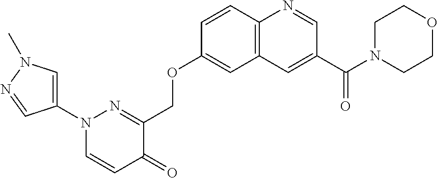 [SMILES: Cn1cc(-n2ccc(=O)c(COc3ccc4ncc(C(=O)N5CCOCC5)cc4c3)n2)cn1]

### Step 2. 1-(1-Methyl-1H-pyrazol-4-yl-3-({[3-morpholin-4-ylcarbonyl)quinolin-6-yl]oxy}methyl)pyridazin-4(1H)-one

To a stirring solution of lithium 6-{[1-(1-methyl-1H-pyrazol-4-yl)-4-oxo-1,4-dihydropyridazin-3-yl]methoxy}quinoline-3-carboxylate (20 mg, 0.05 mmol), HOBT (29 mg, 0.19 mmol) and EDC (35 mg, 0.18 mmol) in DMF (0.5 mL) was added morpholine (8 μL, 0.09 mmol) and DIPEA (0.07 mL, 0.4 mmol). The reaction was stirred at ambient temperature for 17 hours then heated to 100° C. for 2 hours and 20 min. The reaction mixture was cooled to ambient temperature, diluted with water and extracted with EtOAc (3×). The combined organics were washed with brine, dried over magnesium sulfate, filtered and concentrated in vacuo. Purification by silica gel flash chromatography (MPLC, gradient elution, 0-20% MeOH/DCM) gave 1-(1-methyl-1H-pyrazol-4-yl)-3-({[3-(morpholin-4-ylcarbonyl)quinolin-6-yl]oxy}methyl)pyridazin-4(1H)-one as a white solid.

LRMS (ESI) calc'd for C23H23N6O4 [M+H]⁺: 447. Found: 447.

### Scheme #12

### Examples #110 and #111

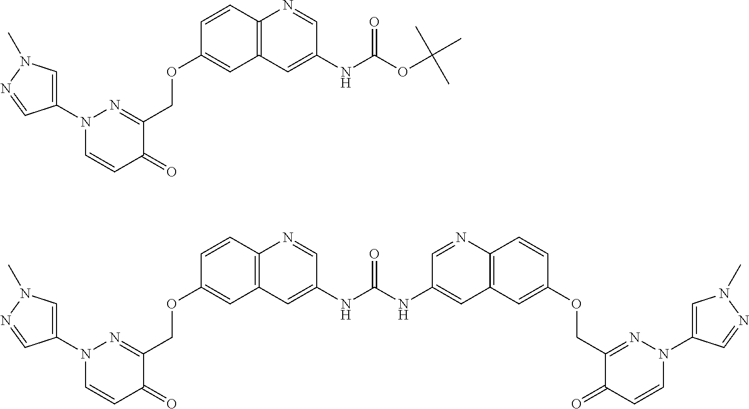 [SMILES: Cn1cc(-n2ccc(=O)c(COc3ccc4ncc(NC(=O)OC(C)(C)C)cc4c3)n2)cn1 ; Cn1cc(-n2ccc(=O)c(COc3ccc4ncc(NC(=O)Nc5cnc6ccc(OCc7nn(-c8cnn(C)c8)ccc7=O)cc6c5)cc4c3)n2)cn1]

### tert-Butyl (6-{[1-(1-methyl-1H-pyrazol-4-yl)-4-oxo-1,4-dihydropyridazin-3-yl]methoxy}quinolin-3-yl)carbamate and 1,3-bis(6-{[1-(1-methyl-1H-pyrazol-4-yl)-4-oxo-1,4-dihydropyridazin-3-yl]methoxy)}quinolin-3-yl)urea

### Step 1. tert-Butyl (6-[1-(1-methyl-1H-pyrazol-4-yl)-4-oxo-1,4-dihydropyridazin-3-yl]methoxyquinolin-3-yl)carbamate and 1,3-bis(6-{[1-(1-methyl-1H-pyrazol-4-yl)-4-oxo-1,4-dihydropyridazin-3-yl]methoxy}quinolin-3-yl)urea

A 20 mL microwave vial was charged with 6-{[1-(1-methyl-1H-pyrazol-4-yl)-4-oxo-1,4-dihydropyridazin-3-yl]methoxy}quinoline-3-carboxylic acid (prepared by HPLC purification of Scheme #12, Step 1, 0.09 g, 0.2 mmol) and tert-butyl alcohol (2 mL). Triethylamine (53 μL, 0.38 mmol) was added to the reaction mixture followed by diphenylphosphoryl azide (66 μL, 0.31 mmol). The reaction mixture was sealed and heated to 100° C. for 18 hours. After cooling to ambient temperature, water was added and the resulting precipitate collected via filtration and dried via lyophilizer. The obtained solid was washed with DCM and the filtrate concentrated in vacuo. The residue obtained was purified by silica gel flash chromatography (MPLC, gradient elution, 0-20% MeOH/DCM) to obtain tert-butyl (6-{[1-(1-methyl-1H-pyrazol-4-yl)-4-oxo-1,4-dihydropyridazin-3-yl]methoxy}quinolin-3-yl)carbamate (Example #110) as a white solid. The DCM-insoluble matter was dried in vacuo to give 1,3-bis(6-{[1-(1-methyl-1H-pyrazol-4-yl)-4-oxo-1,4-dihydropyridazin-3-yl]methoxy}quinolin-3-yl)urea (Example #111).

LRMS (ESI) calc'd for C23H25N6O4 [M+H]: 449. Found: 449.

LRMS (ESI) calc'd for C37H32N12O5 [M+2H]²⁺/2: 362. Found: 362.

### Scheme #12

### Example #112

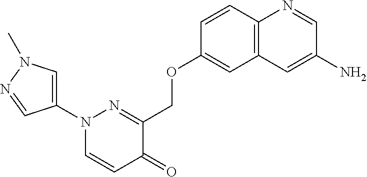 [SMILES: Cn1cc(-n2ccc(=O)c(COc3ccc4ncc(N)cc4c3)n2)cn1]

### 3-{[(3-Aminoquinolin-6-yl)oxy]methyl}-1-(1-methyl-1H-pyrazol-4-yl)pyridazin-4(1H)-one

### Step 1. 3-{[(3-Aminoquinolin-6-yl)oxy]methyl}-1-(1-methyl-1H-pyrazol-4-yl)pyridazin-4(1H)-one

tert-Butyl (6-{[1-(1-methyl-1H-pyrazol-4-yl)-4-oxo-1,4-dihydropyridazin-3-yl]methoxy}quinolin-3-yl)carbamate (Example #110, 25.7 mg, 0.057 mmol) was stirred in 1 N HCl in 1,4-dioxane (1 mL, 1 mmol) at ambient temperature overnight. The reaction mixture was concentrated in vacuo and taken up in MeOH. Filtration of this solution through PL-HCO3 cartridges gave freebased 3-{[(3-aminoquinolin-6-yl)oxy]methyl}-1-(1-methyl-1H-pyrazol-4-yl)pyridazin-4(1H)-one as a white solid.

LRMS (ESI) calc'd for C18H17N6O2 [M+H]⁺: 349. Found: 349.

### Scheme #13

### Examples #113 and #114

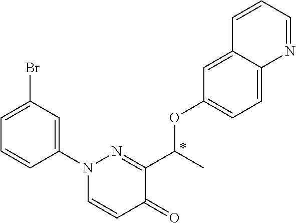 [SMILES: CC(Oc1ccc2ncccc2c1)c1nn(-c2cccc(Br)c2)ccc1=O]

### (R and S)-1-(3-Bromophenyl)-3-[1-(quinolin-6-yloxy)ethyl]pyridazin-4(1H)-one

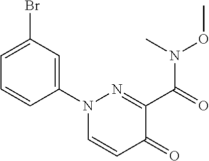 [SMILES: CON(C)C(=O)c1nn(-c2cccc(Br)c2)ccc1=O]

### Step 1. 1-(3-Bromophenyl)-N-methoxy-N-methyl-4-oxo-1,4-dihydropyridazine-3-carboxamide

1-(3-Bromophenyl)-4-oxo-1,4-dihydropyridazine-3-carboxylic acid (Example #13, Step 3, 1.5 g, 5.1 mmol), N,O-dimethylhydroxylamine hydrochloride (0.992 g, 10.2 mmol), DIPEA (3.55 mL, 20.3 mmol), EDC (1.46 g, 7.62 mmol) and HOBt (1.17 g, 7.62 mmol) were stirred in DMF (25 mL) at ambient temperature for 3 days. Saturated aqueous sodium bicarbonate solution was added and the products were extracted into EtOAc (2×). The combined organic extracts were washed with brine, dried over sodium sulfate, filtered and concentrated in vacuo. Purification of the residue by silica gel flash chromatography (MPLC, gradient elution, 0-10% MeOH/EtOAc) gave 1-(3-bromophenyl)-N-methoxy-N-methyl-4-oxo-1,4-dihydropyridazine-3-carboxamide as a pale yellow solid.

LRMS (ESI) calc'd for C13H12BrN3O3 [M+H]⁺: 338. Found: 338.

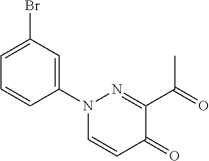 [SMILES: CC(=O)c1nn(-c2cccc(Br)c2)ccc1=O]

### Step 2. 3-Acetyl-1-(3-bromophenyl)pyridazin-4(1H)-one

1-(3-Bromophenyl)-N-methoxy-N-methyl-4-oxo-1,4-dihydropyridazine-3-carboxamide (1.49 g, 4.42 mmol) was taken up in THF (22 mL) and cooled to −78° C. Methylmagnesium bromide solution (4.42 mL, 13.3 mmol) was added and the resulting mixture stirred at −78° C. for 2 hours. While keeping the reaction mixture at −78° C., 15 mL of 2 N HCl was added followed by water. The products were extracted into EtOAc (2×). The combined organic extracts were washed with brine, dried over sodium sulfate, filtered and concentrated in vacuo. The residue was triturated in Et₂O to give 3-acetyl-1-(3-bromophenyl)pyridazin-4(1H)-one as a yellow solid.

LRMS (ESI) calc'd for C12H9BrN2O2 [M+H]⁺: 293. Found: 293.

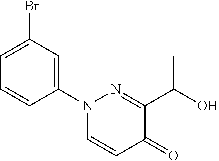 [SMILES: CC(O)c1nn(-c2cccc(Br)c2)ccc1=O]

### Step 3. 1-(3-bromophenyl)-3-(1-hydroxyethyl)pyridazin-4(1H)-one

3-Acetyl-1-(3-bromophenyl)pyridazin-4(1H)-one (304 mg, 1.04 mmol) and sodium borohydride (43 mg, 1.1 mmol) were stirred in MeOH (10 mL) at ambient temperature for 3 hours. Saturated aqueous ammonium chloride solution was added and the products extracted into EtOAc (×2). The combined organic extracts were washed with brine, dried over sodium sulfate, filtered and concentrated in vacuo. Purification of the residue by silica gel flash chromatography (MPLC, gradient elution, 0-10% MeOH/EtOAc) gave 1-(3-bromophenyl)-3-(1-hydroxyethyl)pyridazin-4(1H)-one as a white solid.

LRMS (ESI) calc'd for C12H11BrN2O2 [M+H]⁺: 295. Found: 295.

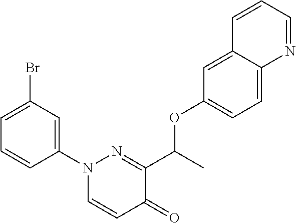 [SMILES: CC(Oc1ccc2ncccc2c1)c1nn(-c2cccc(Br)c2)ccc1=O]

### Step 4. 1-(3-Bromophenyl)-3-[1-(quinolin-6-yloxy)ethyl]pyridazin-4(1H)-one

1-(3-Bromophenyl)-3-(1-hydroxyethyl)pyridazin-4(1H)-one (77 mg, 0.26 mmol), 6-hydroxyquinoline (56.8 mg, 0.391 mmol), triphenylphosphine (103 mg, 0.391 mmol) and DIAD (0.076 mL, 0.39 mmol) were stirred in THF (2.6 mL) at ambient temperature for 5 hours. Additional triphenylphosphine (34.2 mg, 0.130 mmol) and DIAD (0.025 mL, 0.13 mmol) were added and the reaction mixture was stirred at ambient temperature for 3 days. The solvent was removed in vacuo onto silica gel and the residue purified by silica gel flash chromatography (MPLC, gradient elution, 0-10% MeOH/EtOAc) to give racemic 1-(3-bromophenyl)-3-[1-(quinolin-6-yloxy)ethyl]pyridazin-4(1H)-one as a colorless gum.

LRMS (ESI) calc'd for C21H16BrN3O2 [M+H]⁺: 422. Found: 422.

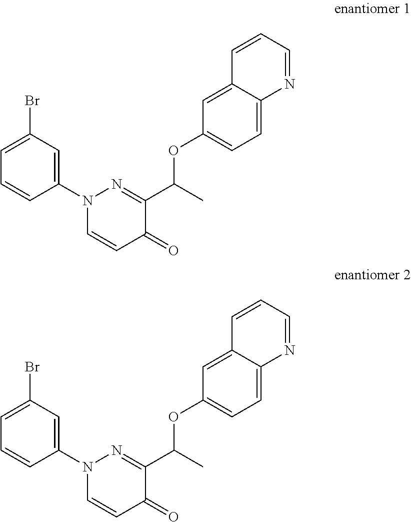 [SMILES: CC(Oc1ccc2ncccc2c1)c1nn(-c2cccc(Br)c2)ccc1=O ; CC(Oc1ccc2ncccc2c1)c1nn(-c2cccc(Br)c2)ccc1=O]

### Step 5. Chiral separation of racemic 1-(3-bromophenyl)-3-[1-(quinolin-6-yloxy)ethyl]pyridazin-4(1H)-one

Chiral separation was performed using chiral SFC as follows:

### Column: Chiral Technology OJ 2.1×25 cm, 10 uM.

MP: 35%/65% Methanol/CO2 (no other modifiers).
Flow rate: 70 mL/Min, 5.5 min run time.

### WL: 320 nm.

Racemic 1-(3-bromophenyl)-3-[1-(quinolin-6-yloxy)ethyl]pyridazin-4(1H)-one (96 mg) was dissolved in 4 mL of methanol. Injections were performed on a Berger Multigram II SFC. Elution of the enantiomers was observed at 2.29 minutes (Enantiomer 1, Example #113) and 3.72 minutes (Enantiomer 2, Example #114). No attempt was made to identify absolute stereochemistry.

LRMS (ESI) calc'd for C21H16BrN3O2 [M+H]⁺: 422. Found: 422.

### Scheme #14

### Examples #115 and #116

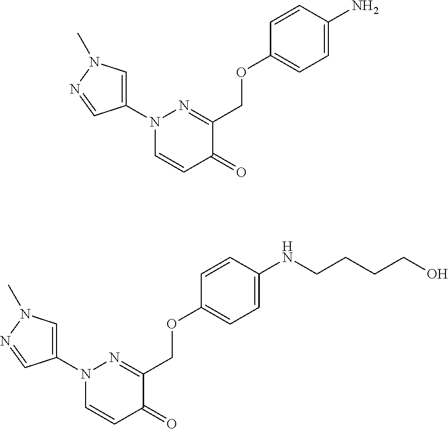 [SMILES: Cn1cc(-n2ccc(=O)c(COc3ccc(N)cc3)n2)cn1 ; Cn1cc(-n2ccc(=O)c(COc3ccc(NCCCCO)cc3)n2)cn1]

### 3-((4-aminophenoxy)methyl)-1-(1-methyl-1H-pyrazol-4-yl)pyridazin-4(1H)-one and 3-((4-(4-hydroxybutylamino)phenoxy)methyl)-1-(1-methyl-1H-pyrazol-4-yl)pyridazin-4(1H)-one

### Step 1. 3-((4-aminophenoxy)methyl)-1-(1-methyl-1H-pyrazol-4-ylpyridazin-4(1H)-one and 3-((4-(4-hydroxybutylamino)phenoxy)methyl)-1-(1-methyl-1H-pyrazol-4-yl)pyridazin-4(1H)-one

A sample of 1-(1-methyl-1H-pyrazol-4-yl)-3-((4-nitrophenoxy)methyl)pyridazin-4(1H)-one (Example #9, 84.6 mg, 0.258 mmol) was taken up in EtOAc (10 mL) and THF (10 mL). Nitrogen was bubbled through the reaction mixture for two minutes and 10% Pd/C (27.5 mg, 0.258 mmol) was added. The reaction mixture was subjected to 45 psi of hydrogen gas on a Parr shaker apparatus for 1 hour. The reaction was removed from the hydrogen atmosphere and filtered through Celite. The filtrate was concentrated in vacuo then purified by silica gel flash chromatography (ISCO, 0-20% MeOH/DCM) to obtain 3-((4-aminophenoxy)methyl)-1-(1-methyl-1H-pyrazol-4-yl)pyridazin-4(1H)-one (Example #115) as a white foam and 3-((4-(4-hydroxybutylamino)phenoxy)methyl)-1-(1-methyl-1H-pyrazol-4-yl)pyridazin-4(1H)-one (Example #116) as a white residue.

LRMS (ESI) calc'd for C15H16N5O2 [M+H]: 298. Found: 298.

LRMS (ESI) calc'd for C19H24N5O3 [M+H]⁺: 370. Found: 370.

The following examples were prepared according to Scheme #15 following similar procedures described for Examples #115 and #116, which can be achieved by those of ordinary skill in the art of organic synthesis.

| | | | Exact Mass |
|---|---|---|---|
| Example | Structure | IUPAC Name | [M + H]+ |
| 117 | 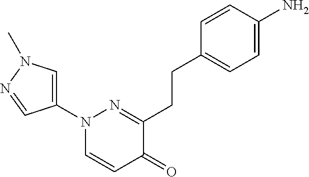 [SMILES: Cn1cc(-n2ccc(=O)c(CCc3ccc(N)cc3)n2)cn1] | 3-[2-(4- aminophenyl)eth- yl]-1-(1- methyl-1H- pyrazol-4- yl)pyridazin- 4(1H)-one | Calc'd 296, found 296 |
| 118 | 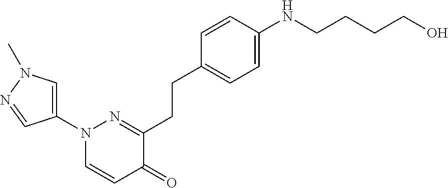 [SMILES: Cn1cc(-n2ccc(=O)c(CCc3ccc(NCCCCO)cc3)n2)cn1] | 3-(2-{4-[(4- hydroxybutyl) amino]phenyl}eth- yl)-1-(1- methyl-1H- pyrazol-4- yl)pyridazin- 4(1H)-one | Calc'd 368, found 368 |

### Scheme #14

### Example #119

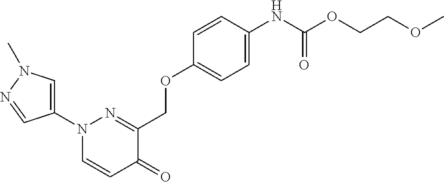 [SMILES: COCCOC(=O)Nc1ccc(OCc2nn(-c3cnn(C)c3)ccc2=O)cc1]

### 2-methoxyethyl (4-{[1-(1-methyl-1H-pyrazol-4-yl)-4-oxo-1,4-dihydropyridazin-3-yl]methoxy}phenyl)carbamate

### Step 1. 2-methoxyethyl (4-{[1-(1-methyl-1H-pyrazol-4-yl)-4-oxo-1,4-dihydropyridazin-3-yl]methoxy}phenyl)carbamate

A sample of 3-((4-aminophenoxy)methyl)-1-(1-methyl-1H-pyrazol-4-yl)pyridazin-4(1H)-one (Example #115, 20. mg, 0.067 mmol) was taken into THF (500 μL). To this solution was added DIPEA (14 μL, 0.09 mmol) followed by slow addition of 2-(methoxy)ethylchloroformate (9 μL, 0.08 mmol). The reaction was allowed to stir at ambient temperature under an atmosphere of nitrogen for 1 hour. The crude reaction mixture was then concentrated in vacuo. The resulting residue was partitioned between EtOAc and water. The resulting organics were dried over sodium sulfate, and volatiles were removed in vacuo. Purification via reverse-phase HPLC (10-100% MeCN/water, 0.1% TFA) gave 2-methoxyethyl (4-{[1-(1-methyl-1H-pyrazol-4-yl)-4-oxo-1,4-dihydropyridazin-3-yl]methoxy}phenyl)carbamate.

LRMS (ESI) calc'd for C19H22N5O5 [M+H]⁺: 398. Found: 398.

The following examples were prepared according to Scheme #15 following similar procedures described for Example #119 using Examples #115 and 117, which can be achieved by those of ordinary skill in the art of organic synthesis.

| | | | Exact Mass |
|---|---|---|---|
| Example | Structure | IUPAC Name | [M + H]+ |
| 120 | 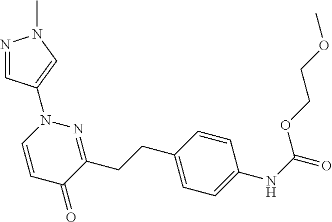 [SMILES: COCCOC(=O)Nc1ccc(CCc2nn(-c3cnn(C)c3)ccc2=O)cc1] | 2-methoxyethyl (4- {2-[1-(1-methyl-1H- pyrazol-4-yl)-4-oxo- 1,4-dihydropyridazin- 3- yl]ethyl}phenyl) carbamate | Calc'd 398, found 398 |
| 121 | 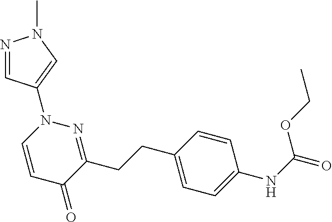 [SMILES: CCOC(=O)Nc1ccc(CCc2nn(-c3cnn(C)c3)ccc2=O)cc1] | ethyl (4-{2-[1-(1- methyl-1H-pyrazol-4- yl)-4-oxo-1,4- dihydropyridazin-3- yl]ethyl}phenyl) carbamate | Calc'd 368, found 368 |
| 122 | 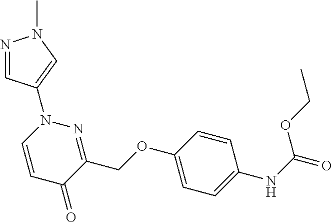 [SMILES: CCOC(=O)Nc1ccc(OCc2nn(-c3cnn(C)c3)ccc2=O)cc1] | ethyl (4-{[1-(1- methyl-1H-pyrazol-4- yl)-4-oxo-1,4- dihydropyridazin-3- yl]methoxy}phenyl)car- bamate | Calc'd 370, found 370 |
| 123 | 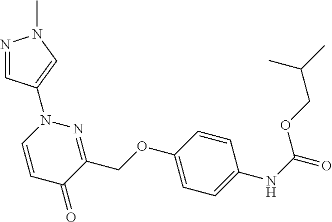 [SMILES: CC(C)COC(=O)Nc1ccc(OCc2nn(-c3cnn(C)c3)ccc2=O)cc1] | 2-methylpropyl (4- {[1-(1-methyl-1H- pyrazol-4-yl)-4-oxo- 1,4-dihydropyridazin- 3- yl]methoxy}phenyl)car- bamate | Calc'd 398, found 398 |

### Scheme #15

### Example #124

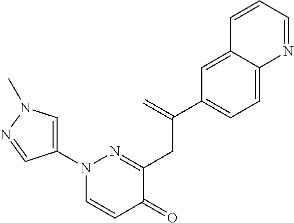 [SMILES: C=C(Cc1nn(-c2cnn(C)c2)ccc1=O)c1ccc2ncccc2c1]

### 1-(1-Methyl-1H-pyrazol-4-yl)-3-(2-quinolin-6-ylprop-2-en-1-yl)pyridazin-4(1H)-one

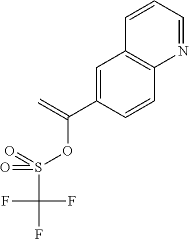 [SMILES: C=C(OS(=O)(=O)C(F)(F)F)c1ccc2ncccc2c1]

### Step 1. 1-Quinolin-6-ylvinyl trifluoromethanesulfonate

A solution of 1-quinolin-6-ylethanone (Intermediate #34, 410 mg, 2.392 mmol) in THF (12 mL) was cooled to −78° C. Lithium hexamethyldisilazide in THF (1 M, 2990 μL, 2.99 mmol) was added drop-wise and the reaction was stirred for 30 minutes, at which point 2-[N,N-bis(trifluoromethylsulfonyl)amino]pyridine (980 mg, 2.63 mmol) was added. After stirring for 10 minutes, the cooling bath was removed and the reaction was warmed to room temperature over hours. The reaction mixture was quenched with saturated aqueous potassium hydrogen sulfate solution, diluted with ethyl acetate and washed with water and ethyl acetate. The combined organics were concentrated in vacuo and purified by silica gel flash chromatography (MPLC, gradient elution, 0-100% EtOAc/hexanes) to obtain 1-quinolin-6-ylvinyl trifluoromethanesulfonate.

LRMS (ESI) calc'd for C12H9NO3 S [M+H]⁺: 304. Found: 304.

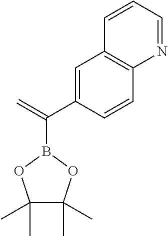 [SMILES: C=C(B1OC(C)(C)C(C)(C)O1)c1ccc2ncccc2c1]

### Step 2. 6-[1-(4,4,5,5-Tetramethyl-1,3,2-dioxaborolan-2-yl)vinyl]quinoline

A microwave vial was charged with 1-quinolin-6-ylvinyl trifluoromethanesulfonate (420 mg, 1.39 mmol), toluene (14 mL), bis(pinacolato)diboron (350 mg, 1.39 mmol), triphenylphosphine (18 mg, 0.069 mmol), sodium phenoxide (230 mg, 1.94 mmol) and bis(triphenylphosphine)palladium(II)dichloride (29 mg, 0.042 mmol), purged with a stream of nitrogen gas for 5 minutes, sealed and heated to 55° C. for 2 hours. The crude reaction mixture was filtered through a column pre-packed with Celite and the combined organics were concentrated in vacuo onto silica gel and purified by silica gel flash chromatography (MPLC, gradient elution, 0-100% EtOAc/hexanes) to obtain 6-[1-(4,4,5,5-tetramethyl-1,3,2-dioxaborolan-2-yl)vinyl]quinoline.

LRMS (ESI) calc'd for C17H21BNO2 [M+H]: 282. Found: 282.

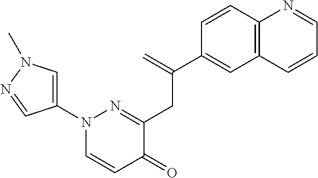 [SMILES: C=C(Cc1nn(-c2cnn(C)c2)ccc1=O)c1ccc2ncccc2c1]

### Step 3. 1-(1-Methyl-1H-pyrazol-4-yl)-3-(2-quinolin-6-ylprop-2-en-1-yl)pyridazin-4(1H)-one

A microwave vial was charged with 6-[1-(4,4,5,5-tetramethyl-1,3,2-dioxaborolan-2-yl)vinyl]quinoline (69 mg, 0.25 mmol), 3-(chloromethyl)-1-(1-methyl-1H-pyrazol-4-yl)pyridazin-4(1H)-one (Intermediate #1, 46 mg, 0.21 mmol), 1,1′-diphenylphosphinoferrocene palladium (II) dichloride dichloromethane adduct (3.0 mg, 4.11 μmol), potassium phosphate tribasic (107 mg, 0.617 mmol), dimethoxyethane (1.9 mL), and water (190 μL), purged with a stream of nitrogen for 5 minutes, sealed and heated to 100° C. for one hour. The crude reaction mixture was filtered through a column pre-packed with Celite and the combined organics were concentrated in vacuo onto silica gel and purified by silica gel flash chromatography (MPLC, gradient elution, 0-15% DCM/MeOH) to obtain 1-(1-methyl-1H-pyrazol-4-yl)-3-(2-quinolin-6-ylprop-2-en-1-yl)pyridazin-4(1B)-one.

LRMS (ESI) calc'd for C20H18N5O [M+H]⁺: 344. Found: 344.

### Scheme #15

### Examples #125 and 126

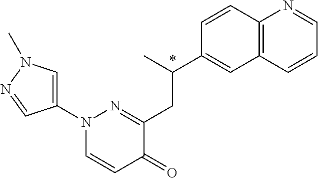 [SMILES: CC(Cc1nn(-c2cnn(C)c2)ccc1=O)c1ccc2ncccc2c1]

### 1-(1-Methyl-1H-pyrazol-4-yl)-3-[(2S)-2-quinolin-6-ylpropyl]pyridazin-4(1H)-one and 1-(1-methyl-1H-pyrazol-4-yl)-3-[(2R)-2-quinolin-6-ylpropyl]pyridazin-4(1H)-one

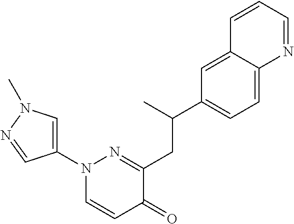 [SMILES: CC(Cc1nn(-c2cnn(C)c2)ccc1=O)c1ccc2ncccc2c1]

### Step 1. 1-(1-Methyl-1H-pyrazol-4-yl-3-(2-quinolin-6-ylpropyl)pyridazin-4(1H)-one

To an oven dried, nitrogen cooled, 5 mL microwave vial equipped with a stir bar was added Pd/C (5.0 mg, 0.049 mmol) and 1-(1-methyl-1H-pyrazol-4-yl)-3-(2-quinolin-6-ylprop-2-en-1-yl)pyridazin-4(1H)-one (Example #124, 56 mg, 0.16 mmol). The vial was sealed under nitrogen, methanol (1.6 mL) was added via syringe and the reaction was stirred under a balloon of hydrogen gas at room temperature for 3.5 hours. The crude reaction mixture was filtered through a column pre-packed with Celite and the combined organics were concentrated in vacuo and purified by silica gel flash chromatography (MPLC, gradient elution, 0-15% DCM/MeOH) to obtain 1-(1-methyl-1H-pyrazol-4-yl)-3-(2-quinolin-6-ylpropyl)pyridazin-4(1H)-one.

LRMS (ESI) calc'd for C20H20N5O [M+H]: 346. Found: 346.

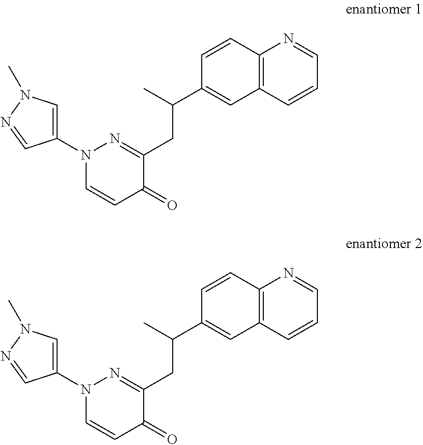 [SMILES: CC(Cc1nn(-c2cnn(C)c2)ccc1=O)c1ccc2ncccc2c1 ; CC(Cc1nn(-c2cnn(C)c2)ccc1=O)c1ccc2ncccc2c1]

### Step 2. 1-(1-Methyl-1H-pyrazol-4-yl)-3-[(2S)-2-quinolin-6-ylpropyl pyridazin-4(1H)-one and 1-(1-methyl-1H-pyrazol-4-yl)-3-(2R)-2-quinolin-6-ylpropyl]pyridazin-4(1H)-one

Chiral separation was performed using chiral SFC as follows:

### Column: Chiral Technology AD-H 2.1×25 cm, 5 uM.

MP: 30%/70% Methanol/CO2 (no other modifiers).
Flow rate: 70 mL/Min, 8 min run time.

### WL: 220 nm.

Racemic 1-(1-methyl-1H-pyrazol-4-yl)-3-(2-quinolin-6-ylpropyl)pyridazin-4(1H)-one (43.8 mg) was dissolved in methanol (2 mL). Injections of 1.0 mL were performed on Berger Multigram II SFC. Enantiomers eluted at 5.25 minutes (enantiomer 1, Example #125) and 6.68 minutes (enantiomer 2, Example #126). No attempt was made to identify absolute stereochemistry.

LRMS (ESI) calc'd for C20H20N5O [M+H]: 346. Found: 346.

### Scheme #16

### Example #127

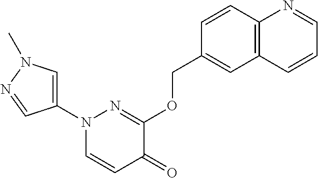 [SMILES: Cn1cc(-n2ccc(=O)c(OCc3ccc4ncccc4c3)n2)cn1]

### 1-(1-Methyl-1H-pyrazol-4-yl)-3-(quinolin-6-ylmethoxy)pyridazin-4(1H)-one

### Step 1. 1-(1-Methyl-1H-pyrazol-4-yl)-3-(quinolin-6-ylmethoxy)pyridazin-4(1H)-one

An oven-dried, nitrogen cooled 2 mL microwave vial was charged with 6-quinolinylmethanol (20 mg, 0.13 mmol) and DMF (0.5 mL) and cooled to 0° C. Sodium hydride (60%, 4.8 mg, 0.120 mmol) was added and the reaction was stirred at 0° C. for 30 min. The reaction was then charged with a solution of 3-chloro-1-(1-methyl-1H-pyrazol-4-yl)pyridazin-4(1H)-one (Intermediate #35, 17 mg, 0.081 mmol) in DMF (1.5 mL) and heated to 50° C. for 16.5 hours. The reaction mixture was diluted with water and extracted with EtOAc (3×). The combined organic layers were washed with brine, dried over magnesium sulfate, filtered and concentrated in vacuo. The aqueous layer was concentrated in vacuo and the resulting solids were extracted with DCM and the organics were concentrated in vacuo. Each residue was purified separately (MPLC, gradient elution, 0-20% MeOH/DCM) and the purified products were combined to give 1-(1-methyl-1H-pyrazol-4-yl)-3-(quinolin-6-ylmethoxy)pyridazin-4(1H)-one.

LRMS (ESI) calc'd for C18H16N5O2 [M+H]⁺: 334. Found: 334.

The following examples were prepared according to Scheme #16 following similar procedures described for Example #127 using Intermediates #35-36 and #37-43, which can be achieved by those of ordinary skill in the art of organic synthesis.

| | | | Exact Mass |
|---|---|---|---|
| Example | Structure | IUPAC Name | [M + H]+ |
| 128 | 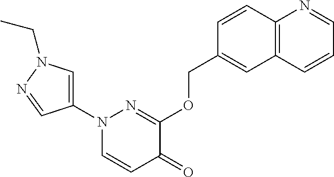 [SMILES: CCn1cc(-n2ccc(=O)c(OCc3ccc4ncccc4c3)n2)cn1] | 1-(1-ethyl-1H- pyrazol-4-yl)-3- (quinolin-6- ylmethoxy)pyridazin- 4(1H)-one | Calc'd 348, found 348 |
| 129 | 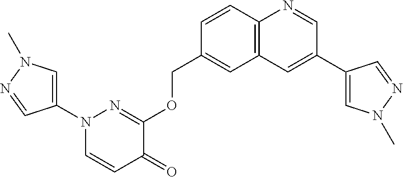 [SMILES: Cn1cc(-c2cnc3ccc(COc4nn(-c5cnn(C)c5)ccc4=O)cc3c2)cn1] | 1-(1-methyl-1H- pyrazol-4-yl)-3-{[3- (1-methyl-1H-pyrazol- 4-yl)quinolin-6- yl]methoxy}pyridazin- 4(1H)-one | Calc'd 414, found 414 |
| 130 | 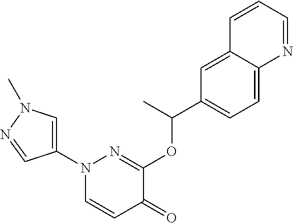 [SMILES: CC(Oc1nn(-c2cnn(C)c2)ccc1=O)c1ccc2ncccc2c1] | 1-(1-methyl-1H- pyrazol-4-yl)-3-(1- quinolin-6- ylethoxy)pyridazin- 4(1H)-one | Calc'd 348, found 348 |
| 131 | 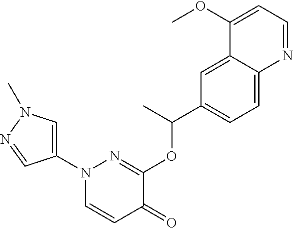 [SMILES: COc1ccnc2ccc(C(C)Oc3nn(-c4cnn(C)c4)ccc3=O)cc12] | 3-[1-(4- methoxyquinolin-6- yl)ethoxy]-1-(1- methyl-1H-pyrazol-4- yl)pyridazin-4(1H)- one | Calc'd, found |
| 132 | 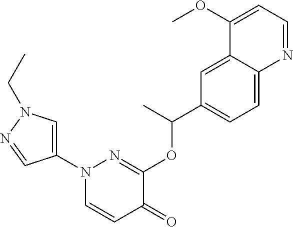 [SMILES: CCn1cc(-n2ccc(=O)c(OC(C)c3ccc4nccc(OC)c4c3)n2)cn1] | 1-(1-ethyl-1H- pyrazol-4-yl)-3-[1-(4- methoxyquinolin-6- yl)ethoxy]pyridazin- 4(1H)-one | Calc'd, found |
| 133 | 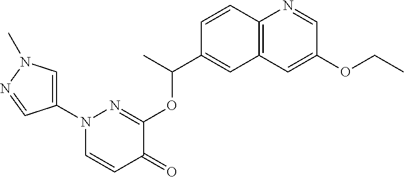 [SMILES: CCOc1cnc2ccc(C(C)Oc3nn(-c4cnn(C)c4)ccc3=O)cc2c1] | 3-[1-(3- ethoxyquinolin-6- yl)ethoxy]-1-(1- methyl-1H-pyrazol-4- yl)pyridazin-4(1H)- one | Calc'd 392, found 392 |
| 134 | 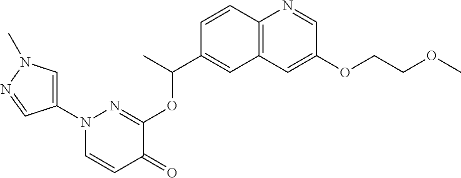 [SMILES: COCCOc1cnc2ccc(C(C)Oc3nn(-c4cnn(C)c4)ccc3=O)cc2c1] | 3-{1-[3-(2- methoxyethoxy)quinolin- 6-yl]ethoxy}-1-(1- methyl-1H-pyrazol-4- yl)pyridazin-4(1H)- one | Calc'd 422, found 422 |
| 135 | 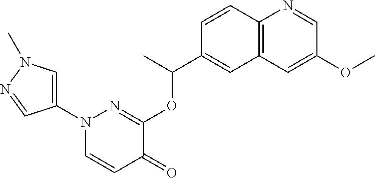 [SMILES: COc1cnc2ccc(C(C)Oc3nn(-c4cnn(C)c4)ccc3=O)cc2c1] | 3-[1-(3- methoxyquinolin-6- yl)ethoxy]-1-(1- methyl-1H-pyrazol-4- yl)pyridazin-4(1H)- one | Calc'd 378, found 378 |
| 136 | 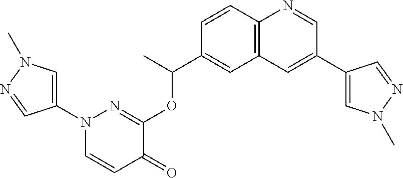 [SMILES: CC(Oc1nn(-c2cnn(C)c2)ccc1=O)c1ccc2ncc(-c3cnn(C)c3)cc2c1] | 1-(1-methyl-1H- pyrazol-4-yl)-3-{1-[3- (1-methyl-1H-pyrazol- 4-yl)quinolin-6- yl]ethoxy}pyridazin- 4(1H)-one | Calc'd 428, found 428 |

### Scheme #16

### Example #137

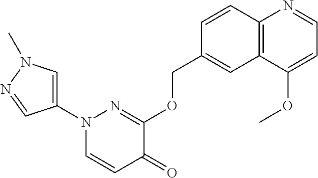 [SMILES: COc1ccnc2ccc(COc3nn(-c4cnn(C)c4)ccc3=O)cc12]

### 3-[(4-methoxyquinolin-6-yl)methoxy]-1-(1-methyl-1H-pyrazol-4-yl)pyridazin-4(1H)-one

### Step 1. 3-[(4-methoxyquinolin-6-yl)methoxy]-1-(1-methyl-1H-pyrazol-4-yl)pyridazin-4(1-one

A mixture of 3-chloro-1-(1-methyl-1H-pyrazol-4-yl)pyridazin-4(1H)-on (Intermediate #35, 15 mg, 0.071 mmol), 4-methoxy-6-{[2-(trimethylsilyl)ethoxy]methyl}quinoline (Intermediate #31, Step 1, 26 mg, 0.090 mmol) and cesium fluoride (16 mg, 0.11 mmol) in DMF (2 mL) was heated in a sealed 5 mL microwave vial to 100° C. for 20.5 hours followed by heating at 200° C. for 1 hour under microwave irradiation. The reaction mixture was allowed to cool to room temperature, diluted with DMSO and purified by mass-triggered reverse-phase HPLC to obtain 3-[(4-methoxyquinolin-6-yl)methoxy]-1-(1-methyl-1H-pyrazol-4-yl)pyridazin-4(1H)-one.

LRMS (ESI) calc'd for C19H18N5O3 [M+H]⁺: 364. Found: 364.

### Scheme #16

### Example #138 and 139

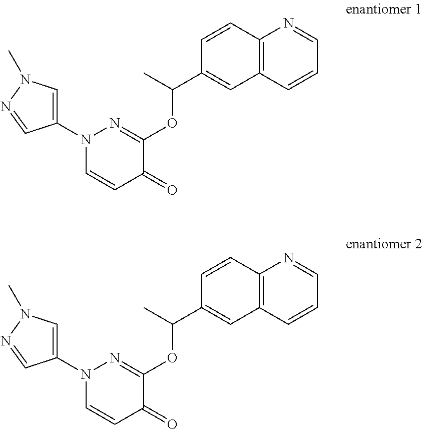 [SMILES: CC(Oc1nn(-c2cnn(C)c2)ccc1=O)c1ccc2ncccc2c1 ; CC(Oc1nn(-c2cnn(C)c2)ccc1=O)c1ccc2ncccc2c1]

### Chiral separation of racemic 1-(1-methyl-1H-pyrazol-4-yl)-3-[1-(quinolin-6-yl)ethoxy]pyridazin-4(1H)-one

### Step 1. 1-(1-Methyl-1H-pyrazol-4-yl)-3-[(1R)-1-quinolin-6-yl)ethoxy]pyridazin-4(1H)-one and 1-(1-methyl-1H-pyrazol-4-yl)-3-[(1S)-1-quinolin-6-ylethoxy]pyridazin-4(1H)-one

Chiral separation was performed using chiral SFC as follows:

### Column: Chiral Technology AD-H 2.1×25 cm, 5 uM.

MP: 35%/65% Ethanol/CO2 (no other modifiers).
Flow rate: 70 mL/Min, 8 min run time.

### WL: 220 nm

Racemic 1-(1-methyl-1H-pyrazol-4-yl)-3-(1-quinolin-6-ylethoxy)pyridazin-4(1H)-one (Example #130, 26.5 mg) was dissolved in methanol (3 mL). Injections of 1.25 mL were performed on the Berger Multigram II SFC. Elution of the enantiomers was observed at 3.66 minutes (enantiomer 1) and 4.84 minutes (enantiomer 2). No attempt was made to confirm the absolute stereochemistry.

LRMS (ESI) calc'd for C19H18N5O [M+H]⁺: 348. Found: 348.

The following examples were prepared according to Scheme #16 following similar procedures described for Examples #138 and #139 using Examples #131-136, which can be achieved by those of ordinary skill in the art of organic synthesis. No attempt was made to confirm the absolute stereochemistry. In all cases, the first eluting enantiomer is labeled “enantiomer 1,” and the second eluting enantiomer is labeled “enantiomer 2.”

| | | | Exact Mass |
|---|---|---|---|
| Example | Structure | IUPAC Name | [M + H]+ |
| 140 | 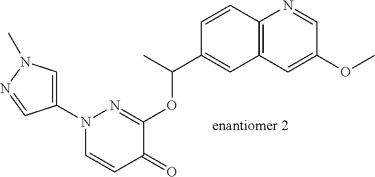 [SMILES: COc1cnc2ccc(C(C)Oc3nn(-c4cnn(C)c4)ccc3=O)cc2c1] | 3-[1-(3- methoxyquinolin-6- yl)ethoxy]-1-(1-methyl- 1H-pyrazol-4- yl)pyridazin-4(1H)-one enantiomer 2 | Calc'd 378, found 378 |
| 141 | 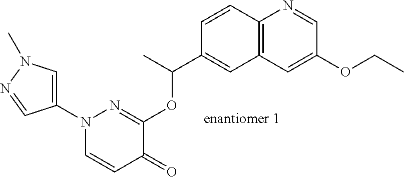 [SMILES: CCOc1cnc2ccc(C(C)Oc3nn(-c4cnn(C)c4)ccc3=O)cc2c1] | 3-[1-(3-ethoxyquinolin- 6-yl)ethoxy]-1-(1- methyl-1H-pyrazol-4- yl)pyridazin-4(1H)-one enantiomer 1 | Calc'd 392, found 392 |
| 142 | 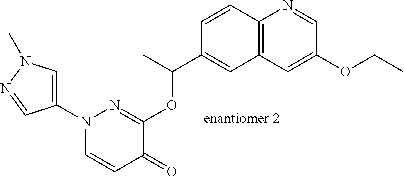 [SMILES: CCOc1cnc2ccc(C(C)Oc3nn(-c4cnn(C)c4)ccc3=O)cc2c1] | 3-[1-(3-ethoxyquinolin- 6-yl)ethoxy]-1-(1- methyl-1H-pyrazol-4- yl)pyridazin-4(1H)-one enantiomer 2 | Calc'd 392, found 392 |
| 143 | 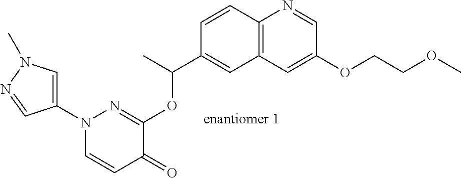 [SMILES: COCCOc1cnc2ccc(C(C)Oc3nn(-c4cnn(C)c4)ccc3=O)cc2c1] | 3-{1-[3-(2- methoxyethoxy)quinolin- 6-yl]ethoxy}-1-(1- methyl-1H-pyrazol-4- yl)pyridazin-4(1H)-one enantiomer 1 | Calc'd 422, found 422 |
| 144 | 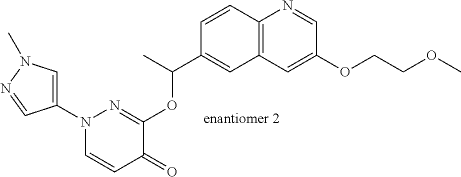 [SMILES: COCCOc1cnc2ccc(C(C)Oc3nn(-c4cnn(C)c4)ccc3=O)cc2c1] | 3-{1-[3-(2- methoxyethoxy)quinolin- 6-yl]ethoxy}-1-(1- methyl-1H-pyrazol-4- yl)pyridazin-4(1H)-one enantiomer 2 | Calc'd 422, found 422 |
| 145 | 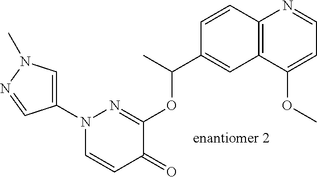 [SMILES: COc1ccnc2ccc(C(C)Oc3nn(-c4cnn(C)c4)ccc3=O)cc12] | 3-[1-(4- methoxyquinolin-6- yl)ethoxy]-1-(1-methyl- 1H-pyrazol-4- yl)pyridazin-4(1H)-one enantiomer 2 | Calc'd 378, found 378 |
| 146 | 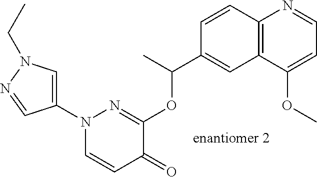 [SMILES: CCn1cc(-n2ccc(=O)c(OC(C)c3ccc4nccc(OC)c4c3)n2)cn1] | 1-(1-ethyl-1H-pyrazol- 4-yl)-3-[1-(4- methoxyquinolin-6- yl)ethoxy]pyridazin- 4(1H)-one enantiomer 2 | Calc'd 392, found 392 |

### Scheme #17

### Example #147

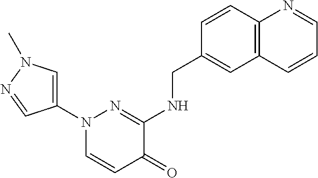 [SMILES: Cn1cc(-n2ccc(=O)c(NCc3ccc4ncccc4c3)n2)cn1]

### 1-(1-methyl-1H-pyrazol-4-yl)-3-[(quinolin-6-ylmethyl)amino]pyridazin-4(1H)-one

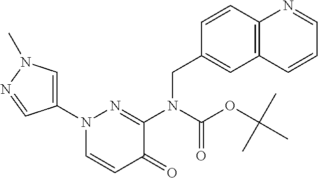 [SMILES: Cn1cc(-n2ccc(=O)c(N(Cc3ccc4ncccc4c3)C(=O)OC(C)(C)C)n2)cn1]

### Step 1. tert-butyl [4-(1-methyl-1H-pyrazol-4-yl)-4-oxo-1,4-dihdropyridazin-3-yl](quinolin-6-ylmethyl)carbamate

To a stirring solution of tert-butyl [1-(1-methyl-1H-pyrazol-4-yl)-4-oxo-1,4-dihydropyridazin-3-yl]carbamate (50 mg, 0.17 mmol, Intermediate #35, Step 1), triphenylphosphine (90 mg, 0.34 mmol) and 6-quinolinylmethanol (55 mg, 0.35 mmol) in THF (1 mL) was added TMAD (59 mg, 0.34 mmol) and the reaction mixture was stirred at room temperature for 20 hours. The reaction mixture was concentrated in vacuo and purified by silica gel flash chromatography (MPLC, gradient elution, 0-20% MeOH/DCM) to obtain tert-butyl [1-(1-methyl-1H-pyrazol-4-yl)-4-oxo-1,4-dihydropyridazin-3-yl](quinolin-6-ylmethyl)carbamate.

LRMS (ESI) calc'd for C23H25N6O3 [M+H]⁺: 433. Found: 433.

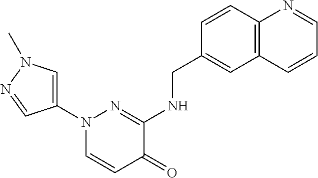 [SMILES: Cn1cc(-n2ccc(=O)c(NCc3ccc4ncccc4c3)n2)cn1]

### Step 2. 1-(1-methyl-1H-pyrazol-4-yl)-3-[(quinolin-6-ylmethyl)amino]pyridazin-4(1-one

A mixture of tert-butyl [1-(1-methyl-1H-pyrazol-4-yl)-4-oxo-1,4-dihydropyridazin-3-yl](quinolin-6-ylmethyl)carbamate (74.2 mg, 0.172 mmol), DCM (1.5 mL) and TFA (0.5 mL, 6.49 mmol) was stirred at ambient temperature overnight, then concentrated in vacuo. The residue was taken up in methanol, filtered through a PL-HCO3 cartridge and the filtrate was concentrated in vacuo. The residue was purified by reverse-phase HPLC (5-40% MeCN/H2O, 0.1% TFA, freebased with PL-HCO3 SPE cartridges) to obtain 1-(1-methyl-1H-pyrazol-4-yl)-3-[(quinolin-6-ylmethyl)amino]pyridazin-4(1H)-one.

LRMS (ESI) calc'd for C18H17N6O [M+H]⁺: 333. Found: 333.

The following examples were prepared according to Scheme #16 following similar procedures described for Example #147 using Intermediates #35 and 38, which can be achieved by those of ordinary skill in the art of organic synthesis.

| | | | Exact Mass |
|---|---|---|---|
| Example | Structure | IUPAC Name | [M + H]+ |
| 148 | 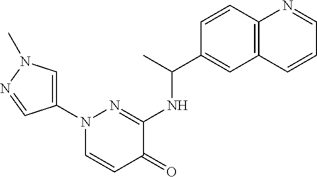 [SMILES: CC(Nc1nn(-c2cnn(C)c2)ccc1=O)c1ccc2ncccc2c1] | 1-(1-methyl-1H- pyrazol-4-yl)-3-{[1- (quinolin-6- yl)ethyl]amino}pyrida- zin-4(1H)-one | Calc'd 347, found 347 |

### Scheme #18

### Example #149

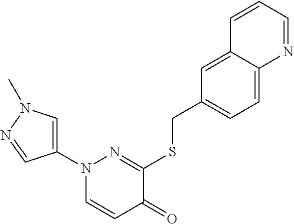 [SMILES: Cn1cc(-n2ccc(=O)c(SCc3ccc4ncccc4c3)n2)cn1]

### 1-(1-methyl-1H-pyrazol-4-yl)-3-[(quinolin-6-ylmethyl)thio]pyridazin-4(1H)-one

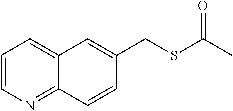

### Step 1. S-(quinolin-6-ylmethyl)ethanethioate

An oven dried microwave vial under nitrogen atmosphere was charged with thioacetic acid (0.902 mL, 12.6 mmol) and 6-quinolinylmethanol (1.00 g, 6.28 mmol) in THF (2.5 mL). A separate oven dried microwave vial under nitrogen atmosphere was charged with di-tert-butyl azodicarboxylate (2.89 g, 12.6 mmol) and triphenylphosphine (3.30 g, 12.6 mmol) in THF (2.5 mL) and the mixture cooled to 0° C. The first mixture was added via syringe to the second mixture at 0° C. and the combined reaction mixtures were stirred together for 30 min at 0° C., warmed to room temperature and stirred for 15 hours. The reaction mixture was then charged with TFA (2.5 mL), stirred for 15 minutes, poured into 60 mL of saturated sodium carbonate at 0° C., stirred for 2 hours and extracted with EtOAc (3×90 mL). The combined organic layers were washed with brine, dried over sodium sulfate, filtered, concentrated in vacuo and purified by silica gel flash chromatography (MPLC, gradient elution, 0-50% EtOAc/hexanes) gave S-(quinolin-6-ylmethyl)ethanethioate.

LRMS (ESI) calc'd for C2H12NOS [M+H]⁺: 218. Found: 218.

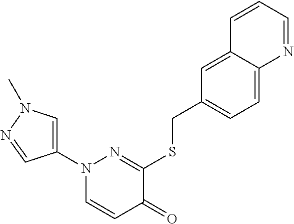 [SMILES: Cn1cc(-n2ccc(=O)c(SCc3ccc4ncccc4c3)n2)cn1]

### Step 2. 1-(1-methyl-1H-pyrazol-4-yl)-3-[(quinolin-6-ylmethyl)thio]pyridazin-4(1H)-one

S-(Quinolin-6-ylmethyl)ethanethioate (99 mg, 0.46 mmol), 2 N sodium hydroxide (0.228 mL, 0.456 mmol) and MeOH (0.018 mL, 0.456 mmol) were stirred in THF (4.1 mL) at room temperature for 5 hours followed by stirring at 50° C. for 18 hours. Ambient temperature was attained and additional 2 N sodium hydroxide (0.228 mL, 0.456 mmol) and MeOH (1 mL) were added. After 1 hour at room temperature, the solvent was removed in vacuo, and the residue was taken up in DMF (4.5 mL). Sodium hydride (18.2 mg, 0.456 mmol) was added, followed by 3-chloro-1-(1-methyl-1H-pyrazol-4-yl)pyridazin-4(1H)-one (Intermediate #35, 80 mg, 0.38 mmol) and the resulting mixture was stirred at room temperature for 2 hours. TFA (0.146 mL, 1.90 mmol) was added and the reaction mixture was purified directly by mass-triggered reverse phase HPLC. The product residue was dissolved in MeOH, eluted through a PS—HCO3 cartridge and concentrated in vacuo to give 1-(1-methyl-1H-pyrazol-4-yl)-3-[(quinolin-6-ylmethyl)thio]pyridazin-4(1H)-one.

LRMS (ESI) calc'd for C18H16N5OS [M+H]⁺: 350. Found: 350.

### Scheme #19

### Example #150

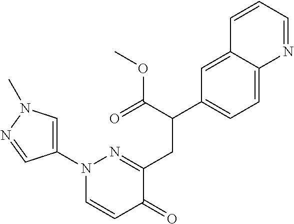 [SMILES: COC(=O)C(Cc1nn(-c2cnn(C)c2)ccc1=O)c1ccc2ncccc2c1]

### Methyl 3-[1-(1-methyl-1H-pyrazol-4-yl)-4-oxo-1,4-dihydropyridazin-3-yl]-2-(quinolin-6-yl)propanoate

### Step 1. Methyl 3-[1-(1-methyl-1H-pyrazol-4-yl)-4-oxo-1,4-dihydropyridazin-3-yl]-2-(quinolin-6-yl)propanoate

A solution of methyl quinolin-6-ylacetate (99 mg, 0.49 mmol) in THF (0.55 mL)/DMF (0.55 mL) was charged with sodium hydride (21.4 mg, 0.534 mmol), added dropwise from a pipette to a solution of 3-(chloromethyl)-1-(1-methyl-1H-pyrazol-4-yl)pyridazin-4(1H)-one (Intermediate #1, 100 mg, 0.445 mmol) in THF (0.55 mL)/DMF (0.55 mL) at room temperature and the resulting mixture was stirred at room temperature for 45 minutes. Additional methyl quinolin-6-ylacetate (99 mg, 0.49 mmol) was taken up in THF (0.55 mL)/DMF (0.55 mL), charged with sodium hydride (21.4 mg, 0.534 mmol), added dropwise from a pipette to the reaction mixture and stirring was continued at room temperature for 45 minutes. The reaction mixture was quenched with methanol, concentrated in vacuo onto silica gel and purified by silica gel flash chromatography (MPLC, gradient elution, 0-20% MeOH/EtOAc and then 0-8% MeOH/DCM) to obtain methyl 3-[1-(1-methyl-1H-pyrazol-4-yl)-4-oxo-1,4-dihydropyridazin-3-yl]-2-(quinolin-6-yl)propanoate.

LRMS (ESI) calc'd for C21H20N5O3 [M+H]⁺: 390. Found: 390.

### Pharmaceutical Composition

As a specific embodiment of this invention, 100 mg of 1-(1-Methyl-1H-pyrazol-4-yl)-3-[(quinolin-6-yloxy)methyl]pyridazin-4(1H)-one is formulated with sufficient finely divided lactose to provide a total amount of 580 to 590 mg to fill a size 0, hard-gelatin capsule.

### Assays

The compounds of the instant invention described in the Examples were tested by the assays described below and were found to have MET inhibitory activity. Other assays are known in the literature and could be readily performed by those of skill in the art (see, for example, U.S. Patent Application Publications US 2005/0075340 A1, Apr. 7, 2005, pages 18-19; and PCT Publication WO 2005/028475, Mar. 31, 2005, pages 236-248).

### I. In Vitro Kinase Assays

Recombinant GST-tagged cytosolic domains of human c-Met and other receptor tyrosine kinases including mouse c-Met, human Ron, KDR, IGFR, EGFR, FGFR, Mer, TrkA and Tie2 are used to determine whether the compounds of the instant invention modulate the enzymatic activities of these kinases.

Soluble recombinant GST-tagged cytosolic domains of c-Met and other receptor tyrosine kinases are expressed in a baculovirus system (Pharmingen) according to a protocol recommended by the manufacturer. The c-DNA encoding each cytosolic domain is subcloned into a baculovirus expression vector (pGcGHLT-A, B or C, Pharmingen) containing an in frame 6× histidine tag and a GST tag. The resulting plasmid construct and BaculoGold baculovirus DNA (Pharmingen) are used to co-transfect Sf9 or Sf21 insect cells. After confirming expression of GST-tagged kinase fusion, a high titer recombinant baculovirus stock is produced, expression conditions are optimized, and a scaled up expression of rat KDR-GST fusion is performed. The fusion kinase is then purified from the insect cell lysate by affinity chromatography using glutathione agarose (Pharmingen). The purified protein is dialyzed against 50% glycerol, 2 mM DTT, 50 mM Tris-HCl (pH 7.4) and stored at −20° C. The protein concentrations of the fusion proteins are determined using Coomassie Plus Protein Assay (Pierce) with BSA as standard.

The kinase activities of c-Met and other kinases are measured using a modified version of the homogeneous time-resolved tyrosine kinase assay described by Park et al. (1999, Anal. Biochem. 269:94-104).

The procedure for determining the potency of a compound to inhibit c-Met kinase comprises the following steps:
1. Prepare 3-fold serial diluted compound solutions in 100% dimethyl sulfoxide (DMSO) at 20× of the desired final concentrations in a 96 well plate.
2. Prepare a master reaction mix containing 6.67 mM MgCl₂, 133.3 mM NaCl, 66.7 mM Tris-HCl (pH 7.4), 0.13 mg/ml BSA, 2.67 mM dithiothreitol, 0.27 nM recombinant c-Met and 666.7 nM biotinylated synthetic peptide substrate (biotin-ahx-EQEDEPEGDYFEWLE-CONH₂) (SEQ.ID.NO.:1).
3. In a black assay plate, add 2.5 μl of compound solution (or DMSO) and 37.5 μl of master reaction mix per well. Initiate the kinase reaction by adding 10 μl of 0.25 mM MgATP per well. Allow the reactions to proceed for 80 min at room temperature. The final conditions for the reaction are 0.2 nM c-Met, 0.5 μM substrate, 50 μM MgATP, mM MgCl₂, 100 mM NaCl, 2 mM DTT, 0.1 mg/ml BSA, 50 mM Tris (pH 7.4) and 5% DMSO.
4. Stop the kinase reaction with 50 μl of Stop/Detection buffer containing 10 mM EDTA, 25 mM HEPES, 0.1% TRITON X-100, 0.126 μg/ml Eu-chelate labeled anti-phosphotyrosine antibody PY20 (cat. #AD0067, PerkinElmer) and 45 μg/ml Streptavidin-allophycocyanin conjugate (cat. #PJ25S, Prozyme).
5. Read HTRF signals on a Victor reader (PerkinElmer) in HTRF mode after 60 min.
6. IC₅₀ is determined by fitting the observed relationship between compound concentration and HTRF signal with a 4-parameter logistic equation.
   Essentially the same procedure was used to determine the potency of compounds to inhibit mouse c-Met, human Ron, KDR, IGFR, EGFR, FGFR, Mer, TrkA and Tie2 except that the concentration of enzyme varied in individual assays (0.2 nM mouse c-Met; 2.5 nM Ron, 8 nM KDR; 0.24 nM IGFR; 0.24 nM EGFR; 0.14 nM FGFR; 16 nM Mer; 8 nM TrkA; 8 nM Tie2).

The compound of the instant invention may be tested in the assay above and inhibitory activity may be determined.

II. GTL-16 pY1349 Cell-Based Assay

The ability of compounds to inhibit the phosphorylation of Met Y1349 in GTL-16 cells (Ponzetto et al. Oncogene 1991; 6:553-559.) was measured using a 384-well AlphaScreen (Perkin Elmer) assay. GTL-16 cells were grown in RPMI Medium 1640 (no phenol red Invitrogen Cat #11835) with 10% FBS, 1% sodium pyruvate and 1% HEPES (pH 7.5). On day one, GTL-16 cells were seeded at a density of 10,000 cells/well in 20 ul of RPMI growth medium on Perkin Elmer CulturePlates. Plates were incubated at 37° C., 5% CO₂ overnight. The next day, 20 nL of serially diluted compounds were added to the cell plate via acoustic dispensing. Final compound concentrations of the 9-point 1:3 serial dilutions ranged from 10 uM to 1.5 nM. Cells were incubated in the presence of compound for 60 min at 37° C., 5% CO₂. After incubation, 20 uL of culture media were removed and 10 uL/well lysis buffer (30 mM Tris-HCL (pH 7.5), 5 mM EDTA, 50 mM NaCl, 30 mM NaPPi, 50 mM NaF, 0.5% (vol/vol) IGEPAL CA-630, 1% (vol/vol) Triton X-100, 10% glycerol, Roche Mini-Complete™ (without EDTA) protease inhibitor cocktail, 0.5 mM Na₃VO₄, 0.1 mg mL⁻¹ potassium bisperoxo (1,10-phenanthroline) oxovanadate (bpV-phen), 1% (vol/vol) phenylmethylsulfonyl fluoride (PMSF) and 0.5 mg mL⁻¹ Microcystin-LR) containing 1 ug/mL biotinylated anti-HGFR (R&D System, Cat#BAF358) was added to each well. Next, 10 uL of 5 ug/ml anti-phospho-Met Tyr1349 (Cell Signaling Technology, Cat#3121) in PBS plus 0.1% BSA was added to each well. Plates were then incubated at room temperature with shaking for 2 h. After incubation, 10 uL/well anti-IgG (Protein A) acceptor and streptavidin donor AlphaScreen bead mixture (50 ug/mL acceptor, 120 ug/mL donor; PerkinElmer, Cat#: 6760617R) in PBS with 0.1% BSA was added and the plates were incubated in the dark for 2 h. The AlphaScreen signal was read on an Envision (Perkin Elmer). After background correction, and normalization to untreated controls, the percent inhibition of Y1349 phosphorylation at each compound concentration was calculated. The plot of percent inhibition vs. the log of compound concentration was fit with a 4-parameter dose response equation to calculate IC₅₀ values.

### III. GTL-16 and HCT116 Proliferation Assay

The ability of compounds to inhibit the growth of GTL-16 cells with constitutively active amplified cMet (Ponzetto et al. Oncogene 1991; 6:553-559.) was assessed using an assay which measures cellular ATP levels as a proxy for viable cell mass. The assay makes use of a bioluminescent method from Lonza (Cat #LT07-321). In the presence of ATP, luciferase converts luciferin to oxyluciferin and light. The amount of light produced (emission at 565 nM) is measured and correlates with a relative amount of proliferation. A negative control cell line, HCT116 (ATCC # CCL-247), the growth of which is not dependent on met activity, was grown in 90% DMEM, 10% FBS, 10 mM HEPES pH 7.5. Two days prior to compound treatment, a 80-90% confluent flask of GTL-16 cells was split 1:4 in Complete Media and incubated in 5% CO₂ at 37° C. overnight. One day prior to compound treatment, GTL-16 cells at 1000 cells/well and HCT116 at 1000 cells/well were seeded in 20 uL complete medium in 384 well Perkin Elmer CulturePlates. Cells were incubated in the cell plates at 37° C., 5% CO₂ overnight. The next day, 100 nL of serially diluted compounds to the cell plate via acoustic dispensing. Cells were then incubated in the presence of compound for 72 hr at 37° C., 5% CO₂. At the end of the incubation, cells were lysed and ATP content was measured following the manufacturer's instructions. Assay plates were read in a luminometer after 2 min (1 sec exposure per well). The highest final compound concentration in the assay plates was 50 uM for test compounds, which were serially diluted 1:3 to give a final concentration series of 50000, 16667, 5556, 1852, 617, 206, 69, 23 and 7.6 nM. Final DMSO concentration was 0.5% in each well. The percentage inhibition of cell viability was calculated relative to untreated controls, plotted as a function of the log of compound concentration and analyzed using a four parameter logistical fit to calculate IC₅₀ values.

### IV. HPAF Scatter Assay

The ability of compounds to inhibit the HGF-dependent scattering phenotype of HPAF-II cells was measured using a modified version of the assay described by Chan et al. 2008 (Chan et al. J. Biomolec. Screening 2008; 13:847-854). Briefly, HPAF-II cells (ATCC #CRL1997) were plated in 50 uL DMEM (#11995)+10% FBS+P/S at a density of 3,000 cells/well in Costar black clear bottom 384-well plates [Product no. 3712] and incubated at 37° C. overnight. The next day, 100 nL of serially diluted compound in DMSO was added to each well in the cell plate to give a nine-point 1:3 dilution series with final concentrations ranging from 20 uM to 3 nM. Cells were preincubated with compound at 37° C. for one hour. To stimulate the scattering phenotype, 10 uL of 24 ng/ml HGF (R&D Systems 294-HGN) was next added to each well, giving a final concentration of 4 ng/ml HGF. Cells were incubated with both compound and HGF at 37° C. for an additional 22 hrs. Control HGF-stimulated wells without compound treatment and control wells without HGF or compound were included on each plate. Next, to visualize the cells, each plate was washed in PBS 1×, fixed in ice cold methanol for 3 min at RT, washed in PBS 3×, stained with Hoechst (1:2500) in PBS/0.1% Triton for 15 min in the dark, and finally washed in PBS 4× before imaging on an INCell Analyzer 1000 (GE Healthcare). Individual cell by cell SOI internuclear distance information was exported and then processed using a Pipeline Pilot (Accelrys) protocol to calculate the percentage of scattered cells. The percent inhibition of the scattering phenotype was calculated relative to cells without compound treatment, plotted against the log of compound concentration and then fit to a four parameter logistic fit to obtain IC₅₀ values.

V. K_{I} and k_{inact} Determination for Time-Dependent Inhibition of CYP3A4

The time-dependant inhibition assay for CYP3A4 was performed in two steps, a preincubation step where the test compound was incubated with human liver microsomes and the secondary incubation period where CYP3A4 substrate, testosterone was added to the preincubate to measure residual CYP3A4 activity. Wells contained human liver microsomes (42.5 μl, 2.35 mg/ml) which were diluted from a stock (20 mg/ml) in potassium phosphate buffer (50 mM, pH 7.4) such that the final concentration in the 50 μl preincubation was 2 mg/ml. The wells also contained test compound (2.5 μl at 20 times the incubation concentration) in a solvent mixture of DMSO:water:methanol (10:50:40) and the same solvent in the absence of the test compound was used as the control. The final concentrations of the test compound in the preincubations were 1.56, 3.13, 6.25, 12.5, 25, 50 and 100 μM. The preincubation times used were 0, 5, 10, 15, and min. Separate preincubations were used for each preincubation time point. The rack containing the wells was pre-warmed for 30 min at 37° C. in an incubator that was gently shaken and the temperature was maintained at 37° C. for the duration of the incubations. The preincubation period was initiated by the addition of NADPH (5 μl, 10 mM) that had been pre-warmed to 37° C. for ten minutes. Following the preincubation step, the secondary incubations were initiated by performing a 10-fold dilution of the preincubate using 450 μl of a pre-warmed (37° C.) solution of NADPH (1 mM) and testosterone (222 μM) in potassium phosphate (50 mM, pH 7.4) The final concentration of NADPH and testosterone in the 500 μl incubation was 1 mM and 200 μM, respectively. After a 10 min incubation, each well was quenched with 1 ml of acetonitrile containing the internal standard, cortisone (0.6 μg/ml) and placed on ice. The rack was centrifuged at 3202 g for 10 min and 200 μl of the supernatant was diluted with 100 μl of water, mixed well and analyzed by LC/MS-MS.

Samples (10 μl) were injected onto a C₁₈ column (2.0 mm×30 mm, 3 μm particle size) and eluted using water containing 0.1% formic acid as the aqueous mobile phase (A), and acetonitrile containing 0.1% formic acid as the organic phase (B), according to the following gradient table:

| Time | Flow Rate | | |
|---|---|---|---|
| (min) | (ml/min) | % A | % B |
| 0.00 | 0.85 | 98 | 2 |
| 0.02 | 0.85 | 98 | 2 |
| 3.02 | 0.85 | 2 | 98 |
| 3.52 | 0.85 | 2 | 98 |
| 3.53 | 0.85 | 98 | 2 |

The eluent from the column was sent to the mass spectrometer and specific multiple reaction monitoring transitions for testosterone metabolite, 6β-OH testosterone (305 m/z>269 m/z) and cortisone (361 m/z>185 m/z) were used for MS/MS detection. Integrated area ratios of the analyte (6β-OH testosterone) to the internal standard (cortisone) were analyzed by nonlinear regression to calculate K_{I} and k_{inact}.

Biological Activity (METKA IC₅₀) generated using
the in vitro kinase assay described herein

| Example | Activity Code |
|---|---|
| 1 | +++ |
| 2 | + |
| 3 | +++ |
| 4 | +++ |
| 5 | +++ |
| 6 | +++ |
| 7 | +++ |
| 8 | +++ |
| 9 | + |
| 10 | +++ |
| 11 | +++ |
| 12 | ++ |
| 13 | ++ |
| 14 | + |
| 15 | + |
| 16 | ++ |
| 17 | + |
| 18 | + |
| 19 | +++ |
| 20 | +++ |
| 21 | +++ |
| 22 | +++ |
| 23 | +++ |
| 24 | +++ |
| 25 | +++ |
| 26 | +++ |
| 27 | ++ |
| 28 | +++ |
| 29 | +++ |
| 30 | +++ |
| 31 | +++ |
| 32 | + |
| 33 | ++ |
| 34 | ++ |
| 35 | +++ |
| 36 | +++ |
| 37 | +++ |
| 38 | +++ |
| 39 | ++ |
| 40 | +++ |
| 41 | ++ |
| 42 | +++ |
| 43 | +++ |
| 44 | +++ |
| 45 | +++ |
| 46 | +++ |
| 47 | +++ |
| 48 | +++ |
| 49 | ++ |
| 50 | +++ |
| 51 | +++ |
| 52 | + |
| 53 | +++ |
| 54 | + |
| 55 | + |
| 56 | + |
| 57 | +++ |
| 58 | +++ |
| 59 | +++ |
| 60 | + |
| 61 | +++ |
| 62 | +++ |
| 63 | +++ |
| 64 | +++ |
| 65 | +++ |
| 66 | +++ |
| 67 | +++ |
| 68 | +++ |
| 69 | +++ |
| 70 | +++ |
| 71 | +++ |
| 72 | +++ |
| 73 | +++ |
| 74 | +++ |
| 75 | +++ |
| 76 | +++ |
| 77 | +++ |
| 78 | +++ |
| 79 | +++ |
| 80 | +++ |
| 81 | +++ |
| 82 | +++ |
| 83 | +++ |
| 84 | +++ |
| 85 | +++ |
| 86 | +++ |
| 87 | +++ |
| 88 | +++ |
| 89 | +++ |
| 90 | +++ |
| 91 | +++ |
| 92 | +++ |
| 93 | +++ |
| 94 | +++ |
| 95 | ++ |
| 96 | +++ |
| 97 | +++ |
| 98 | +++ |
| 99 | +++ |
| 100 | +++ |
| 101 | +++ |
| 102 | +++ |
| 103 | ++ |
| 104 | +++ |
| 105 | +++ |
| 106 | +++ |
| 107 | +++ |
| 108 | +++ |
| 109 | + |
| 110 | +++ |
| 111 | +++ |
| 112 | +++ |
| 113 | + |
| 114 | + |
| 115 | ++ |
| 116 | + |
| 117 | + |
| 118 | ++ |
| 119 | + |
| 120 | + |
| 121 | + |
| 122 | ++ |
| 123 | + |
| 124 | ++ |
| 125 | +++ |
| 126 | ++ |
| 127 | + |
| 128 | ++ |
| 129 | +++ |
| 130 | ++ |
| 131 | ++ |
| 132 | +++ |
| 133 | nd |
| 134 | nd |
| 135 | nd |
| 136 | +++ |
| 137 | + |
| 138 | ++ |
| 139 | ++ |
| 140 | +++ |
| 141 | +++ |
| 142 | + |
| 143 | +++ |
| 144 | ++ |
| 145 | +++ |
| 146 | +++ |
| 147 | ++ |
| 148 | +++ |
| 149 | +++ |
| 150 | + |

+++ IC₅₀ < 100 nM
++ IC₅₀ = 100-1000 nM
+ IC₅₀ = 1000-3000 nM

What is claimed is:

1. A compound of the formula: 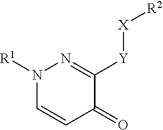
wherein X is CR⁴R^{4′}, C₂₋₃ alkenyl, O, NR⁵ or S;
Y is CR³R^{3′}, O, NR⁵ or S;
R¹ is heteroaryl or aryl, wherein said heteroaryl and aryl groups are optionally substituted with one to three groups independently selected from the group consisting of halo, cyano, C₁₋₆ alkyl, (C₁₋₆ alkyl)R⁷, OR⁷, (C═O)NR⁵R⁶, heterocyclyl, aryl, heteroaryl and (heteroaryl)R⁶;
R² is heteroaryl or phenyl, wherein said heteroaryl and phenyl groups are optionally substituted with one to three groups independently selected from the group consisting of halo, cyano, oxo, C₁₋₆ alkyl, (C₁₋₆ alkyl)OR⁶, OR⁶, R⁷, OR⁷, O(C₁₋₆ alkyl)OR⁶, O(C₁₋₆ alkyl)R⁷, (C═O)R⁶, (C═O)OR⁶, (C═O)NHR⁶, (C═O)R⁷, NHR⁶, NH(C═O)OR⁶, NH(C═O)R⁷, NH(C═O)O(C₁₋₆ alkyl)OR⁶ and NO₂;
R³ is hydrogen, halo or C₁₋₆ alkyl,
R^{3′} is hydrogen, halo or C₁₋₆ alkyl,
R⁴ is hydrogen, halo, C₁₋₆ alkyl or (C═O)OR⁶,
R^{4′} is hydrogen, halo or C₁₋₆ alkyl,
R⁵ is hydrogen or C₁₋₆ alkyl,
R⁶ is hydrogen or C₁₋₆ alkyl, wherein said alkyl is optionally substituted with one to three groups independently selected from the group consisting of halo and hydroxyl;
R⁷ is hydrogen, heterocyclyl, aryl or heteroaryl, wherein said heterocyclyl and heteroaryl groups are optionally substituted with one to two groups independently selected from the group consisting of cyano, halo, hydroxyl, R⁶, R⁸, OR⁶, (C₁₋₆ alkyl)OR⁶, (C₁₋₆ alkyl)OR⁸, SO₂(C₁₋₆ alkyl), (C═O)R⁸;
R⁸ is heterocyclyl or heteroaryl, wherein said heterocyclyl group is optionally substituted with cyano, halo, hydroxyl, R⁶, OR⁶ or (C═O)OR⁶;
or a pharmaceutically acceptable salt thereof.

2. The compound of claim 1 wherein R¹ is heteroaryl, wherein said heteroaryl group is optionally substituted with one to three groups independently selected from the group consisting of halo, cyano or C₁₋₆ alkyl; or a pharmaceutically acceptable salt thereof.

3. The compound of claim 2 wherein R¹ is heteroaryl, wherein said heteroaryl group is optionally substituted with C₁₋₆ alkyl, or a pharmaceutically acceptable salt thereof.

4. The compound of claim 2 wherein R³ is hydrogen, R^{3′} is hydrogen, R⁴ is hydrogen and R^{4′} is hydrogen, or a pharmaceutically acceptable salt thereof.

5. The compound of claim 1 wherein R² is heteroaryl or phenyl, wherein said heteroaryl and phenyl groups are optionally substituted with one to three groups independently selected from the group consisting of halo, oxo, C₁₋₆ alkyl, (C₁₋₆ alkyl)OR⁶, OR⁶, R⁷, OR⁷, O(C₁₋₆ alkyl)OR⁶, O(C₁₋₆ alkyl)R⁷, (C═O)R⁶, (C═O)OR⁶, (C═O)NHR⁶, (C═O)R⁷, NHR⁶, NH(C═O)OR⁶, NH(C═O)R⁷, NH(C═O)O(C₁₋₆ alkyl)OR⁶ and NO₂;
or a pharmaceutically acceptable salt thereof.

6. The compound of claim 5 wherein R² is quinolinyl, wherein said quinolinyl is optionally substituted with one to three groups independently selected from the group consisting of halo, oxo, C₁₋₆ alkyl, (C₁₋₆ alkyl)OR⁶, OR⁶, R⁷, OR⁷, O(C₁₋₆ alkyl)OR⁶, O(C₁₋₆ alkyl)R⁷, (C═O)R⁶, (C═O)OR⁶, (C═O)NHR⁶, (C═O)R⁷, NHR⁶, NH(C═O)OR⁶, NH(C═O)R⁷, NH(C═O)O(C₁₋₆ alkyl)OR⁶ and NO₂; or a pharmaceutically acceptable salt thereof.

7. The compound of claim 1 wherein X is O; or a pharmaceutically acceptable salt thereof.

8. The compound selected from:
1-(1-Methyl-1H-pyrazol-4-yl)-3-[(quinolin-6-yloxy)methyl]pyridazin-4(1H)-one;
3-[(isoquinolin-5-yloxy)methyl]-1-(1-methyl-1H-pyrazol-4-yl)pyridazin-4(1H)-one;
3-{4-oxo-3-[(quinolin-6-yloxy)methyl]pyridazin-1(4H)-yl}benzonitrile;
3-fluoro-5-{4-oxo-3-[(quinolin-6-yloxy)methyl]pyridazin-1(4H)-yl}benzonitrile;
4-{4-oxo-3-[(quinolin-6-yloxy)methyl]pyridazin-1(4H)-yl}benzonitrile;
3-chloro-5-{4-oxo-3-[(quinolin-6-yloxy)methyl]pyridazin-1(4H)-yl}benzonitrile;
1-(3,5-difluorophenyl)-3-[(quinolin-6-yloxy)methyl]pyridazin-4(1H)-one;
1-(3,4-difluorophenyl)-3-[(quinolin-6-yloxy)methyl]pyridazin-4(1H)-one;
1-(1-methyl-1H-pyrazol-4-yl)-3-[(4-nitrophenoxy)methyl]pyridazin-4(1H)-one;
3-[(quinolin-6-yloxy)methyl]-1-(3,4,5-trifluorophenyl)pyridazin-4(1H)-one;
1-(1-methyl-1H-pyrazol-4-yl)-3-[(quinoxalin-6-yloxy)methyl]pyridazin-4(1H)-one;
ethyl 4-{[1-(1-methyl-1H-pyrazol-4-yl)-4-oxo-1,4-dihydropyridazin-3-yl]methoxy}benzoate;
1-(1-methyl-1H-pyrazol-4-yl)-3-{[(2-methylquinolin-6-yl)oxy]methyl}pyridazin-4(1H)-one;
6-{[1-(1-methyl-1H-pyrazol-4-yl)-4-oxo-1,4-dihydropyridazin-3-yl]methoxy}-1,3-benzothiazole-2-carbonitrile;
1-Methyl-N-(3-{[1-(1-methyl-1H-pyrazol-4-yl)-4-oxo-1,4-dihydropyridazin-3-yl]methoxy}phenyl)-1H-pyrazole-4-carboxamide;
N-methyl-4-{[1-(1-methyl-1H-pyrazol-4-yl)-4-oxo-1,4-dihydropyridazin-3-yl]methoxy}benzamide;
4-{[1-(1-methyl-1H-pyrazol-4-yl)-4-oxo-1,4-dihydropyridazin-3-yl]methoxy}benzamide;
4-{[1-(1-methyl-1H-pyrazol-4-yl)-4-oxo-1,4-dihydropyridazin-3-yl]methoxy}-N-(2,2,2-trifluoroethyl)benzamide;
1-(1-methyl-1H-pyrazol-4-yl)-3-{[(2-phenyl-1H-benzimidazol-5-yl)oxy]methyl}pyridazin-4(1H)-one;
1-(3-Bromophenyl)-3-[(quinolin-6-yloxy)methyl]pyridazin-4(1H)-one;
1-(5-bromopyridin-3-yl)-3-[(quinolin-6-yloxy)methyl]pyridazin-4(1H)-one;
1-(1-ethyl-1H-pyrazol-4-yl)-3-[(quinolin-6-yloxy)methyl]pyridazin-4(1H)-one;
3-{[(3-Ethoxyquinolin-6-yl)oxy]methyl}-1-(1-methyl-1H-pyrazol-4-yl)pyridazin-4(1H)-one;
3-({[3-(2-methoxyethoxy)quinolin-6-yl]oxy}methyl)-1-(1-methyl-1H-pyrazol-4-yl)pyridazin-4(1H)-one;
1-(1-methyl-1H-pyrazol-4-yl)-3-({[3-(tetrahydrofuran-3-ylmethoxy)quinolin-6-yl]oxy}methyl)pyridazin-4(1H)-one;
3-[({3-[(3-methyloxetan-3-yl)methoxy]quinolin-6-yl}oxy)methyl]-1-(1-methyl-1H-pyrazol-4-yl)pyridazin-4(1H)-one;
1-(1-methyl-1H-pyrazol-4-yl)-3-[(quinazolin-6-yloxy)methyl]pyridazin-4(1H)-one;
3-fluoro-5-[3-({[3-(2-methoxyethoxy)quinolin-6-yl]oxy}methyl)-4-oxopyridazin-1(4H)-yl]benzonitrile;
3-chloro-5-[3-({[3-(2-methoxyethoxy)quinolin-6-yl]oxy}methyl)-4-oxopyridazin-1(4H)-yl]benzonitrile;
1-(3,4-difluorophenyl)-3-({[3-(2-methoxyethoxy)quinolin-6-yl]oxy}methyl)pyridazin-4(1H)-one;
1-(3,5-difluorophenyl)-3-({[3-(2-methoxyethoxy)quinolin-6-yl]oxy}methyl)pyridazin-4(1H)-one;
3-{[(8-fluoroquinolin-6-yl)oxy]methyl}-1-(1-methyl-1H-pyrazol-4-yl)pyridazin-4(1H)-one;
3-({[4-(2-ethoxyethoxy)quinolin-6-yl]oxy}methyl)-1-(1-methyl-1H-pyrazol-4-yl)pyridazin-4(1H)-one;
3-({[4-(2-methoxyethoxy)quinolin-6-yl]oxy}methyl)-1-(1-methyl-1H-pyrazol-4-yl)pyridazin-4(1H)-one;
3-{[(3-methoxyquinolin-6-yl)oxy]methyl}-1-(1-methyl-1H-pyrazol-4-yl)pyridazin-4(1H)-one;
1-(1-ethyl-1H-pyrazol-4-yl)-3-({[3-(2-methoxyethoxy)quinolin-6-yl]oxy}methyl)pyridazin-4(1H)-one;
3-{[(3-fluoroquinolin-6-yl)oxy]methyl}-1-(1-methyl-1H-pyrazol-4-yl)pyridazin-4(1H)-one;
1-(3-bromophenyl)-3-({[3-(2-methoxyethoxy)quinolin-6-yl]oxy}methyl)pyridazin-4(1H)-one;
1-(1-methyl-1H-pyrazol-4-yl)-3-({[3-(4H-1,2,4-triazol-4-yl)quinolin-6-yl]oxy}methyl)pyridazin-4(1H)-one;
3-[({3-[4-(methoxymethyl)-1H-1,2,3-triazol-1-yl]quinolin-6-yl}oxy)methyl]-1-(1-methyl-1H-pyrazol-4-yl)pyridazin-4(1H)-one;
1-(1-methyl-1H-pyrazol-4-yl)-3-{[(3-{4-[(piperidin-4-yloxy)methyl]-1-(1-methyl-1H-pyrazol-4-yl)pyridazin-4(1H)-one;
1-(1-Methyl-1H-pyrazol-4-yl)-3-({[3-(1H-1,2,3-triazol-1-yl)quinolin-6-yl]oxy}methyl)pyridazin-4(1H)-one;
3-{[(4-Ethoxyquinolin-6-yl)oxy]methyl}-1-(1-methyl-1H-pyrazol-4-yl)pyridazin-4(1H)-one;
3-[3-{[(3-ethoxyquinolin-6-yl)oxy]methyl}-4-oxopyridazin-1(4H)-yl]-5-fluorobenzonitrile;
3-chloro-5-[3-{[(3-ethoxyquinolin-6-yl)oxy]methyl}-4-oxopyridazin-1(4H)-yl]benzonitrile;
1-(3,4-difluorophenyl)-3-{[(3-ethoxyquinolin-6-yl)oxy]methyl}pyridazin-4(1H)-one;
1-(3,5-difluorophenyl)-3-{[(3-ethoxyquinolin-6-yl)oxy]methyl}pyridazin-4(1H)-one;
3-{[(4-Methoxyquinolin-6-yl)oxy]methyl}-1-(1-methyl-1H-pyrazol-4-yl)pyridazin-4(1H)-one;
3-{[(2-methoxyquinolin-6-yl)oxy]methyl}-1-(1-methyl-1H-pyrazol-4-yl)pyridazin-4(1H)-one;
1-(1-methyl-1H-pyrazol-4-yl)-3-{[(4-methylquinolin-6-yl)oxy]methyl}pyridazin-4(1H)-one;
3-{4-Oxo-3-[(quinolin-6-yloxy)methyl]pyridazin-1(4H)-yl}benzamide;
3-[3-({[3-(2-methoxyethoxy)quinolin-6-yl]oxy}methyl)-4-oxopyridazin-1(4H)-yl]-N,N-dimethylbenzamide;
1-(1-Methyl-1H-pyrazol-4-yl)-3-[2-(quinolin-6-yl)ethyl]pyridazin-4(1H)-one;
1-(1-methyl-1H-pyrazol-4-yl)-3-(2-quinolin-4-ylethyl)pyridazin-4(1H)-one;
1-(1-methyl-1H-pyrazol-4-yl)-3-[2-(1H-pyrrolo[2,3-b]pyridin-4-yl)ethyl]pyridazin-4(1H)-one;
methyl 4-{2-[1-(1-methyl-1H-pyrazol-4-yl)-4-oxo-1,4-dihydropyridazin-3-yl]ethyl}benzoate;
3-{2-[3-(2-methoxyethoxy)quinolin-6-yl]ethyl}-1-(1-methyl-1H-pyrazol-4-yl)pyridazin-4(1H)-one;
3-[2-(3-ethoxyquinolin-6-yl)ethyl]-1-(1-methyl-1H-pyrazol-4-yl)pyridazin-4(1H)-one;
3-[2-(4-methoxyquinolin-6-yl)ethyl]-1-(1-methyl-1H-pyrazol-4-yl)pyridazin-4(1H)-one;
1-(1-Methyl-1H-pyrazol-4-yl)-3-[2-(quinoxalin-6-yl)ethyl]pyridazin-4(1H)-one;
3-Hydroxy-6-{[1-(1-methyl-1H-pyrazol-4-yl)-4-oxo-1,4-dihydropyridazin-3-yl]methoxy}quinolinium chloride;
3-({[3-(2-Hydroxy-2-methylpropoxy)quinolin-6-yl]oxy}methyl)-1-(1-methyl-1H-pyrazol-4-yl)pyridazin-4(1H)-one;
1-(1-Methyl-1H-pyrazol-4-yl)-3-({[3-(oxetan-3-yloxy)quinolin-6-yl]oxy}methyl)pyridazin-4(1H)-one;
3-[({3-[(2H5)ethyloxy]quinolin-6-yl}oxy)methyl]-1-(1-methyl-1H-pyrazol-4-yl)pyridazin-4(1H)-one;
3-({[3-(2,2-difluoroethoxy)quinolin-6-yl]oxy}methyl)-1-(1-methyl-1H-pyrazol-4-yl)pyridazin-4(1H)-one;
3-({[3-(difluoromethoxy)quinolin-6-yl]oxy}methyl)-1-(1-methyl-1H-pyrazol-4-yl)pyridazin-4(1H)-one;
1-(1-ethyl-1H-pyrazol-4-yl)-3-({[3-(pyridin-2-yloxy)quinolin-6-yl]oxy}methyl)pyridazin-4(1H)-one;
3-({[3-(2,2-difluoroethoxy)quinolin-6-yl]oxy}methyl)-1-(1-ethyl-1H-pyrazol-4-yl)pyridazin-4(1H)-one;
1-(1-ethyl-1H-pyrazol-4-yl)-3-({[3-(tetrahydrofuran-3-yloxy)quinolin-6-yl]oxy}methyl)pyridazin-4(1H)-one;
1-(1-ethyl-1H-pyrazol-4-yl)-3-[({3-[(5-methyl-1,2,4-oxadiazol-3-yl)methoxy]quinolin-6-yl}oxy)methyl]pyridazin-4(1H)-one;
1-(1-Methyl-1H-pyrazol-4-yl)-3-{[(3-methylquinolin-6-yl)oxy]methyl}pyridazin-4(1H)-one;
3-{[(3-ethylquinolin-6-yl)oxy]methyl}-1-(1-methyl-1H-pyrazol-4-yl)pyridazin-4(1H)-one;
3-({[3-(methoxymethyl)quinolin-6-yl]oxy}methyl)-1-(1-methyl-1H-pyrazol-4-yl)pyridazin-4(1H)-one;
1-(1-Methyl-1H-pyrazol-4-yl)-3-{[(3-pyridin-3-ylquinolin-6-yl)oxy]methyl}pyridazin-4(1H)-one;
1-(1-methyl-1H-pyrazol-4-yl)-3-({[3-(1-methyl-1H-pyrazol-4-yl)quinolin-6-yl]oxy}methyl)pyridazin-4(1H)-one;
6-(1-ethyl-1H-pyrazol-4-yl)-2-[({3-[4-(methylsulfonyl)phenyl]quinolin-6-yl}oxy)methyl]pyridin-3(6H)-one;
1-(1-ethyl-1H-pyrazol-4-yl)-3-[({3-[1-(2-methoxyethyl)-1H-pyrazol-4-yl]quinolin-6-yl}oxy)methyl]pyridazin-4(1H)-one;
1-(1-ethyl-1H-pyrazol-4-yl)-3-({[3-(4-fluorophenyl)quinolin-6-yl]oxy}methyl)pyridazin-4(1H)-one;
1-(1-ethyl-1H-pyrazol-4-yl)-3-({[3-(4-methoxyphenyl)quinolin-6-yl]oxy}methyl)pyridazin-4(1H)-one;
1-(1-ethyl-1H-pyrazol-4-yl)-3-{[(3-pyridin-4-ylquinolin-6-yl)oxy]methyl}pyridazin-4(1H)-one;
5-(6-{[1-(1-ethyl-1H-pyrazol-4-yl)-4-oxo-1,4-dihydropyridazin-3-yl]methoxy}quinolin-3-yl)pyridine-2-carbonitrile;
1-(1-ethyl-1H-pyrazol-4-yl)-3-({[3-(6-methoxypyridin-3-yl)quinolin-6-yl]oxy}methyl)pyridazin-4(1H)-one;
1-(1-ethyl-1H-pyrazol-4-yl)-3-({[3-(2-fluorophenyl)quinolin-6-yl]oxy}methyl)pyridazin-4(1H)-one;
1-(1-ethyl-1H-pyrazol-4-yl)-3-({[3-(2-methylphenyl)quinolin-6-yl]oxy}methyl)pyridazin-4(1H)-one;
1-(1-ethyl-1H-pyrazol-4-yl)-3-{[(3-pyrimidin-5-ylquinolin-6-yl)oxy]methyl}pyridazin-4(1H)-one;
1-(1-ethyl-1H-pyrazol-4-yl)-3-({[3-(1,3-thiazol-4-yl)quinolin-6-yl]oxy}methyl)pyridazin-4(1H)-one;
1-(1-ethyl-1H-pyrazol-4-yl)-3-({[3-(6-morpholin-4-ylpyridin-3-yl)quinolin-6-yl]oxy}methyl)pyridazin-4(1H)-one;
3-({[3-(6-chloropyridin-3-yl)quinolin-6-yl]oxy}methyl)-1-(1-ethyl-1H-pyrazol-4-yl)pyridazin-4(1H)-one;
1-(1-ethyl-1H-pyrazol-4-yl)-3-[({3-[4-(trifluoromethyl)phenyl]quinolin-6-yl}oxy)methyl]pyridazin-4(1H)-one;
1-(1-ethyl-1H-pyrazol-4-yl)-3-({[3-(3-fluorophenyl)quinolin-6-yl]oxy}methyl)pyridazin-4(1H)-one;
1-(1-ethyl-1H-pyrazol-4-yl)-3-{[(3-phenylquinolin-6-yl)oxy]methyl}pyridazin-4(1H)-one;
tert-Butyl-3-[4-(6-{[1-(1-methyl-1H-pyrazol-4-yl)-4-oxo-1,4-dihydropyridazin-3-yl]methoxy}quinolin-3-yl)-1H-pyrazol-1-yl]azetidine-1-carboxylate;
3-[({3-[(1)-1-(Azetidin-3-ylamino)prop-1-en-2-yl]quinolin-6-yl}oxy)methyl]-1-(1-methyl-1H-pyrazol-4-yl)pyridazin-4(1H)-one ammoniate;
1-(1-methyl-1H-pyrazol-4-yl)-3-[({3-[1-(piperidin-4-yl)-1H-pyrazol-4-yl]quinolin-6-yl}oxy)methyl]pyridazin-4(1H)-one;
Methyl 6-{[1-(1-methyl-1H-pyrazol-4-yl)-4-oxo-1,4-dihydropyridazin-3-yl]methoxy}quinoline-3-carboxylate;
1-(1-Methyl-1H-pyrazol-4-yl)-3-[({3-[1-(oxetan-3-yl)-1H-pyrazol-4-yl]quinolin-6-yl}oxy)methyl]pyridazin-4(1H)-one;
3-{[(4-Chloroquinolin-6-yl)oxy]methyl}-1-(1-methyl-1H-pyrazol-4-yl)pyridazin-4(1H)-one;
3-{[(4-Ethylquinolin-6-yl)oxy]methyl}-1-(1-methyl-1H-pyrazol-4-yl)pyridazin-4(1H)-one;
3-({[3-(2-Methoxyethoxy)quinolin-6-yl]sulfanyl}methyl)-1-(1-methyl-1H-pyrazol-4-yl)pyridazin-4(1H)-one;
3-{[(3-ethoxyquinolin-6-yl)sulfanyl]methyl}-1-(1-methyl-1H-pyrazol-4-yl)pyridazin-4(1H)-one;
1-(1-Methyl-1H-pyrazol-4-yl)-3-[(quinolin-6-ylsulfanyl)methyl]pyridazin-4(1H)-one;
3-({[3-(2-Methoxyethoxy)quinolin-6-yl]amino}methyl)-1-(1-methyl-1H-pyrazol-4-yl)pyridazin-4(1H)-one;
3-{[(3-ethoxyquinolin-6-yl)amino]methyl}-1-(1-methyl-1H-pyrazol-4-yl)pyridazin-4(1H)-one;
1-(1-Methyl-1H-pyrazol-4-yl)-3-[(quinolin-6-ylamino)methyl]pyridazin-4(1H)-one;
3-[(1,3-Benzothiazol-6-yloxy)methyl]-1-(1-methyl-1H-pyrazol-4-yl)pyridazin-4(1H)-one;
3-({[3-(2-methoxyethoxy)quinoxalin-6-yl]oxy}methyl)-1-(1-methyl-1H-pyrazol-4-yl)pyridazin-4(1H)-one;
3-[(2,3-dihydro[1,4]dioxino[2,3-c]quinolin-9-yloxy)methyl]-1-(1-methyl-1H-pyrazol-4-yl)pyridazin-4(1H)-one;
1-(1-Methyl-1H-pyrazol-4-yl)-3-{[methyl(quinolin-6-yl)amino]methyl}pyridazin-4(1H)-one;
1-(1-methyl-1H-pyrazol-4-yl)-3-({[3-(morpholin-4-ylcarbonyl)quinolin-6-yl]oxy}methyl)pyridazin-4(1H)-one;
tert-Butyl (6-{[1-(1-methyl-1H-pyrazol-4-yl)-4-oxo-1,4-dihydropyridazin-3-yl]methoxy}quinolin-3-yl)carbamate;
1,3-bis(6-{[1-(1-methyl-1H-pyrazol-4-yl)-4-oxo-1,4-dihydropyridazin-3-yl]methoxy}quinolin-3-yl)urea;
3-{[(3-Aminoquinolin-6-yl)oxy]methyl}-1-(1-methyl-1H-pyrazol-4-yl)pyridazin-4(1H)-one;
1-(3-Bromophenyl)-3-[1-(quinolin-6-yloxy)ethyl]pyridazin-4(1H)-one;
1-(3-Bromophenyl)-3-[1-(quinolin-6-yloxy)ethyl]pyridazin-4(1H)-one;
3-((4-aminophenoxy)methyl)-1-(1-methyl-1H-pyrazol-4-yl)pyridazin-4(1H)-one;
3-((4-(4-hydroxybutylamino)phenoxy)methyl)-1-(1-methyl-1H-pyrazol-4-yl)pyridazin-4(1H)-one;
3-[2-(4-aminophenyl)ethyl]-1-(1-methyl-1H-pyrazol-4-yl)pyridazin-4(1H)-one;
3-(2-{4-[(4-hydroxybutyl)amino]phenyl}ethyl)-1-(1-methyl-1H-pyrazol-4-yl)pyridazin-4(1H)-one;
2-methoxyethyl (4-{[1-(1-methyl-1H-pyrazol-4-yl)-4-oxo-1,4-dihydropyridazin-3-yl]methoxy}phenyl)carbamate;
2-methoxyethyl (4-{2-[1-(1-methyl-1H-pyrazol-4-yl)-4-oxo-1,4-dihydropyridazin-3-yl]ethyl}phenyl)carbamate;
ethyl (4-{2-[1-(1-methyl-1H-pyrazol-4-yl)-4-oxo-1,4-dihydropyridazin-3-yl]ethyl}phenyl)carbamate;
ethyl (4-{[1-(1-methyl-1H-pyrazol-4-yl)-4-oxo-1,4-dihydropyridazin-3-yl]methoxy}phenyl)carbamate;
2-methylpropyl (4-{[1-(1-methyl-1H-pyrazol-4-yl)-4-oxo-1,4-dihydropyridazin-3-yl]methoxy}phenyl)carbamate;
1-(1-Methyl-1H-pyrazol-4-yl)-3-(2-quinolin-6-ylprop-2-en-1-yl)pyridazin-4(1H)-one;
-(1-Methyl-1H-pyrazol-4-yl)-3-(2-quinolin-6-ylpropyl)pyridazin-4(1H)-one;
-(1-Methyl-1H-pyrazol-4-yl)-3-(2-quinolin-6-ylpropyl)pyridazin-4(1H)-one;
1-(1-Methyl-1H-pyrazol-4-yl)-3-(quinolin-6-ylmethoxy)pyridazin-4(1H)-one;
1-(1-ethyl-1H-pyrazol-4-yl)-3-(quinolin-6-ylmethoxy)pyridazin-4(1H)-one;
1-(1-methyl-1H-pyrazol-4-yl)-3-{[3-(1-methyl-1H-pyrazol-4-yl)quinolin-6-yl]methoxy}pyridazin-4(1H)-one;
1-(1-methyl-1H-pyrazol-4-yl)-3-(1-quinolin-6-ylethoxy)pyridazin-4(1H)-one;
3-[1-(4-methoxyquinolin-6-yl)ethoxy]-1-(1-methyl-1H-pyrazol-4-yl)pyridazin-4(1H)-one;
1-(1-ethyl-1H-pyrazol-4-yl)-3-[1-(4-methoxyquinolin-6-yl)ethoxy]pyridazin-4(1H)-one;
3-[1-(3-ethoxyquinolin-6-yl)ethoxy]-1-(1-methyl-1H-pyrazol-4-yl)pyridazin-4(1H)-one;
3-{1-[3-(2-methoxyethoxy)quinolin-6-yl]ethoxy}-1-(1-methyl-1H-pyrazol-4-yl)pyridazin-4(1H)-one;
3-[1-(3-methoxyquinolin-6-yl)ethoxy]-1-(1-methyl-1H-pyrazol-4-yl)pyridazin-4(1H)-one;
1-(1-methyl-1H-pyrazol-4-yl)-3-{1-[3-(1-methyl-1H-pyrazol-4-yl)quinolin-6-yl]ethoxy}pyridazin-4(1H)-one;
3-[(4-methoxyquinolin-6-yl)methoxy]-1-(1-methyl-1H-pyrazol-4-yl)pyridazin-4(1H)-one;
1-(1-methyl-1H-pyrazol-4-yl)-3-[1-(quinolin-6-yl)ethoxy]pyridazin-4(1H)-one;
1-(1-methyl-1H-pyrazol-4-yl)-3-[1-(quinolin-6-yl)ethoxy]pyridazin-4(1H)-one;
3-[1-(3-methoxyquinolin-6-yl)ethoxy]-1-(1-methyl-1H-pyrazol-4-yl)pyridazin-4(1H)-one enantiomer 2;
3-[1-(3-ethoxyquinolin-6-yl)ethoxy]-1-(1-methyl-1H-pyrazol-4-yl)pyridazin-4(1H)-one enantiomer 1;
3-[1-(3-ethoxyquinolin-6-yl)ethoxy]-1-(1-methyl-1H-pyrazol-4-yl)pyridazin-4(1H)-one enantiomer 2;
3-{1-[3-(2-methoxyethoxy)quinolin-6-yl]ethoxy}-1-(1-methyl-1H-pyrazol-4-yl)pyridazin-4(1H)-one enantiomer 1;
3-{1-[3-(2-methoxyethoxy)quinolin-6-yl]ethoxy}-1-(1-methyl-1H-pyrazol-4-yl)pyridazin-4(1H)-one enantiomer 2;
3-[1-(4-methoxyquinolin-6-yl)ethoxy]-1-(1-methyl-1H-pyrazol-4-yl)pyridazin-4(1H)-one enantiomer 2;
1-(1-ethyl-1H-pyrazol-4-yl)-3-[1-(4-methoxyquinolin-6-yl)ethoxy]pyridazin-4(1H)-one enantiomer 2;
1-(1-methyl-1H-pyrazol-4-yl)-3-[(quinolin-6-ylmethyl)amino]pyridazin-4(1H)-one;
1-(1-methyl-1H-pyrazol-4-yl)-3-{[1-(quinolin-6-yl)ethyl]amino}pyridazin-4(1H)-one;
1-(1-methyl-1H-pyrazol-4-yl)-3-[(quinolin-6-ylmethyl)thio]pyridazin-4(1H)-one;
methyl {[1-(1-methyl-1H-pyrazol-4-yl)-4-oxo-1,4-dihydropyridazin-3-yl]oxy}(quinolin-6-yl)acetate;
or a pharmaceutically acceptable salt thereof.

9. A pharmaceutical composition that is comprised of a compound in accordance with claim 1 and a pharmaceutically acceptable carrier.

10. A method of treating or preventing cancer in a mammal in need of such treatment that is comprised of administering to said mammal a therapeutically effective amount of a compound of claim 1.

11. A compound which is: 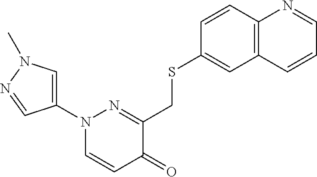 [SMILES: Cn1cc(-n2ccc(=O)c(CSc3ccc4ncccc4c3)n2)cn1]
1-(1-Methyl-1H-pyrazol-4-yl)-3-[(quinolin-6-ylsulfanyl)methyl]pyridazin-4(1H)-one or a pharmaceutically acceptable salt thereof.

12. A compound which is: 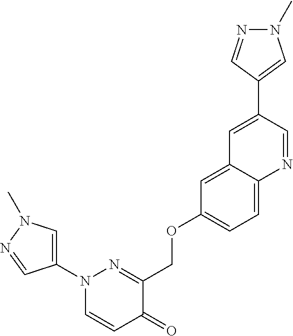 [SMILES: Cn1cc(-c2cnc3ccc(OCc4nn(-c5cnn(C)c5)ccc4=O)cc3c2)cn1]
1-(1-methyl-1H-pyrazol-4-yl)-3-({[3-(1-methyl-1H-pyrazol-4-yl)quinolin-6-yl]oxy}methyl)pyridazin-4(1H)-one
or a pharmaceutically acceptable salt thereof.

13. A compound which is: 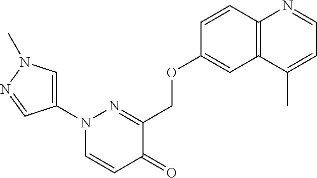 [SMILES: Cc1ccnc2ccc(OCc3nn(-c4cnn(C)c4)ccc3=O)cc12]
1-(1-methyl-1H-pyrazol-4-yl)-3-{[(4-methylquinolin-6-yl)oxy]methyl}pyridazin-4(1H)-one
or a pharmaceutically acceptable salt thereof.

14. A compound which is: 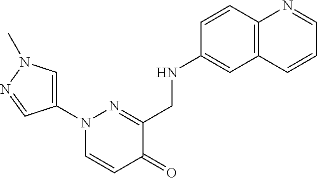 [SMILES: Cn1cc(-n2ccc(=O)c(CNc3ccc4ncccc4c3)n2)cn1]
1-(1-Methyl-1H-pyrazol-4-yl)-3-[(quinolin-6-ylamino)methyl]pyridazin-4(1H)-one or a pharmaceutically acceptable salt thereof.

15. A compound which is: 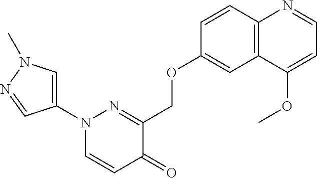 [SMILES: COc1ccnc2ccc(OCc3nn(-c4cnn(C)c4)ccc3=O)cc12]
3-{[(4-Methoxyquinolin-6-yl)oxy]methyl}-1-(1-methyl-1H-pyrazol-4-yl)pyridazin-4(1H)-one
or a pharmaceutically acceptable salt thereof.

16. A compound which is: 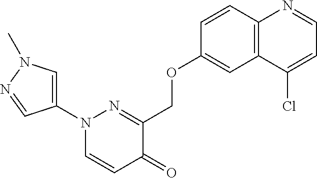 [SMILES: Cn1cc(-n2ccc(=O)c(COc3ccc4nccc(Cl)c4c3)n2)cn1]
3-{[(4-Chloroquinolin-6-yl)oxy]methyl}-1-(1-methyl-1H-pyrazol-4-yl)pyridazin-4(1H)-one
or a pharmaceutically acceptable salt thereof.
